(19) United States
(12) Patent Application Publication
(10) Pub. No.: US 2026/0123608 A1
(43) Pub. Date: May 7, 2026

(54) NON-HUMAN ANIMALS HAVING MODIFIED IMMUNOGLOBULIN HEAVY CHAIN CONSTANT REGION LOCUS AND USES THEREOF

(71) Applicant: Regeneron Pharmaceuticals, Inc., Tarrytown, NY (US)

(72) Inventors: John McWhirter, Sanremo IM (IT); Chunguang Guo, Briarcliff Manor, NY (US); Lynn Macdonald, Harrison, NY (US); Andrew J. Murphy, Croton-on-Hudson, NY (US)

(21) Appl. No.: 19/292,205

(22) Filed: Aug. 6, 2025

Related U.S. Application Data
(60) Provisional application No. 63/679,947, filed on Aug. 6, 2024.

(51) Int. Cl. A01K 67/0275; C07K 16/08; C12N 5/0735

(52) U.S. Cl. CPC A01K 67/0275; C07K 16/08; C12N 5/0606; A01K 2217/075; A01K 2227/105; A01K 2267/01; C07K 2317/52; C12N 2510/00

(57) ABSTRACT

Non-human animals (and/or non-human cells) and methods of using the same are provided, which non-human animals (and/or non-human cells) have a genome comprising human antibody-encoding sequences (i.e., immunoglobulin genes). Non-human animals described herein express antibodies that are of IgA, IgD, or IgM isotypes. Non-human animals provided herein are, in some embodiments, characterized by expression of IgA antibodies that contain human heavy chain and light chain variable domains and rodent constant domains. Methods for producing antibodies from non-human animals are also provided, which antibodies contain human variable regions and rodent constant regions.

### CROSS REFERENCE TO RELATED APPLICATIONS

This application claims the benefit of U.S. Provisional Application No. 63/679,947, filed Aug. 6, 2024, the contents of which is hereby incorporated by reference in its entirety.

### SEQUENCE LISTING

The instant application contains a Sequence Listing in electronic format entitled “2010794-3024_SL.xml” which was created on Jul. 22, 2025, and which has a size of 22,284 bytes. The contents of the file “2010794-3024_SL.xml” are incorporated by reference herein.

### BACKGROUND

Human antibodies are the most rapidly growing class of therapeutics. Of the technologies that are currently used for their production, the development of genetically modified non-human animals (e.g., rodents) engineered with genetic material encoding human antibodies, in whole or in part, has revolutionized the field of human therapeutic monoclonal antibodies for the treatment of various diseases. Still, development of improved in vivo systems for generating human monoclonal antibodies that maximize human antibody repertoires in genetically modified non-human animals is needed.

### SUMMARY

The present disclosure provides, among other things, a non-human animal (e.g., a rodent, e.g., a mouse or rat) whose germline genome has been modified such that the animal produces antibodies of only particular isotypes (e.g., IgA isotype, IgM isotype, and/or IgD isotype). For example, in some embodiments, a non-human animal (e.g., a rodent, e.g., a mouse or rat) described herein comprises a germline genome that comprises an engineered endogenous immunoglobulin heavy chain locus that comprises an immunoglobulin heavy chain constant region comprising a non-human animal (e.g., a rodent, e.g., a mouse or rat) immunoglobulin heavy chain alpha (Igha) constant region, wherein the genetically modified non-human animal produces only immunoglobulin chains that comprise IgA constant domain encoded by a Igha constant region gene.

In another embodiment, a non-human animal (e.g., a rodent, e.g., a mouse or rat) described herein comprises a germline genome that comprises an engineered endogenous immunoglobulin heavy chain locus that comprises immunoglobulin heavy chain constant region genes Ighm, Ighd, and Igha, wherein the animal only produces immunoglobulin heavy chains that comprise an IgA, an IgM, or an IgD constant domain encoded by an Igha constant region gene, an Ighm constant region gene, or an Ighd constant region gene, respectively.

Animals (e.g., rodents, e.g., mice or rats) described herein, when immunized with an antigen of interest, produce specific populations of antibodies. A genetically modified non-human animal (e.g., a rodent, e.g., a mouse or rat) that produces only, primarily, or an increased titer of IgA antibodies provides a new tool for antibody discovery, particularly taking advantage of the unique properties of IgA antibodies, which have traditionally proven difficult to isolate, purify and recombinantly produce. Additionally, IgA antibodies may provide new beneficial properties for therapeutic antibodies, particularly for difficult targets that traditional IgG antibodies have been unsuccessful at targeting.

Antibodies with IgG isotype are most prevalent among circulating antibodies, accounting for approximately 80%. The second most prevalent isotype is monomeric IgA, which accounts for about 15% of circulating antibodies. However, IgA antibodies are the main class of antibodies found in the mucosa of the intestinal lining and is produced at a higher rate than any of the other isotypes (about 40-60 mg/kg per day) (see Bohlander, Fabian. Frontiers in Immunology 14 (2023): 1127339, which is herein incorporated by reference). IgA antibodies have unique structure and function within the immune system, including preventing infection (particularly from invading pathogens at mucosal surfaces) and controlling inflammation, that make them an ideal class of antibody for therapeutic antibody discovery. IgA antibodies have a strong effector function through the IgA-specific receptor FcαRI and are efficacious at killing tumor cells (see e.g., van Tetering, Geert, et al. Antibodies 9.4 (2020): 70, which is herein incorporated by reference). As such, efforts have been made to generate therapeutic IgA. However, challenges exist with developing therapeutic IgA antibodies, which include, among other things, lack of animal models due to differences in IgA antibodies in humans and rodents, difficulty in producing recombinant IgA antibodies due to its complex structure and heavy glycosylation, and its short half-life (see Bohlander, 2023). Consequently, most therapeutic antibodies to date have an IgG isotype. The prevalence of IgG antibodies as therapeutics is in part because of IgG's abundance in serum, long half-life, and body of literature related to IgG antibodies.

The present disclosure addresses the difficulties in identifying, isolating and studying IgA antibodies (e.g., short half-life and lower quantity in serum), and is a promising approach for developing therapeutic IgA antibodies. Additionally, the present disclosure recognizes that antigen binding domains identified from IgA antibodies generated in humans and non-human animals may also have unique properties such as ability to target different types of antigens involved in infection and chronic inflammation, often implicated in diseases such as chronic respiratory and digestive tract diseases. Thus, antigen-binding domains identified or derived from IgA antibodies can be utilized in development of therapeutic antigen-binding proteins of any format, including any antibody isotype.

The present disclosure also provides genetically modified non-human animals (e.g., rodents, e.g., mice or rats) with modified endogenous immunoglobulin heavy chain loci that result in limited antibody class(es) being produced by the rodent. For example, the present disclosure provides genetically modified non-human animals that produce only, primarily, or an increased titer of IgA antibodies, e.g., by introducing one or more modifications that disrupt the function and/or expression of the endogenous immunoglobulin constant region genes Ighm, Ighg, Ighd, and/or Ighe. In other embodiments, the present disclosure provides genetically modified non-human animals that produce only, primarily, or an increased titer of IgA, IgM, and IgD antibodies, e.g., by introducing one or more modifications that disrupt the function and/or expression of the endogenous immunoglobulin constant region genes Ighg and Ighe. The present disclosure also contemplates the humanization of the endogenous immunoglobulin heavy and/or light chain variable region locus such that IgA antibodies produced by the animal in response to antigen stimulation contain human variable domains and rodent IgA constant domains. Human variable domains can be either human heavy or human light chain variable domains. Human antigen-binding domains (e.g., human immunoglobulin heavy and/or light chain variable domains) identified from rodents described herein may be utilized in a therapeutic antibody with a constant domain that is not limited to an IgA constant domain (e.g., an IgG, IgM, IgD, or IgE constant domain). Such an approach would utilize potential beneficial properties of antigen binding domains originating from IgA antibodies, while also benefiting from the longer half-life and ease of production of IgG antibodies.

The present disclosure provides, among other things, a genetically modified rodent. In some embodiments, a genetically modified rodent has a germline genome that comprises an engineered endogenous immunoglobulin heavy chain locus.

In some embodiments, an engineered endogenous immunoglobulin heavy chain locus comprises an immunoglobulin heavy chain constant region comprising a rodent immunoglobulin heavy chain alpha (Igha) constant region gene. In some embodiments, a genetically modified rodent only produces immunoglobulin heavy chains that comprise a rodent Igha constant domain encoded by a rodent Igha constant region gene.

In some embodiments, a genetically modified rodent's germline genome comprises one or more genetic alterations at an endogenous immunoglobulin heavy chain locus, where one or more genetic alterations disrupt expression from rodent immunoglobulin heavy chain constant region genes: mu (Ighm), delta (Ighd), gamma (Ighg), and epsilon (Ighe) constant region genes.

In some embodiments, an Ighg constant region gene comprises gamma 3 (Ighg3), gamma 1 (Ighg1), gamma 2a (Ighg2a), and gamma 2b (Ighg2b) constant region genes. In some embodiments, an Ighg constant region gene comprises gamma 3 (Ighg3), gamma 1 (Ighg1), gamma 2c (Ighg2c), and gamma 2b (Ighg2b) constant region genes.

In some embodiments, one or more genetic alterations comprise one or more deletions, insertions, mutations, and/or inversions.

In some embodiments, one or more genetic alterations comprises one or more deletions. In some embodiments, one or more deletions remove all or part of each of rodent immunoglobulin heavy chain constant region genes: Ighm, Ighd, Ighg and Ighe constant region genes. In some embodiments, one or more deletions remove part of each of rodent immunoglobulin heavy chain constant region genes: Ighm, Ighd, Ighg, and Ighe constant region genes. In some embodiments, one or more deletions remove all of rodent immunoglobulin heavy chain constant region genes: Ighm, Ighd, Ighg, and Ighe constant region genes. In some embodiments, one or more deletions remove a contiguous sequence spanning a region that is upstream of a rodent Ighm gene to a region that is upstream of a rodent Igha gene. In some embodiments, one or more deletions do not remove all or a part of a non-coding region between rodent immunoglobulin heavy chain constant region genes: Ighm, Ighd, Ighg, and Ighe constant region genes.

In some embodiments, one or more genetic alterations comprise one or more insertions. In some embodiments, one or more insertions comprise an insertion of one or more repressor elements that inhibit expression of rodent immunoglobulin heavy chain constant region genes: Ighm, Ighd, Ighg, and Ighe constant region genes.

In some embodiments, one or more genetic alterations comprise one or more mutations. In some embodiments, one or more mutations comprises a frameshift mutation that inhibits expression of rodent immunoglobulin heavy chain constant region genes: Ighm, Ighd, Ighg, and Ighe constant region genes.

In some embodiments, an engineered endogenous immunoglobulin heavy chain locus comprises in 5′ to 3′ order: (i) a rodent intronic enhancer (Eμ); (ii) a rodent switch mu region (Sμ); and (iii) a rodent Igha constant region gene. In some embodiments, an engineered endogenous immunoglobulin heavy chain locus comprises a rodent 3′ IgH regulatory region (3′RR) that is 3′ of a rodent Igha constant region gene.

The present disclosure provides, among other things, a genetically modified rodent. In some embodiments, a genetically modified rodent has a germline genome that comprises an engineered endogenous immunoglobulin heavy chain locus.

In some embodiments, an engineered endogenous immunoglobulin heavy chain locus comprises an immunoglobulin heavy chain constant region comprising rodent immunoglobulin heavy chain constant region genes Ighm, Ighd, and Igha. In some embodiments, a genetically modified rodent only produces immunoglobulin heavy chains that comprise a rodent Igha, a rodent Ighm, or a rodent Ighd constant domain encoded by a rodent Igha constant region gene, a rodent Ighm constant region gene, or a rodent Ighd constant region gene, respectively.

In some embodiments, a genetically modified rodent's germline genome comprises one or more genetic alterations at an endogenous immunoglobulin heavy chain locus, where one or more genetic alterations disrupt expression from rodent immunoglobulin heavy chain constant region genes: Ighg and Ighe constant region genes.

In some embodiments, an Ighg constant region gene comprises gamma 3 (Ighg3), gamma 1 (Ighg1), gamma 2a (Ighg2a), and gamma 2b (Ighg2b) constant region genes. In some embodiments, an Ighg constant region gene comprises gamma 3 (Ighg3), gamma 1 (Ighg1), gamma 2c (Ighg2c), and gamma 2b (Ighg2b) constant region genes.

In some embodiments, one or more genetic alterations comprise one or more deletions, insertions, mutations, and/or inversions.

In some embodiments, one or more genetic alterations comprises one or more deletions. In some embodiments, one or more deletions remove all or part of each of rodent immunoglobulin heavy chain constant region genes: Ighg and Ighe constant region genes. In some embodiments, one or more deletions remove part of each of rodent immunoglobulin heavy chain constant region genes: Ighg and Ighe constant region genes. In some embodiments, one or more deletions remove all of rodent immunoglobulin heavy chain constant region genes: Ighg and Ighe constant region genes. In some embodiments, one or more deletions remove a contiguous sequence spanning a region that is upstream of an Ighg constant region gene to a region that is upstream of a rodent Igha constant region gene. In some embodiments, one or more deletions do not remove one or more of a non-coding sequences adjacent to rodent immunoglobulin heavy chain constant region genes: Ighg and Ighe constant region genes.

In some embodiments, one or more genetic alterations comprise one or more insertions. In some embodiments, one or more insertions comprise an insertion of one or more repressor elements that inhibit expression of rodent immunoglobulin heavy chain constant region genes: Ighg and Ighe constant region genes.

In some embodiments, one or more genetic alterations comprise one or more mutations. In some embodiments, one or more mutations comprise a frameshift mutation that inhibits expression of rodent immunoglobulin heavy chain constant region genes: Ighg and Ighe constant region genes.

In some embodiments, an engineered endogenous immunoglobulin heavy chain locus comprises in 5′ to 3′ order: (i) a rodent intronic enhancer (Eμ); (ii) a rodent switch mu region (Sμ); (iii) a rodent Ighm constant region gene; (iv) a rodent Ighd constant region gene; (v) a rodent switch alpha region (Sα); and (vi) a rodent Igha constant region gene. In some embodiments, an engineered endogenous immunoglobulin heavy chain locus comprises a rodent 3′ IgH regulatory region 3′RR that is 3′ of a rodent Igha constant region gene.

In some embodiments, engineered endogenous immunoglobulin heavy chain locus comprises one or more rodent V_{H} gene segments, one or more rodent D_{H} gene segments, and one or more rodent J_{H} gene segments. In some embodiments, rodent V_{H}, D_{H}, and J_{H} gene segments are operably linked to a rodent immunoglobulin heavy chain constant region.

In some embodiments, an engineered endogenous immunoglobulin heavy chain locus comprises one or more non-rodent mammal V_{H} gene segments, one or more non-rodent mammal D_{H} gene segments, and one or more non-rodent mammal J_{H} gene segments. In some embodiments, non-rodent mammal V_{H}, D_{H}, and J_{H} gene segments are operably linked to a rodent immunoglobulin heavy chain constant region. In some embodiments, a non-rodent mammal is a cat, dog, cow, horse, primate, or human.

In some embodiments, one or more non-rodent mammal V_{H} gene segments, one or more non-rodent mammal D_{H} gene segments, and one or more non-rodent mammal J_{H} gene segments are or comprise one or more human V_{H} gene segments, one or more human D_{H} gene segments, and one or more human J_{H} gene segments.

In some embodiments, an engineered endogenous immunoglobulin heavy chain locus comprises a contiguous human sequence that spans one or more human V_{H} gene segments, one or more human D_{H} gene segments, and one or more human J_{H} gene segments. In some embodiments, a contiguous human sequence is operably linked to a rodent immunoglobulin heavy chain constant region.

In some embodiments, an engineered endogenous immunoglobulin heavy chain locus comprises one or more rodent V_{H} non-coding sequences, each of which is adjacent to one or more human V_{H} gene segments, where the one or more V_{H} non-coding sequences naturally appears adjacent to a rodent V_{H} gene segment in an endogenous rodent immunoglobulin locus.

In some embodiments, an engineered endogenous immunoglobulin heavy chain locus comprises one or more rodent D_{H} non-coding sequences, each of which is adjacent to one or more human D_{H} gene segments, wherein the one or more D_{H} non-coding sequences naturally appears adjacent to a rodent D_{H} gene segment in an endogenous rodent immunoglobulin locus.

In some embodiments, an engineered endogenous immunoglobulin heavy chain locus comprises one or more rodent J_{H} non-coding sequences, each of which is adjacent to one or more human J_{H} gene segments, wherein the one or more J_{H} non-coding sequences naturally appears adjacent to a rodent J_{H} gene segment in an endogenous rodent immunoglobulin locus.

In some embodiments, one or more human V_{H} gene segments, one or more human D_{H} gene segments, and one or more human J_{H} gene segments are operably linked to a rodent immunoglobulin heavy chain constant region at an endogenous rodent immunoglobulin heavy chain locus.

In some embodiments, an engineered endogenous immunoglobulin heavy chain locus comprises in 5′ to 3′ order: (i) one or more human V_{H} gene segments, one or more human DH gene segments, and one or more human J_{H} gene segments; (ii) a rodent intronic enhancer (Eμ); (iii) a rodent switch mu region (Sμ); (iv) a rodent Igha constant region gene; and optionally (v) a rodent 3′ IgH regulatory region 3′RR.

In some embodiments, an engineered endogenous immunoglobulin heavy chain locus comprises in 5′ to 3′ order: (i) one or more human V_{H} gene segments, one or more human D_{H} gene segments, and one or more human J_{H} gene segments; (ii) a rodent intronic enhancer (Eμ); (iii) a rodent switch mu region (Sμ); (iv) a rodent Ighm constant region gene; (v) a rodent Ighd constant region gene; (vi) a rodent switch alpha region (Sα); (vii) a rodent Igha constant region gene; and optionally (viii) a rodent 3′ IgH regulatory region 3′RR.

In some embodiments, one or more human V_{H} gene segments, one or more human D_{H} gene segments, and one or more human J_{H} gene segments replace one or more rodent V_{H} gene segments, one or more rodent D_{H} gene segments, one or more rodent J_{H} gene segments, or any combination thereof.

In some embodiments, one or more human V_{H} gene segments, one or more human D_{H} gene segments, and one or more human J_{H} gene segments are inserted downstream of one or more endogenous rodent V_{H} gene segments, one or more rodent D_{H} gene segments, one or more rodent J_{H} gene segments, or any combination thereof. In some embodiments, one or more endogenous rodent V_{H} gene segments, one or more rodent D_{H} gene segments, one or more rodent J_{H} gene segments, or any combination thereof are non-functional.

In some embodiments, a genetically modified rodent is a mouse. In some embodiments, a genetically modified rodent is a rat.

In some embodiments, a genetically modified rodent is homozygous for an engineered endogenous immunoglobulin heavy chain locus. In some embodiments, a genetically modified rodent is heterozygous for an engineered endogenous immunoglobulin heavy chain locus. In some embodiments, a genetically modified rodent Igha constant region gene is an endogenous rodent Igha constant region gene.

In some embodiments, a genetically modified rodent Igha constant region gene is an endogenous mouse Igha constant region gene. In some embodiments, an endogenous mouse Igha constant region gene comprises as Igha*01 allele. In some embodiments, an endogenous mouse Igha constant region gene comprises as Igha*02 allele.

In some embodiments, one or more human V_{H} gene segments comprise V_{H}3-74, V_{H}3-73, V_{H}3-72, V_{H}2-70, V_{H}1-69, V_{H}3-66, V_{H}3-64, V_{H}4-61, V_{H}4-59, V_{H}1-58, V_{H}3-53, V_{H}5-51, V_{H}3-49, V_{H}3-48, V_{H}1-46, V_{H}1-45, V_{H}3-43, V_{H}4-39, V_{H}4-34, V_{H}3-33, V_{H}4-31, V_{H}3-30, V_{H}4-28, V_{H}2-26, V_{H}1-24, V_{H}3-23, V_{H}3-21, V_{H}3-20, V_{H}1-18, V_{H}3-15, V_{H}3-13, V_{H}3-11, V_{H}3-9, V_{H}1-8, V_{H}3-7, V_{H}2-5, V_{H}7-4-1, V_{H}4-4, V_{H}1-3, V_{H}1-2, V_{H}6-1, or any combination thereof.

In some embodiments, one or more human D_{H} gene segments comprise D_{H}1-1, D_{H}2-2, D_{H}3-3, D_{H}4-4, D_{H}5-5, D_{H}6-6, D_{H}1-7, D_{H}2-8, D_{H}3-9, D_{H}3-10, D_{H}5-12, D_{H}6-13, D_{H}2-15, D_{H}3-16, D_{H}4-17, D_{H}6-19, D_{H}1-20, D_{H}2-21, D_{H}3-22, D_{H}6-25, D_{H}1-26, D_{H}7-27, or any combination thereof.

In some embodiments, one or more human J_{H} gene segments comprise: J_{H}1, J_{H}2, J_{H}3, J_{H}4, J_{H}5, J_{H}6, or any combination thereof.

In some embodiments, one or more human V_{H} gene segments, one or more human D_{H} gene segments, and one or more human J_{H} gene segments comprise all functional human V_{H} gene segments, all functional human D_{H} gene segments, and all functional human J_{H} gene segments.

In some embodiments, one or more human V_{H} gene segments, one or more human D_{H} gene segments, and one or more human J_{H} gene segments comprises 80 V_{H} gene segments, all human D_{H} gene segments, and all human J_{H} gene segments.

In some embodiments, one or more human V_{H} gene segments, one or more human D_{H} gene segments, and one or more human J_{H} gene segments comprises all V_{H} gene segments, all human D_{H} gene segments, and all human J_{H} gene segments.

In some embodiments, a genetically modified rodent is heterozygous for an engineered endogenous immunoglobulin heavy chain locus. In some embodiments, a genetically modified rodent is hemizygous for an engineered endogenous immunoglobulin heavy chain locus. In some embodiments, a genetically modified rodent is homozygous for an engineered endogenous immunoglobulin heavy chain locus.

In some embodiments, an engineered endogenous immunoglobulin heavy chain locus lacks a functional endogenous rodent Adam6 gene.

In some embodiments, a genetically modified rodent's germline genome comprises one or more nucleotide sequences encoding one or more rodent ADAM6 polypeptides, functional orthologs, functional homologs, or functional fragments thereof. In some embodiments, one or more rodent ADAM6 polypeptides, functional orthologs, functional homologs, or functional fragments thereof are expressed.

In some embodiments, one or more nucleotide sequences encoding one or more rodent ADAM6 polypeptides, functional orthologs, functional homologs, or functional fragments thereof are included on the same chromosome as an engineered endogenous immunoglobulin heavy chain locus.

In some embodiments, one or more nucleotide sequences encoding one or more rodent ADAM6 polypeptides, functional orthologs, functional homologs, or functional fragments thereof are included in an engineered endogenous immunoglobulin heavy chain locus.

In some embodiments, one or more nucleotide sequences encoding one or more rodent ADAM6 polypeptides, functional orthologs, functional homologs, or functional fragments thereof are in place of a human Adam6 pseudogene. In some embodiments, one or more nucleotide sequences encoding one or more rodent ADAM6 polypeptides, functional orthologs, functional homologs, or functional fragments thereof replace a human Adam6 pseudogene.

In some embodiments, one or more nucleotide sequences encoding one or more rodent ADAM6 polypeptides, functional orthologs, functional homologs, or functional fragments thereof are between a first human V_{H} gene segment and a second human V_{H} gene segment. In some embodiments, a first human V_{H} gene segment is V_{H}1-2 and a second human V_{H} gene segment is V_{H}6-1.

In some embodiments, one or more nucleotide sequences encoding one or more rodent ADAM6 polypeptides, functional orthologs, functional homologs, or functional fragments thereof are between a human V_{H} gene segment and a human D_{H} gene segment.

In some embodiments, a germline genome of a rodent comprises an engineered endogenous immunoglobulin light chain locus comprising: (a) one or more non-rodent mammal Vκ gene segments, and (b) one or more non-rodent mammal Jκ gene segments. In some embodiments, one or more non-rodent mammal Vκ gene segments and one or more non-rodent mammal Jκ gene segments are operably linked to a CK gene.

In some embodiments, non-rodent mammal is a cat, dog, cow, horse, primate, or human.

In some embodiments, one or more non-rodent mammal Vκ gene segments and one or more non-rodent mammal Jκ gene segments are or comprise one or more human Vκ gene segments and one or more human Jκ gene segments.

In some embodiments, an engineered endogenous immunoglobulin light chain locus comprises a contiguous human sequence that spans one or more human Vκ gene segments and one or more human Jκ gene segments. In some embodiments, a contiguous human sequence spanning one or more human Vκ gene segments and one or more human Jκ gene segments is operably linked to an endogenous rodent immunoglobulin light chain CK gene.

In some embodiments, an engineered endogenous immunoglobulin light chain locus comprises one or more rodent Vκ non-coding sequences, each of which is adjacent to one or more human Vκ gene segments, where the one or more Vκ non-coding sequences naturally appears adjacent to a rodent Vκ gene segment in an endogenous rodent immunoglobulin locus. In some embodiments, an engineered endogenous immunoglobulin light chain locus comprises one or more rodent Jκ non-coding sequences, each of which is adjacent to one or more human Jκ gene segments, where the one or more Jκ non-coding sequences naturally appears adjacent to a rodent Jκ gene segment in an endogenous rodent immunoglobulin locus.

In some embodiments, a CK gene is a rodent CK gene. In some embodiments, a CK gene is an endogenous rodent CK gene.

In some embodiments, one or more human Vκ gene segments and one or more human Jκ gene segments replace one or more rodent Vκ gene segments and one or more rodent Jκ gene segments. In some embodiments, one or more human Vκ gene segments and one or more human Jκ gene segments are inserted downstream of one or more endogenous rodent Vκ gene segments and one or more endogenous rodent Jκ gene segments. In some embodiments, one or more endogenous rodent Vκ gene and one or more endogenous rodent Jκ gene segments are non-functional.

In some embodiments, a genetically modified rodent is homozygous for an engineered endogenous immunoglobulin light chain locus. In some embodiments, a genetically modified rodent is heterozygous for an engineered endogenous immunoglobulin light chain locus. In some embodiments, a genetically modified rodent comprises an inactivated endogenous immunoglobulin κ light chain locus.

In some embodiments, a genetically modified rodent is heterozygous for an inactivated endogenous immunoglobulin κ light chain locus. In some embodiments, a genetically modified rodent is hemizygous for an inactivated endogenous immunoglobulin κ light chain locus. In some embodiments, a genetically modified rodent is homozygous for an inactivated endogenous immunoglobulin κ light chain locus.

In some embodiments, an endogenous Vκ gene segments and an endogenous Jκ gene segments are deleted in whole or in part. In some embodiments, a genetically modified rodent does not detectably express endogenous immunoglobulin κ light chain variable domains.

In some embodiments, one or more human Vκ gene segments and one or more human Jκ gene segments comprise 40 Vκ gene segments and all human Jκ gene segments. In some embodiments, one or more human Vκ gene segments and one or more human Jκ gene segments comprise all functional human Vκ gene segments and all functional human Jκ gene segments. In some embodiments, one or more human Vκ gene segments and one or more human Jκ gene segments comprise all human Vκ gene segments and all human Jκ gene segments.

In some embodiments, one or more human Vκ gene segments comprises a single human Vκ gene segment and one or more human Jκ gene segments comprises a single human Jκ gene segment. In some embodiments, a single human Vκ gene segment and a single Jκ gene segment comprise a single rearranged Vκ/Jκ gene sequence. In some embodiments, all immunoglobulin κ light chains expressed by B cells of a genetically modified rodent include human immunoglobulin κ light chain variable domains expressed from a single rearranged human immunoglobulin κ light chain variable region or a somatically hypermutated version thereof.

In some embodiments, a germline genome of a rodent comprises an engineered endogenous immunoglobulin light chain locus comprising one or more non-rodent mammal Vλ gene segments, and one or more non-rodent mammal Jλ gene segments. In some embodiments, one or more non-rodent mammal Vλ gene segments and one or more non-rodent mammal Jλ gene segments are operably linked to a rodent immunoglobulin light chain constant region gene.

In some embodiments, a non-rodent mammal is a cat, dog, cow, horse, primate, or human.

In some embodiments, one or more non-rodent mammal Vλ gene segments and one or more non-rodent mammal Jλ gene segments are or comprise one or more human Vλ gene segments and one or more human Jλ gene segments.

In some embodiments, an engineered endogenous immunoglobulin light chain locus comprises a contiguous human sequence that spans one or more human mammal Vλ gene segments and one or more human Jλ gene segments. In some embodiments, a contiguous human sequence is operably linked to an endogenous rodent immunoglobulin light chain gene.

In some embodiments, an engineered endogenous immunoglobulin light chain locus comprises one or more rodent Vλ non-coding sequences, each of which is adjacent to one or more human Vλ gene segments, where the one or more Vλ non-coding sequences naturally appears adjacent to a rodent Vλ gene segment in an endogenous rodent immunoglobulin locus. In some embodiments, an engineered endogenous immunoglobulin light chain locus comprises one or more rodent Jλ non-coding sequences, each of which is adjacent to one or more human Jλ gene segments, where the one or more Jκ non-coding sequences naturally appears adjacent to a rodent Jλ gene segment in an endogenous rodent immunoglobulin locus.

In some embodiments, an endogenous rodent immunoglobulin light chain constant region gene is a rodent Cλ gene. In some embodiments, an endogenous rodent immunoglobulin light chain constant region gene is a rodent Cκ gene.

In some embodiments, an engineered endogenous immunoglobulin light chain locus is an engineered endogenous λ immunoglobulin light chain locus. In some embodiments, an engineered endogenous immunoglobulin light chain locus is an engineered endogenous κ immunoglobulin light chain locus.

In some embodiments, one or more human Vλ gene segments and one or more human Jλ gene segments replace one or more rodent Vλ gene segments and one or more rodent Jλ gene segments.

In some embodiments, one or more human Vλ gene segments and one or more human Jλ gene segments are inserted downstream of one or more endogenous rodent Vλ gene segments and one or more endogenous rodent Jλ gene segments. In some embodiments, one or more endogenous rodent Vλ gene and one or more endogenous rodent Jλ gene segments are non-functional.

In some embodiments, a genetically modified rodent is homozygous for an engineered endogenous immunoglobulin light chain locus. In some embodiments, a genetically modified rodent is heterozygous for an engineered endogenous immunoglobulin light chain locus. In some embodiments, a genetically modified rodent is hemizygous for an engineered endogenous immunoglobulin light chain locus.

In some embodiments, a genetically modified rodent comprises an inactivated endogenous immunoglobulin λ light chain locus. In some embodiments, a genetically modified rodent is heterozygous for an inactivated endogenous immunoglobulin λ light chain locus. In some embodiments, a genetically modified rodent is homozygous for an inactivated endogenous immunoglobulin λ light chain locus. In some embodiments, an endogenous rodent Vλ gene segments and Jλ gene segments are deleted in whole or in part. In some embodiments, a genetically modified rodent does not detectably express endogenous immunoglobulin λ light chain variable domains.

In some embodiments, one or more human Vλ gene segments comprise Vλ5-52, Vλ1-51, Vλ9-49, Vλ1-47, Vλ7-46, Vλ5-45, Vλ1-44, Vλ7-43, Vλ1-40, Vλ5-39, Vλ5-37, Vλ1-36, Vλ3-27, Vλ3-25, Vλ2-23, Vλ3-22, Vλ3-21, Vλ3-19, Vλ3-16, Vλ2-14, Vλ3-12, Vλ2-11, Vλ3-10, Vλ3-9, Vλ2-8, Vλ4-3, Vλ3-1, or any combination thereof.

In some embodiments, one or more human Jλ gene segments comprise Jλ1, Jλ2, Jλ3, Jλ6, Jλ7, or any combination thereof.

In some embodiments, one or more human Vλ gene segments comprise at least 12 human Vλ gene segments. In some embodiments, one or more human Vλ gene segments comprise at least 28 human Vλ gene segments. In some embodiments, one or more human Vλ gene segments comprise at least 40 human Vλ gene segments. In some embodiments, one or more human Vλ gene segments and one or more human Jλ gene segments comprise all functional human Vλ gene segments and all functional human Jλ gene segments.

In some embodiments, one or more human Vλ gene segments comprises a single human Vλ gene segment and one or more human Jλ gene segments comprises a single human Jλ gene segment. In some embodiments, an engineered endogenous immunoglobulin light chain locus comprises a single rearranged human immunoglobulin λ light chain variable region.

In some embodiments, a single rearranged human immunoglobulin λ light chain variable region is operably linked to a rodent Cλ gene segment at an endogenous immunoglobulin κ light chain locus.

In some embodiments, all immunoglobulin λ light chains expressed by B cells of a genetically modified rodent include human immunoglobulin λ light chain variable domains expressed from a single rearranged human immunoglobulin λ light chain variable region or a somatically hypermutated version thereof.

The present disclosure also provides method of making a genetically modified rodent. In some embodiments, a method comprises genetically modifying a germline genome of a rodent so that a rodent comprises an immunoglobulin heavy chain constant region that comprises a rodent Igha constant region gene. In some embodiments, a genetically modified rodent only produces immunoglobulin heavy chains that comprise a rodent Igha constant domain encoded by a rodent Igha constant region gene.

In some embodiments, a method comprises introducing one or more genetic alterations at an endogenous immunoglobulin heavy chain locus, where one or more genetic alterations disrupt expression from rodent immunoglobulin heavy chain constant region genes: Ighm, Ighd, Ighg and Ighe constant region genes.

In some embodiments, an Ighg constant region gene comprises gamma 3 (Ighg3), gamma 1 (Ighg1), gamma 2a (Ighg2a) and gamma 2b (Ighg2b) constant region genes. In some embodiments, an Ighg constant region gene comprises gamma 3 (Ighg3), gamma 1 (Ighg1), gamma 2c (Ighg2c), and gamma 2b (Ighg2b) constant region genes.

In some embodiments, one or more genetic alterations comprise one or more deletions, insertions, mutations, and/or inversions.

In some embodiments, one or more genetic alterations comprises one or more deletions. In some embodiments, one or more deletions remove all or part of each of rodent immunoglobulin heavy chain constant region genes: Ighm, Ighd, Ighg, and Ighe constant region genes. In some embodiments, one or more deletions remove part of each of rodent immunoglobulin heavy chain constant region genes: Ighm, Ighd, Ighg, and Ighe constant region genes. In some embodiments, one or more deletions remove all of each of rodent immunoglobulin heavy chain constant region genes: Ighm, Ighd, Ighg, and Ighe constant region genes. In some embodiments, one or more deletions remove a contiguous sequence spanning a region that is upstream of a rodent Ighm constant region gene to a region that is upstream of a rodent Igha constant region gene. In some embodiments, one or more deletions do not remove all or a part of a non-coding region between rodent immunoglobulin heavy chain constant region genes: Ighm, Ighd, Ighg constant region genes and Ighe.

In some embodiments, one or more genetic alterations comprise one or more insertions. In some embodiments, one or more insertions comprise an insertion of one or more repressor elements that inhibit expression of rodent immunoglobulin heavy chain constant region genes: Ighm, Ighd, Ighg, and Ighe constant region genes.

In some embodiments, one or more genetic alterations comprise one or more mutations. In some embodiments, one or more mutations comprise one or more frameshift mutations that inhibit expression of rodent immunoglobulin heavy chain constant region genes: Ighm, Ighd, Ighg and Ighe constant region genes.

In some embodiments, an engineered endogenous immunoglobulin heavy chain locus comprises in 5′ to 3′ order: (i) a rodent intronic enhancer (Eμ); (ii) a rodent switch mu region (Sμ); and (iii) a rodent immunoglobulin heavy constant alpha (Igha) gene. In some embodiments, an engineered endogenous immunoglobulin heavy chain locus comprises a rodent 3′ IgH regulatory region 3′RR that is 3′ of a rodent Igha constant region gene.

In some embodiments, a genetically modified rodent is heterozygous for an engineered endogenous immunoglobulin heavy chain locus. In some embodiments, a genetically modified rodent is hemizygous for an engineered endogenous immunoglobulin heavy chain locus. In some embodiments, a genetically modified rodent is homozygous for an engineered endogenous immunoglobulin heavy chain locus.

The present disclosure further provides a method of making a genetically modified rodent. In some embodiments, a method comprises genetically modifying a germline genome of a rodent so that a rodent comprises an immunoglobulin heavy chain constant region that comprises a constant region comprising rodent immunoglobulin heavy chain constant region genes Ighm, Ighd, and Igha. In some embodiments, a genetically modified rodent only produces immunoglobulin heavy chains that comprise a rodent Igha, a rodent Ighm, or a rodent Ighd constant domain encoded by an Igha constant region gene, a rodent Ighm constant region gene, or a rodent Ighd constant region gene, respectively.

In some embodiments, a genetically modified rodent's germline genome comprises one or more genetic alterations at an endogenous immunoglobulin heavy chain locus. In some embodiments, one or more genetic alterations disrupt expression from rodent immunoglobulin heavy chain constant region genes: Ighg and Ighe constant region genes.

In some embodiments, one or more genetic alterations comprise one or more deletions, insertions, mutations, and/or inversions.

In some embodiments, one or more genetic alternations comprises one or more deletions. In some embodiments, one or more deletions remove all or part of each of rodent immunoglobulin heavy chain constant region genes: Ighg and Ighe constant region genes. In some embodiments, one or more deletions remove part of each of rodent immunoglobulin heavy chain constant region genes: Ighg and Ighe constant region genes. In some embodiments, one or more deletions remove all of each rodent immunoglobulin heavy chain constant region genes: Ighg and Ighe constant region genes. In some embodiments, one or more deletions remove a contiguous sequence spanning a region that is upstream of a rodent Ighg3 constant region gene to a region that is upstream of a rodent Igha constant region gene. In some embodiments, one or more deletions do not remove one or more of a non-coding sequences adjacent to rodent immunoglobulin heavy chain constant region genes: Ighg and Ighe constant region genes.

In some embodiments, one or more genetic alterations comprise one or more insertions. In some embodiments, one or more insertions comprise an insertion of one or more repressor elements that inhibit expression of rodent immunoglobulin heavy chain constant region genes: Ighg and Ighe constant region genes.

In some embodiments, one or more genetic alterations comprise one or more mutations. In some embodiments, one or more mutations comprise one or more frameshift mutations that inhibit expression of each of rodent immunoglobulin heavy chain constant region genes: Ighg and Ighe constant region genes.

In some embodiments, a genetically modified rodent comprises an engineered endogenous immunoglobulin heavy chain locus that comprises in 5′ to 3′ order: (i) a rodent intronic enhancer (Eμ); (ii) a rodent switch mu region (Sμ); (iii) a rodent Ighm constant region gene; (iv) a rodent Ighd constant region gene; (v) a rodent switch alpha region (Sα); (vi) a rodent Igha constant region gene; and (vii) optionally a rodent 3′ IgH regulatory region 3′RR. In some embodiments, an engineered endogenous immunoglobulin heavy chain locus comprises a rodent 3′ IgH regulatory region 3′RR that is 3′ of a rodent Igha constant region gene.

In some embodiments, a genetically modified rodent is heterozygous for an engineered endogenous immunoglobulin heavy chain locus. In some embodiments, a genetically modified rodent is hemizygous for an engineered endogenous immunoglobulin heavy chain locus. In some embodiments, a genetically modified rodent is homozygous for an engineered endogenous immunoglobulin heavy chain locus.

Among other things, the present disclosure provides a method of producing an antibody. In some embodiments, a method comprises immunizing a genetically modified rodent as described herein with an antigen of interest. In some embodiments, a method comprises maintaining a genetically modified rodent under conditions sufficient for the genetically modified rodent to produce an immune response to an antigen of interest. In some embodiments, a method comprises recovering from a genetically modified rodent (i) an antibody that binds an antigen of interest, (ii) a nucleotide that encodes a human light or heavy chain variable domain, a light chain, or a heavy chain of an antibody that binds an antigen of interest, or (iii) a cell that expresses an antibody that binds an antigen of interest.

In some embodiments, a method comprises expressing a first nucleotide sequence that encodes an immunoglobulin heavy chain in a host cell, where a first nucleotide sequence includes a human heavy chain variable region sequence that was identified from a genetically modified rodent as described. In some embodiments, a method comprises expressing a second nucleotide sequence that encodes an immunoglobulin light chain in a host cell. In some embodiments, a method comprises culturing a host cell so that immunoglobulin light chains and immunoglobulin heavy chains are expressed and form an antibody. In some embodiments, a method comprises obtaining an antibody from a host cell or host cell culture.

Additionally, the present disclosure provides a method of making a fully human antibody specific against an antigen. In some embodiments, a method comprises immunizing a genetically modified rodent as described herein with an antigen. In some embodiments, a method comprises determining a nucleotide sequence that encodes a human heavy chain variable domain of an antibody that specifically binds an antigen and that was generated by a genetically modified rodent and/or determining a nucleotide sequence that encodes a human light chain variable domain of an antibody that specifically binds an antigen and that was generated by a genetically modified rodent. In some embodiments, a method comprises expressing a fully human antibody by employing a nucleotide sequence encoding a human heavy chain variable domain from a genetically modified rodent operably linked to a human heavy chain constant region gene. In some embodiments, a method comprises expressing a fully human antibody by employing a nucleotide sequence encoding a human light chain variable domain from a genetically modified rodent operably linked to a human light chain constant region gene. In some embodiments, a human heavy chain constant region gene is a human Ighg (a human Ighg1, Ighg2, Ighg3, or Ighg4) constant region gene. In some embodiments, a human heavy chain constant region gene is a human Igha (a human Igha1 or Igha2) constant region gene.

In some embodiments, a method comprises expressing in a mammalian cell a fully human antibody comprising two human light chains and two human heavy chains, where each human light chain includes a human light chain variable domain encoded by a human light chain variable region and each human heavy chain includes a human heavy chain variable domain encoded by a human heavy chain variable region, where a nucleotide sequence of at least one human heavy or light chain variable region was obtained from a genetically modified rodent as described herein. In some embodiments, a method comprises obtaining a fully human antibody. In some embodiments, a method comprises immunizing a genetically modified rodent as described herein. In some embodiments, a method comprises determining a human heavy chain variable domain sequence of an antibody that specifically binds an antigen and that was generated by a genetically modified rodent and/or determining of a human light chain variable domain sequence of an antibody that specifically binds an antigen and that was generated by a genetically modified rodent. In some embodiments, a method comprises expressing a fully human antibody by employing a human heavy chain variable domain sequence from a genetically modified rodent operably linked to a human heavy chain constant domain sequence. In some embodiments, a method comprises expressing a fully human antibody by employing a human light chain variable domain sequence from a genetically modified rodent operably linked to a human light chain constant domain sequence.

In some embodiments, employing a human heavy chain variable domain sequence from a genetically modified rodent operably linked to a human heavy chain constant domain sequence comprises expressing a nucleotide sequence that encodes a human heavy chain variable domain sequence from a genetically modified rodent and a human heavy chain constant domain sequence. In some embodiments, employing a human light chain variable domain sequence from a genetically modified rodent operably linked to a human light chain constant domain sequence comprises expressing a nucleotide sequence that encodes a human light chain variable domain sequence from a genetically modified rodent and a human light chain constant domain sequence.

In some embodiments, a method comprises expressing in a mammalian cell a fully human antibody comprising two human light chains and two human heavy chains, where each human light chain includes a human light chain variable domain and each human heavy chain includes a human heavy chain variable domain, and where an amino acid sequence of at least one human heavy or light chain variable domain was obtained from a genetically modified rodent as described herein. In some embodiments, a method comprises obtaining a fully human antibody.

The present disclosure provides a method of generating a human heavy chain or light chain variable domain sequence. In some embodiments, a method comprises immunizing a genetically modified rodent as described herein with an antigen. In some embodiments, a method comprises determining a human heavy or light chain variable domain sequence of an antibody that specifically binds an antigen and that was generated by a genetically modified rodent. In some embodiments, a human heavy or light chain variable domain sequence comprises determining a nucleotide sequence that encodes a human heavy or light chain variable domain sequence.

The present disclosure also provides a method of making a fully human immunoglobulin heavy chain or a fully human immunoglobulin light chain. In some embodiments, a method comprises immunizing a genetically modified rodent as described herein with an antigen. In some embodiments, a method comprises determining a human immunoglobulin heavy or light chain variable domain sequence of an antibody that specifically binds to an antigen and that was generated by a genetically modified rodent. In some embodiments, a method comprises operably linking a human immunoglobulin heavy or light chain variable domain sequence to a human immunoglobulin heavy or light chain constant domain sequence, respectively, to form a fully human heavy chain or a fully human light chain.

In some embodiments, a method comprises operably linking a human immunoglobulin heavy or light chain variable domain sequence to a human immunoglobulin heavy or light chain constant domain sequence, respectively, comprises operably linking a nucleotide sequence encoding an immunoglobulin human heavy or light chain variable domain sequence to a nucleotide sequence encoding a human immunoglobulin heavy or light chain constant domain sequence.

Further, the present disclosure provides a method of generating a human immunoglobulin heavy or light chain variable region sequence. In some embodiments, a method comprises immunizing a genetically modified rodent as described herein with an antigen. In some embodiments, a method comprises determining a human immunoglobulin heavy or light chain variable region sequence that encodes a human immunoglobulin heavy or light chain variable domain, respectively, of an antibody that specifically binds an antigen and that was generated by a genetically modified rodent.

The present disclosure additionally provides a method of making a nucleotide sequence encoding a fully human immunoglobulin heavy chain or a fully human immunoglobulin light chain. In some embodiments, a method comprises immunizing a genetically modified rodent as described herein with an antigen. In some embodiments, a method comprises determining a human immunoglobulin heavy or light chain variable region sequence that encodes a human immunoglobulin heavy or light chain variable domain, respectively, of an antibody that specifically binds an antigen and that was generated by a genetically modified rodent. In some embodiments, a method comprises operably linking a human immunoglobulin heavy or light chain variable region sequence to a human immunoglobulin heavy or light chain constant region gene, respectively, to form a nucleotide sequence encoding a fully human immunoglobulin heavy chain or a fully human immunoglobulin light chain.

Also provided in the present disclosure is a method of making a genetically modified rodent embryonic stem (ES) cell. In some embodiments, a method comprises genetically modifying a rodent ES cell so that its genome comprises an engineered endogenous immunoglobulin heavy chain locus comprising a Igha constant region gene, where a genetically modified rodent only produces immunoglobulin heavy chains that comprise a rodent Igha constant domain encoded by a rodent Igha constant region gene. In some embodiments, a method comprises genetically modifying a rodent ES cell so that its genome comprises an engineered endogenous immunoglobulin heavy chain locus comprising rodent Ighm, Ighd, and Igha, where a genetically modified rodent only produces immunoglobulin heavy chains that comprise a rodent Igha, a rodent Ighm, or a rodent Ighd constant domain encoded by rodent Ighm, Ighd, and Igha constant region genes, respectively.

The present disclosure provides a rodent embryonic stem (ES) cell, whose genome comprises an engineered endogenous immunoglobulin heavy chain locus that comprises a rodent Igha constant region gene. In some embodiments, a rodent ES cell genome comprises one or more genetic alterations at an endogenous immunoglobulin heavy chain locus. In some embodiments, one or more genetic alterations disrupt expression from rodent immunoglobulin heavy chain constant region genes: Ighm, Ighd, Ighg and Ighe constant region genes.

The present disclosure provides a rodent embryonic stem (ES) cell, whose genome comprises an engineered endogenous immunoglobulin heavy chain locus that comprises rodent Ighm, Ighd, and Igha constant region genes. In some embodiments, a rodent ES cell genome comprises one or more genetic alterations at an endogenous immunoglobulin heavy chain locus. In some embodiments, one or more genetic alterations disrupt expression from rodent immunoglobulin heavy chain constant region genes: Ighg and Ighe constant region genes.

Also provided by the present disclosure is a rodent embryo generated from a rodent ES cell described herein.

In addition, the present disclosure provides an isolated rodent cell, whose genome comprises an engineered endogenous immunoglobulin heavy chain locus that comprises a rodent Igha constant region gene. In some embodiments, an isolated rodent cell genome comprises one or more genetic alterations at an endogenous immunoglobulin heavy chain locus, wherein one or more genetic alterations disrupt expression from rodent immunoglobulin heavy chain constant region genes: Ighm, Ighd, Ighg and Ighe constant region genes.

Moreover, the present disclosure provides an isolated rodent cell, whose genome comprises an engineered endogenous immunoglobulin heavy chain locus that comprises rodent immunoglobulin heavy chain constant region genes: Ighm, Ighd, and Igha constant region genes. In some embodiments, an isolated rodent cell genome comprises one or more genetic alterations at an endogenous immunoglobulin heavy chain locus. In some embodiments, one or more genetic alterations disrupt expression from rodent immunoglobulin heavy chain constant region genes: Ighg and Ighe constant region genes.

In some embodiments, an isolated rodent cell is a B cell. In some embodiments, a B cell is a plasma cell.

The present disclosure provides an immortalized cell generated from an isolated rodent cell as described herein. The present disclosure also provides hybridoma generated from an isolated B cell or plasma cell as described herein.

Also, the present disclosure provides a targeting vector. In some embodiments, a targeting vector can be used to genetically modify a genome of a rodent or rodent cell described herein. In some embodiments, a targeting vector comprises a 5′ homology arm comprising a nucleotide sequence corresponding to a genomic target sequence that is upstream to a rodent Ighm constant region gene and a 3′ homology arm comprising a nucleotide sequence corresponding to a genomic target sequence that is upstream of a rodent Igha constant region gene. In some embodiments, a targeting vector comprises a 5′ homology arm comprising a nucleotide sequence corresponding to a genomic target sequence that is upstream to a rodent Ighg3 constant region gene and a 3′ homology arm comprising a nucleotide sequence corresponding to a genomic target sequence that is upstream of a rodent Igha constant region gene. In some embodiments, a targeting vector comprises a selection marker.

### BRIEF DESCRIPTION OF THE DRAWING

The Drawing included herein, which is composed of the following Figures, is for illustration purposes only and not for limitation.

FIGS. 1A-1C show a schematic of an exemplary approach for genetically engineering a mouse immunoglobulin locus to generate an embodiment of an IgA rodent as described herein. Unless indicated otherwise, mouse coding sequences are indicated by filled in shapes (e.g., ovals, boxes, and arrows) and mouse non-coding sequences are indicated as single lines; while human coding sequences are indicated by open shapes (e.g., arrowheads) and human non-coding sequences are indicated as double lines. For simplicity and to conserve space, the 5′ end of the Ig locus is depicted as starting with unrearranged human J_{H} gene segments, although it will be understood that unrearranged human V_{H} and D_{H} gene segments and corresponding non-coding sequences also appear upstream (e.g., see FIG. 3 for reference).

FIGS. 2A-2C show a schematic of an exemplary approach for genetically engineering a mouse immunoglobulin locus to generate an embodiment of an IgMDA rodent as described herein. Unless indicated otherwise, mouse coding sequences are indicated by filled in shapes (e.g., ovals, boxes, and arrows) and mouse non-coding sequences are indicated as single lines; while human coding sequences are indicated by open shapes (e.g., arrowheads) and human non-coding sequences are indicated as double lines. For simplicity and to conserve space, the 5′ end of the Ig locus is depicted as starting with unrearranged human J_{H} gene segments, although it will be understood that unrearranged human V_{H} and D_{H} gene segments and corresponding non-coding sequences also appear upstream (e.g., see FIG. 3 for reference).

FIGS. 3A-3C show exemplary heavy chain, kappa light chain, and lambda light chain loci configurations for a VI3 mouse (FIG. 3A), an exemplary IgA mouse comprising human variable region genes (FIG. 3B), and an exemplary IgMDA mouse (FIG. 3C). VI3 mouse heavy chain locus (FIG. 3A) contains an endogenous mouse heavy chain constant region, which also contains other endogenous non-coding regulatory sequences not shown (e.g., the corresponding switch regions (e.g., “Sμ” as shown in FIG. 1A), I exons, and cognate germline promoters of the Ig heavy chain constant region genes). Unless indicated otherwise, mouse coding sequences are indicated by filled in shapes (e.g., ovals, boxes, and arrows) and mouse non-coding sequences are indicated as single lines; while human coding sequences are indicated by open shapes (e.g., arrowheads) and human non-coding sequences are indicated as double lines. “HO” indicates that the exemplified mouse is homozygous for the relevant locus; “Het” indicates that the exemplified mouse is heterozygous or hemizygous for the relevant locus.

FIGS. 4A-4C show exemplary analyses of IgA and IgM antibodies produced by IgA, IgMDA, and VI3 mice. Specifically, a concentration of IgA antibodies produced by IgA mice, IgMDA mice, and VI3 mice was obtained from stool (μg/mg feces) (FIG. 4A) and serum (μg/μl serum) (FIG. 4B), and a concentration of IgM antibodies produced by IgA mice, IgMDA mice, and VI3 mice obtained from serum (μg/μl serum) (FIG. 4C). Each dot represents a mouse (10 weeks old); *p<0.05, **p<0.01, ***p<0.001, ns: not significant, determined by Mann-Whitney test; Data: mean+/−S.E.M.

FIG. 5 shows production of bacteria-specific IgA antibodies in mice. Specifically, production of bacteria-specific IgA antibodies was measured by flow cytometry and the percentage of bacteria-specific IgA antibodies in stool samples of VI3, IgA mice, and IgMDA mice was determined. Each dot represents a mouse (10 weeks old); *p<0.05, ns: not significant, determined by Mann-Whitney test; Data: mean+/−S.E.M.

FIGS. 6A-6F show quantification of splenic T and B cells in IgA, IgMDA, and VI3 mice. FIG. 6A shows percentage of splenic T cells detected as represented by % CD3+ cells among live mCD45+ cells. FIG. 6B shows % of B cells detected as represented by % CD19+ cells among live mCD45+ cells. FIG. 6C shows ratio of splenic T:B cells detected as represented by % of mCD3+ vs. mcCD19+ cells. Each dot represents a mouse. FIGS. 6D-6F show representative flow plots for CD19+ B cells and CD3+ T cells in VI3 mice (FIG. 6D), IgA mice (FIG. 6E), and IgMDA mice (FIG. 6F).

FIGS. 7A-7F show exemplary percentages of kappa and lambda light chain antibodies among splenic B cells in IgA, IgMDA, and VI3 mice. FIG. 7A shows percentage of B cells with κ light chains detected as represented by % kappa (κ) light chain+cells among mC19+ cells (% of B cells with κ light chains). FIG. 7B shows percentage of B cells detected with λ light chains % lambda (λ) light chain+ cells among mC19+ cells. FIG. 7C shows the ratio of κ:λ antibodies detected as represented by % κ vs. λ light chain antibodies. Each dot represents a mouse. FIGS. 7D-7F show representative flow plots for κ light chain+ vs. λ light chain+ B cells in VI3 mice (FIG. 7D), IgA mice (FIG. 7E), and IgMDA mice (FIG. 7F).

FIGS. 8A-8C show representative flow plots for IgM+ and IgA+ B cells in VI3 mice (FIG. 8A), IgA mice (FIG. 8B), and MDA mice (FIG. 8C).

FIG. 9 shows exemplary anti-viral antigen antibody titers with IgA isotype detected in serum of VI3, IgA, and IgMDA mice 27 days post-immunization with a viral antigen. Specifically, mice were immunized with viral antigen for 4 consecutive weeks. After resting for 1 week, serum samples were harvested, and anti-viral antigen IgA antibody titer was quantified by ELISA. Each dot represents the value for one mouse.

FIGS. 10A-10C show representative flow plots of plasma cell subpopulations isolated from bone marrow of IgA mice immunized with antigen. Specifically, FIG. 10A shows a population of plasma cells, FIG. 10B shows the subpopulation of plasma cells that are antigen-positive (Ag+) plasma cells, and FIG. 10C shows the subpopulation of Ag+ plasma cells that have an IgA isotype.

### DEFINITIONS

The scope of the present invention is defined by the claims appended hereto and is not limited by certain embodiments described herein. Those skilled in the art, reading the present specification, will be aware of various modifications that may be equivalent to such described embodiments, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context. Additional definitions for the following and other terms are set forth throughout the specification. Patent and non-patent literature references cited within this specification, or relevant portions thereof, are incorporated herein by reference in their entireties.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a,” “an,” and “the” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of a group is present in, employed in, or otherwise relevant to a given product or process. In some embodiments, more than one, or all group members are present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where embodiments or aspects are referred to as “comprising” particular elements, features, etc., provided are embodiments or aspects that “consist,” or “consist essentially of,” such elements, features, etc. For purposes of simplicity, those embodiments have not in every case been specifically set forth in so many words herein. It should also be understood that any embodiment or aspect can be explicitly excluded from the claims, regardless of whether the specific exclusion is recited in the specification.

Administration: as used herein, includes the administration of a composition (e.g., antigen or antibody) to a subject or system (e.g., to a cell, organ, tissue, organism, or relevant component or set of components thereof). The skilled artisan will appreciate that route of administration may vary depending, for example, on the subject or system to which the composition is being administered, the nature of the composition, the purpose of the administration, etc. For example, in certain embodiments, administration to a subject (e.g., to a human or a rodent) may be bronchial (including by bronchial instillation), buccal, enteral, interdermal, intradermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and/or vitreal. In some embodiments, administration may involve intermittent dosing. In some embodiments, administration may involve continuous dosing (e.g., perfusion) for at least a selected period of time.

Amino acid: as used herein, is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of the foregoing.

The term “antigen” refers to any agent (e.g., protein, peptide, polysaccharide, glycoprotein, glycolipid, nucleotide, portions thereof, or combinations thereof) that, when introduced into an immunocompetent host, is recognized by the immune system, particularly the adaptive immune system, of the host and elicits an immune response in the host that is mediated by an “antigen binding protein” such as an immunoglobulin or T cell receptor (TCR). The T cell receptor recognizes a peptide presented in the context of a major histocompatibility complex (MHC) as part of an immunological synapse. The peptide MHC (pMHC) complex is recognized by TCR, with the peptide (antigenic determinant) and the TCR idiotype providing the specificity of the interaction. Accordingly, the term “antigen” encompasses peptides presented in the context of MHCs, e.g., peptide-MHC complexes, e.g., pMHC complexes. The peptide displayed on MHC may also be referred to as an “epitope” or an “antigenic determinant”. The terms “peptide,” “antigenic determinant,” “epitopes,” etc., encompass not only those presented naturally by antigen presenting cells (APCs), but may be any desired peptide so long as it is recognized by an immune cell of a genetically modified non-human animal, e.g., when presented appropriately to the cells of an immune system. For example, a peptide having an artificially prepared amino acid sequence may also be an antigen.

Antigen binding protein: as used herein, refers to any protein or polypeptide that specifically binds to at least one antigen of interest. Antigen binding proteins include, but are not limited to, antibodies, heavy chains, light chains (e.g., λ or κ light chains), heavy chain variable domains, light chain variable domains (e.g., λ or κ light chain variable domains), and single chain variable fragments (scFv). In some embodiments, antigen binding proteins can be multi-specific and specifically bind to two or more epitopes or antigens.

The terms “antigen-binding protein,” “immunoglobulin, “antibody,” “antibodies,” “binding protein” and the like refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 20070004909, incorporated herein by reference in its entirety, and Ig-DARTS such as those disclosed in U.S. Pat. Appl. Pub. 20090060910, incorporated herein by reference in its entirety. In some embodiments, an antibody utilized in accordance with the present invention is in a format selected from, but not limited to, intact IgA, IgG, IgE or IgM antibodies.

The term “specifically binds,” “binds in a specific manner,” “antigen-specific” or the like, indicates that the molecules involved in the specific binding are (1) able to stably bind, e.g., associate, e.g., form intermolecular non-covalent bonds, under physiological conditions, and are (2) unable to stably bind under physiological conditions to other molecules outside the specified binding pair.

Approximately: as applied to one or more values of interest, includes to a value that is similar to, e.g., within a meaningful range of, a stated reference value. The allowable variation encompassed by the term “about” or “approximately” depends on the particular system under study, and can be readily appreciated by one of ordinary skill in the art. In embodiments, the term “approximately” or “about” refers to a range of values that fall within ±10% (greater than or less than) of the stated reference value unless otherwise stated or otherwise evident from the context (except where such number would exceed 100% of a possible value).

Biologically active: as used herein, refers to a characteristic of any agent that has activity in a biological system, in vitro or in vivo (e.g., in an organism). For instance, an agent that, when present in an organism, has a biological effect within that organism is considered to be biologically active. In particular embodiments, where a protein or polypeptide is biologically active, a portion of that protein or polypeptide that shares at least one biological activity of the protein or polypeptide is typically referred to as a “biologically active” portion.

Chimeric: as used herein, refers to nucleic acids or proteins whose structures (i.e., nucleotide or amino acid sequences, respectively) include portions that are from different sources (e.g., from different species). In some embodiments, the “chimeric” nucleic acids or proteins described herein include nucleotide or amino acid sequences, respectively, that are from both a non-human source and a human source. In such embodiments, the “chimeric” nucleic acids or proteins can also be referred to as “humanized” nucleic acids or proteins, respectively.

Comparable: as used herein, refers to two or more agents, entities, situations, sets of conditions, etc. that may not be identical to one another but that are sufficiently similar to permit comparison there between so that conclusions may reasonably be drawn based on differences or similarities observed. Persons of ordinary skill in the art will understand, in context, what degree of identity is required in any given circumstance for two or more such agents, entities, situations, sets of conditions, etc. to be considered comparable.

Conservative: as used herein, refers to instances when describing a conservative amino acid substitution, including a substitution of an amino acid residue by another amino acid residue having a side chain R group with similar chemical properties (e.g., charge or hydrophobicity). In general, a conservative amino acid substitution will not substantially change the functional properties of interest of a protein, for example, the ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some embodiments, a conservative amino acid substitution can be a substitution of any native residue in a protein with alanine, as used in, for example, alanine scanning mutagenesis. In some embodiments, a conservative substitution is made that has a positive value in the PAM250 log-likelihood matrix disclosed in Gonnet, G. H. et al., 1992, Science 256:1443-1445, which is incorporated herein by reference in its entirety. In some embodiments, a substitution is a moderately conservative substitution wherein the substitution has a nonnegative value in the PAM250 log-likelihood matrix.

Control: as used herein, refers to the art-understood meaning of a “control” being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. A “control” also includes a “control animal.” A “control animal” may have a modification as described herein, a modification that is different as described herein, or no modification (i.e., a wild-type animal). In one experiment, a “test” parameter (e.g., a variable being tested) is applied. In a second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Derived from: when used concerning a rearranged variable region gene or a variable domain “derived from” an unrearranged variable region and/or unrearranged variable region gene segments refers to the ability to trace the sequence of the rearranged variable region gene or variable domain back to a set of unrearranged variable region gene segments that were rearranged to form the rearranged variable region gene that expresses the variable domain (accounting for, where applicable, splice differences and somatic mutations). For example, a rearranged variable region gene that has undergone somatic mutation does not change the fact that it is derived from the unrearranged variable region gene segments.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous locus: as used herein, refers to a genetic locus found in a parent or reference organism prior to introduction of a disruption, deletion, replacement, alteration, or modification as described herein. In some embodiments, an endogenous locus has a sequence found in nature. In some embodiments, an endogenous locus is a wild-type locus. In some embodiments, an endogenous locus is an engineered locus. In some embodiments, a reference organism is a wild-type organism. In some embodiments, a reference organism is an engineered organism. In some embodiments, a reference organism is a laboratory-bred organism (whether wild-type or engineered).

Engineered: as used herein refers, in general, to the aspect of having been manipulated by the hand of man. For example, in some embodiments, a polynucleotide may be considered to be “engineered” when two or more sequences that are not linked together in that order in nature are manipulated by the hand of man to be directly linked to one another in the engineered polynucleotide. In some embodiments, an engineered polynucleotide may comprise a regulatory sequence that is found in nature in operative association with a first coding sequence but not in operative association with a second coding sequence, is linked by the hand of man so that it is operatively associated with the second coding sequence. Alternatively, or additionally, in some embodiments, first and second nucleic acid sequences that each encode polypeptide elements or domains that in nature are not linked to one another may be linked to one another in a single engineered polynucleotide. Comparably, in some embodiments, a cell or organism may be considered to be “engineered” if it has been manipulated so that its genetic information is altered (e.g., new genetic material not previously present has been introduced, or previously present genetic material has been altered or removed). As is common practice and is understood by persons of skill in the art, progeny of an engineered polynucleotide or cell are typically still referred to as “engineered” even though the actual manipulation was performed on a prior entity. Furthermore, as will be appreciated by persons of skill in the art, a variety of methodologies are available through which “engineering” as described herein may be achieved. For example, in some embodiments, “engineering” may involve selection or design (e.g., of nucleic acid sequences, polypeptide sequences, cells, tissues, and/or organisms) through use of computer systems programmed to perform analysis or comparison, or otherwise to analyze, recommend, and/or select sequences, alterations, etc.). Alternatively, or additionally, in some embodiments, “engineering” may involve use of in vitro chemical synthesis methodologies and/or recombinant nucleic acid technologies such as, for example, nucleic acid amplification (e.g., via the polymerase chain reaction) hybridization, mutation, transformation, transfection, etc., and/or any of a variety of controlled mating methodologies. As will be appreciated by those skilled in the art, a variety of established such techniques (e.g., for recombinant DNA, oligonucleotide synthesis, and tissue culture and transformation (e.g., electroporation, lipofection, etc.)) are well known in the art and described in various general and more specific references that are cited and/or discussed throughout the present specification. See e.g., Sambrook et al., Molecular Cloning: A Laboratory Manual 2nd ed., Cold Spring Harbor Laboratory Press, Cold Spring Harbor, N.Y., 1989 and Principles of Gene Manipulation: An Introduction to Genetic Manipulation, 5th Ed., ed. By Old, R. W. and S. B. Primrose, Blackwell Science, Inc., 1994, incorporated herein by reference in their entireties.

Expression: as used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and (4) post-translational modification of a polypeptide or protein.

Functional: as used herein, refers to a form or fragment of an entity (e.g., a gene or gene segment) that exhibits a particular property (e.g., forms part of a coding sequence) and/or activity. For example, in the context of immunoglobulins, variable regions are encoded by unique gene segments (i.e., V, D and/or J) that are assembled (or recombined) to form functional coding sequences. When present in the genome, gene segments are organized in clusters, although variations do occur. A “functional” gene segment is a gene segment represented in an expressed sequence (i.e., a variable region) for which the corresponding genomic DNA has been isolated (i.e., cloned) and identified by sequence. Some immunoglobulin gene segment sequences contain open reading frames and are considered functional although not represented in an expressed repertoire, while other immunoglobulin gene segment sequences contain mutations (e.g., point mutations, insertions, deletions, etc.) resulting in a stop codon and/or truncated sequence which subsequently render(s) such gene segment sequences unable to perform the property/ies and/or activity/ies associated with a non-mutated sequence(s). Such sequences are not represented in expressed sequences and, therefore, categorized as pseudogenes.

Gene: as used herein, refers to a DNA sequence in a chromosome that codes for a product (e.g., an RNA product and/or a polypeptide product). In some embodiments, a gene includes sequence that does not code for a polypeptide. In some particular embodiments, a gene may include both polypeptide coding (e.g., exonic) and polypeptide non-coding (e.g., intronic) sequence. In some embodiments, a gene may include one or more regulatory sequences (e.g., promoters, enhancers, etc.) and/or intron sequences that, for example, may control or impact one or more aspects of gene expression (e.g., cell-type-specific expression, inducible expression, etc.). For the purpose of clarity, we note that, as used in the present disclosure, the term “gene” generally refers to a portion of a nucleic acid that encodes a polypeptide or fragment thereof; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid.

The phrase “gene segment,” or “segment” includes reference to a variable (V) gene segment (e.g., an immunoglobulin light chain variable (V_{L}) gene segment or an immunoglobulin heavy chain variable (V_{H}) gene segment; a TCR Vα gene segment, TCR Vβ gene segment, a TCR Vδ gene segment, or a TCR Vγ gene segment), a diversity gene segment (e.g., an immunoglobulin heavy chain diversity (D_{H}) gene segment, or a TCR Do gene segment), or a joining (J) gene segment (e.g., an immunoglobulin light chain joining (J_{L}) gene segment or an immunoglobulin heavy chain joining (J_{L}) gene segment), which includes unrearranged sequences at immunoglobulin loci that can participate in rearrangement (mediated by, e.g., endogenous recombinases) to form a rearranged light chain V_{L}/J_{L} or rearranged heavy chain V_{H}/D_{H}/J_{H} sequence.

Genetically modified non-human animal or genetically engineered non-human animal: are used interchangeably herein and refer to any non-naturally occurring non-human animal (e.g., a rodent, e.g., a rat or a mouse) in which one or more of the cells of the non-human animal contain a genetic modification in its germline genome (e.g., such as an insertion, deletion, and/or mutation). In some embodiments, a genetically modified non-human animal has one or more cells that contain a heterologous nucleic acid and/or gene encoding a polypeptide of interest, in whole or in part. For example, in some embodiments, a “genetically modified non-human animal” or “genetically engineered non-human animal” refers to non-human animal that contains a transgene or transgene construct as described herein. In some embodiments, a heterologous nucleic acid and/or gene is introduced into the cell, directly or indirectly by introduction into a precursor cell, by way of deliberate genetic manipulation, such as by microinjection or by infection with a recombinant virus. The term genetic manipulation does not include classic breeding techniques, but rather is directed to introduction of recombinant DNA molecule(s). This molecule may be integrated within a chromosome. The phrases “genetically modified non-human animal” or “genetically engineered non-human animal” refers to animals that are heterozygous or homozygous for a heterologous nucleic acid and/or gene, and/or animals that have single or multi-copies of a heterologous nucleic acid and/or gene.

A genome of a non-human animal may be considered a “somatic genome,” e.g., may be the genome found in the somatic cells of the non-human animal, or may be considered a “germline genome,” e.g., may be the genome that is found in the germ cells of the non-human animal and is passed on to the offspring of the non-human animal. A skilled artisan will readily recognize that gene segments in immunoglobulin heavy variable region, and/or immunoglobulin light chain variable region loci that are unrearranged in the germline genome are capable of rearranging in select somatic cells (e.g., a T cell or B cell) of the non-human animal to form a rearranged immunoglobulin variable region gene that encodes an immunoglobulin variable domain. Accordingly, an unrearranged heavy and/or light chain locus in exemplary embodiments may be found in the germline genome of the non-human animal, and the rearranged sequence derived therefrom may be found, e.g., in a B cell, e.g. in a plasma cell, of the non-human animal.

Germline Configuration or Germline Sequence: as used herein, refers to an arrangement of sequences (e.g., gene segments) as found in an endogenous germline genome of a wild-type animal (e.g., mouse, rat, or human), or an RNA or amino acid sequence encoded by a DNA sequence as found in an endogenous germline genome of an animal (e.g., mouse, rat, or human). Examples of germline configurations of immunoglobulin gene segments can be found, e.g., in LeFranc, M-P., The Immunoglobulin Facts Book, Academic Press, May 23, 2001 (referred to herein as “LeFranc 2001”):
An exemplary configuration of human heavy chain variable region gene segments and human heavy chain constant region genes can be found at p. 47 of LeFranc 2001;
An exemplary configuration of human λ light chain variable region gene segments and human λ light chain constant region genes can be found at p. 61 of LeFranc 2001;
An exemplary configuration of human κ light chain variable region gene segments and human κ light chain constant region genes can be found at p. 53 of LeFranc 2001;
An exemplary configuration of mouse heavy chain variable region gene segments and mouse heavy chain constant region genes can be found at Lucas, J. et al., Chapter 1: The Structure and Regulation of the Immunoglobulin Loci, Molecular Biology of B Cells, 2^{nd} Edition, Academic Press, 2015 (Lucas);
An exemplary configuration of mouse λ light chain variable region gene segments and mouse λ light chain constant region genes can be found at LeFranc, M-P et al., Chapter 4: Immunoglobulin Lambda (IGL) Genes of Human and Mouse, Molecular Biology of B Cells, 1^{st} Edition, Academic Press, 2004 (LeFranc 2004); and
An exemplary configuration of mouse κ light chain variable region gene segments and mouse κ light chain constant region genes can be found at Christele, M-J, et al., Nomenclature and Overview of the Mouse (Mus musculus and Mus sp.) Immunoglobulin Kappa (IGK) Genes, Exp Clin Immunogenet 2001, 18:255-279 (Christele).
   Each of the cited sections of LeFranc 2001, Lucas, LeFranc 2004, and Christele are incorporated herein by reference.

Germline Genome: as used herein, refers to the genome found in a germ cell (e.g., a gamete, e.g., a sperm or egg) used in the formation of an animal. A germline genome is a source of genomic DNA for cells in an animal. As such, an animal (e.g., a rodent, e.g., a mouse or rat) having a modification in its germline genome is considered to have the modification in the genomic DNA of all of its cells.

Heterologous: as used herein, refers to an agent or entity from a different source. For example, when used in reference to a polypeptide, gene, or gene product present in a particular cell or organism, the term clarifies that the relevant polypeptide, gene, or gene product: 1) was engineered by the hand of man; 2) was introduced into the cell or organism (or a precursor thereof) through the hand of man (e.g., via genetic engineering); and/or 3) is not naturally produced by or present in the relevant cell or organism (e.g., the relevant cell type or organism type). “Heterologous” also includes a polypeptide, gene or gene product that is normally present in a particular native cell or organism, but has been altered or modified, for example, by mutation or placement under the control of non-naturally associated and, in some embodiments, non-endogenous regulatory elements (e.g., a promoter).

Host cell: as used herein, refers to a cell into which a nucleic acid or protein has been introduced. Persons of skill upon reading this disclosure will understand that such a term refers not only to the particular subject cell, but also is used to refer to the progeny of such a cell. Because certain modifications may occur in succeeding generations due to either mutation or environmental influences, such progeny may not, in fact, be identical to the parent cell, but are still included within the scope of the phrase “host cell.” In some embodiments, a host cell is or comprises a prokaryotic or eukaryotic cell. In general, a host cell is any cell that is suitable for receiving and/or producing a heterologous nucleic acid or protein, regardless of the Kingdom of life to which the cell is designated. Exemplary cells include those of prokaryotes and eukaryotes (single-cell or multiple-cell), bacterial cells (e.g., strains of Escherichia coli, Bacillus spp., Streptomyces spp., etc.), mycobacteria cells, fungal cells, yeast cells (e.g., Saccharomyces cerevisiae, Schizosaccharomyces pombe, Pichia pastoris, Pichia methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a cell is a human, monkey, ape, hamster, rat, or mouse cell. In some embodiments, a cell is eukaryotic and is selected from the following cells: Chinese Hamster Ovarian (CHO) (e.g., CHO K1, DXB-11 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In some embodiments, a cell comprises one or more viral genes, e.g., a retinal cell that expresses a viral gene (e.g., a PER.C6® cell). In some embodiments, a host cell is or comprises an isolated cell. In some embodiments, a host cell is part of a tissue. In some embodiments, a host cell is part of an organism.

Humanized: is used herein in accordance with its art-understood meaning to refer to nucleic acids or proteins whose structures (i.e., nucleotide or amino acid sequences) include portions that are from a non-human source, which are engineered to have a structure and function more similar to true human nucleic acids or proteins than the original source nucleic acids or proteins. For example, humanizing can involve selecting amino acid or nucleic acid substitutions to make a non-human sequence more similar to a human sequence. To give but one example, in the case of a membrane receptor, a “humanized” gene may encode a polypeptide having an extracellular portion having an amino acid sequence as that of a human extracellular portion and the remaining sequence as that of a non-human (e.g., mouse) polypeptide. In some embodiments, a humanized gene comprises at least a portion of a DNA sequence of a human gene. In some embodiments, a humanized protein comprises a sequence having a portion that appears in a human protein. The term “human” is art recognized and refers to nucleic acids or proteins whose structures (i.e., nucleotide or amino acid sequences) are entirely from a human source.

Identity: as used herein in connection with a comparison of sequences, refers to identity as determined by a number of different algorithms known in the art that can be used to measure nucleotide and/or amino acid sequence identity. In some embodiments, identities as described herein are determined using a ClustalW v. 1.83 (slow) alignment employing an open gap penalty of 10.0, an extend gap penalty of 0.1, and using a Gonnet similarity matrix (MACVECTOR™ 10.0.2, MacVector Inc., 2008).

Immune cell: as used herein, refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, plasma cells, or B-lymphocytes.

In vitro: as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and/or a non-human animal. In the context of cell-based systems, the term may be used to refer to events that occur within a living cell (as opposed to, for example, in vitro systems).

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are separated from 10% to 100%, 15%-100%, 20%-100%, 25%-100%, 30%-100%, 35%-100%, 40%-100%, 45%-100%, 50%-100%, 55%-100%, 60%-100%, 65%-100%, 70%-100%, 75%-100%, 80%-100%, 85%-100%, 90%-100%, 95%-100%, 96%-100%, 97%-100%, 98%-100%, or 99%-100% of the other components with which they were initially associated. In some embodiments, isolated agents are separated from 10% to 100%, 10%-99%, 10%-98%, 10%-97%, 10%-96%, 10%-95%, 10%-90%, 10%-85%, 10%-80%, 10%-75%, 10%-70%, 10%-65%, 10%-60%, 10%-55%, 10%-50%, 10%-45%, 10%-40%, 10%-35%, 10%-30%, 10%-25%, 10%-20%, or 10%-15% of the other components with which they were initially associated. In some embodiments, isolated agents are separated from 11% to 99%, 12%-98%, 13%-97%, 14%-96%, 15%-95%, 20%-90%, 25%-85%, 30%-80%, 35%-75%, 40%-70%, 45%-65%, 50%-60%, or 55%-60% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. In some embodiments, isolated agents are 80%-99%, 85%-99%, 90%-99%, 95%-99%, 96%-99%, 97%-99%, or 98%-99% pure. In some embodiments, isolated agents are 80%-99%, 80%-98%, 80%-97%, 80%-96%, 80%-95%, 80%-90%, or 80%-85% pure. In some embodiments, isolated agents are 85%-98%, 90%-97%, or 95%-96% pure. In some embodiments, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when: a) by virtue of its origin or source of derivation is not associated with some or all of the components that accompany it in its native state in nature; b) it is substantially free of other polypeptides or nucleic acids of the same species from the species that produces it in nature; or c) is expressed by or is otherwise in association with components from a cell or other expression system that is not of the species that produces it in nature. Thus, for instance, in some embodiments, a polypeptide that is chemically synthesized, or is synthesized in a cellular system different from that which produces it in nature, is considered to be an “isolated” polypeptide. Alternatively, or additionally, in some embodiments, a polypeptide that has been subjected to one or more purification techniques may be considered to be an “isolated” polypeptide to the extent that it has been separated from other components: a) with which it is associated in nature; and/or b) with which it was associated when initially produced.

Locus as used herein, refers to a location of a gene, DNA sequence, polypeptide-encoding sequence, or position on a chromosome of the genome of an organism. For example, an “immunoglobulin locus” may refer to the location of an immunoglobulin gene segment (e.g., V, D, J or C), immunoglobulin gene segment DNA sequence, immunoglobulin gene segment-encoding sequence, or immunoglobulin gene segment position on a chromosome of the genome of an organism that has been identified as to where such a sequence resides. It is understood in the art that a locus can encompass more than a single nucleotide position (e.g., a single coordinate on a chromosome). For instance, a person of ordinary skill in the art understands that an “immunoglobulin locus” can encompass hundreds of thousands of nucleotides (represented by associated chromosomal coordinates). An “immunoglobulin locus” may comprise a regulatory element of an immunoglobulin gene segment, including, but not limited to, an enhancer, a promoter, 5′ and/or 3′ regulatory sequence or region, or a combination thereof. An “immunoglobulin locus” may comprise intergenic DNA, e.g., DNA that normally resides or appears between gene segments in a wild-type locus. Persons of ordinary skill in the art will appreciate that chromosomes may, in some embodiments, contain hundreds or even thousands of genes and demonstrate physical co-localization of similar genetic loci when comparing between different species. Such genetic loci can be described as having shared synteny.

Naturally appears: as used herein in reference to a biological element (e.g., a nucleic acid sequence) means that the biological element can be found in a specified context and/or location, absent engineering (e.g., genetic engineering), in a cell or organism (e.g., an animal). In other words, a sequence that naturally appears in a specified context and/or location is not in the specified context and/or location as the result of engineering (e.g., genetic engineering). For example, a sequence that naturally appears adjacent to a human Jκ1 gene segment in an endogenous human immunoglobulin kappa light chain locus is a sequence that can be found adjacent to a human Jκ1 gene segment in an endogenous human immunoglobulin kappa light chain locus, absent genetic engineering, in a human. In some embodiments, a sequence can be obtained, derived, and/or isolated from where it naturally appears in a cell or organism. In some embodiments, a cell or organism is not a direct source of a sequence that naturally appears in the cell or organism. For example, a corresponding sequence in a cell or organism could be identified and then produced or replicated by mechanisms known in the art.

Non-human animal: as used herein, refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human animal is a mammal. In some embodiments, a non-human mammal is a primate, a goat, a sheep, a pig, a dog, a cow, or a rodent. In some embodiments, a non-human animal is a rodent such as a rat or a mouse.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

Operably linked: as used herein, refers to a juxtaposition of components, where the components described are in a relationship permitting them to function as linked (e.g., when the components are present in the proper tissue, cell type, cellular activity, etc.). For example, one or more V_{H} gene segments, one or more D gene segments, and one or more J_{H} gene segments are “operably linked” to a heavy chain constant region if the V_{H}, D, and J_{H} gene segments can be spliced to the heavy chain constant region at the proper time in B cell development, regardless of whether such splicing occurs in, e.g., a cell outside the immune system (e.g., a germ cell). A control sequence “operably linked” to a coding sequence is ligated in such a way that expression of the coding sequence is achieved under conditions compatible with the control sequences. “Operably linked” sequences include both expression control sequences that are contiguous with a gene of interest and expression control sequences that act in trans or at a distance to control a gene of interest (or sequence of interest). The term “expression control sequence” includes polynucleotide sequences, which are necessary to affect the expression and processing of coding sequences to which they are ligated. “Expression control sequences” include: appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance polypeptide stability; and when desired, sequences that enhance polypeptide secretion. The nature of such control sequences differs depending upon the host organism. For example, in prokaryotes, such control sequences generally include promoter, ribosomal binding site and transcription termination sequence, while in eukaryotes typically such control sequences include promoters and transcription termination sequence. The term “control sequences” is intended to include components whose presence is essential for expression and processing and can also include additional components whose presence is advantageous, for example, leader sequences and fusion partner sequences.

Polypeptide or Protein: as used herein, refers to any polymeric chain of amino acids and encompasses naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.). In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. In some embodiments, a polypeptide has an amino acid sequence encoded by a sequence that does not occur in nature (e.g., a sequence that is engineered in that it is designed and/or produced through action of the hand of man to encode said polypeptide).

Promoter: as used herein includes a DNA sequence operably linked to a nucleic acid sequence to be transcribed, such as a nucleic acid sequence encoding a desired molecule. A promoter is generally positioned upstream of a nucleic acid sequence to be transcribed and provides a site for specific binding by RNA polymerase and other transcription factors. The phrase “endogenous promoter” refers to a promoter that is naturally associated, e.g., in a wild-type organism, with an endogenous gene.

Rearranged: as used herein, describes a DNA sequence that includes two or more immunoglobulin gene segments joined (directly or indirectly) together, such that the joined gene segments together have a DNA sequence that encodes a variable region of an immunoglobulin. The two or more immunoglobulin gene segments of a rearranged DNA sequence are no longer associated with functioning recombination signal sequences (RSS), and as such cannot undergo further rearrangement. Those of skill in the art will recognize that, while two or more immunoglobulin gene segments of a rearranged DNA sequence may not be able to rearrange further, it does not mean that other immunoglobulin gene segments within the same locus cannot undergo, e.g., secondary rearrangement. Those of skill in the art will appreciate that rearranged gene segments (e.g., in a rearranged immunoglobulin variable region) can be joined together via a natural VDJ recombination process. Those of skill in the art will also appreciate that rearranged gene segments (e.g., in a rearranged immunoglobulin variable region) can be engineered to be joined together, e.g., by joining the gene segments using standard recombinant techniques. Rearranged immunoglobulin variable regions typically include two or more joined immunoglobulin gene segments. For example, a rearranged immunoglobulin λ light chain variable region can include a Vλ gene segment joined with a Jλ gene segment. A rearranged immunoglobulin heavy chain variable region can include a V_{H} gene segment, a D gene segment, a J_{H} gene segment that are joined. Those of skill in the art will also appreciate that all or substantially all intergenic sequence is generally removed between immunoglobulin gene segments in a rearranged immunoglobulin variable region. Those of skill in the art will further appreciate that a rearranged sequence can include, among other things, introns in the gene segments.

Recombinant: as used herein, refers to molecules (e.g., DNA, RNA, or polypeptides) formed by laboratory methods of genetic recombination (e.g., cloning) to bring together genetic material from multiple sources (e.g., organisms, tissues, cells, genomes, or portions of a genome). In some embodiments, recombinant polypeptides are designed, engineered, prepared, expressed, created, or isolated by recombinant means, such as polypeptides expressed using a recombinant expression vector transfected into a host cell, polypeptides isolated from a recombinant, combinatorial human polypeptide library (Hoogenboom, H. R., 1997, TIB Tech. 15:62-70; Azzazy, H. and W. E. Highsmith, 2002, Clin. Biochem. 35:425-45; Gavilondo, J. V. and J. W. Larrick, 2002, BioTechniques 29:128-45; Hoogenboom H., and P. Chames, 2000, Immunol. Today 21:371-8, incorporated herein by reference in their entireties), antibodies isolated from a non-human animal (e.g., a rodent, e.g., a mouse or rat) that has been genetically modified to include human immunoglobulin genes (see e.g., Taylor, L. D. et al., 1992, Nucl. Acids Res. 20:6287-95; Kellermann, S-A. and L. L. Green, 2002, Curr. Opin. Biotechnol. 13:593-7; Little, M. et al., 2000, Immunol. Today 21:364-70; Osborn, M. J. et al., 2013, J. Immunol. 190:1481-90; Lee, E-C. et al., 2014, Nat. Biotech. 32(4):356-63; Macdonald, L. E. et al., 2014, Proc. Natl. Acad. Sci. U.S.A. 111(14):5147-52; Murphy, A. J. et al., 2014, Proc. Natl. Acad. Sci. U.S.A. 111(14):5153-8, each of which is incorporated herein by reference in its entirety), or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements result from mutagenesis (e.g., in vivo or in vitro) of a known sequence element, e.g., from a natural or synthetic (e.g., man-made) source. For example, in some embodiments, a recombinant polypeptide is comprised of sequences found in the genome of a source organism of interest (e.g., human, mouse, etc.). In some embodiments, a recombinant polypeptide has an amino acid sequence that resulted from mutagenesis (e.g., in vitro or in vivo, for example, in a non-human animal), so that the amino acid sequences of the recombinant polypeptides are sequences that, while originating from and related to polypeptides sequences, may not naturally exist within the genome of a non-human animal in vivo.

Reference: as used herein, refers to a standard or control agent, animal, cohort, individual, population, sample, sequence, or value against which an agent, animal, cohort, individual, population, sample, sequence or value of interest is compared. In some embodiments, a reference agent, animal, cohort, individual, population, sample, sequence, or value is tested and/or determined substantially simultaneously with the testing or determination of an agent, animal, cohort, individual, population, sample, sequence or value of interest. In some embodiments, a reference agent, animal, cohort, individual, population, sample, sequence, or value is a historical reference, optionally embodied in a tangible medium. In some embodiments, a reference may refer to a control. A “reference” also includes a “reference animal.” A “reference animal” may have a modification as described herein, a modification that is different as described herein or no modification (i.e., a wild-type animal). Typically, as would be understood by persons of skill in the art, a reference agent, animal, cohort, individual, population, sample, sequence, or value is determined or characterized under conditions comparable to those utilized to determine or characterize an agent, animal (e.g., a mammal), cohort, individual, population, sample, sequence or value of interest.

Replacement: as used herein, refers to a process through which a “replaced” nucleic acid sequence (e.g., a gene) found in a host locus (e.g., in a genome) is removed from its original position within that locus, and a different, “replacement” nucleic acid is located in its place. In some embodiments, the replaced nucleic acid sequence and the replacement nucleic acid sequences are comparable to one another in that, for example, they are homologous to one another, contain corresponding elements (e.g., protein-coding elements, regulatory elements, etc.), and/or have similar or identical sequences. In some embodiments, a replaced nucleic acid sequence includes one or more of a promoter, an enhancer, a splice donor site, a splice acceptor site, an intron, an exon, an untranslated region (UTR); in some embodiments, a replacement nucleic acid sequence includes one or more coding sequences. In some embodiments, a replacement nucleic acid sequence is a homolog or variant (e.g., mutant) of the replaced nucleic acid sequence. In some embodiments, a replacement nucleic acid sequence is an ortholog or homolog of the replaced sequence. In some embodiments, a replacement nucleic acid sequence is or comprises a human nucleic acid sequence. In some embodiments, including where the replacement nucleic acid sequence is or comprises a human nucleic acid sequence, the replaced nucleic acid sequence is or comprises a rodent sequence (e.g., a mouse or rat sequence). In some embodiments, including where the replacement nucleic acid sequence is or comprises a human nucleic acid sequence, the replaced nucleic acid sequence is or comprises a human sequence. In some embodiments, a replacement nucleic acid sequence is a variant or mutant (i.e., a sequence that contains one or more sequence differences, e.g., substitutions, as compared to the replaced sequence) of the replaced sequence. The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, a replacement is a substitution of an endogenous sequence with a heterologous sequence that results in the production of a gene product from the nucleic acid sequence so placed (comprising the heterologous sequence), but not expression of the endogenous sequence; a replacement is of an endogenous genomic sequence with a nucleic acid sequence that encodes a polypeptide that has a similar function as a polypeptide encoded by the endogenous sequence. In various embodiments, an endogenous gene or fragment thereof is replaced with a corresponding human gene or fragment thereof. A corresponding human gene or fragment thereof is a human gene or fragment that is an ortholog of or is substantially similar or the same in structure and/or function, as the endogenous gene or fragment thereof that is replaced.

Substantially: as used herein, refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial similarity: as used herein, refers to a comparison between amino acid or nucleic acid sequences. As will be appreciated by those of ordinary skill in the art, two sequences are generally considered to be “substantially similar” if they contain similar residues (e.g., amino acids or nucleotides) in corresponding positions. As is understood in the art, while similar residues may be identical residues (see also Substantial Identity, below), similar residues may also be non-identical residues with appropriately comparable structural and/or functional characteristics. For example, as is well known by those of ordinary skill in the art, certain amino acids are typically classified as “hydrophobic” or “hydrophilic” amino acids, and/or as having “polar” or “non polar” side chains. Substitution of one amino acid for another of the same type may often be considered a “conservative” substitution. Typical amino acid categorizations are summarized in Table 1 below.

TABLE 1
Amino acid categorizations

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Se | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | 1.3 |
| Valine | Val | V | Nonpolar | Neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, S. F. et al., 1990, J. Mol. Biol., 215(3): 403-10; Altschul, S. F. et al., 1996, Meth. Enzymol. 266:460-80; Altschul, S. F. et al., 1997, Nucleic Acids Res., 25:3389-402; Baxevanis, A. D. and B. F. F. Ouellette (eds.) Bioinformatics: A Practical Guide to the Analysis of Genes and Proteins, Wiley, 1998; and Misener et al. (eds.) Bioinformatics Methods and Protocols, Methods in Molecular Biology, Vol. 132, Humana Press, 1998, incorporated herein by reference in their entireties. In addition to identifying similar sequences, the programs mentioned above typically provide an indication of the degree of similarity. In some embodiments, two sequences are considered to be substantially similar if at least, e.g., but not limited to, 80%, 85%, 90%, 91%, 92%, 93%, 94%, 95%, 96%, 97%, 98%, 99% or more of their corresponding residues are similar (e.g., identical or include a conservative substitution) over a relevant stretch of residues. In some embodiments, the relevant stretch is a complete sequence (e.g., a sequence of a gene, a gene segment, a sequence encoding a domain, a polypeptide, or a domain). In some embodiments, the relevant stretch is at least 9, 10, 11, 12, 13, 14, 15, 16, 17 or more residues. In some embodiments, the relevant stretch is at least 10, 15, 20, 25, 30, 35, 40, 45, 50, or more residues. In some embodiments, the relevant stretch includes contiguous residues along a complete sequence. In some embodiments, the relevant stretch includes discontinuous residues along a complete sequence, for example, noncontiguous residues brought together by the folded conformation of a polypeptide or a portion thereof.

Substantial identity: as used herein, refers to a comparison between amino acid or nucleic acid sequences. As will be appreciated by those of ordinary skill in the art, two sequences are generally considered to be “substantially identical” if they contain identical residues (e.g., amino acids or nucleotides) in corresponding positions. As is well-known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, S. F. et al., 1990, J. Mol. Biol., 215(3): 403-10; Altschul, S. F. et al., 1996, Meth. Enzymol. 266:460-80; Altschul, S. F. et al., 1997, Nucleic Acids Res., 25:3389-402; Baxevanis, A. D. and B. F. F. Ouellette (eds.) Bioinformatics: A Practical Guide to the Analysis of Genes and Proteins, Wiley, 1998; and Misener et al. (eds.) Bioinformatics Methods and Protocols, Methods in Molecular Biology, Vol. 132, Humana Press, 1998, each of which is incorporated herein by reference in its entirety. In addition to identifying identical sequences, the programs mentioned above typically provide an indication of the degree of identity. In some embodiments, two sequences are considered to be substantially identical if at least 80%, 85%, 90%, 91%, 92%, 93%, 94%, 95%, 96%, 97%, 98%, 99% or more of their corresponding residues are identical over a relevant stretch of residues. In some embodiments, a relevant stretch of residues is a complete sequence. In some embodiments, a relevant stretch of residues is, e.g., but not limited to, at least 10, 15, 20, 25, 30, 35, 40, 45, 50, or more residues.

Targeting construct or targeting vector: as used herein, refers to a polynucleotide molecule that comprises a targeting region. A targeting region comprises a sequence that is identical or substantially identical to a sequence in a target cell, tissue or animal and provides for integration of the targeting construct into a position within the genome of the cell, tissue, or animal via homologous recombination. Targeting regions that target using site-specific recombinase recognition sites (e.g., loxP or Frt sites) are also included and described herein. In some embodiments, a targeting construct as described herein further comprises a nucleic acid sequence or gene of particular interest, a selectable marker, control and/or regulatory sequences, and other nucleic acid sequences that allow for recombination mediated through exogenous addition of proteins that aid in or facilitate recombination involving such sequences. In some embodiments, a targeting construct as described herein further comprises a gene of interest in whole or in part, wherein the gene of interest is a heterologous gene that encodes a polypeptide, in whole or in part, that has a similar function as a protein encoded by an endogenous sequence. In some embodiments, a targeting construct as described herein further comprises a humanized gene of interest, in whole or in part, wherein the humanized gene of interest encodes a polypeptide, in whole or in part, that has a similar function as a polypeptide encoded by an endogenous sequence. In some embodiments, a targeting construct (or targeting vector) may comprise a nucleic acid sequence manipulated by the hand of man. For example, in some embodiments, a targeting construct (or targeting vector) may be constructed to contain an engineered or recombinant polynucleotide that contains two or more sequences that are not linked together in that order in nature yet manipulated by the hand of man to be directly linked to one another in the engineered or recombinant polynucleotide.

Transgene or transgene construct: as used herein, refers to a nucleic acid sequence (encoding e.g., a polypeptide of interest, in whole or in part) that has been introduced into a cell by the hand of man such as by the methods described herein. A transgene could be partly or entirely heterologous, i.e., foreign, to the genetically modified animal or cell into which it is introduced. A transgene can include one or more transcriptional regulatory sequences and any other nucleic acid, such as introns or promoters, which may be necessary for expression of a selected nucleic acid sequence.

Unrearranged: as used herein, describes a DNA sequence that includes two or more immunoglobulin gene segments that have not undergone a recombination event or otherwise been joined, and therefore, include intergenic sequence(s) between them. Those of skill in the art will appreciate that unrearranged V gene segments and J gene segments can be associated with an intact recombination signal sequence (RSS). Unrearranged D gene segments can be flanked by two intact recombination signal sequences (RSSs). Those of skill in the art will further appreciate that unrearranged gene segments can include, among other things, introns.

Variant: as used herein, includes a nucleic acid sequence or a peptide sequence that differs in sequence from a reference nucleic acid sequence or peptide sequence respectively. In some embodiments, a variant retains one or more biological properties of a reference nucleic acid sequence or peptide sequence. In some embodiments, changes in the sequence of a nucleic acid variant may not alter the amino acid sequence of a peptide encoded by the reference nucleic acid, e.g., due to the degeneracy of the genetic code, or may result in amino acid substitutions, additions, deletions, fusions, and truncations. In some embodiments, changes in the sequence of peptide variants are limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant or can be a variant that is not known to occur naturally. Non-naturally occurring variants of nucleic acids and peptides may be made by mutagenesis techniques or by direct synthesis.

Vector: as used herein, refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operably linked genes are referred to herein as “expression vectors.”

Wild-type: as used herein, refers to an entity having a structure and/or activity as found in nature in a “normal” (as contrasted with mutant, diseased, altered, engineered, etc.) state or context. Those of ordinary skill in the art will appreciate that wild-type genes and polypeptides often exist in multiple different forms (e.g., alleles).

### DETAILED DESCRIPTION

The present disclosure provides genetically modified non-human animals (e.g., rodents, e.g., mice or rats) with modified endogenous immunoglobulin heavy chain loci so that the class(es) of antibodies produced by the animal are limited. For example, the present disclosure provides genetically modified non-human animals (e.g., rodents, e.g., mice or rats) that produce only, primarily, or an increased titer of IgA antibodies, e.g., by introducing one or more modifications that disrupt the function and/or expression of the endogenous immunoglobulin constant region genes Ighm, Ighg, Ighd, and/or Ighe. In other embodiments, the present disclosure provides animals (e.g., rodents, e.g., mice or rats) that produce only, primarily, or an increased titer of IgA, IgM, and IgD antibodies, e.g., by introducing one or more modifications that disrupt the function and/or expression of the endogenous immunoglobulin constant region genes Ighg and Ighe. The present disclosure also contemplates the humanization of the endogenous immunoglobulin heavy and/or light chain variable region locus in a non-human animal such that IgA antibodies produced by the animal in response to antigen stimulation contain human variable domains and non-human IgA constant domains. Human antigen-binding domains (e.g., human immunoglobulin heavy and/or light chain variable domains) identified from genetically modified non-human animals described herein may be utilized in a therapeutic antibody with a constant domain that is not limited to an IgA constant domain (e.g., an IgG constant domain). Such an approach would utilize potential beneficial properties of antigen binding domains originating from IgA antibodies, while also benefiting from the longer half-life and ease of production of IgG antibodies, for example. Human antigen-binding domains (e.g., human immunoglobulin heavy and/or light chain variable domains) identified from genetically modified non-human animals described herein may be utilized in various antigen-binding protein formats.

Without wishing to be bound by any particular theory, it is contemplated that genetically modified non-human animals as described herein provide an improved in vivo system that exploits the expression of antibodies containing IgA constant domains for the production of therapeutic antibodies. Thus, provided non-human animals are particularly useful for the development of human antibodies and human antibody-based molecules (e.g., multi-specific binding agents, scFvs, fusion polypeptides, etc.) against targets associated with infection and chronic inflammation. The present disclosure also recognizes that an in vivo system producing only IgA antibodies may allow for generation of antibodies with higher rates of somatic hypermutation (SHM) (and thus, provide a diverse pool of antigen positive antibodies), and for the identification of therapeutic antibodies that are able to bind to specific targets (e.g., viral antigens).

Several studies have suggested that plasma cells are a source of high-affinity antibodies. In addition, in humans and wild type mice, IgA antibodies are known to be expressed on the surface of plasma cells. Pinto et al. (2013) Blood 121(20):4110-14. By demonstrating that genetically modified mice disclosed herein (which comprise disrupted function and/or expression of the endogenous immunoglobulin constant region genes Ighm, Ighg, Ighd, and/or Ighe) comprise plasma cells that express cell surface bound IgA, the present disclosure recognizes that these plasma cells can be a convenient source for obtaining sequences of high-affinity antigen-positive antibodies.

The present disclosure describes, among other things, an immunoglobulin heavy chain locus that comprises a deletion or disruption of certain constant region gene sequences (e.g., Ighg, Ighe, Ighm, and/or Ighd). In particular, the present disclosure describes the production of a non-human animal (e.g., a rodent, e.g., a mouse or rat) having a germline genome that contains an engineered immunoglobulin heavy chain (IgH) locus that is, in some embodiments, able to produce exclusively IgA antibodies.

In various embodiments, a humanized immunoglobulin heavy chain locus contains at least one human V_{H}, at least one human D_{H} and at least one human J_{H} gene segment operably linked to a non-human immunoglobulin heavy chain constant region (e.g., an endogenous non-human immunoglobulin heavy chain constant region that includes one or more immunoglobulin heavy chain constant region genes such as, for example, IgM, IgD, and/or IgA), e.g., a plurality of human V_{H}, D_{H} and J_{H} gene segments operably linked to a non-human immunoglobulin heavy chain constant region. In some embodiments, non-human animals as described herein that contain human immunoglobulin heavy chain and/or light chain variable region gene segments, are characterized by their ability to produce antibodies that comprise human immunoglobulin variable domains and rodent IgA constant domains. In some embodiments, provided non-human animals have a germline genome that includes one or more immunoglobulin loci depicted in the Drawings. Such engineered non-human animals provide a source of human antibodies and human antibody fragments and provide an improved in vivo system suitable for exploiting human heavy chain and light chain variable region sequences originating in IgA antibodies for the production of human therapeutic antibodies.

Various aspects of certain embodiments are described in detail in the following sections, each of which can apply to any aspect or embodiment as described herein. The use of sections is not for limitation.

### Antibody Repertoires in Non-Human Animals

Immunoglobulins (also called antibodies) are large (˜150 kD), Y-shaped glycoproteins that are produced by B cells of a host immune system to neutralize pathogens (e.g., viruses, bacteria, etc.). Each immunoglobulin (Ig) is composed of two identical heavy chains and two identical light chains, each of which has two structural components: a variable domain and a constant domain. The heavy and light chain variable regions differ in antibodies produced by different B cells but are the same for all antibodies produced by a single B cell or B cell clone. The heavy and light chain variable regions of each antibody together comprise the antigen-binding region (or antigen-binding site).

Immunoglobulins can exist in different varieties that are referred to as isotypes or classes based on the heavy chain constant regions (or domains) that they contain. The heavy chain constant region is identical in all antibodies of the same isotype but differs in antibodies of different isotypes. Table 2 below summarizes the nine antibody isotypes in mouse and human.

TABLE 2
Antibody Isotypes in Mouse and Human

| Mouse | Human |
|---|---|
| IgM | IgM |
| IgD | IgD |
| IgG1 | IgG1 |
| IgG2a | IgG2 |
| IgG2b | IgG3 |
| IgG2c | IgG4 |
| IgG3 | IgE |
| IgE | IgA1 |
| IgA | IgA2 |

Additional isotypes have been identified in other species. Isotypes confer specialized biological properties on the antibody due to the different structural characteristics among the different isotypes and are found in different locations (cells, tissues, etc.) within an animal body. Initially, B cells produce IgM and IgD with identical antigen-binding regions. Upon activation, B cells switch to different isotypes by a process referred to as class switching, which involves a change of the constant region of the antibody produced by the B cell while the variable regions remain the same, thereby preserving antigen specificity of the original antibody (B cell).

Early in antibody development, antibody heavy chains undergo a selection process wherein nature chooses, through a variety of selection schemes, suitable heavy chains to undergo further selection to eventually form functional and affinity-matured antibodies. Diversity arising from heavy and light chain variable gene rearrangement occurs in the bone marrow and precedes class switching. Antibody heavy chains expressed from recombined heavy chain gene segments in progenitor B cells (or, pro-B cells) are paired with a surrogate light chain for presentation on the surface of the pro-B cell in an IgM isotype to form a structure (which includes other co-receptors) referred to as a pre-B cell receptor, or pre-BCR. Once the pre-BCR is presented on the cell surface, the pre-BCR signals its appropriate formation of the complex to the cell, effectively instructing the cell that the heavy chain has passed this early selection step. Thus, the cell is informed that the heavy chain may undergo further selection. If the heavy chain contains a defect that is deleterious to the formation of a pre-BCR when presented in the context of an IgM and a surrogate light chain, the cell will undergo apoptosis. If the cell undergoes apoptosis, the usefulness, or contribution to diversity, of the heavy chain variable region of the heavy chain will be lost. Thus, a very early step in antibody selection requires presentation of the heavy chain together with a surrogate light chain in the context of an IgM isotype.

After B-cells exit the bone marrow, engagement with antigen (which requires a low affinity interaction between the rearranged antibody expressed as a cell-surface IgM) stimulates concerted induction of somatic hypermutation and class switching. Class switching, also referred to as isotype switching, isotypic commutation or class-switch recombination (CSR), is the process by which a mature B cells switches production of one isotype of immunoglobulin to another.

During isotype switching, the constant region of an antibody is changed, retaining the particular variable region, thereby retaining antigen specificity. However, the antibody will interact with different effector molecules depending on the isotype (i.e., type of constant domain). Mature B cells may produce IgM or IgD antibodies. Once a B cell becomes activated by an antigen, it proliferates and interacts with different signaling molecules. When these B cells interact with certain signaling molecules such T cell cytokines (e.g., IL-4, IL-5, IFNγ, TGFβ, and IL-10), they undergo class switching recombination to IgA, IgE, or IgG isotypes. Depending on the signaling molecule, a B cell may undergo class switching recombination to a particular antibody isotype. After class switching, differential antigen recognition by the surface B-cell receptor allows antibodies of increased affinity to be selected from a pool of hyper-mutated derivatives of the original IgM.

Development of therapeutic antibodies for the treatment of various human diseases has largely been centered on the creation of engineered non-human animals, in particular, engineered rodents, harboring varying amounts of genetic material in their genomes corresponding to human immunoglobulin genes (reviewed in, e.g., Bruggemann, M. et al., 2015, Arch. Immunol. Ther. Exp. 63:101-8, which is incorporated herein by reference in its entirety). Initial efforts in creating such genetically modified rodent lines focused on integration of portions of human immunoglobulin loci that could, by themselves, support recombination of gene segments and production of heavy and/or light chains that were entirely human while having endogenous immunoglobulin loci inactivated (see e.g., Bruggemann, M. et al., 1989, Proc. Nat. Acad. Sci. U.S.A. 86:67-09-13; Bruggemann, M. et al., 1991, Eur. J. Immunol. 21:1323-6; Taylor, L. D. et al., 1992, Nucl. Acids Res. 20:6287-6295; Davies, N. P. et al., 1993, Biotechnol. 11:911-4; Green, L. L. et al., 1994, Nat. Genet. 7:13-21; Lonberg, N. et al., 1994, Nature 368:856-9; Taylor, L. D. et al., 1994, Int. Immunol. 6:579-91; Wagner, S. D. et al., 1994, Eur. J. Immunol. 24:2672-81; Fishwild, D. M. et al., 1996, Nat. Biotechnol. 14:845-51; Wagner, S. D. et al., 1996, Genomics 35:405-14; Mendez, M. J. et al., 1997, Nat. Genet. 15:146-56; Green, L. L. et al., 1998, J. Exp. Med. 188:483-95; Xian, J. et al., 1998, Transgenics 2:333-43; Little, M. et al., 2000, Immunol. Today 21:364-70; Kellermann, S. A. and L. L. Green, 2002, Cur. Opin. Biotechnol. 13:593-7, each of which is incorporated by reference in their entirety).

The present disclosure provides, among other things, genetically modified non-human animals (e.g., rodents, e.g., mice or rats) that produce antibodies with particular isotypes. In some embodiments, genetically modified non-human animals produce only IgA antibodies. In some embodiments, genetically modified non-human animals produce only IgM, IgD, and IgA antibodies. In some embodiments, genetically modified non-human animals (e.g., rodents, e.g., mice or rats) described herein contain one or more genetic alterations at the endogenous immunoglobulin heavy chain locus, wherein the one or more genetic alterations disrupt expression from the immunoglobulin heavy chain constant region genes of the non-human animal: e.g., mu (Ighm), delta (Ighd), gamma 3 (Ighg3), gamma 1 (Ighg1), gamma 2a (Ighg2a), gamma 2b (Ighg2b), and epsilon (Ighe) constant region genes.

Antibodies with IgG isotype are most prevalent among circulating antibodies in humans, accounting for approximately 80%. The second most prevalent isotype in human serum is monomeric IgA, which accounts for about 15% of circulating antibodies (see Bohländer, Fabian. Frontiers in Immunology 14 (2023): 1127339, which is herein incorporated by reference). Dimeric and other multimeric forms of IgA is prevalent in mucosal linings in the body. Multimeric IgA formation is facilitated by a small molecule called the J-chain (i.e., “IgJ”) through covalent linkage. IgJ linkage in multimeric formats is required for binding of the polymeric immunoglobulin receptor (pIgR), which causes transport to the mucosa. Mice have one subclass of IgA, which exists as a dimer in serum. Humans have two subclasses of IgA, IgA1 and IgA2. Rodents also lack the main IgA receptor FcαRI.

IgA antibodies in the mucosal lining of the gut serve as a first line of defense against antigens (e.g., pathogenic bacteria and viruses) by e.g., coating bacteria or viruses by dimeric IgA, thereby blocking the antigens. Human IgA interacts with the Fcα receptor in humans to activate effector cells e.g., polymorphonuclear neutrophils (PMNs), neutrophil extracellular traps (NETs), and macrophages, leading to e.g., trogoptosis, phagocytosis and killing. Binding of IgA with polymeric immunoglobulin receptor (pIgR) causes transcytosis of IgA across mucosal barriers, and activates inflammatory signaling pathways, e.g., in ovarian cancer cells, e.g., by upregulating IFNg receptors and facilitating T cell killing of cancer cells (see Biswas, S., et al, Nature 2021). IgA antibodies have been shown to have a relatively high number of somatic hypermutations compared to other isotypes in mice (e.g., IgM and IgG). See Liu, Xin, et al., Nature immunology 23.11 (2022): 1564-1576.

The present disclosure recognizes, among other things, that IgA antibodies provide better protection at mucosal sites but present therapeutic challenges. Isolation of antigen-specific IgA antibodies is challenging. Additionally, systemic antigen administration leads to clones that are primarily IgG isotypes. Mucosal immunization leads to more antigen-specific IgA antibodies, however, harvesting B cells from the mucosa is difficult, and a higher dose or subsequent doses of antigen do not increase antigen-specific IgA antibody titer (Li, H., et al, Nature 2020, Sheikh-Mohamed, S., Mucosal Immunol 2022). Generating and purifying recombinant secretory IgA (SIgA) is challenging due to its complex structure, and SIgA also has a short half-life due to lack of FcRn binding.

The present disclosure exemplifies the successful production of a non-human animal that is capable of producing exclusively or primarily IgA antibodies. In some embodiments, genetically modified non-human animals described herein are capable of producing antigen-specific IgA antibodies when systemically immunized with antigen. Non-human mammals that produce only IgA antibodies, as provided herein, presents an opportunity to identify new therapeutic antibodies that would otherwise be undiscoverable.

### Genetically Modified Animals

The present disclosure provides genetically modified non-human animals with modified endogenous immunoglobulin heavy chain loci, where the modification controls the class of antibodies produced by the non-human animal. For example, the present disclosure provides genetically modified non-human animals that produce only, primarily, or an increased titer of IgA antibodies, e.g., by introducing one or more modifications that disrupt the function and/or expression of the endogenous immunoglobulin constant region genes Ighm, Ighg, Ighd, and/or Ighe. In other embodiments, the present disclosure provides genetically modified non-human animals that produce only, primarily, or an increased titer of IgA, IgM, and IgD antibodies, e.g., by introducing one or more modifications that disrupt the function and/or expression of the endogenous immunoglobulin constant region genes Ighg and Ighe.

Various embodiments of the genetically modified non-human animals (e.g., rodents, e.g., rats or mice) are described in more detail herein below. Nucleic acid and amino acid sequences of immunoglobulin genes and polypeptides are available from the International Immunogenetics Information System website, www.imgt.org.

### A. Engineered Heavy Chain Loci

Genetically modified non-human animals (e.g., rodents, e.g., rats or mice) provided herein include one or more genetic modifications that alter an endogenous immunoglobulin heavy chain locus and/or comprise introduction of an exogenous nucleic acid sequence that encodes a genetically modified immunoglobulin heavy chain locus. In some embodiments, one or more genetic modifications that alter an endogenous immunoglobulin heavy chain locus comprise one or more genetic modifications in an immunoglobulin heavy chain constant region (as described further below). In some embodiments, one or more genetic modifications that alter an endogenous immunoglobulin heavy chain locus comprise (i) one or more genetic modifications in an immunoglobulin heavy chain constant region (as described further below) and (ii) one or more genetic modifications in an immunoglobulin heavy chain variable region as described further below.

### 1. Engineered Constant Regions

The constant domain of an antibody can be encoded by one of various immunoglobulin constant region genes. The mouse immunoglobulin genome comprises the following constant region genes: Ighm, Ighd, Ighg1, Ighg2a, Ighg2b, Ighg2c, Ighg3, Ighe, and Igha, which encode constant region domains of IgM, IgD, IgG1, IgG2a, IgG2b, IgG2c, IgG3, IgE, and IgA antibodies, respectively. The rat immunoglobulin genome comprises the following constant region genes: Ighm, Ighd, Ighg1, Ighg2a, Ighg2b, Ighg2c, Ighe, and Igha, which encode constant region domains of IgM, IgD, IgG1, IgG2a, IgG2b, IgG2c, IgE, and IgA antibodies, respectively. The human immunoglobulin genome comprises the following constant region genes: Ighm, Ighd, Ighg1, Ighg2, Ighg3, Ighg4, Ighe, Igha1, Igha2, which encode constant region domains of IgM, IgD, IgG1, IgG2, IgG3, IgG4, IgE, IgA1, and IgA2 antibodies, respectively.

Each Ighd, Ighg, and Igha constant region gene comprises a CH1, CH2, and CH3 region, which encode the CH1 domain, CH2 domain, and CH3 domain of an antibody. Each Ighm and Ighe constant region gene comprises a CH1, CH2, CH3, and CH4 region, which encode the CH1 domain, CH2 domain, CH3 domain, and CH4 domain of an antibody.

In some embodiments, the present disclosure provides genetically modified non-human animals (e.g., rodents, e.g., mice or rats) that comprise one or more genetic alterations in the immunoglobulin heavy chain constant region. In some embodiments, a modification in an immunoglobulin constant region is made in order to control the isotype of the antibodies produced by the genetically modified non-human animal. In some embodiments, a modification in an immunoglobulin heavy chain constant region is made to generate different antibody formats (e.g., heavy-chain only antibodies).

a. IgA Constant Regions

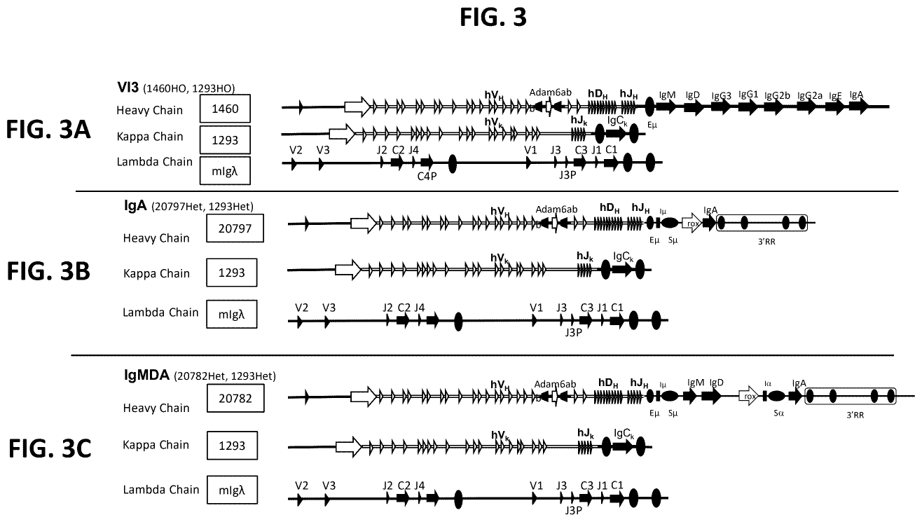

The present disclosure provides genetically modified non-human animals (e.g., rodents, e.g., mice or rats) with modified endogenous immunoglobulin heavy chain loci which results in the expression of a limited number of antibody classes being produced by the animal. For example, the present disclosure provides genetically modified non-human animals that produce only, primarily, or an increased titer of IgA antibodies, e.g., by introducing one or more modifications that disrupt the function and/or expression of the endogenous immunoglobulin constant region genes Ighm, Ighg, Ighd, and/or Ighe. Such animals are referred to herein as “IgA animals” (e.g., IgA rodents, e.g., IgA mice). Such animals further comprising human immunoglobulin variable region gene segments are referred to herein as “hIgA animals” (e.g., hIgA rodents, e.g., hIgA mice) as shown in FIG. 3B.

In some embodiments, a genetically modified non-human animal (e.g., a rodent, e.g., a mouse or rat) comprises an engineered endogenous immunoglobulin heavy chain locus that comprises an immunoglobulin heavy chain constant region comprising an animal (e.g., rodent, e.g., mouse or rat) immunoglobulin heavy chain alpha (Igha) constant region that is modified such that the genetically modified non-human animal produces only, primarily, or an increased titer of IgA antibodies. In some embodiments, the genetically modified non-human animals have an increased titer of IgA antibodies that is at least 5%, 10%, 20%, 30%, 40%, 50%, 60%, 70%, 80%, 90% or more greater that the titer of IgA antibodies in a corresponding non-human animal without the modification. In some embodiments, genetically modified non-human animals produce antigen-specific IgA antibodies in response to antigen stimulation.

As described herein, IgA antibodies produced have been shown to have a relatively high number of somatic hypermutations compared to other isotypes in mice (e.g., IgM and IgG). See Liu, Xin, et al., Nature immunology 23.11 (2022): 1564-1576. The present disclosure provides, among other things, genetically modified non-human animals that produce an increased titer of IgA antibodies, and, in some embodiments, produce antibodies having a higher rate of somatic hypermutation (SHM). In some embodiments, a non-human animal is provided whose genome comprises an engineered immunoglobulin heavy chain locus, where the non-human animal includes a B cell (e.g., a plasma cell) that includes a human heavy variable region sequence, a human λ light chain variable region sequence, and/or a human κ light chain variable region sequence that is somatically hypermutated. In some embodiments, a human heavy chain variable region sequence, a human λ light chain variable region sequence, and/or a human κ light chain variable region sequence present in a B cell (e.g., a plasma cell) of a genetically modified non-human animal of the present disclosure has 1, 2, 3, 4, 5, or more somatic hypermutations. Those skilled in the art are aware of methods for identifying source gene segments in a mature antibody sequence. For example, various tools are available to aid in this analysis, such as, for example, DNAPLOT, IMGT/V-QUEST, JOINSOLVER, SoDA, and Ab-origin.

In some embodiments, a human heavy chain variable region sequence, a human λ light chain variable region sequence, and/or a human κ light chain variable region sequence produced by a genetically modified non-human animal as described herein is somatically hypermutated. In some embodiments, genetically modified non-human animal of the present disclosure (e.g., in response to antigen stimulation) have at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% of the B cells (e.g., plasma cells) in a population of B cells (e.g., plasma cells) produced in response to an antigen include a human heavy chain variable region sequence, a human λ light chain variable region sequence, and/or a human κ light chain variable region sequence that is somatically hypermutated.

In some embodiments, a genetically modified non-human animal (e.g., rodent, e.g., mouse or rat) comprises in its germline genome one or more genetic alterations at the endogenous immunoglobulin heavy chain locus that disrupt expression from one or more immunoglobulin constant region genes (e.g., one or more constant region genes selected from Ighm, Ighd, Ighg, and/or Ighe). In some embodiments, one or more genetic alterations at the endogenous immunoglobulin heavy chain locus disrupt the expression from immunoglobulin constant region genes Ighm, Ighd, Ighg, and Ighe. In some embodiments, a genetically modified non-human animal is a mouse, and the one or more genetic alterations disrupt expression from mouse Ighm, Ighd, Ighg1, Ighg2a, Ighg2b, Ighg2c, Ighg3, and Ighe genes. In some embodiments, a genetically modified non-human animal is a rat, and the one or more genetic alterations disrupt expression from rat Ighm, Ighd, Ighg1, Ighg2a, Ighg2b, Ighg2c, and Ighe genes.

In some embodiments, a genetic alteration comprises a deletion, insertion, mutation and/or inversion. In some embodiments, a genetic alteration comprises a deletion to remove all or part of one or more endogenous constant region genes.

In some embodiments, a genetic alteration comprises a deletion to remove all or part of one or more endogenous rodent (e.g., mouse or rat) constant region genes.

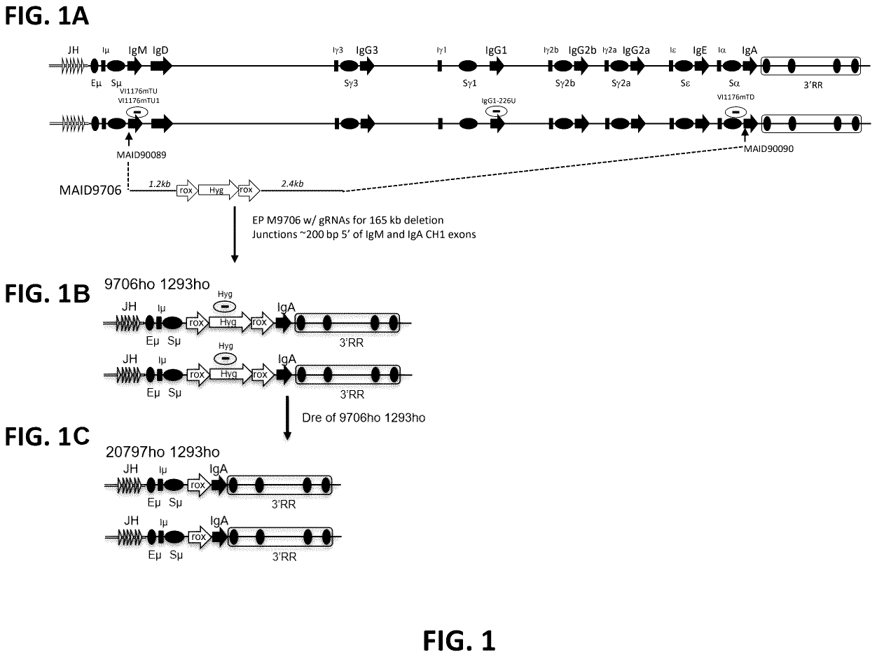

In some embodiments, a deletion removes all or part of rodent (e.g., mouse or rat) immunoglobulin heavy chain constant region genes such as Ighm, Ighd, Ighg, and Ighe constant regions genes. In some embodiments, a deletion is a contiguous sequence spanning a region in the immunoglobulin heavy chain constant region upstream of a rodent Ighm gene to a region that is upstream of a rodent immunoglobulin heavy chain constant region Igha gene. In some embodiments the deleted region is between about 10-1000 bp upstream of a rodent Ighm gene (e.g., between about 100-900 bp, 100-800 bp, 100-700 bp, 100-600 bp, 100-500 bp, 100-400 bp, 100-300 bp upstream of a rodent Ighm gene). In some embodiments the deleted region is between about 200 bp upstream of a rodent Ighm gene (e.g., the CH1 exon of the Ighm gene). In some embodiments the deleted region is between about 10-1000 bp upstream of a rodent Igha gene (e.g., between about 100-900 bp, 100-800 bp, 100-700 bp, 100-600 bp, 100-500 bp, 100-400 bp, 100-300 bp upstream of a rodent Ighm gene). In some embodiments the deleted region is between about 200 bp upstream of a rodent Igha gene (e.g., the CH1 exon of the Igha gene). In some embodiments, a genetically modified rodent (e.g., a mouse or rat) comprises an immunoglobulin heavy chain constant region comprising from 5′ to 3′ Eμ-Sμ-IgA (e.g., from 5′ to 3′ Eμ-Sμ-IgA-3′RR), where the mu intronic enhancer Eμ and mu switch region Sμ are directly 5′ of the immunoglobulin constant alpha gene sequence (see FIGS. 1B-1C). In some embodiments, a rodent IgA gene in an IgA rodent genome is operably linked to an IgM switch region Sμ. In some embodiments, immunoglobulin heavy chain constant region genes in IgA rodents described herein are associated with other regulatory elements (e.g., I exons and their cognate germline promoters). In some embodiments, a rodent IgA gene in an IgA rodent genome is associated with IgM regulatory elements, and/or IgA regulatory elements are deleted. In some embodiments, a rodent IgA gene is driven by an IgM germline promoter and IgM switch region (Sμ). The present disclosure recognizes that an IgA switch region in IgA rodents described herein may be deleted along with Ighm, Ighd, Ighg, and Ighe constant regions genes, and an IgM switch region and/or other regulatory elements may be retained and drive IgA expression.

In some embodiments, an alteration comprises one or more deletions remove part of each of non-human animal (e.g., rodent, e.g., mouse or rat) Ighm, Ighd, Ighg, and Ighe constant region genes, such that expression of the constant region genes is reduced or disrupted, e.g., reduced by at least 10%, 20%, 30%, 40%, 50%, 60%, 70%, 80%, 90% or more compared to a comparable animal that does not have the constant region modification. In some embodiments, an alteration comprises one or more deletions that do not remove all or part of the non-coding regions between the animal (e.g., rodent, e.g., mouse or rat) Ighm, Ighd, Ighg, and Ighe constant region genes. In some embodiments, an alteration comprises one or more deletions that remove all of the animal (e.g., rodent, e.g., mouse or rat) Ighm, Ighd, Ighg, and Ighe constant region genes.

In some embodiments, an alteration comprises one or more insertions in the endogenous non-human animal (e.g., rodent, e.g., mouse or rat) immunoglobulin heavy chain constant region. In some embodiments, an insertion comprises an insertion of a transgene. In some embodiments, a transgene comprises a limited set of constant region genes. In some embodiments, a transgene comprises a single constant region gene. In some embodiments, a single constant region gene is an Igha constant region gene. In some embodiments, an Igha constant region gene is a non-human animal (e.g., a rodent, e.g., a mouse or rodent) Igha constant region gene. In some embodiments, an Igha constant region gene is a human Igha (e.g., Igha1 or Igha2) constant region gene. In some embodiments, a transgene comprises only a human Igha1 constant region gene and a human Igha2 constant region gene.

In some embodiments, an alteration comprises one or more insertions in the endogenous non-human animal (e.g., rodent, e.g., mouse or rat) immunoglobulin heavy chain constant region. In some embodiments, an insertion comprises an insertion of one or more repressor elements that inhibit expression of one or more animal (e.g., rodent, e.g., mouse or rat) immunoglobulin constant region genes (e.g., Ighm, Ighd, Ighg, and Ighe constant region genes). In some embodiments, an insertion comprises an insertion of one or more repressor elements that inhibit expression of animal (e.g., rodent, e.g., mouse or rat) immunoglobulin constant region genes Ighm, Ighd, Ighg, and Ighe.

In some embodiments, an alteration comprises one or more mutations. In some embodiments, a mutation comprises a frameshift mutation that inhibits expression of one or more animal (e.g., rodent, e.g., mouse or rat) immunoglobulin constant region genes (e.g., Ighm, Ighd, Ighg, and Ighe constant region genes). In some embodiments, a mutation comprises a frameshift mutation that inhibits expression of animal (e.g., rodent, e.g., mouse or rat) immunoglobulin constant region genes Ighm, Ighd, Ighg, and Ighe.

In some embodiments, genetically modified non-human animals (e.g., rodents, e.g., mice or rats) described herein are modified to be heterozygous for the engineered endogenous immunoglobulin heavy chain IgA constant region locus. In some embodiments, genetically modified non-human animals (e.g., rodents, e.g., mice or rats) described herein are modified to be homozygous for the engineered endogenous immunoglobulin heavy chain IgA constant region locus. In some embodiments, genetically modified non-human animals (e.g., rodents, e.g., mice or rats) described herein are modified to be hemizygous for the engineered endogenous immunoglobulin heavy IgA chain constant region locus.

b. IgMDA Constant Regions

The present disclosure provides genetically modified non-human animals (e.g., rodents, e.g., mice or rats) with modified endogenous immunoglobulin heavy chain loci such as to control the class of antibodies produced by the animal. For example, the present disclosure provides genetically modified non-human animals (e.g., rodents, e.g., mice or rats) that produce only, primarily, or an increased titer of IgA, IgM, and/or IgD antibodies, e.g., by introducing one or more modifications that disrupt the function and/or expression of the endogenous immunoglobulin constant region genes Ighg and/or Ighe. In some embodiments, a genetically modified non-human animals (e.g., rodents, e.g., mice or rats) are modified to produce only, primarily, or an increased titer of IgA, IgM, and IgD antibodies, e.g., by introducing one or more modifications that disrupt the function and/or expression of the endogenous immunoglobulin constant region genes Ighg and Ighe. Such animals are referred to herein as “IgMDA animals” (e.g., IgMDA mice). Such animals further comprising human immunoglobulin variable region gene segments are referred to herein as “hIgMDA animals” (e.g., hIgMDA mice) as shown in FIG. 3C.

In some embodiments, a genetically modified non-human animal (e.g., rodent, e.g., a mouse or rat) comprises an engineered endogenous immunoglobulin heavy chain locus that comprises an immunoglobulin heavy chain constant region genes Ighm, Ighd, and/or Igha such that the genetically modified non-human animal produces only, primarily, or an increased titer of IgA, IgD, and/or IgM antibodies. In some embodiments, the genetically modified non-human animal (e.g., rodent, e.g., a mouse or rat) have an increased titer of IgA, IgD, and/or IgM antibodies that is at least 5%, 10%, 20%, 30%, 40%, 50%, 60%, 70%, 80%, 90% or more that the titer of IgA, IgD, and/or IgM antibodies in a corresponding mouse without the modification. In some embodiments, genetically modified non-human animal (e.g., rodent, e.g., a mouse or rat) produce antigen-specific IgA, IgM, and/or IgD antibodies in response to antigen stimulation.

In some embodiments, a genetically modified non-human animal (e.g., rodent, e.g., a mouse or rat) comprises in its germline genome one or more genetic alterations at the endogenous immunoglobulin heavy chain locus that disrupt expression from one or more immunoglobulin constant region genes (e.g., one or more constant region genes selected from Ighg, and/or Ighe). In some embodiments, one or more genetic alterations at the endogenous immunoglobulin heavy chain locus disrupt the expression from immunoglobulin constant region genes Ighg and Ighe. In some embodiments, a genetically modified rodent is a mouse, and the one or more genetic alterations disrupt expression from mouse Ighg1, Ighg2a, Ighg2b, Ighg2c, Ighg3, and Ighe genes. In some embodiments, a genetically modified non-human animal is a rat, and the one or more genetic alterations disrupt expression from rat Ighg1, Ighg2a, Ighg2b, Ighg2c, and Ighe genes.

In some embodiments, a genetic alteration comprises a deletion, insertion, mutation and/or inversion. In some embodiments, a genetic alteration comprises a deletion to remove all or part of one or more endogenous constant region genes.

In some embodiments, a genetic alteration comprises a deletion to remove all or part of one or more endogenous rodent (e.g., mouse or rat) constant region genes.

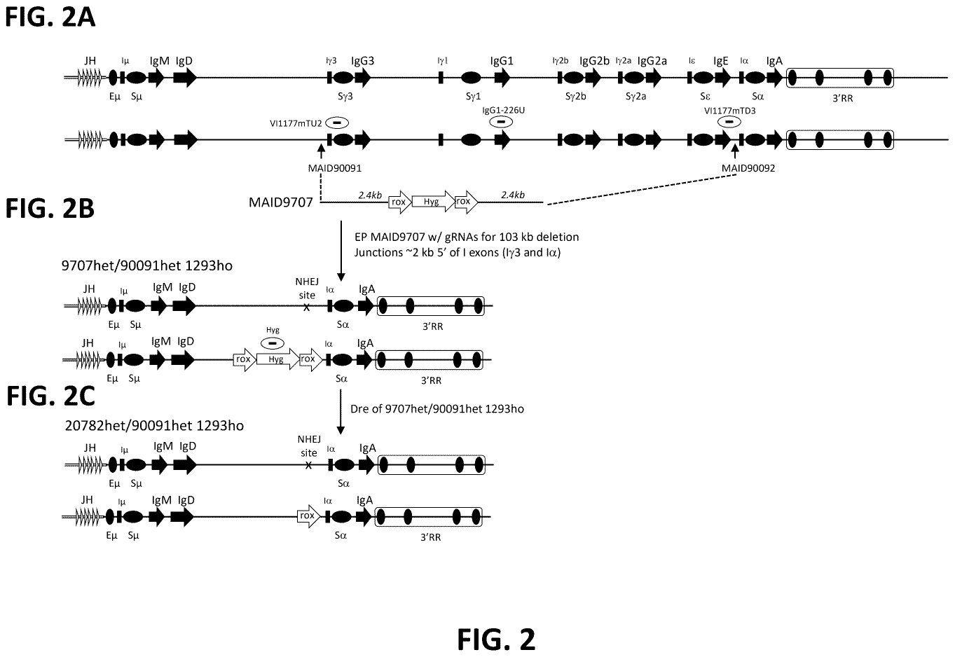

In some embodiments, a deletion removes all or part of a rodent immunoglobulin heavy chain constant region genes Ighg and Ighe. In some embodiments, a deletion is a contiguous sequence spanning a region in the immunoglobulin heavy chain constant region upstream of a rodent Ighg gene to a region that is upstream of a rodent immunoglobulin heavy chain constant region Igha gene. In some embodiments, a rodent is a mouse, and a deletion in the mouse genome is a contiguous sequence spanning a region upstream of the mouse Ighg3 gene (e.g., exon 1 of the Ighg3) to a region that is upstream of the mouse Igha gene (e.g., exon 1 of the Igha gene). In some embodiments the region is between about 10 bp-10 kb upstream of a rodent Ighg gene (e.g., between about 100-9000 bp, 100-8000 bp, 100-7000 bp, 100-6000 bp, 100-5000 bp, 100-4000 bp, 1000-3000 bp upstream of a rodent Ighm gene). In some embodiments the region is about 2000 bp upstream of a rodent Ighg gene (e.g., the CH1 exon of the Ighg3 gene). In some embodiments the region is between about 10 bp-10 kb upstream of a rodent Igha gene (e.g., between about 1000-9000b.p, 1000-8000 bp, 1000-7000 bp, 1000-6000 bp, 1000-5000 bp, 1000-4000 bp, 1000-3000 bp upstream of a rodent Ighm gene). In some embodiments the region is between about 2000 bp upstream of a rodent Igha gene (e.g., the CH1 exon of the Igha gene). In some embodiments, a genetically modified rodent (e.g., mouse) comprises an immunoglobulin heavy chain constant region that comprises from 5′ to 3′ Eμ-Sμ-IgM-IgD-Sa-IgA-3′RR, where the IgA gene is driven by its cognate germline promoter and switch region (see FIGS. 2B-2C).

In some embodiments, an alteration comprises one or more deletions that remove part of each of the non-human animal (e.g., a rodent, e.g., a mouse or rat) Ighg, and Ighe constant region genes, such that expression of the constant region genes is reduced or disrupted, e.g., reduced by at least 10%, 20%, 30%, 40%, 50%, 60%, 70%, 80%, 90% or greater compared to a comparable animal that does not have the constant region modification. In some embodiments, an alteration comprises one or more deletions that do not remove all or part of the non-coding regions between the non-human animal (e.g., a rodent, e.g., a mouse or rat) Ighg and Ighe constant region genes. In some embodiments, an alteration comprises one or more deletions remove all of the non-human animal (e.g., a rodent, e.g., a mouse or rat) Ighg, and Ighe constant region genes.

In some embodiments, an alteration comprises one or more insertions in the endogenous non-human animal (e.g., rodent, e.g., mouse or rat) genome (e.g., germline genome). In some embodiments, an insertion comprises an insertion of a transgene. In some embodiments, a transgene comprises a limited set of constant region genes. In some embodiments, a limited set of constant region genes comprises Ighm, Ighd, and Igha constant region genes. In some embodiments, Ighm, Ighd, and Igha constant region genes are human and/or non-human animal (e.g., rodent, e.g., mouse or rat) constant region genes. In some embodiments, Ighm, Ighd, and Igha constant region genes are non-human animal (e.g., rodent, e.g., mouse or rat) constant region genes. In some embodiments, Ighm, Ighd, and Igha (e.g., Igha1 and/or Igha2) constant region genes are human constant region genes.

In some embodiments, an Igha constant region gene is a human Igha (e.g., Igha1 or Igha2) constant region gene. In some embodiments, a transgene comprises only a human Igha1 constant region gene and a human Igha2 constant region gene.

In some embodiments, an alteration comprises one or more insertions in the endogenous non-human animal (e.g., a rodent, e.g., a mouse or rat) immunoglobulin heavy chain constant region. In some embodiments, an insertion comprises an insertion of one or more repressor elements that inhibit expression of one or more non-human animal (e.g., a rodent, e.g., a mouse or rat) immunoglobulin constant region genes (e.g., Ighg and Ighe constant region genes). In some embodiments, an insertion comprises an insertion of one or more repressor elements that inhibit expression of non-human animal (e.g., a rodent, e.g., a mouse or rat) immunoglobulin constant region genes Ighg and Ighe.

In some embodiments, an alteration comprises one or more mutations. In some embodiments, a mutation comprises a frameshift mutation that inhibits expression of one or more non-human animal (e.g., a rodent, e.g., a mouse or rat) immunoglobulin constant region genes (e.g., Ighg and Ighe constant region genes). In some embodiments, a mutation comprises a frameshift mutation that inhibits expression of non-human animal (e.g., a rodent, e.g., a mouse or rat) immunoglobulin constant region genes Ighg and Ighe.

In some embodiments, genetically modified non-human animal (e.g., a rodent, e.g., a mouse or rat) described herein is modified to be heterozygous for the engineered endogenous immunoglobulin heavy chain IgMDA constant region locus. In some embodiments, genetically modified non-human animal (e.g., a rodent, e.g., a mouse or rat) described herein is modified to be homozygous for the engineered endogenous immunoglobulin heavy chain IgMDA constant region locus. In some embodiments, genetically modified non-human animal (e.g., a rodent, e.g., a mouse or rat) described herein is modified to be hemizygous for the engineered endogenous immunoglobulin heavy IgMDA chain constant region locus.

### 2. Engineered Variable Domains

In some embodiments, a genetically modified non-human animal (e.g., a rodent, e.g., a mouse or rat) provided herein comprises an immunoglobulin locus comprising IgA constant region (as described above) operably linked to an immunoglobulin variable region. In some embodiments, a genetically modified non-human animal (e.g., a rodent, e.g., a mouse or rat) provided herein comprises an immunoglobulin comprising IgMDA constant region (as described above) and an immunoglobulin variable region.

In some embodiments, an immunoglobulin variable region is a heavy chain variable region. In some embodiments, an immunoglobulin variable region is a light chain variable region. In some embodiments, an immunoglobulin variable region comprises human, rodent or both variable region gene segments. In some embodiments, an immunoglobulin variable region comprises rodent (e.g., mouse) gene segments. In some embodiments, an immunoglobulin variable region comprises rodent (e.g., mouse) heavy chain variable region gene segments. In some embodiments, an immunoglobulin variable region comprises rodent (e.g., mouse) light chain variable region gene segments. In some embodiments, an immunoglobulin variable region comprises rodent (e.g., mouse) kappa light chain variable region gene segments. In some embodiments, an immunoglobulin variable region comprises rodent (e.g., mouse) lambda light chain variable region gene segments. In some embodiments, an immunoglobulin variable region comprises human gene segments. In some embodiments, an immunoglobulin variable region comprises human heavy chain variable region gene segments. In some embodiments, an immunoglobulin variable region comprises human light chain variable region gene segments. In some embodiments, an immunoglobulin variable region comprises human kappa light chain variable region gene segments. In some embodiments, an immunoglobulin variable region comprises human lambda light chain variable region gene segments.

Various human heavy chain variable regions can be used with an IgA constant region or an IgMDA constant region in genetically modified non-human animals (e.g., rodents, e.g., mice or rats) as provided herein. The ability to generate antibodies has been harnessed in genetically modified animals, which are able to generate therapeutic antibodies or antibody fragments against human targets. Exemplary genetically modified rodents comprising human V(D)J gene segments (with, e.g., endogenous heavy chain constant region loci) for generation of therapeutic antibodies are those described in U.S. Pat. Nos. 5,633,425, 5,770,429, 5,814,318, 6,075,181, 6,114,598, 6,150,584, 6,998,514, 7,795,494, 7,910,798, 8,232,449, 8,703,485, 8,907,157, and 9,145,588, each of which is hereby incorporated by reference in its entirety, as well as in U.S. Pat. Pub. Nos. 2008/0098490, 2010/0146647, 2013/0145484, 2012/0167237, 2013/0167256, 2013/0219535, 2012/0207278, 2015/0113668, 2019/0127757, and 2021/0059229 each of which is hereby incorporated by reference in its entirety, and in PCT Pub. Nos. WO2007117410, WO2008151081, WO2009157771, WO2010039900, WO2011004192, WO2011123708, WO2013045916, WO2015049517, WO2014093908, WO2014093908, WO2006008548, WO2010109165, WO2016062990, WO2018039180, WO2011158009, WO2013041844, WO2013041846, WO2013079953, WO2013061098, WO2013144567, WO2013144566, WO2013171505, WO2012018610, WO2022126113, WO2020132557, WO2017035274, WO2019236670, WO2019236671, WO2019008123, WO2021123090, WO2020169022, and WO2021244522, each of which are hereby incorporated by reference in its entirety. Other exemplary genetically modified rodents comprising human V(D)J gene segments (with, e.g., endogenous heavy chain constant region loci) for generation of therapeutic antibodies are those described in U.S. Pat. Nos. 6,596,541, 6,586,251, 8,642,835, 9,706,759, 10,238,093, 8,754,287, 10,143,186, 9,796,788, 10,130,081, 9,226,484, 9,012,717, 10,246,509, 9,204,624, and 9,686,970, each of which is hereby incorporated by reference in its entirety, as well as in U.S. Pat. Pub. Nos. 2013/0212719, 2015/0289489, 2017/0347633, 2019/0223418, 2018/0125043, 2019/0261612, and 2019/0380316, each of which is hereby incorporated by reference in its entirety, in PCT Pub. Nos. WO2013138680, WO2013138712, WO2013138681, WO2015042250, WO2012148873, WO2013134263, WO2013184761, WO2014160179, WO2017214089, WO2016149678, and WO2017123808, WO 2012018764, WO2019241692, WO2017123804, WO2022140219 and Murphy, A., “VelocImmune: Immunoglobulin Variable Region Humanized Mouse,” in Recombinant Antibodies for Immunotherapy, New York, NY, Cambridge University Press, 101-107 (2009), each of which are hereby incorporated by reference in its entirety. Additional detailed embodiments of certain exemplary genetically engineered non-human animals (e.g., rodents, e.g., rats or mice) are described below.

Various embodiments of specific heavy chain variable region locus configurations, which can be used in IgA or IgMDA animals (e.g., mice or rats), include but are not limited to the locus configurations described in more detail herein below.

a. Human Heavy Chain (HOH)

In some embodiments, a genetically modified non-human animal (e.g., rodent, e.g., mouse or rat) comprises in its genome (e.g., germline genome) an immunoglobulin heavy chain variable region comprising one or more human heavy chain V gene segments, one or more human D gene segments, and one or more human heavy chain J gene segments.

In embodiments, an immunoglobulin heavy chain variable region is located at the endogenous animal (e.g., rodent, e.g., mouse or rat) heavy chain locus. Genetically modified non-human animals comprising an engineered immunoglobulin heavy chain locus that comprise human heavy chain variable region genes operably linked to a heavy chain constant region, particularly an endogenous animal (e.g., rodent, e.g., mouse or rat) heavy chain constant region, are referred to herein as an “HoH locus.” Rodents including human heavy chain variable region genes operably linked to an endogenous rodent heavy chain constant region are exemplified in, e.g., U.S. Pat. Nos. 6,596,541; 8,642,835; and 8,697,940, and Murphy, A., “VelocImmune: Immunoglobulin Variable Region Humanized Mouse,” in Recombinant Antibodies for Immunotherapy, New York, NY, Cambridge University Press, 101-107 (2009), and Macdonald et al, Proc. Natl. Acad. Sci. USA 111:5147-52 and supporting information (www.pnas.org/cgi/content/short/1323896111), each of which is incorporated by reference in its entirety.

In some embodiments, a genetically modified non-human animal (e.g., rodent, e.g., mouse or rat) comprises in its genome (e.g., its germline genome) an engineered immunoglobulin heavy chain locus (e.g., an engineered endogenous animal (e.g., rodent, e.g., mouse or rat) immunoglobulin heavy chain locus). In some embodiments, an engineered immunoglobulin heavy chain locus comprises one or more unrearranged human V_{H} gene segments, one or more unrearranged human D_{H} gene segments, and one or more unrearranged human J_{H} gene segments.

In some embodiments, a genetically modified non-human animal (e.g., rodent, e.g., mouse or rat) is homozygous at an HoH locus. In some embodiments, a genetically modified non-human animal (e.g., rodent, e.g., mouse or rat) is heterozygous at an HoH locus.

In some embodiments, one or more unrearranged human V_{H} gene segments includes at least six human V_{H} gene segments. In some embodiments, one or more unrearranged human V_{H} gene segments includes at least 18 human V_{H} gene segments. In some embodiments, one or more unrearranged human V_{H} gene segments includes at least 39 human V_{H} gene segments. In some embodiments, one or more unrearranged human V_{H} gene segments includes at least 80 human V_{H} gene segments. In some embodiments, one or more unrearranged human D_{H} gene segments includes at least 27 human D_{H} gene segments. In some embodiments, one or more unrearranged human J_{H} gene segments includes at least six human J_{H} gene segments.

In some embodiments, one or more unrearranged human V_{H} gene segments includes at least 22 human V_{H} gene segments. In some embodiments, one or more unrearranged human D_{H} gene segments includes all human D_{H} gene segments. In some embodiments, one or more unrearranged human J_{H} gene segments includes all human J_{H} gene segments.

In some embodiments, one or more unrearranged human V_{H} gene segments includes all functional human V_{H} gene segments. In some embodiments, one or more unrearranged human V_{H} gene segments includes less than 80 human V_{H} gene segments. In some embodiments, one or more unrearranged human V_{H} gene segments includes less than 39 human V_{H} gene segments. In some embodiments, one or more unrearranged human V_{H} gene segments includes less than 18 human V_{H} gene segments. In some embodiments, one or more unrearranged human V_{H} gene segments includes less than 10 human V_{H} gene segments. In some embodiments, one or more unrearranged human V_{H} gene segments includes all human V_{H} gene segments. In some embodiments, one or more unrearranged human V_{H} gene segments includes a contiguous (e.g., unmodified) human sequence including human IGHV(III)-82 to human IGHV6-1 of a human heavy chain immunoglobulin gene locus.

In some embodiments, one or more unrearranged human D_{H} gene segments and one or more unrearranged human J_{H} include all human D_{H} and J_{H} gene segments. In some embodiments, one or more unrearranged human D_{H} gene segments includes a contiguous (e.g., unmodified) human sequence including human IGHD1-1 to human IGHJ-6 of a human heavy chain immunoglobulin gene locus.

In some embodiments, a genetically modified non-human animal is a genetically modified mouse. In some embodiments, a genetically modified mouse comprises in its genome (e.g., its germline genome) an engineered immunoglobulin heavy chain locus (e.g., an engineered endogenous mouse immunoglobulin heavy chain locus) that comprises a deletion of all endogenous mouse immunoglobulin heavy chain variable region gene segments (e.g., deletion of a contiguous mouse immunoglobulin heavy chain gene sequence from mouse IGHV1-86 to IGHJ4 on mouse chromosome 12). In some embodiments, an endogenous mouse immunoglobulin heavy chain variable region sequence is replaced (e.g., by homologous recombination) with a contiguous sequence of human immunoglobulin heavy chain variable region sequence. In some embodiments, a human immunoglobulin heavy chain variable region sequence comprises all human immunoglobulin heavy chain variable gene segments (i.e., a contiguous sequence from human IGHV(III)-82 to human IGHV6-1 on chromosome 14). In some embodiments, the human immunoglobulin heavy chain variable region sequence is at least 500 kb in size (i.e., at least 500 kb, at least 100 kb, at least 1,000 kb). In some embodiments, the replacement of the endogenous mouse immunoglobulin heavy chain locus is performed with a single recombination step. Exemplary embodiments of mice including loci with such human heavy chain variable region configurations can be found, e.g., in PCT Publication No. WO2020169022 (which is hereby incorporated by reference in its entirety).

In some embodiments, one or more unrearranged human V_{H} gene segments includes at least 18 human V_{H} gene segments, one or more unrearranged human D_{H} gene segments includes 27 human D_{H} gene segments, and one or more unrearranged human J_{H} gene segments includes six human J_{H} gene segments. Such an engineered immunoglobulin heavy chain locus can be referred to as a “VelocImmune® 1 HoH locus.” In some embodiments, one or more unrearranged human V_{H} gene segments includes at least 39 human V_{H} gene segments, one or more unrearranged human D_{H} gene segments includes 27 human D_{H} gene segments, and one or more unrearranged human J_{H} gene segments includes six human J_{H} gene segments. Such an engineered immunoglobulin heavy chain locus is referred to herein as a “VelocImmune® 2 HoH locus.” In some embodiments, one or more unrearranged human V_{H} gene segments includes at least 80 human V_{H} gene segments, one or more unrearranged human D_{H} gene segments includes 27 human D_{H} gene segments, and one or more unrearranged human J_{H} gene segments includes six human J_{H} gene segments. Such an engineered immunoglobulin heavy chain locus is referred to herein as a “VelocImmune® 3 HoH locus.”

In some embodiments, a genetically modified non-human animal (e.g., rodent, e.g., mouse or rat) comprises in its genome (e.g., its germline genome) an engineered immunoglobulin heavy chain locus (e.g., an engineered endogenous non-human animal (e.g., rodent, e.g., mouse or rat) immunoglobulin heavy chain locus) comprising one or more unrearranged human V_{H} gene segments, one or more unrearranged human D_{H} gene segments, and one or more unrearranged human J_{H} gene segments, which further comprises substitution or insertion of at least one histidine for a non-histidine residue, such that the unrearranged immunoglobulin heavy chain variable gene sequence comprises in a complementarity determining region 3 (CDR3) encoding sequence a substitution of at least one non histidine codon with a histidine codon or an insertion of at least one histidine codon (see, e.g., PCT Pub. Nos. WO2013/138712 and WO2013/138681, incorporated herein by reference in their entireties).

i. Exemplary HoH-IgA Locus Configuration

In some embodiments, a genetically modified non-human animal (e.g., rodent, e.g., mouse or rat) comprises in its genome (e.g., its germline genome) an engineered immunoglobulin heavy chain locus (e.g., an engineered endogenous non-human animal (e.g., rodent, e.g., mouse or rat) immunoglobulin heavy chain locus) comprising one or more unrearranged human V_{H} gene segments, one or more unrearranged human D_{H} gene segments, and one or more unrearranged human J_{H} gene segments that are upstream of (e.g., operably linked to) an IgA constant region locus (referred to herein as an “HoH-IgA locus”). In some embodiments, a genetically modified non-human animal (e.g., rodent, e.g., mouse or rat) is homozygous for an HoH-IgA locus. In some embodiments, a genetically modified non-human animal (e.g., rodent, e.g., mouse or rat) is heterozygous for an HoH-IgA locus.

In some embodiments, one or more unrearranged human V_{H} gene segments at a HoH locus includes at least six human V_{H} gene segments. In some embodiments, one or more unrearranged human V_{H} gene segments at an HoH-IgA locus includes at least 18 human V_{H} gene segments. In some embodiments, one or more unrearranged human V_{H} gene segments at an HoH-IgA locus includes at least 39 human V_{H} gene segments. In some embodiments, one or more unrearranged human V_{H} gene segments at an HoH-IgA locus includes at least 80 human V_{H} gene segments. In some embodiments, one or more unrearranged human D_{H} gene segments at an HoH-IgA locus includes at least 27 human D_{H} gene segments. In some embodiments, one or more unrearranged human J_{H} gene segments at an HoH-IgA locus includes at least six human J_{H} gene segments.

In some embodiments, one or more unrearranged human V_{H} gene segments at an HoH-IgA locus includes at least 18 human V_{H} gene segments, one or more unrearranged human D_{H} gene segments at an HoH-IgA locus includes 27 human D_{H} gene segments, and one or more unrearranged human J_{H} gene segments at an HoH-IgA locus includes six human J_{H} gene segments.

In some embodiments, one or more unrearranged human V_{H} gene segments at an HoH-IgA locus includes at least 39 human V_{H} gene segments, one or more unrearranged human D_{H} gene segments at an HoH-IgA locus includes 27 human D_{H} gene segments, and one or more unrearranged human J_{H} gene segments at an HoH-IgA locus includes six human J_{H} gene segments.

In some embodiments, one or more unrearranged human V_{H} gene segments at an HoH-IgA locus includes at least 80 human V_{H} gene segments, one or more unrearranged human D_{H} gene segments at an HoH-IgA locus includes 27 human D_{H} gene segments, and one or more unrearranged human J_{H} gene segments at an HoH-IgA locus includes six human J_{H} gene segments.

In some embodiments, a genetically modified non-human animal (e.g., rodent, e.g., mouse or rat) with an HoH-IgA locus comprises an engineered endogenous region locus that comprises an IgA constant region as described herein. In some embodiments, such a genetically modified non-human animal (e.g., rodent, e.g., mouse or rat) produces only, primarily, or an increased titer of IgA antibodies. In some embodiments, a genetically modified non-human animal (e.g., rodent, e.g., mouse or rat) comprises in its germline genome one or more genetic alterations at the endogenous immunoglobulin heavy chain locus that disrupt expression from endogenous immunoglobulin constant region genes Ighm, Ighd, Ighg, and Ighe. In some embodiments, a genetic alteration comprises a deletion, insertion, mutation and/or inversion.

In some embodiments, a genetically modified non-human animal (e.g., rodent, e.g., mouse or rat), which comprises a HoH-IgA locus, produces antibodies comprising, inter alia, heavy chains, wherein each heavy chain comprises a human heavy chain variable domain operably linked to an IgA constant region, and wherein the animal (e.g., rodent, e.g., mouse or rat) produces antibodies with a human variable domain and IgA constant domain, e.g., in response to antigenic stimulation.

ii. Exemplary HoH-IgMDA Locus Configuration

In some embodiments, a genetically modified non-human animal (e.g., rodent, e.g., mouse or rat) comprises in its genome (e.g., its germline genome) an engineered immunoglobulin heavy chain locus (e.g., an engineered endogenous animal (e.g., rodent, e.g., mouse or rat) immunoglobulin heavy chain locus) comprising one or more unrearranged human V_{H} gene segments, one or more unrearranged human D_{H} gene segments, and one or more unrearranged human J_{H} gene segments that are upstream of (e.g., operably linked to) an IgMDA constant region (referred to herein as an “HoH-IgMDA locus”). In some embodiments, a genetically modified non-human animal (e.g., rodent, e.g., mouse or rat) is homozygous for an HoH-IgMDA locus. In some embodiments, a genetically modified non-human animal (e.g., rodent, e.g., mouse or rat) is heterozygous for an HoH-IgMDA locus.

In some embodiments, one or more unrearranged human V_{H} gene segments at a HoH-IgMDA locus includes at least six human V_{H} gene segments. In some embodiments, one or more unrearranged human V_{H} gene segments at a HoH-IgMDA locus includes at least 18 human V_{H} gene segments. In some embodiments, one or more unrearranged human V_{H} gene segments at a HoH-IgMDA locus includes at least 39 human V_{H} gene segments. In some embodiments, one or more unrearranged human V_{H} gene segments at a HoH-IgMDA locus includes at least 80 human V_{H} gene segments. In some embodiments, one or more unrearranged human D_{H} gene segments at a HoH-IgMDA locus includes at least 27 human D_{H} gene segments. In some embodiments, one or more unrearranged human J_{H} gene segments at a HoH-IgMDA locus includes at least six human J_{H} gene segments.

In some embodiments, one or more unrearranged human V_{H} gene segments at a HoH-IgMDA locus includes at least 18 human V_{H} gene segments, one or more unrearranged human D_{H} gene segments at a HoH-IgMDA locus includes 27 human D_{H} gene segments, and one or more unrearranged human J_{H} gene segments at a HoH-IgMDA locus includes six human J_{H} gene segments.

In some embodiments, one or more unrearranged human V_{H} gene segments at a HoH-IgMDA locus includes at least 39 human V_{H} gene segments, one or more unrearranged human D_{H} gene segments at a HoH-IgMDA locus includes 27 human D_{H} gene segments, and one or more unrearranged human J_{H} gene segments at a HoH-IgMDA locus includes six human J_{H} gene segments.

In some embodiments, one or more unrearranged human V_{H} gene segments at a HoH-IgMDA locus includes at least 80 human V_{H} gene segments, one or more unrearranged human D_{H} gene segments at a HoH-IgMDA locus includes 27 human D_{H} gene segments, and one or more unrearranged human J_{H} gene segments at a HoH-IgMDA locus includes six human J_{H} gene segments.

In some embodiments, a genetically modified non-human animal (e.g., rodent, e.g., mouse or rat) with an HoH-IgMDA locus comprises a heavy chain constant region locus that comprises an IgMDA constant region as described herein. In some embodiments, such genetically modified animal (e.g., rodent, e.g., mouse or rat) produces only, primarily, or an increased titer of IgM, IgD, and/or IgA antibodies. In some embodiments, a genetically modified non-human animal (e.g., rodent, e.g., mouse or rat) comprises in its germline genome one or more genetic alterations at the endogenous immunoglobulin heavy chain locus that disrupt expression from endogenous immunoglobulin constant region genes Ighg and Ighe. In some embodiments, a genetic alteration comprises a deletion, insertion, mutation and/or inversion.

In some embodiments, a genetically modified non-human animal (e.g., rodent, e.g., mouse or rat), which comprises a HoH-IgMDA locus, produces antibodies comprising, inter alia, heavy chains, where each heavy chain comprises a human heavy chain variable domain operably linked to an heavy chain constant domain encoded by an Ighm, Ighd, or an Igha constant region gene, and wherein the non-human animal (e.g., rodent, e.g., mouse or rat) produces antibodies with a human variable domain and an IgM, IgD, or IgA constant domain, e.g., in response to antigenic stimulation.

b. Universal Heavy Chain

In some embodiments, a genetically modified non-human animal (e.g., rodent, e.g., mouse or rat) comprises in its genome (e.g., its germline genome) an engineered immunoglobulin heavy chain locus, such as comprising a restricted heavy chain variable region sequence, comprising a limited human heavy chain variable region repertoire.

In some embodiments, a genetically modified non-human animal (e.g., rodent, e.g., mouse or rat) comprises in its genome (e.g., its germline genome) an engineered immunoglobulin heavy chain locus (e.g., an engineered endogenous non-human animal (e.g., rodent, e.g., mouse or rat) immunoglobulin heavy chain locus) comprising a single human V_{H} gene segment, one or more unrearranged human D_{H} gene segments, and one or more unrearranged human J_{H} gene segments. A genetically modified rodent having an immunoglobulin heavy chain loci comprising a limited number of human heavy chain variable region gene segments is exemplified in, e.g., U.S. Patent Publication No. 2019/0261612 and U.S. Pat. No. 10,238,093, each of which is incorporated by reference in its entirety.

In some embodiments, a genetically modified non-human animal (e.g., rodent, e.g., mouse or rat) comprises in its genome (e.g., its germline genome) an engineered immunoglobulin heavy chain locus (e.g., an engineered endogenous non-human animal (e.g., rodent, e.g., mouse or rat) immunoglobulin heavy chain locus) comprising a single rearranged human heavy chain variable region. Such an engineered immunoglobulin heavy chain locus is referred to herein as a “UHC locus” or a “universal heavy chain locus” or a “common heavy chain locus.” Rodents including loci with a single rearranged human heavy chain variable region are exemplified in, e.g., U.S. Pat. No. 9,204,624, which is incorporated by reference in its entirety.

In some embodiments, a single rearranged human heavy chain variable region comprises a single human V_{H} gene segment, a single human D_{H} gene segment, and a single human J_{H} gene segment. In some embodiments, a single human V_{H} gene segment is a human V_{H}3-23, a single human D_{H} gene segment is a human D_{H}4-4, and a single human J_{H} gene segment is a human J_{H}4.

In some embodiments, a single rearranged human heavy chain variable region comprises a single human V_{H} gene segment and a single human J_{H} gene segment, which are separated by two amino acids. In some embodiments, a single human V_{H} gene segment is a human V_{H}3-23, a single human J_{H} gene segment is a human J_{H}4, and two amino acids are glycine and tyrosine.

i. Exemplary UHC-IgA Locus Configuration

In some embodiments, a genetically modified non-human animal (e.g., rodent, e.g., mouse or rat) comprises in its genome (e.g., its germline genome) an engineered immunoglobulin heavy chain locus (e.g., an engineered endogenous rodent immunoglobulin heavy chain locus) comprising a single rearranged human heavy chain variable region upstream of (e.g., operably linked to) an IgA constant region locus (referred to herein as an “UHC-IgA locus”). In some embodiments, a genetically modified animal (e.g., rodent, e.g., mouse or rat) is homozygous for an UHC-IgA locus. In some embodiments, a genetically modified animal (e.g., rodent, e.g., mouse or rat) is heterozygous for an UHC-IgA locus.

In some embodiments, a single rearranged human heavy chain variable region at an UHC-IgA locus comprises a single human V_{H} gene segment, a single human D_{H} gene segment, and a single human J_{H} gene segment. In some embodiments, a single human V_{H} gene segment is a human V_{H}3-23, a single human D_{H} gene segment is a human D_{H}4-4, and a single human J_{H} gene segment is a human J_{H}4.

In some embodiments, a single rearranged human heavy chain variable region at an UHC-IgA locus comprises a single human V_{H} gene segment and a single human J_{H} gene segment, which are separated by two amino acids. In some embodiments, a single human V_{H} gene segment is a human V_{H}3-23, a single human J_{H} gene segment is a human J_{H}4, and two amino acids are glycine and tyrosine.

In some embodiments, a genetically modified animal (e.g., rodent, e.g., mouse or rat) with an UHC-IgA locus comprises a heavy chain constant region locus that comprises an IgA constant region as described herein. In some embodiments, such a genetically modified animal (e.g., rodent, e.g., mouse or rat) produces only, primarily, or an increased titer of IgA antibodies. In some embodiments, a genetically modified animal (e.g., rodent, e.g., mouse or rat) comprises in its germline genome one or more genetic alterations at the endogenous immunoglobulin heavy chain locus that disrupt expression from endogenous immunoglobulin constant region genes Ighm, Ighd, Ighg, and Ighe. In some embodiments, a genetic alteration comprises a deletion, insertion, mutation and/or inversion.

In some embodiments, a genetically modified animal (e.g., rodent, e.g., mouse or rat), which comprises a UHC-IgA locus, produces antibodies comprising, inter alia, heavy chains, where each heavy chain comprises a human heavy chain variable domain operably linked to an IgA constant domain, e.g., in response to antigenic stimulation. In some embodiments, all heavy chains expressed by a genetically modified animal (e.g., rodent, e.g., mouse or rat) comprise human heavy chain variable domains expressed from the single rearranged human heavy chain variable region or a somatically hypermutated version thereof.

ii. Exemplary UHC-IgMDA Locus Configuration

In some embodiments, a genetically modified animal (e.g., rodent, e.g., mouse or rat) comprises in its genome (e.g., its germline genome) an engineered immunoglobulin heavy chain locus (e.g., an engineered endogenous rodent immunoglobulin heavy chain locus) comprising a single rearranged human heavy chain variable region upstream of (e.g., operably linked to) an IgMDA constant region (referred to herein as an “UHC-IgMDA locus”). In some embodiments, a genetically modified animal (e.g., rodent, e.g., mouse or rat) is homozygous for an UHC-IgMDA locus. In some embodiments, a genetically modified animal (e.g., rodent, e.g., mouse or rat) is heterozygous for an UHC-IgMDA locus.

In some embodiments, a single rearranged human heavy chain variable region at an UHC-IgMDA locus comprises a single human V_{H} gene segment, a single human D_{H} gene segment, and a single human J_{H} gene segment. In some embodiments, a single human V_{H} gene segment is a human V_{H}3-23, a single human D_{H} gene segment is a human D_{H}4-4, and a single human J_{H} gene segment is a human J_{H}4.

In some embodiments, a single rearranged human heavy chain variable region at an UHC-IgMDA locus comprises a single human V_{H} gene segment and a single human J_{H} gene segment, which are separated by two amino acids. In some embodiments, a single human V_{H} gene segment is a human V_{H}3-23, a single human J_{H} gene segment is a human J_{H}4, and two amino acids are glycine and tyrosine.

In some embodiments, a genetically modified animal (e.g., rodent, e.g., mouse or rat) with an UHC-IgMDA locus comprises a heavy chain constant region locus that comprises an IgMDA constant region as described herein. In some embodiments, such genetically modified animal (e.g., rodent, e.g., mouse or rat) produces only, primarily, or an increased titer of IgM, IgD, and/or IgA antibodies. In some embodiments, a genetically modified animal (e.g., rodent, e.g., mouse or rat) comprises in its germline genome one or more genetic alterations at the endogenous immunoglobulin heavy chain locus that disrupt expression from endogenous immunoglobulin constant region genes Ighg and Ighe. In some embodiments, a genetic alteration comprises a deletion, insertion, mutation and/or inversion.

In some embodiments, a genetically modified animal (e.g., rodent, e.g., mouse or rat), which comprises an UHC-IgMDA locus, produces antibodies comprising, inter alia, heavy chains, where each heavy chain comprises a human heavy chain variable domain operably linked to an IgM, IgD, or IgA constant domain, e.g., in response to antigenic stimulation. In some embodiments, all heavy chains expressed by a genetically modified animal (e.g., rodent, e.g., mouse or rat) comprise human heavy chain variable domains expressed from the single rearranged human heavy chain variable region or a somatically hypermutated version thereof.

c. Light on Heavy

In some embodiments, a genetically modified non-human animal (e.g., rodent, e.g., mouse or rat) comprises in its genome (e.g., its germline genome) an engineered immunoglobulin heavy chain locus (e.g., an engineered endogenous non-human animal (e.g., rodent, e.g., mouse or rat) immunoglobulin heavy chain locus) comprising one or more unrearranged human V_{L} gene segments and one or more unrearranged human J_{L} gene segments. In other words, such a genetically modified non-human animal (e.g., rodent, e.g., mouse or rat) comprises a hybrid immunoglobulin chain locus comprising human light chain variable region gene segments operably linked to a heavy chain constant region (e.g., an IgA or IgMDA heavy chain constant region). Such an engineered immunoglobulin chain locus is referred to herein as an “LoH locus.” Rodents including an immunoglobulin locus having human light chain variable region gene segments operably linked to a heavy chain constant region are exemplified in, e.g., U.S. Pat. No. 9,686,970 and U.S. Patent Publication No. 2013/0212719, each of which is incorporated by reference in its entirety.

In some embodiments, one or more unrearranged human V_{L} gene segments and one or more unrearranged human J_{L} gene segments are one or more unrearranged human Vκ gene segments and one or more unrearranged human Jκ gene segments. In other words, such a genetically modified non-human animal (e.g., rodent, e.g., mouse or rat) comprises a hybrid immunoglobulin chain locus comprising human kappa light chain variable region gene segments and a heavy chain constant region (e.g., an IgA or IgMDA heavy chain constant region). Such engineered immunoglobulin heavy chain locus is referred to herein as a “KoH” locus. Rodents including an immunoglobulin locus having human kappa light chain variable region gene segments operably linked to a heavy chain constant region are exemplified in, e.g., PCT Publication No. WO 2012018764.

In some embodiments, one or more unrearranged human V_{L} gene segments and one or more unrearranged human J_{L} gene segments are one or more unrearranged human Vλ gene segments and one or more unrearranged human Jλ gene segments.

In some embodiments, a genetically modified non-human animal (e.g., rodent, e.g., mouse or rat) is homozygous at an LoH locus. In some embodiments, a genetically modified non-human animal (e.g., rodent, e.g., mouse or rat) is heterozygous at an LoH locus.

i. Exemplary LoH-IgA Locus Configuration

In some embodiments, a genetically modified non-human animal (e.g., rodent, e.g., mouse or rat) comprises in its genome (e.g., its germline genome) an engineered immunoglobulin heavy chain locus (e.g., an engineered endogenous rodent immunoglobulin heavy chain locus) comprising human light chain variable region gene segments upstream of (e.g., operably linked to) an IgA constant region locus (referred to herein as an “LoH-IgA locus”). In some embodiments, a genetically modified animal (e.g., rodent, e.g., mouse or rat) is homozygous for an LoH-IgA locus. In some embodiments, a genetically modified animal (e.g., rodent, e.g., mouse or rat) is heterozygous for an LoH-IgA locus.

In some embodiments, human light chain variable region gene segments at an LoH-IgA locus comprise one or more unrearranged human V_{L} gene segments and one or more unrearranged human J_{L} gene segments. In some embodiments, one or more unrearranged human V_{L} gene segments and one or more unrearranged human J_{L} gene segments comprise one or more unrearranged human Vκ gene segments and one or more unrearranged human Jκ gene segments. In some embodiments, one or more unrearranged human V_{L} gene segments and one or more unrearranged human J_{L} gene segments are one or more unrearranged human Vλ gene segments and one or more unrearranged human Jλ gene segments.

In some embodiments, a genetically modified non-human animal (e.g., rodent, e.g., mouse or rat) that comprises an LoH-IgA locus comprises a heavy chain constant region locus that comprises an IgA constant region as described herein. In some embodiments, such a genetically modified animal (e.g., rodent, e.g., mouse or rat) produces only, primarily, or an increased titer of IgA antibodies. In some embodiments, a genetically modified animal (e.g., rodent, e.g., mouse or rat) comprises in its germline genome one or more genetic alterations at the endogenous immunoglobulin heavy chain locus that disrupt expression from endogenous immunoglobulin constant region genes Ighm, Ighd, Ighg, and Ighe. In some embodiments, a genetic alteration comprises a deletion, insertion, mutation and/or inversion.

In some embodiments, a genetically modified animal (e.g., rodent, e.g., mouse or rat), which comprises a LoH-IgA locus, produces antibodies comprising, inter alia, immunoglobulin chains comprising a human light chain variable domain operably linked to an IgA constant domain, e.g., in response to antigenic stimulation.

ii. Exemplary LoH-IgMDA Locus Configuration

In some embodiments, a genetically modified non-human animal (e.g., rodent, e.g., mouse or rat) comprises in its genome (e.g., its germline genome) an engineered immunoglobulin heavy chain locus (e.g., an engineered endogenous rodent immunoglobulin heavy chain locus) comprising human light chain variable region gene segments upstream of (e.g., operably linked to) an IgMDA constant region locus (referred to herein as an “LoH-IgMDA locus”). In some embodiments, a genetically modified animal (e.g., rodent, e.g., mouse or rat) is homozygous for an LoH-IgMDA locus. In some embodiments, a genetically modified animal (e.g., rodent, e.g., mouse or rat) is heterozygous for an LoH-IgMDA locus.

In some embodiments, human light chain variable region gene segments at an LoH-IgMDA locus comprise one or more unrearranged human V_{L} gene segments and one or more unrearranged human J_{L} gene segments. In some embodiments, one or more unrearranged human V_{L} gene segments and one or more unrearranged human J_{L} gene segments comprise one or more unrearranged human Vκ gene segments and one or more unrearranged human Jκ gene segments. In some embodiments, one or more unrearranged human V_{L} gene segments and one or more unrearranged human J_{L} gene segments are one or more unrearranged human Vλ gene segments and one or more unrearranged human Jλ gene segments.

In some embodiments, a genetically modified non-human animal (e.g., rodent, e.g., mouse or rat) that comprises an LoH-IgMDA locus comprises a heavy chain constant region locus that comprises an IgMDA constant region as described herein. In some embodiments, such a genetically modified animal (e.g., rodent, e.g., mouse or rat) produces only, primarily, or an increased titer of IgM, IgD, and/or IgA antibodies. In some embodiments, a genetically modified animal (e.g., rodent, e.g., mouse or rat) comprises in its germline genome one or more genetic alterations at the endogenous immunoglobulin heavy chain locus that disrupt expression from endogenous immunoglobulin constant region genes Ighg and Ighe. In some embodiments, a genetic alteration comprises a deletion, insertion, mutation and/or inversion.

In some embodiments, a genetically modified non-human animal (e.g., rodent, e.g., mouse or rat), which comprises a LoH-IgMDA locus, produces antibodies comprising, inter alia, immunoglobulin chains comprising a human light chain variable domain operably linked to an IgM, IgD, or IgA constant domain, e.g., in response to antigenic stimulation.

### B. Exemplary Engineered Immunoglobulin Light Chain Loci

The present disclosure provides that a genetically modified non-human animal (e.g., rodent, e.g., mouse or rat) comprising an IgA or IgMDA constant region, as described herein, can further comprise modifications at one or both the kappa and lambda light chain loci. In some embodiments, a genetically modified non-human animal (e.g., rodent, e.g., mouse or rat) comprises in its genome (e.g., its germline genome) an engineered immunoglobulin light chain locus (e.g., an engineered endogenous immunoglobulin light chain locus) comprising one or more unrearranged human V_{L} gene segments and one or more unrearranged human J_{L} gene segments that are upstream of (e.g., operably linked to) one or more immunoglobulin light chain constant region genes. In some embodiments, one or more unrearranged human V_{L} gene segments and one or more unrearranged human J_{L} gene segments are one or more unrearranged human Vκ gene segments and one or more unrearranged human Jκ gene segments. In some embodiments, one or more unrearranged human V_{L} gene segments and one or more unrearranged human J_{L} gene segments are one or more unrearranged human Vλ gene segments and one or more unrearranged human Jλ gene segments. In some embodiments, one or more unrearranged immunoglobulin light chain constant region genes is or comprises a Cκ. In some embodiments, one or more unrearranged immunoglobulin light chain constant region genes is or comprises a Cλ.

### 1. Engineered Kappa Light Chain

In some embodiments, a genetically modified non-human animal (e.g., rodent, e.g., mouse or rat) comprises in its genome (e.g., its germline genome) an engineered immunoglobulin light chain locus (e.g., an engineered endogenous immunoglobulin light chain locus) comprising one or more unrearranged human Vκ gene segments and one or more unrearranged human Jκ gene segments that are upstream of (e.g., operably linked to) a Cκ gene. Such an engineered immunoglobulin light chain locus is referred to herein as a “KoK locus.” Rodents including a KoK locus are exemplified in, e.g., U.S. Pat. Nos. 6,596,541; 8,642,835; and 8,697,940, each of which is incorporated by reference in its entirety. In some embodiments, an immunoglobulin κ light chain constant region gene of a KoK locus is a rodent (e.g., rat or mouse) Cκ gene. In some embodiments, an immunoglobulin κ light chain constant region gene of a KoK locus is an endogenous non-human animal (e.g., rodent, e.g., mouse or rat) Cκ gene. In some embodiments, an immunoglobulin κ light chain constant region gene of a KoK locus is an endogenous rodent (e.g., rat or mouse) Cκ gene, e.g., at an endogenous immunoglobulin κ light chain locus. In some embodiments, a genetically modified non-human animal (e.g., rodent, e.g., mouse or rat) is homozygous at a KoK locus. In some embodiments, a genetically modified non-human animal (e.g., rodent, e.g., mouse or rat) is heterozygous at a KoK locus.

In some embodiments, a genetically modified non-human animal (e.g., rodent, e.g., mouse or rat), which comprises a KoK locus, produces an antibody comprising, inter alia, κ light chains, where each κ light chain comprises a human κ light chain variable domain operably linked to a rodent (e.g., rat or mouse) κ light chain constant domain, e.g., in response to antigenic stimulation.

In some embodiments, one or more unrearranged human Vκ gene segments comprise Vκ3D-7, Vκ1D-8, Vκ1D-43, Vκ3D-11, Vκ3D-12, Vκ3D-13, Vκ3D-15, Vκ3D-16, Vκ3D-17, Vκ3D-20, Vκ6D-21, Vκ2D-26, Vκ2D-28, Vκ2D-29, Vκ2D-30, Vκ1D-33, Vκ1D-39, Vκ2D-40, Vκ2-40, Vκ1-39, Vκ1-33, Vκ2-33, Vκ2-29, Vκ2-28, Vκ1-27, Vκ2-24, Vκ6-21, Vκ3-20, Vκ1-17, Vκ1-16, Vκ3-15, Vκ1-13, Vκ1-12, Vκ3-11, Vκ1-9, Vκ1-8, Vκ1-6, Vκ1-5, Vκ5-2, and/or Vκ4-1. In some embodiments, one or more unrearranged human Jκ gene segments comprise Jκ1, Jκ2, Jκ3, Jκ4, and/or Jκ5. In some embodiments, one or more unrearranged human Vκ gene segments includes at least six human Vκ gene segments. In some embodiments, one or more unrearranged human Vκ gene segments includes at least 16 human Vκ gene segments. In some embodiments, one or more unrearranged human Vκ gene segments includes at least 30 human Vκ gene segments. In some embodiments, one or more unrearranged human Vκ gene segments includes at least 40 human Vκ gene segments. In some embodiments, one or more unrearranged human Jκ gene segments includes at least five human Jκ gene segments.

In some embodiments, one or more unrearranged human Vκ gene segments includes 12 human Vκ gene segments. In some embodiments, one or more unrearranged human Jκ gene segments includes all human Jκ gene segments.

In some embodiments, one or more unrearranged human Vκ gene segments includes at least 16 human Vκ gene segments, and one or more unrearranged human Jκ gene segments includes at least five human Jκ gene segments. Such an engineered immunoglobulin light chain locus is referred to herein as a “VelocImmune® 1 KoK locus.” In some embodiments, one or more unrearranged human Vκ gene segments includes at least 30 human Vκ gene segments, and one or more unrearranged human Jκ gene segments includes at least five human Jκ gene segments. Such an engineered immunoglobulin light chain locus is referred to herein as a “VelocImmune® 2 KoK locus.” In some embodiments, one or more unrearranged human Vκ gene segments includes at least 40 human Vκ gene segments, and one or more unrearranged human Jκ gene segments includes at least five human Jκ gene segments. Such an engineered immunoglobulin light chain locus is referred to herein as a “VelocImmune® 3 KoK locus.”

In some embodiments, one or more unrearranged human Vκ gene segments includes all human Vκ gene segments. In some embodiments, one or more unrearranged human Vκ gene segments includes all functional human Vκ gene segments. In some embodiments, one or more unrearranged human Vκ gene segments includes a contiguous (e.g., unmodified) human sequence including human IGKV3D-7 to human IGKV4-1 from a human kappa light immunoglobulin gene locus.

In some embodiments, one or more unrearranged human Jκ gene segments includes all human Jκ gene segments. In some embodiments, one or more unrearranged human Jκ gene segments includes a contiguous (e.g., unmodified) human sequence including human IGKJ1 to human IGKJ5 from a human kappa light immunoglobulin gene locus.

In some embodiments, a genetically modified non-human animal (e.g., rodent, e.g., mouse or rat) comprises in its genome (e.g., its germline genome) an engineered immunoglobulin light chain locus (e.g., an engineered endogenous immunoglobulin light chain locus) that comprises a deletion of all endogenous mouse immunoglobulin κ variable region gene segments (e.g., deletion of a contiguous mouse immunoglobulin κ gene sequence from mouse IGKV2-137 to IGKJ5 on mouse chromosome 6). In some embodiments, an endogenous mouse immunoglobulin κ sequence is replaced (e.g., by homologous recombination) with a contiguous sequence of human immunoglobulin κ sequence. In some embodiments, the human immunoglobulin κ sequence comprises all human immunoglobulin κ variable gene segment sequence (i.e., a contiguous sequence from human IGKV3D-7 to IGKJ5 on chromosome 2). In some embodiments, the human immunoglobulin κ sequence is at least 500 kb in size (i.e., at least 500 kb, at least 100 kb, at least 1,000 kb). In some embodiments, the replacement of the endogenous mouse immunoglobulin light chain locus is performed with a single recombination step. Exemplary embodiments can be found, e.g., in PCT Publication No. WO2020169022, which is hereby incorporated by reference in its entirety).

### 2. Engineered Lambda Light Chain

In some embodiments, a genetically modified non-human animal (e.g., rodent, e.g., mouse or rat) comprises in its genome (e.g., its germline genome) an engineered immunoglobulin light chain locus (e.g., an engineered endogenous non-human animal (e.g., rodent, e.g., mouse or rat) immunoglobulin light chain locus) comprising one or more unrearranged human Vλ gene segments upstream of (e.g., operably linked to) one or more unrearranged human Jλ gene segments and one or more Cλ genes. Such an engineered immunoglobulin light chain locus is referred to herein as an “LoL locus.” Rodents including an LoL locus are exemplified in, e.g., U.S. Pat. Nos. 9,012,717; 9,226,484; 9,029,628, and U.S. Patent Publication No. 2018/0125043, each of which is incorporated by reference in its entirety. Rats including an LoL locus are exemplified in, e.g., WO2014093908, which is herein incorporated by reference in its entirety.

In some embodiments, the one or more unrearranged human Jλ gene segments and one or more Cλ genes of an LoL locus are present in Jλ-Cλ clusters. In some embodiments, one or more Cλ genes of an LoL locus comprise one or more human Cλ genes. In some embodiments, one or more Cλ genes of an LoL locus comprise one or more mouse Cλ genes. In some embodiments, one or more Cλ genes of an LoL locus comprise one or more rat Cλ genes. In some embodiments, one or more Cλ genes of an LoL locus comprise one or more human Cλ genes and one or more mouse or rat Cλ genes. In some embodiments, one or more mouse Cλ genes of an LoL locus comprise a mouse Cλ1 gene. In some embodiments, a genetically modified non-human animal (e.g., rodent, e.g., mouse or rat) is homozygous at an LoL locus. In some embodiments, a genetically modified non-human animal (e.g., rodent, e.g., mouse or rat) is heterozygous at an LoL locus.

In some embodiments, a genetically modified non-human animal (e.g., rodent, e.g., mouse or rat), which comprises an LoL locus, produces an antibody comprising, inter alia, λ light chains, where each λ light chain comprises a human λ light chain variable domain operably linked to a non-human animal (e.g., rodent, e.g., mouse or rat) λ light chain constant domain, e.g., in response to antigenic stimulation. In some embodiments, a genetically modified non-human animal (e.g., rodent, e.g., mouse or rat), which comprises an LoL locus, produces an antibody comprising, inter alia, λ light chains, where each λ light chain comprises a human λ light chain variable domain operably linked to a human λ light chain constant domain, e.g., in response to antigenic stimulation.

In some embodiments, a genetically modified non-human animal (e.g., rodent, e.g., mouse or rat) comprises in its genome (e.g., its germline genome) an engineered immunoglobulin light chain locus comprising one or more unrearranged human Vλ gene segments and one or more unrearranged human Jλ gene segments upstream of (e.g., operably linked to) a Cκ gene. Such an engineered immunoglobulin light chain locus is referred to herein as an “LoK locus.” Rodents including an LoK locus are exemplified in, e.g., U.S. Pat. Nos. 9,006,511 and 9,035,128, each of which is incorporated by reference in its entirety. In some embodiments, a Cκ gene of an LoK locus is a rodent (e.g., rat or mouse) Cκ gene. In some embodiments, a Cκ gene of an LoK locus is an endogenous rodent (e.g., rat or mouse) Cκ gene. In some embodiments, a Cκ gene of an LoK locus is an endogenous rodent (e.g., rat or mouse) Cκ gene at an endogenous immunoglobulin κ light chain locus. In some embodiments, a genetically modified non-human animal (e.g., rodent, e.g., mouse or rat) is homozygous at an LoK locus. In some embodiments, a genetically modified non-human animal (e.g., rodent, e.g., mouse or rat) is heterozygous at an LoK locus.

In some embodiments, a genetically modified non-human animal (e.g., rodent, e.g., mouse or rat), which comprises an LoK locus, produces an antibody comprising, inter alia, light chains, where each light chain comprises a human λ light chain variable domain operably linked to a non-human animal (e.g., rodent, e.g., mouse or rat) κ light chain constant domain, e.g., in response to antigenic stimulation.

In some embodiments, a genetically modified non-human animal (e.g., rodent, e.g., mouse or rat) comprises in its genome (e.g., its germline genome) an engineered immunoglobulin κ light chain locus (e.g., an engineered endogenous immunoglobulin κ light chain locus) comprising one or more unrearranged human Vλ gene segments and one or more unrearranged human Jλ gene segments upstream of (e.g., operably linked to) a Cλ gene. Such an engineered immunoglobulin light chain locus is referred to herein as an “LiK locus.” Rodents including an LiK locus are exemplified in, e.g., U.S. Patent Publication No. 2019/0223418, which is incorporated by reference in its entirety. In some embodiments, a Cλ gene of an LiK locus is a rodent (e.g., rat or mouse) Cλ gene. In some embodiments, a Cλ gene of an LiK locus is a mouse Cλ1 gene. In some embodiments, a genetically modified non-human animal (e.g., rodent, e.g., mouse or rat) is homozygous at an LiK locus. In some embodiments, a genetically modified non-human animal (e.g., rodent, e.g., mouse or rat) is heterozygous at an LiK locus.

In some embodiments, a genetically modified non-human animal (e.g., rodent, e.g., mouse or rat), which comprises an LiK locus, produces an antibody comprising, inter alia, λ light chains, where each λ light chain comprises a human λ light chain variable domain operably linked to a non-human animal (e.g., rodent, e.g., mouse or rat) λ light chain constant domain, e.g., in response to antigenic stimulation.

In some embodiments, a genetically modified non-human animal (e.g., rodent, e.g., mouse or rat) comprises in its genome (e.g., its germline genome) an engineered immunoglobulin κ light chain locus (e.g., an engineered endogenous rodent immunoglobulin κ light chain locus) comprising one or more unrearranged human Vλ gene segments upstream of (e.g., operably linked to) one or more unrearranged human Jλ gene segments and one or more human Cλ genes. In some embodiments, the one or more unrearranged human Jκ gene segments and one or more Cλ genes of such an engineered immunoglobulin κ light chain locus are present in Jλ-Cλ clusters. In some embodiments, a genetically modified non-human animal (e.g., rodent, e.g., mouse or rat) is homozygous for such an engineered immunoglobulin κ light chain locus. In some embodiments, a genetically modified non-human animal (e.g., rodent, e.g., mouse or rat) is heterozygous for such an engineered immunoglobulin κ light chain locus. In some embodiments, a genetically modified non-human animal (e.g., rodent, e.g., mouse or rat), which comprises such an engineered immunoglobulin κ light chain locus, produces an antibody comprising, inter alia, λ light chains, where each λ light chain comprises a human λ light chain variable domain operably linked to a human λ light chain constant domain, e.g., in response to antigenic stimulation.

In some embodiments, a genetically modified non-human animal (e.g., rodent, e.g., mouse or rat) comprises in its genome (e.g., its germline genome) an engineered immunoglobulin λ light chain locus comprising all human Vλ gene segments, human Jλ gene segments, and human Cλ gene segments (e.g., at the endogenous κ or λ light chain locus). In some embodiments, a genetically modified non-human animal (e.g., rodent, e.g., mouse or rat) comprises in its genome (e.g., its germline genome) a deletion of all or part of its endogenous λ light chain locus.

In some embodiments, an endogenous non-human animal (e.g., rodent, e.g., mouse or rat) immunoglobulin λ light chain locus sequence is replaced (e.g., by homologous recombination) with a contiguous sequence of human immunoglobulin λ light chain locus sequence. In some embodiments, the human immunoglobulin λ light chain locus sequence comprises all of the human immunoglobulin λ light chain locus (i.e., a contiguous sequence). In some embodiments, the human immunoglobulin λ light chain locus sequence is at least 500 kb in size (i.e., at least 500 kb, at least 100 kb, at least 1,000 kb). In some embodiments, the replacement of the endogenous non-human animal (e.g., rodent, e.g., mouse or rat) immunoglobulin lambda light chain locus is performed with a single recombination step. Exemplary embodiments can be found, e.g., in PCT Publication No. WO2020169022, which is hereby incorporated by reference in its entirety).

### 3. Universal Light Chain

In some embodiments, a genetically modified non-human animal (e.g., rodent, e.g., mouse or rat) has a germline genome comprising a limited human light chain variable region repertoire. Exemplary genetically modified rodents, comprising human V(D)J gene segments having a germline genome comprising a limited human light chain variable region repertoire are described in, e.g., U.S. Pat. Nos. 9,796,788; 10,130,081; 10,143,186; 10,167,344; 10,412,940; and 10,130,081; as well as WO2019008123, WO2020247623, WO2020132557, WO2017035274, WO2019236670, WO2019236671, WO2021244522, and Harris, et al., Frontiers in immunology 9 (2018): 889, each of which is hereby incorporated by reference in its entirety. In some embodiments, a limited human light chain variable region repertoire comprises a limited number of human V_{L} gene segments. In some embodiments, a limited number of human V_{L} gene segments comprises two human V_{L} gene segments. In some embodiments, a limited number of human V_{L} gene segments is one human V_{L} gene segment. For example, in some embodiments a limited number of human V_{L} gene segments is one human Vκ gene segment. One human Vκ gene segment can be, e.g., a human Vκ1-39 gene segment, a human Vκ3-15 gene segment, a human Vκ 3-11 gene segment, or a human Vκ3-20 gene segment. In some embodiments a limited number of human V_{L} gene segments is one human Vλ gene segment. One human Vλ gene segment can be, e.g., a human Vλ1-51 gene segment, a human Vλ5-45 gene segment, a human Vλ1-44 gene segment, a human Vλ1-40 gene segment, a human Vλ3-21 gene segment, or a human Vλ2-14 gene segment.

In some embodiments, a limited human light chain variable region repertoire comprises one or more J_{L} gene segments. In some embodiments, a limited human light chain variable region repertoire comprises one J_{L} gene segment. In some embodiments, one J_{L} gene segment is a Jκ gene segment. In some embodiments, one J_{L} gene segment is a Jλ gene segment. In some embodiments, one J_{L} gene segment is a human J_{L} gene segment. In some embodiments, one J_{L} gene segment is a mouse J_{L} gene segment.

In some embodiments, a limited human light chain variable region repertoire comprises (i) a human Vκ gene segment and a human Jκ gene segment, (ii) a human Vκ gene segment and a mouse Jκ gene segment, (iii) a human Vκ gene segment and a human Jλ gene segment, or (iv) a human Vκ gene segment and a mouse Jλ gene segment.

In some embodiments, a limited human light chain variable region repertoire comprises (i) a human Vλ gene segment and a human Jλ gene segment, (ii) a human Vκ gene segment and a mouse Jλ gene segment, (iii) a human Vλ gene segment and a human Jκ gene segment, or (iv) a human Vλ gene segment and a mouse Jκ gene segment.

In some embodiments, a limited human light chain variable region repertoire comprises (i) a human Vκ1-39 gene segment and a human Jκ5 gene segment, (ii) a human Vκ1-39 gene segment and a human Jκ1 gene segment, (iii) a human Vκ3-20 gene segment and a human Jκ1 gene segment, (iv) a human Vκ3-20 gene segment and a human Jκ5 gene segment, (v) a human Vκ1-39 gene segment and a human Jκ4 gene segment, (vi) a human Vκ3-11 gene segment and a human Jκ1 gene segment, or (v) a human Vκ3-15 gene segment and a human Jκ1 gene segment.

In some embodiments, a limited human light chain variable region repertoire comprises (i) a human Vκ1-39 gene segment and a mouse Jκ2 gene segment, (ii) a human Vκ3-20 gene segment and a mouse Jκ2 gene segment, or (iii) a human Vκ3-15 gene segment and a mouse Jκ2 gene segment.

In some embodiments, a limited human light chain variable region repertoire comprises (i) a human Vλ1-51 gene segment and a human Jλ2 gene segment, (ii) a human Vλ5-45 gene segment and a human Jλ2 gene segment, (iii) a human Vλ1-44 gene segment and a human Jλ2 gene segment, (iv) a human Vλ1-40 gene segment and a human Jλ2 gene segment, (v) a human Vλ3-21 gene segment and a human Jλ2 gene segment, or (vi) a human Vλ2-14 gene segment and a human Jλ2 gene segment.

In some embodiments, a limited human light chain variable region repertoire is operably linked to a Cκ gene segment. In some embodiments, a Cκ gene segment is human. In some embodiments, a Cκ gene segment is mouse. In some embodiments, a mouse Cκ gene segment is an endogenous mouse Cκ gene segment, e.g., at an endogenous mouse immunoglobulin κ light chain locus. In some embodiments, a mouse Cκ gene segment is at an endogenous mouse immunoglobulin λ light chain locus.

In some embodiments, a Cκ gene segment is rat. In some embodiments, a rat Cκ gene segment is an endogenous rat Cκ gene segment, e.g., at an endogenous rat immunoglobulin κ light chain locus. In some embodiments, a rat Cκ gene segment is at an endogenous rat immunoglobulin λ light chain locus.

In some embodiments, a limited human light chain variable region repertoire is operably linked to a Cλ gene segment. In some embodiments, a Cλ gene segment is human. In some embodiments, a Cλ gene segment is mouse. In some embodiments, a mouse Cλ gene segment is an endogenous mouse Cλ gene segment, e.g., at an endogenous mouse immunoglobulin λ light chain locus. In some embodiments, a mouse Cλ gene segment is at an endogenous mouse immunoglobulin κ light chain locus. In some embodiments, a Cλ gene segment is rat. In some embodiments, a rat Cλ gene segment is an endogenous rat Cλ gene segment, e.g., at an endogenous rat immunoglobulin λ light chain locus. In some embodiments, a rat Cλ gene segment is at an endogenous rat immunoglobulin κ light chain locus.

In some embodiments, a genetically modified non-human animal (e.g., rodent, e.g., mouse or rat) is heterozygous for a limited human light chain variable region repertoire. In some embodiments, a genetically modified non-human animal (e.g., rodent, e.g., mouse or rat) is homozygous for a limited human light chain variable region repertoire.

In some embodiments, a genetically modified non-human animal (e.g., rodent, e.g., mouse or rat) comprises an engineered immunoglobulin light chain locus (e.g., an engineered endogenous immunoglobulin light chain locus) comprising a restricted light chain variable region sequence, comprising a limited human light chain variable region repertoire. In some embodiments, a limited human light chain variable region repertoire comprises one or two human light chain V gene segments and one or more human light chain J gene segments. In some embodiments, a limited human light chain variable region repertoire is operably linked to a light chain constant region gene segment. In some embodiments, a genetically modified non-human animal (e.g., rodent, e.g., mouse or rat) comprising a limited human light chain variable region repertoire comprises in its genome (e.g., its germline genome) exactly two unrearranged human light chain V gene segments and one or more unrearranged human light chain J gene segments operably linked to a light chain constant region sequence. Such an engineered immunoglobulin light chain locus is referred to herein as a “DLC locus.” In some embodiments, a genetically modified non-human animal (e.g., rodent, e.g., mouse or rat) comprising a limited human light chain variable region repertoire comprises in its genome (e.g., its germline genome) a single rearranged light chain variable region locus comprising a single human light chain V gene segment rearranged to a single human light chain J gene segment. In some embodiments, a genetically modified non-human animal (e.g., rodent, e.g., mouse or rat) comprising a limited human light chain variable region repertoire comprises in its genome (e.g., its germline genome) a single rearranged light chain variable region locus operably linked to a light chain constant region sequence, where the single rearranged light chain variable region locus comprises a single human light chain V gene segment rearranged to a single human light chain J gene segment. Such an engineered immunoglobulin light chain locus is referred to herein as “ULC locus.” As used herein, the phrase “ULC locus” is interchangeable with “universal light chain locus” or “common light chain locus.”

In some embodiments, a genetically modified non-human animal (e.g., rodent, e.g., mouse or rat) comprises a limited light chain antibody repertoire that is at least 90% pure for a single human V_{L} domain (e.g., a single rearranged human V_{L} domain) or a somatically hypermutated version thereof. In some embodiments, a genetically modified non-human animal (e.g., rodent, e.g., mouse or rat) comprises a limited light chain antibody repertoire that is at least 95% pure for a single human V_{L} domain (e.g., a single rearranged human V_{L} domain) or a somatically hypermutated version thereof. In some embodiments, a genetically modified non-human animal (e.g., rodent, e.g., mouse or rat) comprises a limited light chain antibody repertoire that is at least 99% pure for a single human V_{L} domain (e.g., a single rearranged human V_{L} domain) or a somatically hypermutated version thereof.

In some embodiments, a genetically modified non-human animal (e.g., rodent, e.g., mouse or rat) has a germline genome comprising a limited human κ light chain variable region repertoire. In some embodiments, a genetically modified non-human animal (e.g., rodent, e.g., mouse or rat) comprises an engineered immunoglobulin κ light chain locus (e.g., an engineered endogenous immunoglobulin κ light chain locus) comprising a limited human κ light chain variable region repertoire. In some embodiments, a limited human κ light chain variable region repertoire comprises one or two human Vκ gene segments and one or more human Jκ gene segments. In some embodiments, a limited human κ light chain variable region repertoire operably linked to a light chain constant region gene segment. In some embodiments, a genetically modified rodent as provided comprises a limited human κ light chain variable region repertoire operably linked to a Cκ gene segment.

In some embodiments, a genetically modified non-human animal (e.g., rodent, e.g., mouse or rat) comprises in its genome (e.g., its germline genome) a limited human κ light chain variable region repertoire, wherein the limited human κ light chain variable region repertoire comprises a single rearranged human κ light chain variable region (Vκ/Jκ). A single rearranged human κ light chain variable region comprises a human Vκ gene segment joined to a human Jκ gene segment. In some embodiments, a genetically modified non-human animal (e.g., rodent, e.g., mouse or rat) comprises in its genome (e.g., its germline genome) an engineered immunoglobulin κ light chain locus (e.g., an engineered endogenous rodent immunoglobulin κ light chain locus) comprising a single rearranged human κ light chain variable region upstream of (e.g., operably linked to) a Cκ gene. Such an engineered immunoglobulin light chain locus is referred to as a “κULC locus” and is an example of a ULC locus. Rodents including a κULC locus are exemplified in, e.g., U.S. Pat. Nos. 10,130,081 and 10,143,186, each of which is incorporated by reference in its entirety.

In some embodiments, a single rearranged human κ light chain variable region comprises a human Vκ gene segment and a human Jκ gene segment. In some embodiments, a human Vκ gene segment is a human Vκ1-39 gene segment, a human Vκ3-11 gene segment, a human Vκ3-15 gene segment, or a human Vκ3-20 gene segment. In some embodiments, a human Jκ gene segment is a human Jκ1 gene segment, a human Jκ2 gene segment, a human Jκ3 gene segment, a human Jκ4 gene segment, or a human Jκ5 gene segment. In some embodiments, a human Vκ gene segment is a human Vκ1-39 gene segment, and a human Jκ gene segment is a human Jκ5 gene segment. In some embodiments, a single rearranged human κ light chain variable region is a human Vκ1-39/Jκ5. In some embodiments, a human Vκ gene segment is a human Vκ3-20 gene segment, and a human Jκ gene segment is a human Jκ1 gene segment. In some embodiments, a human Vκ gene segment is a human Vκ1-39 gene segment, and a human Jκ gene segment is a human Jκ4 gene segment. In some embodiments, a human Vκ gene segment is a human Vκ3-11 gene segment, and a human Jκ gene segment is a human Jκ1 gene segment. In some embodiments, a human Vκ gene segment is a human Vκ3-15 gene segment, and a human Jκ gene segment is a human Jκ1 gene segment. In some embodiments, a single rearranged human κ light chain variable region is a human Vκ3-20/Jκ1. In some embodiments, a single rearranged human κ light chain variable region is a human Vκ3-15/Jκ1.

In some embodiments, a Cκ gene of a κULC locus is a rodent (e.g., rat or mouse) Cκ gene. In some embodiments, a genetically modified non-human animal (e.g., rodent, e.g., mouse or rat) is homozygous at a κULC locus. In some embodiments, a genetically modified non-human animal (e.g., rodent, e.g., mouse or rat) is heterozygous at a κULC locus.

In some embodiments, a genetically modified non-human animal (e.g., rodent, e.g., mouse or rat), which comprises a κULC locus, lacks endogenous Vκ and/or Jκ gene segments that are capable of rearranging to form an endogenous κ light chain variable region. In some embodiments, a genetically modified non-human animal (e.g., rodent, e.g., mouse or rat), which comprises a κULC locus, lacks endogenous Vλ and/or Jλ gene segments that are capable of rearranging to form an endogenous λ light chain variable region.

In some embodiments, a genetically modified non-human animal (e.g., rodent, e.g., mouse or rat), which comprises a κULC locus, produces an antibody comprising, inter alia, κ light chains, where each κ light chain comprises a human κ light chain variable domain operably linked to a non-human animal (e.g., rodent, e.g., mouse or rat) κ light chain constant domain, e.g., in response to antigenic stimulation. In some embodiments, all κ light chains expressed by B cells (e.g., plasma cells) of a genetically modified non-human animal (e.g., rodent, e.g., mouse or rat), which comprises a κULC locus, comprise human κ light chain variable domains expressed from the single rearranged human κ light chain variable region or a somatically hypermutated version thereof.

In some embodiments, a genetically modified non-human animal (e.g., rodent, e.g., mouse or rat) comprises in its genome (e.g., its germline genome) an engineered immunoglobulin κ light chain locus (e.g., an engineered endogenous immunoglobulin κ light chain locus) comprising exactly two unrearranged human Vκ gene segments and one or more unrearranged human Jκ gene segments operably linked to a κ light chain constant region sequence of (e.g., operably linked to) a Cκ gene. Such an engineered immunoglobulin κ light chain locus is referred to herein as a “κDLC locus,” and is an example of a DLC locus. Rodents including a κDLC locus are exemplified in, e.g., U.S. Pat. Nos. 9,796,788; 10,167,344; 10,412,940; and 10,130,081, each of which is incorporated by reference in its entirety.

In some embodiments, exactly two unrearranged human Vκ gene segments comprise a human Vκ1-39 gene segment and a human Vκ3-20 gene segment. In some embodiments, one or more unrearranged human Jκ gene segments comprises two human Jκ gene segments. In some embodiments, one or more unrearranged human Jκ gene segments comprises three human Jκ gene segments. In some embodiments, one or more unrearranged human Jκ gene segments comprises four human Jκ gene segments. In some embodiments, one or more unrearranged human Jκ gene segments comprises five human Jκ gene segments. In some embodiments, one or more unrearranged human Jκ gene segments comprises a human Jκ1 gene segment, a human Jκ2 gene segment, a human Jκ3 gene segment, a human Jκ4 gene segment, a human Jκ5 gene segment, or a combination thereof.

In some embodiments, a genetically modified non-human animal (e.g., rodent, e.g., mouse or rat), which comprises a κDLC locus, comprises in its genome (e.g., germline genome) exactly two unrearranged human Vκ gene segments and five unrearranged human Jκ gene segments. In some embodiments, exactly two unrearranged human Vκ gene segments comprises a human Vκ1-39 gene segment and a human Vκ3-20 gene segment, and five unrearranged human Jκ gene segments comprise a human Jκ1 gene segment, a human Jκ2 gene segment, a human Jκ3 gene segment, a human Jκ4 gene segment, and a human Jκ5 gene segment.

In some embodiments, a Cκ gene of a κDLC locus is a rodent (e.g., rat or mouse) Cκ gene. In some embodiments, a genetically modified non-human animal (e.g., rodent, e.g., mouse or rat) is homozygous at a κDLC locus. In some embodiments, a genetically modified non-human animal (e.g., rodent, e.g., mouse or rat) is heterozygous at a κDLC locus.

In some embodiments, a genetically modified non-human animal (e.g., rodent, e.g., mouse or rat), which comprises a κDLC locus, lacks endogenous immunoglobulin Vκ and/or Jκ gene segments that are capable of rearranging to form an endogenous immunoglobulin K light chain variable region. In some embodiments, a genetically modified non-human animal (e.g., rodent, e.g., mouse or rat), which comprises a κDLC locus, lacks endogenous Vλ and/or Jκ gene segments that are capable of rearranging to form an endogenous λ light chain variable region.

In some embodiments, a genetically modified non-human animal (e.g., rodent, e.g., mouse or rat), which comprises a κDLC locus, produces an antibody comprising, inter alia, κ light chains, where each κ light chain comprises a human κ light chain variable domain operably linked to a non-human animal (e.g., rodent, e.g., mouse or rat) κ light chain constant domain, e.g., in response to antigenic stimulation.

In some embodiments, a genetically modified non-human animal (e.g., rodent, e.g., mouse or rat) has a genome (e.g., germline genome) comprising a limited human λ light chain variable region repertoire. In some embodiments, a genetically modified non-human animal (e.g., rodent, e.g., mouse or rat) has a genome (e.g., germline genome) comprising an engineered immunoglobulin κ light chain locus (e.g., an engineered endogenous immunoglobulin κ light chain locus) comprising a limited human λ light chain variable region repertoire. In some embodiments, a genetically modified non-human animal (e.g., rodent, e.g., mouse or rat) comprises an engineered immunoglobulin κ light chain locus (e.g., an engineered endogenous immunoglobulin κ light chain locus) comprising a limited human λ light chain variable region repertoire, wherein the limited human λ light chain variable region repertoire comprises one or two human Vλ gene segments and one or more human Jλ gene segments. In some embodiments, a genetically modified non-human animal (e.g., rodent, e.g., mouse or rat) comprises a limited human λ light chain variable region repertoire operably linked to a light chain constant region gene segment. In some embodiments, a genetically modified non-human animal (e.g., rodent, e.g., mouse or rat) as provided comprises a limited human λ light chain variable region repertoire operably linked to a non-human animal (e.g., rodent, e.g., mouse or rat) Cκ gene segment. In some embodiments, a genetically modified non-human animal (e.g., rodent, e.g., mouse or rat) as provided comprises a limited human λ light chain variable region repertoire operably linked to a non-human animal (e.g., rodent, e.g., mouse or rat) Cλ gene segment.

In some embodiments, a genetically modified non-human animal (e.g., rodent, e.g., mouse or rat) has a genome (e.g., germline genome) comprising an engineered immunoglobulin κ light chain locus (e.g., an engineered endogenous immunoglobulin κ light chain locus) that comprises a limited human λ light chain variable region repertoire, wherein the limited human λ light chain variable region repertoire comprises a single rearranged human immunoglobulin λ light chain variable region (V/Jλ). A single rearranged human λ light chain variable region comprises a human Vλ gene segment joined to a human Jλ gene segment. In some embodiments, a genetically modified rodent comprises a limited human λ light chain variable region repertoire operably linked to a non-human animal (e.g., rodent, e.g., mouse or rat) Cκ or Cλ gene segment (e.g., a mouse Cλ1 gene segment). Such an engineered immunoglobulin light chain locus is an example of a ULC locus and is referred to herein as a “ULCiK locus.” Rodents including a ULCiK locus are exemplified in, e.g., WO2020/247623, which is incorporated by reference in its entirety.

In some embodiments, a human Vλ gene segment is selected from a group consisting of: Vλ4-69, Vλ8-61, Vλ4-60, Vλ6-57, Vλ10-54, Vλ5-52, Vλ1-51, Vλ9-49, Vλ1-47, Vλ7-46, Vλ5-45, Vλ1-44, Vλ7-43, Vλ1-40, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3, and Vλ3-1. In some embodiments, a human Vλ gene segment is selected from a group consisting of: Vλ5-52, Vλ1-51, Vλ9-49, Vλ1-47, Vλ7-46, Vλ5-45, Vλ1-44, Vλ7-43, Vλ1-40, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3, and Vλ3-1. In some embodiments, a human Vλ gene segment is selected from a group consisting of: Vλ1-51, Vλ5-45, Vλ1-44, Vλ1-40, Vλ3-21, and Vλ2-14. In some embodiments, a human Vλ gene segment is Vλ1-51 or Vλ2-14. In some embodiments, a human Jλ gene segment is selected from a group consisting of: Jλ1, Jλ2, Jλ3, Jλ6, and Jλ7. In some embodiments, a human Jλ gene segment is selected from a group consisting of: Jλ1, Jλ2, Jλ3, and Jλ7. In some embodiments, a human Jλ gene segment is Jλ2.

In some embodiments, a genetically modified non-human animal (e.g., rodent, e.g., mouse or rat), which comprises a ULCiK locus, lacks endogenous Vκ and/or Jκ gene segments that are capable of rearranging to form an endogenous κ light chain variable region. In some embodiments, a genetically modified non-human animal (e.g., rodent, e.g., mouse or rat), which comprises a ULCiK locus, lacks endogenous Vλ and/or Jλ gene segments that are capable of rearranging to form an endogenous λ light chain variable region.

In some embodiments, a genetically modified non-human animal (e.g., rodent, e.g., mouse or rat), which comprises a ULCiK locus, produces an antibody comprising, inter alia, light chains, wherein each light chain comprises a human λ light chain variable domain operably linked to a non-human animal (e.g., rodent, e.g., mouse or rat) light chain constant domain (e.g., a Cλ or Cκ domain), e.g., in response to antigenic stimulation. In some embodiments, all light chains expressed by B cells (e.g., plasma cells) of a genetically modified non-human animal (e.g., rodent, e.g., mouse or rat), which comprises a ULCiK locus, comprise human λ light chain variable domains expressed from the single rearranged human λ light chain variable region or a somatically hypermutated version thereof.

In some embodiments, a genetically modified non-human animal (e.g., rodent, e.g., mouse or rat) has a genome (e.g., germline genome) comprising an engineered immunoglobulin κ light chain locus (e.g., an engineered endogenous immunoglobulin κ light chain locus) that comprises a limited human λ light chain variable region repertoire, wherein the limited human λ light chain variable region repertoire comprises two unrearranged human Vλ gene segments and one or more unrearranged human Jλ gene segments. In some embodiments, a limited human λ light chain variable region repertoire comprises two unrearranged human Vλ gene segments and four unrearranged human Jλ gene segments. In some embodiments, a limited human λ light chain variable region repertoire comprises two unrearranged human Vλ gene segments and five unrearranged human Jλ gene segments. In some embodiments, a genetically modified rodent comprises a limited human λ light chain variable region repertoire operably linked to a rodent (e.g., rat or mouse) Cλ gene segment (e.g., a mouse Cλ1 gene segment). Such an engineered immunoglobulin light chain locus is an example of a DLC locus and is referred to herein as a “DLCiK locus.” Rodents including a DLCiK locus are exemplified in, e.g., WO2020/247623, which is incorporated by reference in its entirety.

In some embodiments, a germline genome of the genetically modified rodent is homozygous for an engineered immunoglobulin κ light chain locus comprising a limited human λ light chain variable region repertoire. In some embodiments, a germline genome of the genetically modified non-human animal (e.g., rodent, e.g., mouse or rat) is heterozygous for a engineered immunoglobulin κ light chain locus comprising a limited human λ light chain variable region repertoire.

In some embodiments, a genetically modified non-human animal (e.g., rodent, e.g., mouse or rat), which comprises a DLCiK locus, lacks endogenous immunoglobulin Vκ and/or Jκ gene segments that are capable of rearranging to form an endogenous immunoglobulin κ light chain variable region. In some embodiments, a genetically modified non-human animal (e.g., rodent, e.g., mouse or rat), which comprises a DLCiK locus, lacks endogenous Vλ and/or Jλ gene segments that are capable of rearranging to form an endogenous λ light chain variable region.

In some embodiments, a genetically modified non-human animal (e.g., rodent, e.g., mouse or rat), which comprises a DLCiK locus, produces an antibody comprising, inter alia, light chains, where each light chain comprises a human λ light chain variable domain operably linked to a light chain constant domain (e.g., a Cλ or Cκ domain), e.g., in response to antigenic stimulation.

In some embodiments, a genetically modified rodent comprises a ULC locus that is modified to reduce or eliminate somatic hypermutation (SHM). In some embodiments, a ULC locus is modified to increase the distance of the human rearranged V/J gene sequence from the transcription start site to reduce or eliminate SHM. In some embodiments, a ULC locus is modified to comprise an intronic enhancer (e.g., Eiκ enhancer) upstream (i.e., 5′) of the rearranged V/J gene sequence to reduce or eliminate SHM.

### C. Other Exemplary Genetic Modifications

### 1. Adam6

In some embodiments, a genetically modified non-human animal (e.g., rodent, e.g., mouse or rat) as provided herein has a genome (e.g., a germline genome) comprising an engineered immunoglobulin heavy chain (e.g., HoH, UHC, LoH) locus (e.g., an engineered endogenous immunoglobulin heavy chain locus) lacking a functional endogenous Adam6 gene. In some embodiments, a genetically modified non-human animal is a rodent (e.g., mouse or rat). In some embodiments, a genetically modified non-human animal is a mouse.

In some embodiments, a genetically modified rodent (e.g., rat or mouse) as provided herein has a genome (e.g., a germline genome) comprising one or more nucleotide sequences encoding one or more rodent ADAM6 polypeptides, functional orthologs, functional homologs, or functional fragments thereof. In some embodiments, one or more rodent ADAM6 polypeptides is or comprises mouse ADAM6a. In some embodiments, one or more rodent ADAM6 polypeptides is or comprises mouse ADAM6b. In some embodiments, one or more rodent ADAM6 polypeptides is or comprises mouse ADAM6a and mouse ADAM6b. In some embodiments, one or more rodent ADAM6 polypeptides comprises only one mouse ADAM6 polypeptide (i.e., either a mouse ADAM6a polypeptide or a mouse ADAM6b polypeptide). Rodents including one or more nucleotide sequences encoding one or more rodent ADAM6 polypeptides, functional orthologs, functional homologs, or functional fragments thereof are exemplified in, e.g., U.S. Pat. Nos. 8,642,835; 8,697,940; 9,706,759; 10,130,081; 10,238,093, U.S. Patent Publication No. 2013/0212719, and US Publication No. 2021/0059229, each of which is incorporated by reference in its entirety. In some embodiments, a genetically modified rodent (e.g., rat or mouse) as provided expresses one or more rodent (e.g., rat or mouse) ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof that are included on the same chromosome as an engineered immunoglobulin heavy chain (e.g., HoH, UHC, LoH) locus. In some embodiments, a genetically modified rodent (e.g., rat or mouse) as provided has a genome (e.g., a germline genome) comprising an engineered immunoglobulin heavy chain (e.g., HoH, UHC, LoH) locus comprising one or more nucleotide sequences encoding one or more rodent ADAM6 polypeptides, functional orthologs, functional homologs, or functional fragments thereof. In some embodiments, a genetically modified rodent (e.g., rat or mouse) as provided has a genome (e.g., a germline genome) comprising one or more nucleotide sequences encoding one or more rodent (e.g., rat or mouse) ADAM6 polypeptides, functional orthologs, functional homologs, or functional fragments thereof in place of a human Adam6 pseudogene. In some embodiments, a genetically modified rodent (e.g., rat or mouse) as provided has a genome (e.g., germline genome) comprising one or more nucleotide sequences encoding one or more rodent (e.g., rat or mouse) ADAM6 polypeptides, functional orthologs, functional homologs, or functional fragments thereof that replace a human Adam6 pseudogene.

In some embodiments, a genetically modified rodent as provided has a genome (e.g., a germline genome) comprising one or more human V_{H} gene segments comprising a first and a second human V_{H} gene segment, and one or more nucleotide sequences encoding one or more rodent (e.g., rat or mouse) ADAM6 polypeptides, functional orthologs, functional homologs, or functional fragments thereof between the first human V_{H} gene segment and the second human V_{H} gene segment. In some embodiments, a first human V_{H} gene segment is V_{H}1-2 and a second human V_{H} gene segment is V_{H}6-1.

In some embodiments, a genetically modified rodent as provided has a genome (e.g., a germline genome) comprising one or more human V_{H} gene segments, and one or more nucleotide sequences encoding one or more rodent (e.g., rat or mouse) ADAM6 polypeptides, functional orthologs, functional homologs, or functional fragments thereof upstream (i.e., 5′) of the one or more human V_{H} gene segments.

In some embodiments, one or more nucleotide sequences encoding one or more rodent (e.g., a rat or mouse) ADAM6 polypeptides, functional orthologs, functional homologs, or functional fragments thereof are between a human V_{H} gene segment and a human D_{H} gene segment.

In some embodiments, one or more nucleotide sequences encoding one or more rodent ADAM6 polypeptides restore or enhance fertility in a male rodent.

### 2. TdT

In some embodiments, a genetically modified non-human animal (e.g., rodent, e.g., mouse or rat) comprises an exogenous terminal deoxynucleotidyl transferase (TdT) gene. Rodents including an exogenous TdT are exemplified in, e.g., U.S. Patent Publication No. 2019/0223418 and PCT Publication No. WO 2017/210586, each of which is incorporated by reference in its entirety. In some embodiments, a non-human animal (e.g., rodent, e.g., mouse or rat) that comprises an exogenous TdT gene can have increased antigen receptor diversity when compared to a rodent without an exogenous TdT gene.

In some embodiments, a non-human animal (e.g., rodent, e.g., mouse or rat) as described herein has a genome comprising an exogenous TdT gene operably linked to a transcriptional control element.

In some embodiments, a transcriptional control element includes a RAG1 transcriptional control element, a RAG2 transcriptional control element, an immunoglobulin heavy chain transcriptional control element, an immunoglobulin κ light chain transcriptional control element, an immunoglobulin λ light chain transcriptional control element, or any combination thereof.

In some embodiments, an exogenous TdT is located at an immunoglobulin κ light chain locus, an immunoglobulin λ light chain locus, an immunoglobulin heavy chain locus, a RAG1 locus, or a RAG2 locus.

In some embodiments, a TdT is a human TdT. In some embodiments, a TdT is a short isoform of TdT (TdTS).

### 3. Engineered DH Gene Segments

In some embodiments, a non-human animal (e.g., rodent, e.g., mouse or rat) as described herein has a genome comprising an engineered or recombinant diversity cluster with an immunoglobulin heavy chain variable region.

In some embodiments, an engineered or recombinant heavy chain diversity (DH) cluster comprises an insertion of one or more DH segments that are each operably linked to a 23-mer recombination signal sequence (RSS). In some embodiments, an engineered or recombinant DH cluster comprises an engineered DH region comprising at least one DH gene segment operably linked to a 23-mer RSS and an unrearranged DH gene segment flanked on one side by a 12-mer RSS and on the other side by another 12-mer RSS, which DH gene segments are operably linked such that they are able to join in a DH-DH recombination event according to the 12/23 rule. In some embodiments, the engineered or recombinant DH gene segments are human. Exemplary engineered or recombinant DH clusters are shown in e.g., WO 2019241692).

In some embodiments, an engineered or recombinant DH cluster comprises at least two DH gene segments that are fused (i.e., a D-D fusion), wherein the DH gene segments, when recombined with V and J gene segments in the heavy chain locus, generate a rearranged VDDJ transcript that encodes an immunoglobulin heavy chain variable domain. Exemplary recombinant DH gene segments are shown in e.g., U.S. Patent Pub. No. 2019/0127757.

In some embodiments, a non-human animal (e.g., rodent, e.g., mouse or rat) as described herein has a genome comprising an engineered or recombinant diversity cluster with an immunoglobulin heavy chain variable region such that the rodent produces human antibodies that comprise HCDR3 sequences that are least 10 amino acids in length (e.g., at least 10, at least 11, at least 12, at least 13, at least 14, at least 15, at least 16, at least 17, at least 18, at least 19, at least 20, at least 21, at least 22, at least 23, at least 24, at least 25, or more amino acids in length).

In some embodiments, a non-human animal (e.g., rodent, e.g., mouse or rat) described herein comprises an immunoglobulin heavy chain variable region that includes an engineered DH region, wherein the engineered DH region includes one or more nucleotide sequences that each encode a non-immunoglobulin polypeptide of interest, or portion thereof (e.g., a chemokine receptor (e.g., atypical chemokine receptor (ACKR), e.g., a D6 chemokine decoy receptor), a conotoxin, or a tarantula toxin), wherein the immunoglobulin heavy chain variable region is operably linked to an IgA or IgMDA constant region. Exemplary engineered DH regions are shown in e.g., WO 2017123804.

In some embodiments, a non-human animal (e.g., rodent, e.g., mouse or rat) described herein comprises an engineered immunoglobulin locus capable of making anchor-modified immunoglobulin polypeptides. In some embodiments, an immunoglobulin locus comprises a nucleic acid sequence comprising a modified immunoglobulin variable (V) gene segment that encodes an anchor-modified immunoglobulin polypeptide. In some embodiments, a modified immunoglobulin V gene segment comprises a nucleic acid sequence encoding an anchor between a nucleic acid sequence encoding an immunoglobulin signal peptide and a nucleic acid sequence encoding the framework region (FR)1, complementarity determining region (CDR)1, FR2, CDR2, FR3, and CDR3 of a germline immunoglobulin V gene segment, or a variant thereof. In some embodiments, an anchor modified immunoglobulin polypeptide comprises in operable linkage: (i) an immunoglobulin signal peptide, (ii) an anchor, and (iii) an FR1, CDR1, FR2, CDR2, FR3, and CDR3 of the germline immunoglobulin V segment, or a variant thereof. In some embodiments, an anchor comprises a receptor-binding portion of a non-immunoglobulin polypeptide of interest that binds a cognate receptor. Exemplary engineered V regions are shown in e.g., WO 2022140219.

### D. Exemplary Combinations of Immunoglobulin Locus Modification

In some embodiments, a genetically modified non-human animal (e.g., rodent, e.g., mouse or rat) comprises in its genome a modified (e.g., humanized) immunoglobulin heavy and/or light chain locus as described herein. Such combinations of modified immunoglobulin heavy and/or light chain loci may be present in the genome of non-human animal (e.g., rodent, e.g., mouse or rat). In some embodiments, a genetically modified non-human animal (e.g., rodent, e.g., mouse or rat) comprises in its genome (e.g., its germline genome) an IgA or IgMDA locus described herein, and additionally comprises any of the following combination of heavy and/or light chain loci.

In some embodiments, a genetically modified non-human animal (e.g., rodent, e.g., mouse or rat) comprises in its genome (e.g., its germline genome) a HoH locus and a KoK locus. In some embodiments, a genetically modified non-human animal (e.g., rodent, e.g., mouse or rat) comprises in its genome (e.g., its germline genome) a HoH locus and a LoL locus. In some embodiments, a genetically modified non-human animal (e.g., rodent, e.g., mouse or rat) comprises in its genome (e.g., its germline genome) a HoH locus, a KoK locus, and a LoL locus. In some embodiments, a genetically modified non-human animal (e.g., rodent, e.g., mouse or rat) is homozygous at a HoH locus, a KoK locus, a LoL locus, or a combination thereof.

In some embodiments, a genetically modified non-human animal (e.g., rodent, e.g., mouse or rat) comprises in its genome (e.g., its germline genome) a HoH locus, a KoK locus, and a LoK locus. In some embodiments, a genetically modified non-human animal (e.g., rodent, e.g., mouse or rat) comprises in its genome (e.g., its germline genome) a HoH locus, a KoK locus, and a LiK locus.

In some embodiments, a genetically modified non-human animal (e.g., rodent, e.g., mouse or rat) comprises in its genome (e.g., its germline genome) a HoH locus and a LoK locus. In some embodiments, a genetically modified non-human animal (e.g., rodent, e.g., mouse or rat) is homozygous at a HoH locus, a LoK locus, or a combination thereof.

In some embodiments, a genetically modified non-human animal (e.g., rodent, e.g., mouse or rat) comprises in its genome (e.g., its germline genome) a HoH locus and a LiK locus. In some embodiments, a genetically modified non-human animal (e.g., rodent, e.g., mouse or rat) is homozygous at a HoH locus, a LiK locus, or a combination thereof.

In some embodiments, a genetically modified non-human animal (e.g., rodent, e.g., mouse or rat) comprises in its genome (e.g., its germline genome) a HoH locus and a ULC locus. In some embodiments, a genetically modified non-human animal (e.g., rodent, e.g., mouse or rat) is homozygous at a HoH locus, a ULC locus, or a combination thereof.

In some embodiments, a genetically modified non-human animal (e.g., rodent, e.g., mouse or rat) comprises in its genome (e.g., its germline genome) a HoH locus and a DLC locus. In some embodiments, a genetically modified non-human animal (e.g., rodent, e.g., mouse or rat) is homozygous at a HoH locus, a DLC locus, or a combination thereof.

In some embodiments, a genetically modified non-human animal (e.g., rodent, e.g., mouse or rat) comprises in its genome (e.g., its germline genome) a HoH locus and a κULC locus. In some embodiments, a genetically modified non-human animal (e.g., rodent, e.g., mouse or rat) is homozygous at a HoH locus, a κULC locus, or a combination thereof.

In some embodiments, a genetically modified non-human animal (e.g., rodent, e.g., mouse or rat) comprises in its genome (e.g., its germline genome) a HoH locus and a κDLC locus. In some embodiments, a genetically modified non-human animal (e.g., rodent, e.g., mouse or rat) is homozygous at a HoH locus, a κDLC locus, or a combination thereof.

In some embodiments, a genetically modified non-human animal (e.g., rodent, e.g., mouse or rat) comprises in its genome (e.g., its germline genome) a HoH locus and a ULCiK locus. In some embodiments, a genetically modified non-human animal (e.g., rodent, e.g., mouse or rat) is homozygous at a HoH locus, a ULCiK locus, or a combination thereof.

In some embodiments, a genetically modified non-human animal (e.g., rodent, e.g., mouse or rat) comprises in its genome (e.g., its germline genome) a HoH locus and a DLCiK locus. In some embodiments, a genetically modified non-human animal (e.g., rodent, e.g., mouse or rat) is homozygous at a HoH locus, a DLCiK locus, or a combination thereof.

In some embodiments, a genetically modified non-human animal (e.g., rodent, e.g., mouse or rat) comprises in its genome (e.g., its germline genome) a HoH locus and a HULC locus. In some embodiments, a genetically modified non-human animal (e.g., rodent, e.g., mouse or rat) is homozygous at a HoH locus, a HULC locus, or a combination thereof.

In some embodiments, a genetically modified non-human animal (e.g., rodent, e.g., mouse or rat) comprises in its genome (e.g., its germline genome) a UHC locus and a KoK locus. In some embodiments, a genetically modified non-human animal (e.g., rodent, e.g., mouse or rat) comprises in its genome (e.g., its germline genome) a UHC locus and a LoL locus. In some embodiments, a genetically modified non-human animal (e.g., rodent, e.g., mouse or rat) comprises in its genome (e.g., its germline genome) a UHC locus, a KoK locus, and a LoL locus. In some embodiments, a genetically modified non-human animal (e.g., rodent, e.g., mouse or rat) is homozygous at a UHC locus, a KoK locus, a LoL locus, or a combination thereof.

In some embodiments, a genetically modified non-human animal (e.g., rodent, e.g., mouse or rat) comprises in its genome (e.g., its germline genome) a UHC locus, a KoK locus, and a LoK locus. In some embodiments, a genetically modified non-human animal (e.g., rodent, e.g., mouse or rat) comprises in its genome (e.g., its germline genome) a UHC locus, a KoK locus, and a LiK locus.

In some embodiments, a genetically modified non-human animal (e.g., rodent, e.g., mouse or rat) comprises in its genome (e.g., its germline genome) a UHC locus and a LoK locus. In some embodiments, a genetically modified non-human animal (e.g., rodent, e.g., mouse or rat) is homozygous at a UHC locus, a LoK locus, or a combination thereof.

In some embodiments, a genetically modified non-human animal (e.g., rodent, e.g., mouse or rat) comprises in its genome (e.g., its germline genome) a UHC locus and a LiK locus. In some embodiments, a genetically modified non-human animal (e.g., rodent, e.g., mouse or rat) is homozygous at a UHC locus, a LiK locus, or a combination thereof.

In some embodiments, a genetically modified non-human animal (e.g., rodent, e.g., mouse or rat) comprises in its genome (e.g., its germline genome) a UHC locus and a ULC locus. In some embodiments, a genetically modified non-human animal (e.g., rodent, e.g., mouse or rat) is homozygous at a UHC locus, a ULC locus, or a combination thereof.

In some embodiments, a genetically modified non-human animal (e.g., rodent, e.g., mouse or rat) comprises in its genome (e.g., its germline genome) a UHC locus and a DLC locus. In some embodiments, a genetically modified non-human animal (e.g., rodent, e.g., mouse or rat) is homozygous at a UHC locus, a DLC locus, or a combination thereof.

In some embodiments, a genetically modified non-human animal (e.g., rodent, e.g., mouse or rat) comprises in its genome (e.g., its germline genome) a UHC locus and a κULC locus. In some embodiments, a genetically modified non-human animal (e.g., rodent, e.g., mouse or rat) is homozygous at a UHC locus, a κULC locus, or a combination thereof.

In some embodiments, a genetically modified non-human animal (e.g., rodent, e.g., mouse or rat) comprises in its genome (e.g., its germline genome) a UHC locus and a κDLC locus. In some embodiments, a genetically modified non-human animal (e.g., rodent, e.g., mouse or rat) is homozygous at a UHC locus, a κDLC locus, or a combination thereof.

In some embodiments, a genetically modified non-human animal (e.g., rodent, e.g., mouse or rat) comprises in its genome (e.g., its germline genome) a UHC locus and a ULCiK locus. In some embodiments, a genetically modified non-human animal (e.g., rodent, e.g., mouse or rat) is homozygous at a UHC locus, a ULCiK locus, or a combination thereof.

In some embodiments, a genetically modified non-human animal (e.g., rodent, e.g., mouse or rat) comprises in its genome (e.g., its germline genome) a UHC locus and a DLCiK locus. In some embodiments, a genetically modified non-human animal (e.g., rodent, e.g., mouse or rat) is homozygous at a UHC locus, a DLCiK locus, or a combination thereof.

In some embodiments, a genetically modified non-human animal (e.g., rodent, e.g., mouse or rat) comprises in its genome (e.g., its germline genome) a UHC locus and a HULC locus. In some embodiments, a genetically modified non-human animal (e.g., rodent, e.g., mouse or rat) is homozygous at a UHC locus, a HULC locus, or a combination thereof.

In some embodiments, a genetically modified non-human animal (e.g., rodent, e.g., mouse or rat) comprises in its genome (e.g., its germline genome) a LoH locus and a KoK locus. In some embodiments, a genetically modified non-human animal (e.g., rodent, e.g., mouse or rat) comprises in its genome (e.g., its germline genome) a LoH locus and a LoL locus. In some embodiments, a genetically modified non-human animal (e.g., rodent, e.g., mouse or rat) comprises in its genome (e.g., its germline genome) a LoH locus, a KoK locus, and a LoL locus. In some embodiments, a genetically modified non-human animal (e.g., rodent, e.g., mouse or rat) is homozygous at a LoH locus, a KoK locus, a LoL locus, or a combination thereof.

In some embodiments, a genetically modified non-human animal (e.g., rodent, e.g., mouse or rat) comprises in its genome (e.g., its germline genome) a LoH locus, a KoK locus, and a LoK locus. In some embodiments, a genetically modified non-human animal (e.g., rodent, e.g., mouse or rat) comprises in its genome (e.g., its germline genome) a LoH locus, a KoK locus, and a LiK locus.

In some embodiments, a genetically modified non-human animal (e.g., rodent, e.g., mouse or rat) comprises in its genome (e.g., its germline genome) a LoH locus and a LoK locus. In some embodiments, a genetically modified non-human animal (e.g., rodent, e.g., mouse or rat) is homozygous at a LoH locus, a LoK locus, or a combination thereof.

In some embodiments, a genetically modified non-human animal (e.g., rodent, e.g., mouse or rat) comprises in its genome (e.g., its germline genome) a LoH locus and a LiK locus. In some embodiments, a genetically modified non-human animal (e.g., rodent, e.g., mouse or rat) is homozygous at a LoH locus, a LiK locus, or a combination thereof.

In some embodiments, a genetically modified non-human animal (e.g., rodent, e.g., mouse or rat) comprises in its genome (e.g., its germline genome) a LoH locus and a κULC locus. In some embodiments, a genetically modified non-human animal (e.g., rodent, e.g., mouse or rat) is homozygous at a LoH locus, a κULC locus, or a combination thereof.

In some embodiments, a genetically modified non-human animal (e.g., rodent, e.g., mouse or rat) comprises in its genome (e.g., its germline genome) a LoH locus and a κDLC locus. In some embodiments, a genetically modified non-human animal (e.g., rodent, e.g., mouse or rat) is homozygous at a LoH locus, a κDLC locus, or a combination thereof.

In some embodiments, a genetically modified non-human animal (e.g., rodent, e.g., mouse or rat) comprises in its genome (e.g., its germline genome) a LoH locus and a ULCiK locus. In some embodiments, a genetically modified non-human animal (e.g., rodent, e.g., mouse or rat) is homozygous at a LoH locus, a ULCiK locus, or a combination thereof.

In some embodiments, a genetically modified non-human animal (e.g., rodent, e.g., mouse or rat) comprises in its genome (e.g., its germline genome) a LoH locus and a DLCiK locus. In some embodiments, a genetically modified non-human animal (e.g., rodent, e.g., mouse or rat) is homozygous at a LoH locus, a DLCiK locus, or a combination thereof.

In some embodiments, a genetically modified non-human animal (e.g., rodent, e.g., mouse or rat) comprises in its genome (e.g., its germline genome) a LoH locus and a HULC locus. In some embodiments, a genetically modified non-human animal (e.g., rodent, e.g., mouse or rat) is homozygous at a LoH locus, a HULC locus, or a combination thereof.

In some embodiments, a genetically modified non-human animal (e.g., rodent, e.g., mouse or rat) comprises an exogenous terminal deoxynucleotidyl transferase (TdT) gene. Rodents including an exogenous TdT are exemplified in, e.g., U.S. Patent Publication No. 2019/0223418 and PCT Publication No. WO 2017/210586, each of which is incorporated by reference in its entirety. In some embodiments, a rodent (e.g., rat or mouse) that comprises an exogenous TdT gene can have increased antigen receptor diversity when compared to a rodent without an exogenous TdT gene.

In some embodiments, a rodent as described herein has a genome comprising an exogenous TdT gene operably linked to a transcriptional control element.

In some embodiments, a transcriptional control element includes a RAG1 transcriptional control element, a RAG2 transcriptional control element, an immunoglobulin heavy chain transcriptional control element, an immunoglobulin κ light chain transcriptional control element, an immunoglobulin λ light chain transcriptional control element, or any combination thereof.

In some embodiments, an exogenous TdT is located at an immunoglobulin κ light chain locus, an immunoglobulin λ light chain locus, an immunoglobulin heavy chain locus, a RAG1 locus, or a RAG2 locus.

In some embodiments, a TdT is a human TdT. In some embodiments, a TdT is a short isoform of TdT (TdTS).

In some embodiments, a rodent as described herein has a genome comprising an engineered or recombinant diversity cluster with an immunoglobulin heavy chain variable region.

In some embodiments, an engineered or recombinant heavy chain diversity (DH) cluster comprises an insertion of one or more DH segments that are each operably linked to a 23-mer recombination signal sequence (RSS). In some embodiments, an engineered or recombinant DH cluster comprises an engineered DH region comprising at least one DH gene segment operably linked to a 23-mer RSS and an unrearranged DH gene segment flanked on one side by a 12-mer RSS and on the other side by another 12-mer RSS, which DH gene segments are operably linked such that they are able to join in a DH-DH recombination event according to the 12/23 rule. In some embodiments, the engineered or recombinant DH gene segments are human. Exemplary engineered or recombinant DH clusters are shown in e.g., WO 2019241692).

In some embodiments, an engineered or recombinant DH cluster comprises at least two DH gene segments that are fused (i.e., a D-D fusion), wherein the DH gene segments, when recombined with V and J gene segments in the heavy chain locus, generate a rearranged VDDJ transcript that encodes an immunoglobulin heavy chain variable domain. Exemplary recombinant DH gene segments are shown in e.g., U.S. Patent Pub. No. 2019/0127757.

In some embodiments, a rodent as described herein has a genome comprising an engineered or recombinant diversity cluster with an immunoglobulin heavy chain variable region such that the rodent produces human antibodies that comprise HCDR3 sequences that are least 10 amino acids in length (e.g., at least 10, at least 11, at least 12, at least 13, at least 14, at least 15, at least 16, at least 17, at least 18, at least 19, at least 20, at least 21, at least 22, at least 23, at least 24, at least 25, or more amino acids in length).

In some embodiments, a rodent described herein comprises an immunoglobulin heavy chain variable region that includes an engineered DH region, wherein the engineered DH region includes one or more nucleotide sequences that each encode a non-immunoglobulin polypeptide of interest, or portion thereof (e.g., a chemokine receptor (e.g., atypical chemokine receptor (ACKR), e.g., a D6 chemokine decoy receptor), a conotoxin, or a tarantula toxin), wherein the immunoglobulin heavy chain variable region is operably linked to an IgA or IgMDA constant region. Exemplary engineered DH regions are shown in e.g., WO 2017123804.

In some embodiments, rodents described herein comprise an engineered immunoglobulin locus capable of making anchor-modified immunoglobulin polypeptides. In some embodiments, an immunoglobulin locus comprises a nucleic acid sequence comprising a modified immunoglobulin variable (V) gene segment that encodes an anchor-modified immunoglobulin polypeptide. In some embodiments, a modified immunoglobulin V gene segment comprises a nucleic acid sequence encoding an anchor between a nucleic acid sequence encoding an immunoglobulin signal peptide and a nucleic acid sequence encoding the framework region (FR)1, complementarity determining region (CDR)1, FR2, CDR2, FR3, and CDR3 of a germline immunoglobulin V gene segment, or a variant thereof. In some embodiments, an anchor modified immunoglobulin polypeptide comprises in operable linkage: (i) an immunoglobulin signal peptide, (ii) an anchor, and (iii) an FR1, CDR1, FR2, CDR2, FR3, and CDR3 of the germline immunoglobulin V segment, or a variant thereof. In some embodiments, an anchor comprises a receptor-binding portion of a non-immunoglobulin polypeptide of interest that binds a cognate receptor. Exemplary engineered V regions are shown in e.g., WO 2022140219.

In some embodiments, a genetically modified rodent (e.g., a rat or mouse) comprises in its genome a modified (e.g., humanized) immunoglobulin heavy and/or light chain locus as described herein. Such combinations of modified immunoglobulin heavy and/or light chain loci may be present in the genome of a rodent, such as a rat or mouse.

In some embodiments, a genetically modified non-human animal (e.g., rodent, e.g., mouse or rat) comprises in its genome (e.g., its germline genome) a HoH locus and a KoK locus. In some embodiments, a genetically modified non-human animal (e.g., rodent, e.g., mouse or rat) comprises in its genome (e.g., its germline genome) a HoH locus and a LoL locus. In some embodiments, a genetically modified non-human animal (e.g., rodent, e.g., mouse or rat) comprises in its genome (e.g., its germline genome) a HoH locus, a KoK locus, and a LoL locus. In some embodiments, a genetically modified non-human animal (e.g., rodent, e.g., mouse or rat) is homozygous at a HoH locus, a KoK locus, a LoL locus, or a combination thereof.

In some embodiments, a genetically modified non-human animal (e.g., rodent, e.g., mouse or rat) comprises in its genome (e.g., its germline genome) a HoH locus, a KoK locus, and a LoK locus. In some embodiments, a genetically modified non-human animal (e.g., rodent, e.g., mouse or rat) comprises in its genome (e.g., its germline genome) a HoH locus, a KoK locus, and a LiK locus.

In some embodiments, a genetically modified non-human animal (e.g., rodent, e.g., mouse or rat) comprises in its genome (e.g., its germline genome) a HoH locus and a LoK locus. In some embodiments, a genetically modified non-human animal (e.g., rodent, e.g., mouse or rat) is homozygous at a HoH locus, a LoK locus, or a combination thereof.

In some embodiments, a genetically modified non-human animal (e.g., rodent, e.g., mouse or rat) comprises in its genome (e.g., its germline genome) a HoH locus and a LiK locus. In some embodiments, a genetically modified non-human animal (e.g., rodent, e.g., mouse or rat) is homozygous at a HoH locus, a LiK locus, or a combination thereof.

In some embodiments, a genetically modified non-human animal (e.g., rodent, e.g., mouse or rat) comprises in its genome (e.g., its germline genome) a HoH locus and a ULC locus. In some embodiments, a genetically modified non-human animal (e.g., rodent, e.g., mouse or rat) is homozygous at a HoH locus, a ULC locus, or a combination thereof.

In some embodiments, a genetically modified non-human animal (e.g., rodent, e.g., mouse or rat) comprises in its genome (e.g., its germline genome) a HoH locus and a DLC locus. In some embodiments, a genetically modified non-human animal (e.g., rodent, e.g., mouse or rat) is homozygous at a HoH locus, a DLC locus, or a combination thereof.

In some embodiments, a genetically modified non-human animal (e.g., rodent, e.g., mouse or rat) comprises in its genome (e.g., its germline genome) a HoH locus and a κULC locus. In some embodiments, a genetically modified non-human animal (e.g., rodent, e.g., mouse or rat) is homozygous at a HoH locus, a κULC locus, or a combination thereof.

In some embodiments, a genetically modified non-human animal (e.g., rodent, e.g., mouse or rat) comprises in its genome (e.g., its germline genome) a HoH locus and a κDLC locus. In some embodiments, a genetically modified non-human animal (e.g., rodent, e.g., mouse or rat) is homozygous at a HoH locus, a κDLC locus, or a combination thereof.

In some embodiments, a genetically modified non-human animal (e.g., rodent, e.g., mouse or rat) comprises in its genome (e.g., its germline genome) a HoH locus and a ULCiK locus. In some embodiments, a genetically modified non-human animal (e.g., rodent, e.g., mouse or rat) is homozygous at a HoH locus, a ULCiK locus, or a combination thereof.

In some embodiments, a genetically modified non-human animal (e.g., rodent, e.g., mouse or rat) comprises in its genome (e.g., its germline genome) a HoH locus and a DLCiK locus. In some embodiments, a genetically modified non-human animal (e.g., rodent, e.g., mouse or rat) is homozygous at a HoH locus, a DLCiK locus, or a combination thereof.

In some embodiments, a genetically modified non-human animal (e.g., rodent, e.g., mouse or rat) comprises in its genome (e.g., its germline genome) a HoH locus and a HULC locus. In some embodiments, a genetically modified non-human animal (e.g., rodent, e.g., mouse or rat) is homozygous at a HoH locus, a HULC locus, or a combination thereof.

In some embodiments, a genetically modified non-human animal (e.g., rodent, e.g., mouse or rat) comprises in its genome (e.g., its germline genome) a UHC locus and a KoK locus. In some embodiments, a genetically modified non-human animal (e.g., rodent, e.g., mouse or rat) comprises in its genome (e.g., its germline genome) a UHC locus and a LoL locus. In some embodiments, a genetically modified non-human animal (e.g., rodent, e.g., mouse or rat) comprises in its genome (e.g., its germline genome) a UHC locus, a KoK locus, and a LoL locus. In some embodiments, a genetically modified non-human animal (e.g., rodent, e.g., mouse or rat) is homozygous at a UHC locus, a KoK locus, a LoL locus, or a combination thereof.

In some embodiments, a genetically modified non-human animal (e.g., rodent, e.g., mouse or rat) comprises in its genome (e.g., its germline genome) a UHC locus, a KoK locus, and a LoK locus. In some embodiments, a genetically modified non-human animal (e.g., rodent, e.g., mouse or rat) comprises in its genome (e.g., its germline genome) a UHC locus, a KoK locus, and a LiK locus.

In some embodiments, a genetically modified non-human animal (e.g., rodent, e.g., mouse or rat) comprises in its genome (e.g., its germline genome) a UHC locus and a LoK locus. In some embodiments, a genetically modified non-human animal (e.g., rodent, e.g., mouse or rat) is homozygous at a UHC locus, a LoK locus, or a combination thereof.

In some embodiments, a genetically modified non-human animal (e.g., rodent, e.g., mouse or rat) comprises in its genome (e.g., its germline genome) a UHC locus and a LiK locus. In some embodiments, a genetically modified non-human animal (e.g., rodent, e.g., mouse or rat) is homozygous at a UHC locus, a LiK locus, or a combination thereof.

In some embodiments, a genetically modified non-human animal (e.g., rodent, e.g., mouse or rat) comprises in its genome (e.g., its germline genome) a UHC locus and a ULC locus. In some embodiments, a genetically modified non-human animal (e.g., rodent, e.g., mouse or rat) is homozygous at a UHC locus, a ULC locus, or a combination thereof.

In some embodiments, a genetically modified non-human animal (e.g., rodent, e.g., mouse or rat) comprises in its genome (e.g., its germline genome) a UHC locus and a DLC locus. In some embodiments, a genetically modified non-human animal (e.g., rodent, e.g., mouse or rat) is homozygous at a UHC locus, a DLC locus, or a combination thereof.

In some embodiments, a genetically modified non-human animal (e.g., rodent, e.g., mouse or rat) comprises in its genome (e.g., its germline genome) a UHC locus and a κULC locus. In some embodiments, a genetically modified non-human animal (e.g., rodent, e.g., mouse or rat) is homozygous at a UHC locus, a κULC locus, or a combination thereof.

In some embodiments, a genetically modified non-human animal (e.g., rodent, e.g., mouse or rat) comprises in its genome (e.g., its germline genome) a UHC locus and a κDLC locus. In some embodiments, a genetically modified non-human animal (e.g., rodent, e.g., mouse or rat) is homozygous at a UHC locus, a κDLC locus, or a combination thereof.

In some embodiments, a genetically modified non-human animal (e.g., rodent, e.g., mouse or rat) comprises in its genome (e.g., its germline genome) a UHC locus and a ULCiK locus. In some embodiments, a genetically modified non-human animal (e.g., rodent, e.g., mouse or rat) is homozygous at a UHC locus, a ULCiK locus, or a combination thereof.

In some embodiments, a genetically modified non-human animal (e.g., rodent, e.g., mouse or rat) comprises in its genome (e.g., its germline genome) a UHC locus and a DLCiK locus. In some embodiments, a genetically modified non-human animal (e.g., rodent, e.g., mouse or rat) is homozygous at a UHC locus, a DLCiK locus, or a combination thereof.

In some embodiments, a genetically modified non-human animal (e.g., rodent, e.g., mouse or rat) comprises in its genome (e.g., its germline genome) a UHC locus and a HULC locus. In some embodiments, a genetically modified non-human animal (e.g., rodent, e.g., mouse or rat) is homozygous at a UHC locus, a HULC locus, or a combination thereof.

In some embodiments, a genetically modified non-human animal (e.g., rodent, e.g., mouse or rat) comprises in its genome (e.g., its germline genome) a LoH locus and a KoK locus. In some embodiments, a genetically modified non-human animal (e.g., rodent, e.g., mouse or rat) comprises in its genome (e.g., its germline genome) a LoH locus and a LoL locus. In some embodiments, a genetically modified non-human animal (e.g., rodent, e.g., mouse or rat) comprises in its genome (e.g., its germline genome) a LoH locus, a KoK locus, and a LoL locus. In some embodiments, a genetically modified non-human animal (e.g., rodent, e.g., mouse or rat) is homozygous at a LoH locus, a KoK locus, a LoL locus, or a combination thereof.

In some embodiments, a genetically modified non-human animal (e.g., rodent, e.g., mouse or rat) comprises in its genome (e.g., its germline genome) a LoH locus, a KoK locus, and a LoK locus. In some embodiments, a genetically modified non-human animal (e.g., rodent, e.g., mouse or rat) comprises in its genome (e.g., its germline genome) a LoH locus, a KoK locus, and a LiK locus.

In some embodiments, a genetically modified non-human animal (e.g., rodent, e.g., mouse or rat) comprises in its genome (e.g., its germline genome) a LoH locus and a LoK locus. In some embodiments, a genetically modified non-human animal (e.g., rodent, e.g., mouse or rat) is homozygous at a LoH locus, a LoK locus, or a combination thereof.

In some embodiments, a genetically modified non-human animal (e.g., rodent, e.g., mouse or rat) comprises in its genome (e.g., its germline genome) a LoH locus and a LiK locus. In some embodiments, a genetically modified non-human animal (e.g., rodent, e.g., mouse or rat) is homozygous at a LoH locus, a LiK locus, or a combination thereof.

In some embodiments, a genetically modified non-human animal (e.g., rodent, e.g., mouse or rat) comprises in its genome (e.g., its germline genome) a LoH locus and a κULC locus. In some embodiments, a genetically modified non-human animal (e.g., rodent, e.g., mouse or rat) is homozygous at a LoH locus, a κULC locus, or a combination thereof.

In some embodiments, a genetically modified non-human animal (e.g., rodent, e.g., mouse or rat) comprises in its genome (e.g., its germline genome) a LoH locus and a κDLC locus. In some embodiments, a genetically modified non-human animal (e.g., rodent, e.g., mouse or rat) is homozygous at a LoH locus, a κDLC locus, or a combination thereof.

In some embodiments, a genetically modified non-human animal (e.g., rodent, e.g., mouse or rat) comprises in its genome (e.g., its germline genome) a LoH locus and a ULCiK locus. In some embodiments, a genetically modified non-human animal (e.g., rodent, e.g., mouse or rat) is homozygous at a LoH locus, a ULCiK locus, or a combination thereof.

In some embodiments, a genetically modified non-human animal (e.g., rodent, e.g., mouse or rat) comprises in its genome (e.g., its germline genome) a LoH locus and a DLCiK locus. In some embodiments, a genetically modified non-human animal (e.g., rodent, e.g., mouse or rat) is homozygous at a LoH locus, a DLCiK locus, or a combination thereof.

In some embodiments, a genetically modified non-human animal (e.g., rodent, e.g., mouse or rat) comprises in its genome (e.g., its germline genome) a LoH locus and a HULC locus. In some embodiments, a genetically modified non-human animal (e.g., rodent, e.g., mouse or rat) is homozygous at a LoH locus, a HULC locus, or a combination thereof.

In some embodiments, a genetically modified rodent comprises an immunoglobulin heavy chain locus, an immunoglobulin kappa light chain locus, and an immunoglobulin lambda light chain locus as shown in Table 3 below.

TABLE 3
Shows exemplary combinations of modified
immunoglobulin loci in a rodent genome

| | Heavy Chain | Kappa | |
|---|---|---|---|
| VH | Constant | Light Chain | Lambda Light Chain |
| HOH | IgA | KOK | WT or Disrupted or LOL |
| HOH | IgMDA | KOK | WT or Disrupted or LOL |
| HOH | IgA | WT or Disrupted | LOL |
| HOH | IgMDA | WT or Disrupted | LOL |
| HOH | IgA | LoK | WT or Disrupted |
| HOH | IgMDA | LoK | WT or Disrupted |
| HOH | IgA | LiK | WT or Disrupted |
| HOH | IgMDA | LiK | WT or Disrupted |
| HOH | IgA | κULC | WT or Disrupted or LOL |
| HOH | IgMDA | κULC | WT or Disrupted or LOL |
| HOH | IgA | κDLC | WT or Disrupted or LOL |
| HOH | IgMDA | κDLC | WT or Disrupted or LOL |
| HOH | IgA | ULCiK | WT or Disrupted |
| HOH | IgMDA | ULCiK | WT or Disrupted |
| HOH | IgA | DLCiK | WT or Disrupted |
| HOH | IgMDA | DLCiK | WT or Disrupted |
| UHC | IgA | KOK | WT or Disrupted or LOL |
| UHC | IgMDA | KOK | WT or Disrupted or LOL |
| UHC | IgA | WT or Disrupted | LOL |
| UHC | IgMDA | WT or Disrupted | LOL |
| UHC | IgA | LoK | WT or Disrupted |
| UHC | IgMDA | LoK | WT or Disrupted |
| UHC | IgA | LiK | WT or Disrupted |
| UHC | IgMDA | LiK | WT or Disrupted |
| UHC | IgA | κULC | WT or Disrupted or LOL |
| UHC | IgMDA | κULC | WT or Disrupted or LOL |
| UHC | IgA | κDLC | WT or Disrupted or LOL |
| UHC | IgMDA | κDLC | WT or Disrupted or LOL |
| UHC | IgA | ULCiK | WT or Disrupted |
| UHC | IgMDA | ULCiK | WT or Disrupted |
| UHC | IgA | DLCiK | WT or Disrupted |
| UHC | IgMDA | DLCiK | WT or Disrupted |
| LOH | IgA | KOK | WT or Disrupted or LOL |
| LOH | IgMDA | KOK | WT or Disrupted or LOL |
| LOH | IgA | WT or Disrupted | LOL |
| LOH | IgMDA | WT or Disrupted | LOL |
| LOH | IgA | LoK | WT or Disrupted |
| LOH | IgMDA | LoK | WT or Disrupted |
| LOH | IgA | LiK | WT or Disrupted |
| LOH | IgMDA | LiK | WT or Disrupted |
| LOH | IgA | κULC | WT or Disrupted or LOL |
| LOH | IgMDA | κULC | WT or Disrupted or LOL |
| LOH | IgA | κDLC | WT or Disrupted or LOL |
| LOH | IgMDA | κDLC | WT or Disrupted or LOL |
| LOH | IgA | ULCiK | WT or Disrupted |
| LOH | IgMDA | ULCiK | WT or Disrupted |
| LOH | IgA | DLCiK | WT or Disrupted |
| LOH | IgMDA | DLCiK | WT or Disrupted |

### Targeting Vectors

In some embodiments, the present disclosure provides one or more targeting vectors that are used to modify an endogenous immunoglobulin locus of a non-human animal. Such modifications include, among other things, genetic alterations that disrupt the function and/or expression of one or more endogenous immunoglobulin constant region genes (e.g., Ighg, Ighe, Ighm, and/or Ighd).

Targeting vectors can be employed to introduce a nucleic acid construct into a genomic target locus and comprise a nucleic acid construct and homology arms that flank said nucleic acid construct; those skilled in the art will be aware of a variety of options and features generally applicable to the design, structure, and/or use of targeting vectors. For example, targeting vectors can be in linear form or in circular form, and they can be single-stranded or double-stranded. Targeting vectors can be deoxyribonucleic acid (DNA) or ribonucleic acid (RNA). For ease of reference, homology arms are referred to herein as 5′ and 3′ (i.e., upstream and downstream) homology arms. This terminology relates to the relative position of the homology arms to a nucleic acid construct within a targeting vector. 5′ and 3′ homology arms correspond to regions within a targeted locus or to a region within another targeting vector, which are referred to herein as “5′ target sequence” and “3′ target sequence,” respectively. In some embodiments, homology arms can also function as a 5′ or a 3′ target sequence.

In some embodiments, methods described herein employ two, three or more targeting vectors that are capable of recombining with each other. In various embodiments, targeting vectors are large targeting vectors (LTVEC) as described elsewhere herein. In such embodiments, first, second, and third targeting vectors each comprise a 5′ and a 3′ homology arm. The 3′ homology arm of the first targeting vector comprises a sequence that overlaps with the 5′ homology arm of the second targeting vector (i.e., overlapping sequences), which allows for homologous recombination between first and second LTVECs.

In the case of double targeting methods, a 5′ homology arm of a first targeting vector and a 3′ homology arm of a second targeting vector can be similar to corresponding segments within a target genomic locus (i.e., a target sequence), which can promote homologous recombination of the first and the second targeting vectors with corresponding genomic segments and modifies the target genomic locus.

In the case of triple targeting methods, a 3′ homology arm of a second targeting vector can comprise a sequence that overlaps with a 5′ homology arm of a third targeting vector (i.e., overlapping sequences), which can allow for homologous recombination between the second and the third LTVEC. The 5′ homology arm of the first targeting vector and the 3′ homology arm of the third targeting vector are similar to corresponding segments within the target genomic locus (i.e., the target sequence), which can promote homologous recombination of the first and the third targeting vectors with the corresponding genomic segments and modifies the target genomic locus.

A homology arm and a target sequence or two homology arms “correspond” or are “corresponding” to one another when the two regions share a sufficient level of sequence identity to one another to act as substrates for a homologous recombination reaction. The sequence identity between a given target sequence and the corresponding homology arm found on a targeting vector (i.e., overlapping sequence) or between two homology arms can be any degree of sequence identity that allows for homologous recombination to occur. To give but one example, an amount of sequence identity shared by a homology arm of a targeting vector (or a fragment thereof) and a target sequence of another targeting vector or a target sequence of a target genomic locus (or a fragment thereof) can be, e.g., but not limited to, at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

Moreover, a corresponding region of similarity (e.g., identity) between a homology arm and a corresponding target sequence can be of any length that is sufficient to promote homologous recombination at the target genomic locus. For example, a given homology arm and/or corresponding target sequence can comprise corresponding regions of similarity that are, e.g., but not limited to, about 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5-75 kb, 5-80 kb, 5-85 kb, 5-90 kb, 5-95 kb, 5- 100 kb, 100-200 kb, or 200-300 kb in length (such as described elsewhere herein) such that a homology arm has sufficient similarity to undergo homologous recombination with a corresponding target sequence(s) within a target genomic locus of the cell or within another targeting vector. In some embodiments, a given homology arm and/or corresponding target sequence comprise corresponding regions of similarity that are, e.g., but not limited to, about 10-100 kb, 15-100 kb, 20-100 kb, 25-100 kb, 30-100 kb, 35-100 kb, 40-100 kb, 45-100 kb, 50-100 kb, 55-100 kb, 60-100 kb, 65-100 kb, 70-100 kb, 75-100 kb, 80-100 kb, 85-100 kb, 90-100 kb, or 95-100 kb in length (such as described elsewhere herein) such that a homology arm has sufficient similarity to undergo homologous recombination with a corresponding target sequence(s) within a target genomic locus of the cell or within another targeting vector.

Overlapping sequences of a 3′ homology arm of a first targeting vector and a 5′ homology arm of a second targeting vector or of a 3′ homology arm of a second targeting vector and a 5′ homology arm of a third targeting vector can be of any length that is sufficient to promote homologous recombination between said targeting vectors. For example, a given overlapping sequence of a homology arm can comprise corresponding overlapping regions that are about 1-5 kb, 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5-75 kb, 5-80 kb, 5-85 kb, 5-90 kb, 5-95 kb, 5-100 kb, 100-200 kb, or 200-300 kb in length such that an overlapping sequence of a homology arm has sufficient similarity to undergo homologous recombination with a corresponding overlapping sequence within another targeting vector. In some embodiments, a given overlapping sequence of a homology arm comprises an overlapping region that is about 1-100 kb, 5-100 kb, 10-100 kb, 15-100 kb, 20-100 kb, 25-100 kb, 30-100 kb, 35-100 kb, 40-100 kb, 45-100 kb, 50-100 kb, 55-100 kb, 60-100 kb, 65-100 kb, 70-100 kb, 75-100 kb, 80-100 kb, 85-100 kb, 90-100 kb, or 95-100 kb in length such that an overlapping sequence of a homology arm has sufficient similarity to undergo homologous recombination with a corresponding overlapping sequence within another targeting vector. In some embodiments, an overlapping sequence is from 1-5 kb, inclusive. In some embodiments, an overlapping sequence is from about 1 kb to about 70 kb, inclusive. In some embodiments, an overlapping sequence is from about 10 kb to about 70 kb, inclusive. In some embodiments, an overlapping sequence is from about 10 kb to about 50 kb, inclusive. In some embodiments, an overlapping sequence is at least 10 kb. In some embodiments, an overlapping sequence is at least 20 kb. For example, an overlapping sequence can be from about 1 kb to about 5 kb, inclusive, from about 5 kb to about 10 kb, inclusive, from about 10 kb to about 15 kb, inclusive, from about 15 kb to about 20 kb, inclusive, from about 20 kb to about 25 kb, inclusive, from about 25 kb to about 30 kb, inclusive, from about 30 kb to about 35 kb, inclusive, from about 35 kb to about 40 kb, inclusive, from about 40 kb to about 45 kb, inclusive, from about 45 kb to about 50 kb, inclusive, from about 50 kb to about 60 kb, inclusive, from about 60 kb to about 70 kb, inclusive, from about 70 kb to about 80 kb, inclusive, from about 80 kb to about 90 kb, inclusive, from about 90 kb to about 100 kb, inclusive, from about 100 kb to about 120 kb, inclusive, from about 120 kb to about 140 kb, inclusive, from about 140 kb to about 160 kb, inclusive, from about 160 kb to about 180 kb, inclusive, from about 180 kb to about 200 kb, inclusive, from about 200 kb to about 220 kb, inclusive, from about 220 kb to about 240 kb, inclusive, from about 240 kb to about 260 kb, inclusive, from about 260 kb to about 280 kb, inclusive, or about 280 kb to about 300 kb, inclusive. To give but one example, an overlapping sequence can be from about 20 kb to about 60 kb, inclusive. Alternatively, an overlapping sequence can be at least 1 kb, at least 5 kb, at least 10 kb, at least 15 kb, at least 20 kb, at least 25 kb, at least 30 kb, at least 35 kb, at least 40 kb, at least 45 kb, at least 50 kb, at least 60 kb, at least 70 kb, at least 80 kb, at least 90 kb, at least 100 kb, at least 120 kb, at least 140 kb, at least 160 kb, at least 180 kb, at least 200 kb, at least 220 kb, at least 240 kb, at least 260 kb, at least 280 kb, or at least 300 kb. In some embodiments, an overlapping sequence can be at most 400 kb, at most 350 kb, at most 300 kb, at most 280 kb, at most 260 kb, at most 240 kb, at most 220 kb, at most 200 kb, at most 180 kb, at most 160 kb, at most 140 kb, at most 120 kb, at most 100 kb, at most 90 kb, at most 80 kb, at most 70 kb, at most 60 kb or at most 50 kb.

Homology arms can, in some embodiments, correspond to a locus that is native to a cell (e.g., a targeted locus), or alternatively they can correspond to a region of a heterologous or exogenous segment of DNA that was integrated into the genome of the cell, including, for example, transgenes, expression cassettes, or heterologous or exogenous regions of DNA. In some embodiments, homology arms can, in some embodiments, correspond to a region on a targeting vector in a cell. In some embodiments, homology arms of a targeting vector may correspond to a region of a yeast artificial chromosome (YAC), a bacterial artificial chromosome (BAC), a human artificial chromosome, or any other engineered region contained in an appropriate host cell. Still further, homology arms of a targeting vector may correspond to or be derived from a region of a BAC library, a cosmid library, or a P1 phage library. In some certain embodiments, homology arms of a targeting vector correspond to a locus that is native, heterologous, or exogenous to a prokaryote, a yeast, a bird (e.g., chicken), a non-human mammal, a rodent, a human, a rat, a mouse, a hamster a rabbit, a pig, a bovine, a deer, a sheep, a goat, a cat, a dog, a ferret, a primate (e.g., marmoset, rhesus monkey), a domesticated mammal, an agricultural mammal, or any other organism of interest. In some embodiments, homology arms correspond to a locus of the cell that shows limited susceptibility to targeting using a conventional method or that has shown relatively low levels of successful integration at a targeted site, and/or significant levels of off-target integration, in the absence of a nick or double-strand break induced by a nuclease agent (e.g., a Cas protein). In some embodiments, homology arms are designed to include engineered DNA.

In some embodiments, 5′ and 3′ homology arms of a targeting vector(s) correspond to a targeted genome. Alternatively, homology arms correspond to a related genome. For example, a targeted genome is a mouse genome of a first strain, and targeting arms correspond to a mouse genome of a second strain, wherein the first strain and the second strain are different. In certain embodiments, homology arms correspond to the genome of the same animal or are from the genome of the same strain, e.g., the targeted genome is a mouse genome of a first strain, and the targeting arms correspond to a mouse genome from the same mouse or from the same strain.

A homology arm of a targeting vector can be of any length that is sufficient to promote a homologous recombination event with a corresponding target sequence, including, for example, 1-5 kb, inclusive, 5-10 kb, inclusive, 5-15 kb, inclusive, 5-20 kb, inclusive, 5-25 kb, inclusive, 5-30 kb, inclusive, 5-35 kb, inclusive, 5-40 kb, inclusive, 5-45 kb, inclusive, 5-50 kb, inclusive, 5-55 kb, inclusive, 5-60 kb, inclusive, 5-65 kb, inclusive, 5-70 kb, inclusive, 5-75 kb, inclusive, 5-80 kb, inclusive, 5-85 kb, inclusive, 5-90 kb, inclusive, 5-95 kb, inclusive, 5-100 kb, inclusive, 100-200 kb, inclusive, or 200-300 kb, inclusive, in length. In some embodiments, a homology arm of a targeting vector has a length that is sufficient to promote a homologous recombination event with a corresponding target sequence that is 1-100 kb, inclusive, 5-100 kb, inclusive, 10-100 kb, inclusive, 15-100 kb, inclusive, 20-100 kb, inclusive, 25-100 kb, inclusive, 30-100 kb, inclusive, 35-100 kb, inclusive, 40-100 kb, inclusive, 45-100 kb, inclusive, 50-100 kb, inclusive, 55-100 kb, inclusive, 60-100 kb, inclusive, 65-100 kb, inclusive, 70-100 kb, inclusive, 75-100 kb, inclusive, 80-100 kb, inclusive, 85-100 kb, inclusive, 90-100 kb, inclusive, or 95-100 kb, inclusive, in length. As described herein, large targeting vectors can employ targeting arms of greater length.

Nuclease agents (e.g., CRISPR/Cas systems) can be employed in combination with targeting vectors to facilitate the modification of a target locus (e.g., modification of immunoglobulin heavy chain locus). Such nuclease agents may promote homologous recombination between a targeting vector and a target locus. When nuclease agents are employed in combination with a targeting vector, the targeting vector can comprise 5′ and 3′ homology arms corresponding to 5′ and 3′ target sequences located in sufficient proximity to a nuclease cleavage site so as to promote the occurrence of a homologous recombination event between target sequences and homology arms upon a nick or double-strand break at the nuclease cleavage site. The term “nuclease cleavage site” includes a DNA sequence at which a nick or double-strand break is created by a nuclease agent (e.g., a Cas9 cleavage site). Target sequences within a targeted locus that correspond to 5′ and 3′ homology arms of a targeting vector are “located in sufficient proximity” to a nuclease cleavage site if the distance is such as to promote the occurrence of a homologous recombination event between 5′ and 3′ target sequences and homology arms upon a nick or double-strand break at the recognition site. Thus, in certain embodiments, target sequences corresponding to 5′ and/or 3′ homology arms of a targeting vector are within at least one nucleotide of a given recognition site or are within at least 10 nucleotides to about 14 kb of a given recognition site. In some embodiments, a nuclease cleavage site is immediately adjacent to at least one or both of the target sequences.

The spatial relationship of target sequences that correspond to homology arms of a targeting vector and a nuclease cleavage site can vary. For example, target sequences can be located 5′ to a nuclease cleavage site, target sequences can be located 3′ to a recognition site, or target sequences can flank a nuclease cleavage site.

Combined use of a targeting vector (including, for example, a large targeting vector) with a nuclease agent can result in an increased targeting efficiency compared to use of a targeting vector alone. For example, when a targeting vector is used in conjunction with a nuclease agent, targeting efficiency of a targeting vector can be increased by at least two-fold, at least three-fold, at least four-fold, at least five-fold, at least six-fold, at least seven-fold, at least eight-fold, at least nine-fold, at least ten-fold or within a range formed from these integers, such as 2-10-fold when compared to use of a targeting vector alone.

Some targeting vectors are “large targeting vectors” or “LTVECs,” which includes targeting vectors that comprise homology arms that correspond to and are derived from nucleic acid sequences larger than those typically used by other approaches intended to perform homologous recombination in cells. A LTVEC can be, for example, at least 10 kb in length, or the sum total of a 5′ homology arm and a 3′ homology arm can be, for example, at least 10 kb. LTVECs also include targeting vectors comprising nucleic acid constructs larger than those typically used by other approaches intended to perform homologous recombination in cells. For example, LTVECs make possible the modification of large loci that cannot be accommodated by traditional plasmid-based targeting vectors because of their size limitations. For example, a targeted locus can be (i.e., 5′ and 3′ homology arms can correspond to) a locus of a cell that is not targetable using a conventional method or that can be targeted only incorrectly or only with significantly low efficiency in the absence of a nick or double-strand break induced by a nuclease agent (e.g., a Cas protein).

In some embodiments, methods described herein employ two or three LTVECs that are capable of recombining with each other and with a target genomic locus in a three-way or a four-way recombination event. Such methods make possible the modification of large loci that cannot be achieved using a single LTVEC.

Examples of LTVECs include vectors derived from a bacterial artificial chromosome (BAC), a human artificial chromosome, or a yeast artificial chromosome (YAC). LTVECs can be in linear form or in circular form. Examples of LTVECs and methods for making them are described, e.g., in U.S. Pat. Nos. 6,586,251, 6,596,541 and 7,105,348; and International Patent Application Publication No. WO 2002/036789, each of which is incorporated herein by reference in their entireties.

In some embodiments, a provided targeting vector, when introduced into a rodent cell, produces a disruption at the endogenous rodent immunoglobulin heavy chain locus. In some embodiments, a provided targeting vector, when introduced into a rodent cell, produces a genetic alteration in the endogenous rodent immunoglobulin heavy chain constant region locus, which results in the disruption of expression of one or more endogenous rodent immunoglobulin constant region genes (e.g., Ighg, Ighe, Ighm, and/or Ighd). In some embodiments, a provided targeting vector, when introduced into a rodent cell, produces a genetic alteration that disrupts the endogenous rodent immunoglobulin heavy chain constant region locus, which results in the disruption of expression of endogenous rodent immunoglobulin constant region genes Ighg and Ighe. In some embodiments, a provided targeting vector, when introduced into a rodent cell, produces a genetic alteration that disrupts at the endogenous rodent immunoglobulin heavy chain constant region locus, which results in the disruption of expression of endogenous rodent immunoglobulin constant region genes Ighg, Ighe, Ighm, and Ighd.

In some embodiments, a genetic alteration comprises one or more genetic deletions in an endogenous rodent immunoglobulin locus. In some embodiments, a genetic alteration comprises one or more genetic insertions in an endogenous rodent immunoglobulin locus. In some embodiments, a genetic alteration comprises one or more genetic mutations (e.g., substitutions) in an endogenous rodent immunoglobulin locus. In some embodiments, a genetic alteration comprises one or more genetic inversions in an endogenous rodent immunoglobulin locus.

In some embodiments, a genetic alteration comprises one or more genetic deletions in an endogenous rodent immunoglobulin locus, such that expression of endogenous Ighg, Ighm, Ighd, and Ighe is disrupted. In some embodiments, a targeting vector targets a region on an endogenous rodent immunoglobulin constant region locus that is upstream of the rodent Ighm CH1 exon and/or upstream of the rodent Igha CH1 exon. In some embodiments, an altered immunoglobulin locus in a rodent cell, after a targeting vector is introduced comprises from 5′ to 3′ Eμ-Sμ-IgA-3′RR, where the mu intronic enhancer Eμ and mu switch region Sμ are directly 5′ of the immunoglobulin constant alpha gene sequence (see FIGS. 1B-1C). In some embodiments, the altered immunoglobulin locus includes other IgM regulatory elements (e.g., I exon and cognate germline promoter) and/or does not contain one or more IgA regulatory elements (e.g., I exon and cognate germline promoter). In some embodiments, a targeting vector produces a deletion at an endogenous rodent immunoglobulin locus that comprises a region corresponding to 200 bp prior to the IgM CH1 exon to 193 bp prior to the IgA CH1 exon on mouse chromosome 12, e.g., corresponding to coordinates Chr12:113,260,430-113,421,730 (GRCm38 assembly) (see FIG. 1A).

In some embodiments, a genetic alteration comprises one or more genetic deletions in an endogenous rodent immunoglobulin locus, such that expression of endogenous Ighg (e.g., endogenous Ighg3, Ighg1, Ighg2b and/or Ighg2a), and Ighe, is disrupted. In some embodiments, a targeting vector targets a region on an endogenous rodent immunoglobulin constant region locus that is upstream of the rodent Ighg3 CH1 exon and/or upstream of the rodent Igha CH1 exon. In some embodiments, an altered immunoglobulin locus in a rodent cell, after a targeting vector is introduced comprises a mouse immunoglobulin heavy chain constant region that comprises from 5′ to 3′ Eμ-Sμ-IgM-IgD-Sa-IgA-3′RR, where the IgA gene is driven by its cognate germline promoter and switch region (see FIGS. 2B-2C). In some embodiments, a targeting vector produces a deletion at an endogenous rodent immunoglobulin locus that comprises a region corresponding to about 2 kb prior to the Ighg3 CH1 exon to 2 kb prior to the Igha CH1 exon on mouse chromosome 12, corresponding to coordinates Chr12:113,266,985-113,368,400 (GRCm38 assembly) (see FIG. 2A).

### Nucleic Acid Constructs

In addition to genetic alterations that target an endogenous rodent immunoglobulin constant region locus, the present disclosure provides nucleic acid constructs that comprise targeting vectors described herein. Additionally, the present disclosure provides nucleic acid constructs containing one or more human immunoglobulin heavy and/or light chain variable region gene segments.

Typically, a polynucleotide molecule containing one or more targeting vectors and/or human immunoglobulin heavy and/or light chain sequences (e.g., human V_{H} and/or human V_{L} gene segments) or portion(s) thereof linked with (e.g., is inserted into) a vector, preferably a DNA vector, in order to replicate the polynucleotide molecule in a host cell.

The encoded sequences can be cloned directly from known sequences or sources (e.g., libraries) or synthesized from germline sequences designed in silico based on published sequences available from GenBank or other publicly available databases (e.g., IMGT). Alternatively, bacterial artificial chromosome (BAC) libraries can provide immunoglobulin DNA sequences of interest (e.g., human immunoglobulin heavy and/or light chain variable region sequences and combinations thereof). BAC libraries can contain an insert size of 100-150 kb and are capable of harboring inserts as large as 300 kb (Shizuya, et al., 1992, Proc. Natl. Acad. Sci., USA 89:8794-8797; Swiatek, et al., 1993, Genes and Development 7:2071-2084; Kim, et al., 1996, Genomics 34 213-218; incorporated herein by reference in their entireties). For example, a human BAC library harboring average insert sizes of 164-196 kb has been described (Osoegawa, K. et al., 2001, Genome Res. 11(3):483-96; Osoegawa, K. et al., 1998, Genomics 52:1-8, Article No. GE985423, each of which is incorporated herein by reference in its entirety). Human and mouse genomic BAC libraries have been constructed and are commercially available (e.g., ThermoFisher). Genomic BAC libraries can also serve as a source of immunoglobulin DNA sequences as well as transcriptional control regions.

Alternatively, immunoglobulin DNA sequences may be isolated, cloned and/or transferred from yeast artificial chromosomes (YACs). For example, the nucleotide sequence of the human Igλ light chain locus has been determined (see, e.g., Dunham, I. et al., 1999, Nature 402:489-95, which is incorporated herein by reference in its entirety). Further, YACs have previously been employed to assemble a human Igλ light chain locus transgene (see, e.g., Popov, A. V. et al., 1996, Gene 177:195-201; Popov, A. V et al., 1999, J. Exp. Med. 189(10):1611-19, each of which is incorporated herein by reference in its entirety). An entire Igλ light chain locus (human or rodent) can be cloned and contained within several YACs. If multiple YACs are employed and contain regions of overlapping similarity, they can be recombined within yeast host strains to produce a single construct representing the entire locus or desired portions of the locus (e.g., a region to targeted with a targeting vector). YAC arms can be additionally modified with mammalian selection cassettes by retrofitting to assist in introducing the constructs into embryonic stems cells or embryos by methods known in the art and/or described herein.

DNA and amino acid sequences of human immunoglobulin heavy chain and light chain variable region and constant region gene segments for use in constructing an engineered immunoglobulin locus as described herein may be obtained from published databases (e.g., GenBank, IMGT, etc.) and/or published antibody sequences.

In some certain embodiments, nucleic acid constructs containing human immunoglobulin heavy or light chain variable region gene segments comprise human VH, DH, and JH sequences operably linked to a human or non-human IgH heavy chain constant region gene locus. In some embodiments, an IgH constant region gene locus comprises one or more genetic alterations as described herein. For example, in some embodiments, an IgH constant region locus comprises one or more genetic alterations (e.g., a deletion) that disrupts expression of one or more endogenous rodent IgH constant region gene segments (e.g., Ighg, Ighe, Ighd, and/or Ighm). In some embodiments, a nucleic acid construct described herein comprises a rodent immunoglobulin constant region that comprises from 5′ to 3′ Eμ-Sμ-IgA-3′RR, where the mu intronic enhancer Eμ and mu switch region Sμ are directly 5′ of the immunoglobulin constant alpha gene sequence (see FIGS. 1B-1C). In some embodiments, a nucleic acid construct described herein includes other IgM regulatory elements (e.g., I exon and cognate germline promoter) and/or does not contain one or more IgA regulatory elements (e.g., I exon and cognate germline promoter). In some embodiments, a nucleic acid construct described herein comprises a rodent immunoglobulin constant region that comprises from 5′ to 3′ Eμ-Sμ-IgM-IgD-Sa-IgA-3′RR, where the IgA gene is driven by its cognate germline promoter and switch region (see FIGS. 2B-2C).

In some certain embodiments, nucleic acid constructs containing human Igλ light chain gene segments comprise human Vλ and Jλ sequences operably linked to a non-human or human Cλ region gene and non-human Igκ light chain enhancer regions (or enhancer sequences). In some certain embodiments, nucleic acid constructs containing human Igκ light chain gene segments comprise human Vκ and Jκ sequences operably linked to a non-human or human Cκ region gene and non-human Igκ light chain enhancer regions (or enhancer sequences).

In some embodiments, nucleic acid constructs containing human variable region gene sequences further comprises intergenic DNA that is of human and/or murine origin. In some embodiments, intergenic DNA between one or more human heavy chain variable region genes is or comprises non-coding murine IgH variable region sequence or non-coding human IgH variable region sequence, or combinations thereof. In some embodiments, intergenic DNA between one or more human light chain variable region sequences is or comprises non-coding murine Igκ light chain sequence, non-coding human Igκ light chain sequence, non-coding murine Igλ light chain sequence, non-coding human Igλ light chain sequence, or combinations thereof.

Nucleic acid constructs can be prepared using methods known in the art. For example, a nucleic acid construct can be prepared as part of a larger plasmid. Such preparation allows the cloning and selection of the correct construction in an efficient manner as is known in the art. Nucleic acid constructs containing human or rodent immunoglobulin heavy and/or light chain sequences, in whole or in part, as described herein can be located between restriction sites on the plasmid so that they can be isolated from the remaining plasmid sequences for incorporation into a desired non-human animal.

Various methods employed in preparation of nucleic acid constructs (e.g., plasmids) and transformation of host organisms are known in the art. For other suitable expression systems for both prokaryotic and eukaryotic cells, as well as general recombinant procedures, see Principles of Gene Manipulation: An Introduction to Genetic Manipulation, 5th Ed., ed. By Old, R. W. and S. B. Primrose, Blackwell Science, Inc., 1994 and Molecular Cloning: A Laboratory Manual, 2nd Ed., ed. by Sambrook, J. et al., Cold Spring Harbor Laboratory Press: 1989, each of which is incorporated herein by reference in its entirety.

### Methods of Making Provided Non-Human Animals

The present disclosure provides compositions and methods for making non-human animals (e.g., rodents, e.g., mice or rats) whose germline genome comprises an engineered immunoglobulin locus comprising one or more modifications that disrupt expression of the endogenous immunoglobulin constant region genes Ighm, Ighg, Ighd, and/or Ighe, including compositions and methods for making non-human animals that express antibodies comprising certain isotypes, e.g., IgA antibodies. In other embodiments, the present disclosure provides genetically modified rodents that produce only, primarily, or an increased titer of IgA, IgM, and IgD antibodies, e.g., by introducing one or more modifications that disrupt the function and/or expression of the endogenous rodent immunoglobulin constant region genes Ighg and/or Ighe.

In some embodiments, the present disclosure provides compositions and methods for making genetically modified rodents that additionally comprise an immunoglobulin heavy chain variable region comprising one or more human heavy chain V gene segments, one or more human D gene segments, and one or more human heavy chain J gene segments, wherein the immunoglobulin heavy chain variable region is operably linked to a constant region comprising one or more genetic alterations described herein.

In some embodiments of a method of making a non-human animal, insertion of one or more human V_{H} gene segments, one or more human D_{H} gene segments and one or more human J_{H} gene segments includes human non-coding DNA that naturally appears adjacent to the human V_{H} gene segments, human non-coding DNA that naturally appears adjacent to the human D_{H} gene segments and human non-coding DNA that naturally appears adjacent to the human J_{H} gene segments in an endogenous human immunoglobulin locus.

In some embodiments, the genome of a non-human animal as described herein further comprises one or more human immunoglobulin heavy variable regions as described in U.S. Pat. Nos. 8,502,018, 8,642,835, 8,697,940 and 8,791,323, each of which is incorporated herein by reference in its entirety. Alternatively, an engineered immunoglobulin κ light chain locus as described herein can be engineered into an embryonic stem cell of a different modified strain such as, e.g., a VELOCIMMUNE® strain (see, e.g., U.S. Pat. Nos. 8,502,018 and/or 8,642,835; incorporated herein by reference in their entireties). Homozygosity of the engineered heavy and/or light chain locus as described herein can subsequently be achieved by breeding. In some embodiments, a VELOCIMMUNE® mouse can be a VELOCIMMUNE®1 (“VI-1” or VI1”) mouse, which includes eighteen human V_{H} gene segments, all of the human D_{H} gene segments, and all of the J_{H} gene segments. A VI-1 mouse can also include sixteen human Vκ gene segments and all of the human Jκ gene segments. In some embodiments, a VELOCIMMUNE® mouse can be a VELOCIMMUNE® 2 (“VI-2” or “VI2”) mouse, which includes thirty-nine human V_{H} gene segments, all of the human D_{H} gene segments, and all of the J_{H} gene segments. A VI-2 mouse can also include human thirty Vκ gene segments and all of the human Jκ gene segments. In some embodiments, a VELOCIMMUNE® mouse can be a VELOCIMMUNE® 3 (“VI-3” or “VI3”) mouse, which includes eighty human V_{H} gene segments, all of the human D_{H} gene segments, and all of the J_{H} gene segments. A VI-3 mouse can also include human forty Vκ gene segments and all of the human Jκ gene segments.

In some embodiments, the present disclosure provides compositions and methods for making genetically modified rodents that additionally comprise an immunoglobulin light chain variable region comprising one or more human light chain V gene segments and one or more human light chain J gene segments, wherein the light chain is operably linked to a light chain constant region.

Where appropriate, a human immunoglobulin variable region sequences may separately be modified to include codons that are optimized for expression in a non-human animal (e.g., see U.S. Pat. Nos. 5,670,356 and 5,874,304). Codon optimized sequences are engineered sequences, and preferably encode the identical polypeptide (or a biologically active fragment of a full-length polypeptide which has substantially the same activity as the full-length polypeptide) encoded by the non-codon optimized parent polynucleotide. In some embodiments, a human immunoglobulin variable region sequence may separately include an altered sequence to optimize codon usage for a particular cell type (e.g., a rodent cell). For example, the codons of each nucleotide sequence to be inserted into the genome of a non-human animal (e.g., a rodent) may be optimized for expression in a cell of the non-human animal. Such a sequence may be described as a codon-optimized sequence.

Insertion of nucleotide sequences encoding human immunoglobulin variable domains employs a minimal modification of the germline genome of a non-human animal as described herein and results in expression of antibodies comprising immunoglobulin chains having human variable domains, which human variable domains are expressed from endogenous engineered immunoglobulin heavy and/or light chain locus. Methods for generating engineered non-human animals, including knockouts and knock-ins, are known in the art (see, e.g., Gene Targeting: A Practical Approach, Joyner, ed., Oxford University Press, Inc., 2000; incorporated herein by reference in its entirety). For example, generation of genetically modified rodents may optionally involve disruption of the genetic loci of one or more endogenous rodent genes (or gene segments) and introduction of one or more heterologous genes (or gene segments or nucleotide sequences) into the rodent genome, in some embodiments, at the same location as an endogenous rodent gene (or gene segments). In some embodiments, nucleotide sequences encoding human variable domains are introduced upstream of a murine or human immunoglobulin chain constant region gene (e.g., an engineered endogenous rodent immunoglobulin constant region locus described herein) in the germline genome of a rodent.

Schematic illustrations (not to scale) of exemplary methods for constructing an engineered immunoglobulin heavy chain constant region locus as described herein are provided in FIGS. 1-3. In particular, FIGS. 1A-1C and FIGS. 2A-2C sets forth an exemplary strategy for construction of an engineered immunoglobulin heavy chain constant region locus characterized by disruption (e.g., deletion) of nucleotide sequences containing one or more mouse constant region genes (e.g., Ighg, Ighd, Ighm, and/or Ighd).

Targeting vectors for constructing an engineered immunoglobulin heavy and/or light chain locus as described herein may be incorporated into the germline genome of a non-human cell (e.g., a rodent embryonic stem cell). In some embodiments, targeting vectors as described herein are incorporated into a wild-type immunoglobulin heavy chain locus in the germline genome of a non-human cell. In some embodiments, targeting vectors as described herein are incorporated into a modified or engineered immunoglobulin heavy chain constant region locus in the germline genome of a non-human cell such that targeted homologous recombination occurs resulting in a deletion of one or more endogenous constant region genes. In some embodiments, human V_{H}, D_{H} and J_{H} genomic DNA (e.g., containing a plurality of human V_{H}, D_{H} and J_{H} gene segments) is incorporated into the germline genome of a nonhuman cell (e.g., a rodent embryonic stems cell) such that the human V_{H}, D_{H} and J_{H} genomic DNA is operably linked to the engineered immunoglobulin constant region (e.g., see U.S. Pat. Nos. 8,502,018, 8,642,835, 8,697,940, 8,791,323, 9,006,511, 9,012,717, 9,029,628, 9,035,128, 9,066,502, 9,150,662 and 9,163,092, each of which is incorporated herein by reference in its entirety).

A targeting vector is introduced into rodent (e.g., mouse) embryonic stem cells by electroporation so that the sequence contained in the targeting vector results in the capacity of a non-human cell or non-human animal (e.g., a mouse) that expresses antibodies having immunoglobulin heavy chains comprising IgA, IgD, and/or IgM constant domains, and which heavy chains are expressed from an endogenous engineered immunoglobulin heavy chain locus. As described herein, a genetically modified rodent is generated where an engineered immunoglobulin heavy chain constant region locus has been created in the germline genome of the rodent e.g., an endogenous immunoglobulin heavy chain locus, optionally containing a human immunoglobulin heavy chain variable region sequence (i.e., a plurality of human V_{H}, D_{H}, and J_{H} gene segments, wherein the human immunoglobulin heavy chain variable region sequence is operably linked to the engineered immunoglobulin heavy chain constant region sequence. Antibodies are expressed on the surface of rodent B cells and in the serum of said rodent, which antibodies are characterized by having IgA, IgM, and/or IgD constant domains. When an endogenous immunoglobulin heavy chain locus in the germline genome of the rodent is not targeted by the targeting vector, an engineered immunoglobulin heavy chain transgene is preferably inserted at a location other than that of an endogenous rodent immunoglobulin heavy chain locus (e.g., randomly inserted transgene).

Creation of an engineered immunoglobulin heavy chain constant region locus in a non-human animal as described above provides an engineered rodent strain that produces antibodies that IgA, IgD, and/or IgM constant domains expressed from such an engineered immunoglobulin heavy chain constant region locus having one or more genetic alterations that disrupt the expression of one or more constant region genes (e.g., Ighg, Ighm, Ighe, and/or Ighd genes). Leveraged with the presence of an engineered immunoglobulin heavy chain variable locus that includes a plurality of human V_{H}, D_{H} and J_{H} gene segments operably linked to the engineered immunoglobulin heavy chain constant region locus, an engineered rodent strain that produces antibodies and antibody components for the development of human antibody-based therapeutics is created. Thus, a single engineered rodent strain is realized that has the capacity to provide an alternative in vivo system for exploiting human heavy chain variable domains derived from IgA antibodies for the development of new antibody-based medicines to treat human disease.

The present disclosure provides a method of making a genetically modified rodent (e.g., an IgA rodent), the method comprising genetically modifying the germline genome of a rodent so that the rodent comprises an immunoglobulin heavy chain constant region comprising a rodent Igha constant region gene and only produces immunoglobulin heavy chains that comprise a rodent Igha constant domain encoded by the rodent Igha constant region gene. In some embodiments, methods comprise introducing one or more genetic alterations at the endogenous immunoglobulin heavy chain locus, wherein the one or more genetic alterations disrupt expression from rodent immunoglobulin heavy chain constant region genes: Ighm, Ighd, Ighg and Ighe constant region genes. A genetic alteration may be introduced by e.g., deletions, insertion, mutation, and/or inversion. In some embodiments, one or more deletions remove all or part of each of the rodent immunoglobulin heavy chain constant region genes: Ighm, Ighd, Ighg, and Ighe constant region genes. In some embodiments, one or more deletions remove part of each of the rodent immunoglobulin heavy chain constant region genes: Ighm, Ighd, Ighg and Ighe constant region genes. In some embodiments, one or more deletions remove all of each of the rodent immunoglobulin heavy chain constant region genes: Ighm, Ighd, Ighg and Ighe constant region genes. In some embodiments, one or more deletions remove a contiguous sequence spanning a region that is upstream of the rodent Ighm constant region gene to a region that is upstream of the rodent Igha constant region gene. In some embodiments, one or more deletions do not remove all or a part of the non-coding region between the rodent immunoglobulin heavy chain constant region genes: Ighm, Ighd, Ighg constant region genes and Ighe. In some embodiments, one or more genetic alterations comprise one or more insertions.

In some embodiments, a genetic alteration comprises one or more insertions comprises, e.g., an insertion of one or more repressor elements that inhibit expression of the rodent immunoglobulin heavy chain constant region genes: Ighm, Ighd, Ighg, and Ighe constant region genes.

In some embodiments, one or more genetic alterations comprise one or more mutations. In some embodiments, one or more mutations comprise one or more frameshift mutations that inhibit expression of rodent immunoglobulin heavy chain constant region genes: Ighm, Ighd, Ighg and Ighe constant region genes.

In some embodiments, provided methods include genetically modifying a rodent (e.g., mouse) endogenous immunoglobulin heavy chain constant region locus so that it comprises in 5′ to 3′ order: a rodent intronic enhancer (Eμ), a rodent switch mu region (Sμ), and a rodent immunoglobulin heavy constant alpha (Igha) gene, e.g., upstream of a rodent 3′ IgH regulatory region 3′RR that is 3′ of the rodent Igha constant region gene.

The present disclosure also provides a method of making a genetically modified rodent (e.g., an IgMDA rodent), the method comprising genetically modifying the germline genome of a rodent so that the rodent comprises an immunoglobulin heavy chain constant region comprising constant region comprising rodent immunoglobulin heavy chain constant region genes Ighm, Ighd, and Igha, and only produces immunoglobulin heavy chains that comprise a rodent Igha, a rodent Ighm, or a rodent Ighd constant domain encoded by the Igha constant region gene, the rodent Ighm constant region gene, or the rodent Ighd constant region gene, respectively.

In some embodiments, methods include modifying a rodent's germline genome to comprise one or more genetic alterations at the endogenous immunoglobulin heavy chain locus, wherein the one or more genetic alterations disrupt expression from rodent immunoglobulin heavy chain constant region genes: Ighg and Ighe constant region genes. In some embodiments, one or more genetic alterations comprise one or more deletions, insertions, mutations, and/or inversions.

In some embodiments, one or more genetic alternations comprises one or more deletions. In some embodiments, one or more deletions remove all or part of each of the rodent immunoglobulin heavy chain constant region genes: Ighg and Ighe constant region genes. In some embodiments, one or more deletions remove part of each of the rodent immunoglobulin heavy chain constant region genes: Ighg and Ighe constant region genes. In some embodiments, one or more deletions remove all of each the rodent immunoglobulin heavy chain constant region genes: Ighg and Ighe constant region genes. In some embodiments, one or more deletions remove a contiguous sequence spanning a region that is upstream of the rodent Ighg3 constant region gene to a region that is upstream of the rodent Igha constant region gene. In some embodiments, one or more deletions do not remove one or more of the non-coding sequences adjacent to the rodent immunoglobulin heavy chain constant region genes: Ighg and Ighe constant region genes.

In some embodiments, one or more genetic alterations comprise one or more insertions. In some embodiments, one or more insertions comprise an insertion of one or more repressor elements that inhibit expression of the rodent immunoglobulin heavy chain constant region genes: Ighg and Ighe constant region genes.

In some embodiments, one or more genetic alterations comprise one or more mutations. In some embodiments, one or more mutations comprise one or more frameshift mutations that inhibit expression of each of the rodent immunoglobulin heavy chain constant region genes: Ighg and Ighe constant region genes.

In some embodiments, methods of making a genetically modified rodent (e.g., an IgMDA rodent) comprises genetically engineering an endogenous immunoglobulin heavy chain constant region locus so that it comprises in 5′ to 3′ order: a rodent intronic enhancer (Eμ), a rodent switch mu region (Sμ), a rodent Ighm constant region gene, a rodent Ighd constant region gene, a rodent switch alpha region (Sα); a rodent Igha constant region gene; and e.g., upstream of a rodent 3′ IgH regulatory region 3′RR.

In some embodiments, genetically modified rodents described herein are modified to be heterozygous for the engineered endogenous immunoglobulin heavy chain constant region locus. In some embodiments, genetically modified rodents described herein are modified to be homozygous for the engineered endogenous immunoglobulin heavy chain constant region locus. In some embodiments, genetically modified rodents described herein are modified to be hemizygous for the engineered endogenous immunoglobulin heavy chain constant region locus.

In some embodiments of a method of making a non-human animal, a DNA fragment further comprises one or more selection markers. In some embodiments of a method of making a non-human animal, a DNA fragment further comprises one or more site-specific recombination sites. In some certain embodiments of a method of making a non-human animal, a DNA fragment further comprises one or more sets of site-specific recombination sites that recombine with the same recombinase. In some certain embodiments of a method of making a non-human animal, a DNA fragment further comprises one or more sets of site-specific recombination sites that recombine with different recombinases.

In some embodiments of a method of making a non-human animal, a DNA fragment is introduced into a non-human embryonic stem cell whose germline genome comprises one or more engineered immunoglobulin loci (e.g., immunoglobulin heavy chain, immunoglobulin κ light chain, immunoglobulin λ light chain, and combinations thereof). In some certain embodiments, engineered immunoglobulin loci are endogenous engineered immunoglobulin loci.

Guidance for germline configuration of human immunoglobulin variable region gene segments and sequences necessary for proper recombination can be found in, e.g., Molecular Biology of B Cells, London: Elsevier Academic Press, 2004, Ed. Honjo, T., Alt, F. W., Neuberger, M. Chapters 4 (pp. 37-59) and 5 (61-82); incorporated herein by reference in their entireties.

In some embodiments, methods include multiple insertions in a single ES cell clone. In some embodiments, methods include sequential insertions made in a successive ES cell clone. In some embodiments, methods include a single insertion made in an engineered ES cell clone.

Alternatively, and/or additionally, in some embodiments, the germline genome of a non-human animal as described herein further comprises a deleted, inactivated, functionally silenced or otherwise non-functional endogenous immunoglobulin kappa or lambda light chain locus. Genetic modifications to delete or render non-functional a gene or genetic locus may be achieved using methods described herein and/or methods known in the art.

A genetically modified founder non-human animal can be identified based upon the presence of an engineered immunoglobulin locus as described herein (e.g., an engineered endogenous rodent immunoglobulin constant region locus) in its germline genome and/or expression of antibodies having an IgA constant domain in tissues or cells of the non-human animal. A genetically modified founder non-human animal can then be used to breed additional non-human animals carrying the engineered immunoglobulin locus thereby creating a cohort of non-human animals each carrying one or more copies of an engineered immunoglobulin locus. Moreover, genetically modified non-human animals carrying an engineered immunoglobulin locus as described herein can further be bred to other genetically modified non-human animals carrying other transgenes (e.g., human immunoglobulin genes) or engineered immunoglobulin loci as desired.

Genetically modified non-human animals may also be produced to contain selected systems that allow for regulated, directed, inducible and/or cell-type specific expression of the transgene or integrated sequence(s). For example, non-human animals as described herein may be engineered to contain one or more sequences encoding a targeting vector described herein, an engineered rodent immunoglobulin heavy chain constant domain, and/or human immunoglobulin heavy and/or light chain variable domain of an antibody that is/are conditionally expressed. Exemplary systems include the Cre/loxP recombinase system of bacteriophage P1 (see, e.g., Lakso, M. et al., 1992, Proc. Natl. Acad. Sci. U.S.A. 89:6232-6, incorporated herein by reference in its entirety) and the FLP/Frt recombinase system of S. cerevisiae (O'Gorman, S. et al, 1991, Science 251:1351-5, incorporated herein by reference in its entirety). Such animals can be provided through the construction of “double” genetically modified non-human animals, e.g., by mating two genetically modified non-human animals, one containing a transgene comprising a selected modification and the other containing a transgene encoding a recombinase (e.g., a Cre recombinase).

Non-human animals as described herein may be prepared as described above, or using methods known in the art, to comprise additional human, humanized or otherwise engineered genes, oftentimes depending on the intended use of the non-human animal. Genetic material of such human, humanized or otherwise engineered genes may be introduced through the further alteration of the genome of cells (e.g., embryonic stem cells) having the genetic modifications or alterations as described above or through breeding techniques known in the art with other genetically modified or engineered strains as desired. In some embodiments, non-human animals as described herein are prepared to further comprise human IgH and/or Igκ light chain genes or gene segments (see e.g., Murphy, A. J. et al., (2014) Proc. Natl. Acad. Sci. U.S.A. 111(14):5153-5158; U.S. Pat. Nos. 8,502,018, 8,642,835, 8,697,940 and 8,791,323; 8,791,323; and U.S. Patent Application Publication No. 2013/0096287 A1; each of which is incorporated herein by reference in its entirety).

In some embodiments, non-human animals as described herein may be prepared by introducing a targeting vector described herein into a cell from a modified or engineered strain. For example, a targeting vector as described herein may be introduced into a VELOCIMMUNE® mouse. VELOCIMMUNE® mice express antibodies that have fully human variable regions and mouse constant regions. In another example, a targeting vector as described herein may be introduced into an engineered mouse as described in any one of U.S. Pat. Nos. 9,006,511, 9,012,717, 9,029,628, 9,035,128, 9,066,502, 9,150,662 and 9,163,092, incorporated herein by reference in their entireties. In some embodiments, non-human animals as described herein are prepared to further comprise human immunoglobulin genes (variable and/or constant region genes). In some embodiments, non-human animals as described herein comprise an engineered Igκ light chain locus as described herein and genetic material from a heterologous species (e.g., humans), wherein the genetic material encodes, in whole or in part, one or more human heavy and/or Igκ light chain variable regions.

For example, as described herein, non-human animals comprising an engineered Igκ light chain locus as described herein may further comprise (e.g., via cross-breeding or multiple gene targeting strategies) one or more modifications as described in Murphy, A. J. et al., (2014) Proc. Natl. Acad. Sci. U.S.A. 111(14):5153-8; Macdonald, L. E. et al., 2014, Proc. Natl. Acad. Sci. U.S.A. 111(14):5147-52; U.S. Pat. Nos. 8,502,018, 8,642,835, 8,697,940 and 8,791,323; all of which are incorporated herein by reference in their entireties. In some embodiments, a rodent comprising an engineered immunoglobulin constant region locus as described herein is crossed to a rodent comprising a humanized immunoglobulin heavy chain variable region locus. In some embodiments, a rodent comprising an engineered immunoglobulin constant region locus as described herein is crossed to a rodent comprising a humanized immunoglobulin light chain variable region locus. Methods for modifying multiple loci in a non-human animal are described in e.g., U.S. Pat. Nos. 8,502,018, 8,642,835, 8,697,940, 9,006,511, 9,012,717, 9,029,628, 9,035,128, 9,066,502, 9,150,662 and 9,163,092 and/or 8,791,323; incorporated herein by reference).

Although embodiments describing the construction of an engineered immunoglobulin constant region locus in a rodent (i.e., a mouse with an engineered immunoglobulin constant region locus characterized by the disruption (e.g., deletion) of one or more constant region gene segments (e.g., Ighg, Ighm, Ighe, and/or Ighe), so that antibodies containing certain constant domains (e.g., IgA) are expressed) are extensively discussed herein, other non-human animals that comprise an engineered immunoglobulin constant region locus are also provided. Such non-human animals include any of those which can be genetically modified to express antibodies as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. For example, for those non-human animals for which suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing somatic cell nuclear transfer (SCNT) to transfer the genetically modified genome to a suitable cell, e.g., an enucleated oocyte, and gestating the modified cell (e.g., the modified oocyte) in a non-human animal under suitable conditions to form an embryo.

Methods for modifying the germline genome of a non-human animal (e.g., a pig, cow, rodent, chicken, etc. genome) include, e.g., employing a zinc finger nuclease (ZFN), a transcription activator-like effector nuclease (TALEN), or a Cas protein (i.e., a CRISPR/Cas system) to include an engineered immunoglobulin κ light chain locus as described herein. Guidance for methods for modifying the germline genome of a non-human animal can be found in, e.g., U.S. patent application Ser. No. 14/747,461 (filed Jun. 23, 2015), Ser. No. 14/948,221 (filed Nov. 20, 2015) and Ser. No. 14/974,623 (filed Dec. 18, 2015); incorporated herein by reference in their entireties.

In some embodiments, a non-human animal as described herein is a mammal. In some embodiments, a non-human animal as described herein is a small mammal, e.g., of the superfamily Dipodoidea or Muroidea. In some embodiments, a genetically modified non-human animal as described herein is a rodent. In some embodiments, a rodent as described herein is selected from a mouse, a rat, and a hamster. In some embodiments, a rodent as described herein is selected from the superfamily Muroidea. In some embodiments, a genetically modified non-human animal as described herein is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some certain embodiments, a genetically modified rodent as described herein is selected from a true mouse or rat (family Muridae), a gerbil, a spiny mouse, and a crested rat. In some certain embodiments, a genetically modified mouse as described herein is from a member of the family Muridae. In some embodiment, a non-human animal as described herein is a rodent. In some certain embodiments, a rodent as described herein is selected from a mouse and a rat. In some embodiments, a non-human animal as described herein is a mouse.

In some embodiments, a non-human animal as described herein is a rodent that is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some certain embodiments, a mouse as described herein is a 129-strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129/SvJae, 129S6 (129/SvEvTac), 129S7, 129S8, 129T1, 129T2 (see, e.g., Festing et al., 1999, Mammalian Genome 10:836; Auerbach, W. et al., 2000, Biotechniques 29(5):1024-1028, 1030, 1032, each of which is incorporated herein by reference in its entirety). In some certain embodiments, a genetically modified mouse as described herein is a mix of an aforementioned 129 strain and an aforementioned C57BL/6 strain. In some certain embodiments, a mouse as described herein is a mix of aforementioned 129 strains, or a mix of aforementioned BL/6 strains. In some certain embodiments, a 129 strain of the mix as described herein is a 129S6 (129/SvEvTac) strain. In some embodiments, a mouse as described herein is a BALB strain, e.g., BALB/c strain. In some embodiments, a mouse as described herein is a mix of a BALB strain and another aforementioned strain.

In some embodiments, a non-human animal as described herein is a rat. In some certain embodiments, a rat as described herein is selected from a Wistar rat, an LEA strain, a Sprague Dawley strain, a Fischer strain, F344, F6, and Dark Agouti. In some certain embodiments, a rat strain as described herein is a mix of two or more strains selected from the group consisting of Wistar, LEA, Sprague Dawley, Fischer, F344, F6, and Dark Agouti.

A rat pluripotent and/or totipotent cell can be from any rat strain, including, for example, an ACI rat strain (an inbred strain originally derived from August and Copenhagen strains), a Dark Agouti (DA) rat strain, a Wistar rat strain, a LEA rat strain, a Sprague Dawley (SD) rat strain, or a Fischer rat strain such as Fisher F344 or Fisher F6. Rat pluripotent and/or totipotent cells can also be obtained from a strain derived from a mix of two or more strains recited above. For example, the rat pluripotent and/or totipotent cell can be from a DA strain or an ACI strain. The ACI rat strain is characterized as having black agouti, with white belly and feet and an RTlavl haplotype. Such strains are available from a variety of sources including Harlan Laboratories. An example of a rat ES cell line from an ACI rat is an ACI.G1 rat ES cell. The DA rat strain is characterized as having an agouti coat and an RTlavl haplotype. Such rats are available from a variety of sources including Charles River and Harlan Laboratories. Examples of a rat ES cell line from a DA rat are the DA.2B rat ES cell line and the DA.2C rat ES cell line. In some embodiments, the rat pluripotent and/or totipotent cells are from an inbred rat strain (see, e.g., U.S. Patent Application Publication No. 2014-0235933 A1, published Aug. 21, 2014, incorporated herein by reference in its entirety). Guidance for making modifications in a rat genome (e.g., in a rat ES cell) using methods and/or constructs as described herein can be found in, e.g., in U.S. Patent Application Publication Nos. 2014-0310828 and 2017-0204430; both of which are incorporated herein by reference in their entireties.

### Methods of Using Provided Non-Human Animals, Cells or Tissues

Non-human animals (e.g., rodents, e.g., mice or rats) as described herein can be used as a platform for the development of antibodies. In particular, the non-human animals described herein represent a particularly advantageous platform for the generation and identification of antibodies (e.g., human antibodies) of particular isotypes (e.g., IgA, IgM, and/or IgD isotypes).

Accordingly, the present disclosure provides that non-human animals (e.g., rodents, e.g., mice or rats) described herein can be used in methods of making antibodies. Antibodies made in accordance with the present disclosure can include, for example, human antibodies, chimeric antibodies, reverse chimeric antibodies, fragments of any of these antibodies, or combinations thereof.

In some embodiments, non-human animals as described herein may be employed for making a human antibody (e.g., a fully human antibody), which human antibody comprises variable regions derived from nucleic acid sequences encoded by genetic material of a cell of a non-human animal (e.g., rodent) as described herein. In some embodiments, a non-human animal as described herein is immunized with an antigen of interest under conditions and for a time sufficient that the non-human animal develops an immune response to said antigen of interest. Antibodies and/or antibody sequences (i.e., sequences that encode for part of an antibody, e.g., a variable region sequence) are isolated and/or identified from the non-human animal (or one or more cells, for example, one or more B cells, e.g., one or more plasma cells) so immunized and characterized using various assays measuring, for example, affinity, specificity, epitope mapping, ability for blocking ligand-receptor interaction, inhibition receptor activation, etc. In various embodiments, antibodies produced by non-human animals (e.g., rodents, e.g., mice) as described herein comprise one or more human variable regions that are derived from one or more human variable region nucleotide sequences isolated from the non-human animal. In some embodiments, anti-drug antibodies (e.g., anti-idiotype antibody) may be raised in non-human animals as described herein. In various embodiments, antibodies produced by non-human animals as described herein are reverse chimeric antibodies that include a human light chain variable domain and a non-human (e.g., rodent, e.g., mouse) light chain constant domain and/or a human heavy chain variable domain and a non-human (e.g., rodent, e.g., mouse) heavy chain constant domain (e.g., an IgA, IgM, or IgD constant domain).

In various embodiments, antibodies produced by non-human animals include heavy and light chains having a human variable domain and a non-human constant domain. In some embodiments, antibodies produced by non-human animals as described herein are reverse chimeric antibodies that include a human light chain variable domain and a non-human (e.g., rodent, e.g., mouse) light chain constant domain. In some embodiments, antibodies produced by non-human animals as described herein are reverse chimeric antibodies that include a human heavy chain variable domain and a non-human (e.g., rodent, e.g., mouse) heavy chain constant domain (e.g., an IgA, IgM, or IgD constant domain).

In some embodiments, provided methods include immunizing a non-human animal as described herein with an antigen of interest. In some embodiments, provided methods include identifying a lymphocyte (e.g., a clonally selected lymphocyte) from said non-human animal, where the lymphocyte expresses an antibody that binds (e.g., specifically binds) the antigen of interest. In some embodiments, a lymphocyte is a B cell. In some embodiments, the B cell is a plasma cell. In some embodiments, a human heavy chain variable region sequence, a human lambda light chain variable region sequence, and/or a human kappa light chain variable region sequence is obtained from the lymphocyte (e.g., B cell, e.g., plasma cell) and/or identified (e.g., genotyped, e.g., sequenced). In some embodiments, an amino acid sequence of a human heavy chain variable domain, a human lambda light chain variable domain, and/or a human kappa light chain variable domain is obtained from the lymphocyte (e.g., B cell, e.g., plasma cell) and/or identified (e.g., sequenced). In some embodiments, a human heavy chain variable region sequence, a human lambda light chain variable region sequence, and/or a human kappa light chain variable region sequence from a B cell (e.g., plasma cell) of a non-human animal is expressed in a host cell. In some embodiments, a variant of a human heavy chain variable region sequence, a human lambda light chain variable region sequence, and/or a human kappa light chain variable region sequence from a B cell (e.g., plasma cell) of a non-human animal is expressed in a host cell. In some embodiments, a variant includes one or more mutations. In some embodiments, one or more mutations can improve a pharmacokinetic and/or a pharmacodynamic property of an antibody including a variant. In some embodiments, one or more mutations can improve the specificity, the affinity, and/or the immunogenicity of an antibody including a variant.

In some embodiments, methods of making a human antibody include identifying a nucleotide sequence encoding a human immunoglobulin heavy chain variable domain and/or a human immunoglobulin light chain variable domain from a non-human animal described herein; and (i) joining or ligating the nucleotide sequence encoding the human immunoglobulin heavy chain variable domain to a nucleotide sequence encoding a human immunoglobulin heavy chain constant domain (e.g., a constant domain of the same or different isotype, e.g., a human IgG constant domain (e.g., human IgG1, IgG2, IgG3, or IgG4) or a human IgA (e.g., IgA1 or IgA2) constant domain), thereby forming a human immunoglobulin heavy chain sequence encoding a fully human immunoglobulin heavy chain, (ii) joining or ligating the nucleotide sequence encoding the human immunoglobulin λ light chain variable domain to a nucleotide sequence encoding a human immunoglobulin λ light chain constant domain, thereby forming a human immunoglobulin λ light chain sequence encoding a fully human immunoglobulin λ light chain, and/or (iii) joining or ligating the nucleotide sequence encoding the human immunoglobulin κ light chain variable domain to a nucleotide sequence encoding a human immunoglobulin κ light chain constant domain, thereby forming a human immunoglobulin κ light chain sequence encoding a fully human immunoglobulin κ light chain. In certain embodiments, a human immunoglobulin heavy chain sequence, and (i) a human immunoglobulin λ light chain sequence, or (ii) a human immunoglobulin κ light chain sequence are expressed in a cell (e.g., a host cell, a mammalian cell) so that fully human immunoglobulin heavy chains and (i) fully human immunoglobulin λ light chains or (ii) fully human immunoglobulin κ light chains are expressed and form human antibodies. In some embodiments, human antibodies are isolated from the cell or culture media including the cell.

Non-human animals as described herein may be employed for identifying a nucleotide or nucleic acid sequence encoding a human variable domain generated by a non-human animal described herein, e.g., as part of an antibody against an epitope or antigen.

Non-human animals as described herein may be employed for identifying an amino acid sequence of a human variable domain generated by a non-human animal described herein, e.g., as part of an antibody against an epitope or antigen.

Non-human animals as described herein provide an improved in vivo system and source of biological materials (e.g., cells, nucleotides, polypeptides, protein complexes) for producing human antibodies that are useful for a variety of assays. In various embodiments, non-human animals as described herein are used to develop therapeutics that target a polypeptide of interest (e.g., a transmembrane or secreted polypeptide) and/or modulate one or more activities associated with said polypeptide of interest and/or modulate interactions of said polypeptide of interest with other binding partners (e.g., a ligand or receptor polypeptide). For example, in various embodiments, non-human animals as described herein are used to develop therapeutics that target one or more receptor polypeptides, modulate receptor polypeptide activity and/or modulate receptor polypeptide interactions with other binding partners. In various embodiments, non-human animals as described herein are used to identify, screen and/or develop candidate therapeutics (e.g., antibodies) that bind one or more polypeptides of interest. In various embodiments, non-human animals as described herein are used to screen and develop candidate therapeutics (e.g., antibodies) that block activity of one or more polypeptides of interest or that block the activity of one or more receptor polypeptides of interest. In various embodiments, non-human animals as described herein are used to determine the binding profile of antagonists and/or agonists of one or more polypeptides of interest. In some embodiments, non-human animals as described herein are used to determine the epitope or epitopes of one or more candidate therapeutic antibodies that bind one or more polypeptides of interest. In some embodiments, methods used to identify, screen and/or develop candidate therapeutics (e.g., antibodies) include immunizing a genetically modified non-human animal as described herein with an antigen of interest, e.g., one or more polypeptides of interest, and obtaining one or more B lymphocytes (e.g., plasma cells) to identify antigen-binding domains that bind to the one or more polypeptides of interest.

In various embodiments, non-human animals are used to determine the pharmacokinetic profiles of one or more human antibody candidates identified from non-human animals described herein. In various embodiments, one or more non-human animals (e.g., non-human animals as described herein) and one or more control or reference non-human animals are each exposed to one or more human antibody candidates at various doses (e.g., 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/mg, 7.5 mg/kg, 10 mg/kg, 15 mg/kg, 20 mg/kg, 25 mg/kg, 30 mg/kg, 40 mg/kg, or 50 mg/kg or more). Candidate therapeutic antibodies may be dosed via any desired route of administration including parenteral and non-parenteral routes of administration. Parenteral routes include, e.g., intravenous, intraarterial, intraportal, intramuscular, subcutaneous, intraperitoneal, intraspinal, intrathecal, intracerebroventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection. Blood is isolated from non-human animals (humanized and control) at various time points (e.g., 0 hr, 6 hr, 1 day, 2 days, 3 days, 4 days, 5 days, 6 days, 7 days, 8 days, 9 days, 10 days, 11 days, or up to 30 or more days). Various assays may be performed to determine the pharmacokinetic profiles of administered candidate therapeutic antibodies using samples obtained from non-human animals (e.g., non-human animals as described herein) including, but not limited to, total IgG, anti-therapeutic antibody response, agglutination, etc.

In various embodiments, non-human animals (e.g., non-human animals as described herein) are used to measure the therapeutic effect of blocking or modulating the activity of a polypeptide of interest and the effect on gene expression as a result of cellular changes or, in the context of a receptor polypeptide, the density of a receptor polypeptide on the surface of cells in the non-human animals. In various embodiments, a non-human animal (e.g., a non-human animal as described herein) or cells isolated therefrom are exposed to a candidate therapeutic that binds a polypeptide of interest and, after a subsequent period of time, analyzed for effects on specific cellular processes that are associated with said polypeptide of interest, for example, ligand-receptor interactions or signal transduction.

Non-human animals as described herein express human antibody variable regions, thus cells, cell lines, and cell cultures can be generated to serve as a source of human antibody variable regions for use in binding and functional assays, e.g., to assay for binding or function of an antagonist or agonist, particularly where the antagonist or agonist is specific for a human antigen of interest or specific for an epitope that functions in ligand-receptor interaction (binding). In various embodiments, epitopes bound by a candidate therapeutic (e.g., a candidate therapeutic antibody) can be determined using cells isolated from non-human animals as described herein.

Cells from provided non-human animals can be isolated and used on an ad hoc basis or can be maintained in culture for many generations. In various embodiments, cells from a provided non-human animal are immortalized (e.g., via use of a virus) and maintained in culture indefinitely (e.g., in serial cultures).

In some embodiments, a non-human cell is a non-human lymphocyte. In some embodiments, a non-human cell is selected from a B cell, dendritic cell, macrophage, monocyte and a T cell. In some embodiments, a non-human cell is an immature B cell, a mature naïve B cell, an activated B cell, a memory B cell, and/or a plasma cell. In one embodiment, the cell is a plasma cell.

In some embodiments, a non-human cell is a non-human embryonic stem (ES) cell. In some embodiments, a non-human ES cell is a rodent ES cell. In some certain embodiments, a rodent ES cell is a mouse ES cell and is from a 129 strain, C57BL strain, BALB/c or a mixture thereof. In some certain embodiments, a rodent embryonic stem cell is a mouse embryonic stem cell and is a mixture of 129 and C57BL strains. In some certain embodiments, a rodent embryonic stem cell is a mouse embryonic stem cell and is a mixture of 129, C57BL and BALB/c strains.

In some embodiments, use of a non-human ES cell as described herein to make a non-human animal is provided. In some certain embodiments, a non-human ES cell is a mouse ES cell and is used to make a mouse comprising engineered immunoglobulin κ light chain locus as described herein. In some certain embodiments, a non-human ES cell is a rat ES cell and is used to make a rat comprising engineered immunoglobulin κ light chain locus as described herein.

In some embodiments, a non-human tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof.

In some embodiments, an immortalized cell made, generated, produced or obtained from an isolated non-human cell or tissue as described herein is provided.

In some embodiments, a non-human embryo made, generated, produced, or obtained from a non-human ES cell as described herein is provided. In some certain embodiments, a non-human embryo is a rodent embryo; in some embodiments, a mouse embryo; in some embodiments, a rat embryo.

Non-human animals as described herein provide an in vivo system for the generation of variants of human antibody variable regions that binds a polypeptide of interest (e.g., human V_{H} or V_{L} domain variants). Such variants include human antibody variable regions having a desired functionality, specificity, low cross-reactivity to a common epitope shared by two or more variants of a polypeptide of interest. In some embodiments, non-human animals as described herein are employed to generate panels of human antibody variable regions that contain a series of variant variable regions that are screened for a desired or improved functionality.

Non-human animals as described herein provide an in vivo system for generating human antibody variable region libraries (e.g., human V_{H} or V_{L} domain library). Such libraries provide a source for heavy and/or light chain variable region sequences that may be grafted onto different Fc regions based on a desired effector function, used as a source for affinity maturation of the variable region sequence using techniques known in the art (e.g., site-directed mutagenesis, error-prone PCR, etc.) and/or used as a source of antibody components for the generation of antibody-based therapeutic molecules such as, for example, chimeric antigen receptors (i.e., a molecule engineered using antibody components, e.g., an scFv), multi-specific binding agents (e.g., bi-specific binding agents) and fusion proteins (e.g., single domain antibodies, scFvs, etc.).

In some embodiments, a method of producing an antibody in a non-human animal is provided, the method comprising the steps of (a) immunizing a non-human animal as described herein with an antigen of interest; (b) maintaining the non-human animal under conditions sufficient that the non-human animal produces an immune response to the antigen of interest; and (c) recovering an antibody from the non-human animal, or a non-human cell, that binds the antigen of interest.

In some embodiments of a method of producing an antibody in a non-human animal, a non-human cell is a B cell. In one embodiment, the B cell is a plasma cell. In some embodiments of a method of producing an antibody in a non-human animal, a non-human cell is a hybridoma, e.g., hybridoma generated from B cell or plasma cell obtained from the non-human animal.

In some embodiments, an antibody prepared by a method is provided, comprising the steps of: (a) providing a non-human animal as described herein; (b) immunizing the non-human animal with an antigen of interest; (c) maintaining the non-human animal under conditions sufficient that the non-human animal produces an immune response to the antigen of interest; and (d) recovering an antibody from the non-human animal, or a non-human cell, that binds the antigen of interest, wherein the antibody of (d) includes human V_{H} and Vλ domains.

In some embodiments of an antibody prepared by a method described herein, a human V_{H} domain is encoded by a rearranged human heavy chain variable region comprising a human V_{H}3-74, V_{H}3-73, V_{H}3-72, V_{H}2-70, V_{H}1-69, V_{H}3-66, V_{H}3-64, V_{H}4-61, V_{H}4-59, V_{H}1-58, V_{H}3-53, V_{H}5-51, V_{H}3-49, V_{H}3-48, V_{H}1-46, V_{H}1-45, V_{H}3-43, V_{H}4-39, V_{H}4-34, V_{H}3-33, V_{H}4-31, V_{H}3-30, V_{H}4-28, V_{H}2-26, V_{H}1-24, V_{H}3-23, V_{H}3-21, V_{H}3-20, V_{H}1-18, V_{H}3-15, V_{H}3-13, V_{H}3-11, V_{H}3-9, V_{H}1-8, V_{H}3-7, V_{H}2-5, V_{H}7-4-1, V_{H}4-4, V_{H}1-3, V_{H}1-2 V_{H}6-1, or somatically hypermutated variant thereof.

In some embodiments of an antibody prepared by a method described herein, a human Vλ domain is encoded by a rearranged human λ light chain variable region comprising a human Vλ4-69, Vλ8-61, Vλ4-60, Vλ6-57, Vλ10-54, Vλ5-52, Vλ1-51, Vλ9-49, Vλ1-47, Vλ7-46, Vλ5-45, Vλ1-44, Vλ7-43, Vλ1-40, Vλ5-39, Vλ5-37, Vλ1-36, Vλ3-27, Vλ3-25, Vλ2-23, Vλ3-22, Vλ3-21, Vλ3-19, Vλ2-18, Vλ3-16, Vλ2-14, Vλ3-12, Vλ2-11, Vλ3-10, Vλ3-9, Vλ2-8, Vλ4-3 Vλ3-1, or somatically hypermutated variant thereof.

In some embodiments, an antibody prepared by a method is provided, comprising the steps of: (a) immunizing a non-human animal as described herein with an antigen of interest; (b) maintaining the non-human animal under conditions sufficient that the non-human animal produces an immune response to the antigen of interest; and (c) recovering an antibody from the non-human animal, or a non-human cell, that binds the antigen of interest, wherein the antibody of (c) includes human V_{H} and Vκ domains.

In some embodiments of an antibody prepared by a method described herein, a human V_{H} domain is encoded by a rearranged human heavy chain variable region comprising a human V_{H}3-74, V_{H}3-73, V_{H}3-72, V_{H}2-70, V_{H}1-69, V_{H}3-66, V_{H}3-64, V_{H}4-61, V_{H}4-59, V_{H}1-58, V_{H}3-53, V_{H}5-51, V_{H}3-49, V_{H}3-48, V_{H}1-46, V_{H}1-45, V_{H}3-43, V_{H}4-39, V_{H}4-34, V_{H}3-33, V_{H}4-31, V_{H}3-30, V_{H}4-28, V_{H}2-26, V_{H}1-24, V_{H}3-23, V_{H}3-21, V_{H}3-20, V_{H}1-18, V_{H}3-15, V_{H}3-13, V_{H}3-11, V_{H}3-9, V_{H}1-8, V_{H}3-7, V_{H}2-5, V_{H}7-4-1, V_{H}4-4, V_{H}1-3, V_{H}1-2 V_{H}6-1, or somatically hypermutated variant thereof.

In some embodiments of an antibody prepared by a method described herein, a human Vκ domain is encoded by a rearranged human κ light chain variable region comprising a human Vκ3D-7, Vκ1D-8, Vκ1D-43, Vκ3D-11, Vκ3D-12, Vκ3D-13, Vκ3D-15, Vκ3D-16, Vκ3D-17, Vκ3D-20, Vκ6D-21, Vκ2D-26, Vκ2D-28, Vκ2D-29, Vκ2D-30, Vκ1D-33, Vκ1D-39, Vκ2D-40, Vκ2-40, Vκ1-39, Vκ1-33, Vκ2-33, Vκ2-29, Vκ2-28, Vκ1-27, Vκ2-24, Vκ6-21, Vκ3-20, Vκ1-17, Vκ1-16, Vκ3-15, Vκ1-13, Vκ1-12, Vκ3-11, Vκ1-9, Vκ1-8, Vκ1-6, Vκ1-5, Vκ5-2, or Vκ4-1 gene segment, or somatically hypermutated variant thereof.

In some embodiments, a non-human animal, non-human cell or non-human tissue as described herein is provided for use in the manufacture and/or development of a drug (e.g., an antibody or fragment thereof) for therapy or diagnosis.

In some embodiments, a non-human animal, non-human cell or non-human tissue as described herein is provided for use in the manufacture of a medicament for the treatment, prevention or amelioration of a disease, disorder or condition.

In some embodiments, use of a non-human animal, non-human cell or non-human tissue as described herein in the manufacture and/or development of a drug or vaccine for use in medicine, such as use as a medicament, is provided.

In some embodiments, use of a non-human animal or cell as described herein in the manufacture and/or development of an antibody or fragment thereof is provided.

Non-human animals as described herein provide an in vivo system for the analysis and testing of a drug or vaccine. In various embodiments, a candidate drug or vaccine may be delivered to one or more non-human animals as described herein, followed by monitoring of the non-human animals to determine one or more of the immune responses to the drug or vaccine, the safety profile of the drug or vaccine, or the effect on a disease or condition and/or one or more symptoms of a disease or condition. Exemplary methods used to determine the safety profile include measurements of toxicity, optimal dose concentration, antibody (i.e., anti-drug) response, efficacy of the drug or vaccine and possible risk factors. Such drugs or vaccines may be improved and/or developed in such non-human animals.

Vaccine efficacy may be determined in a number of ways. Briefly, non-human animals as described herein are vaccinated using methods known in the art and then challenged with a vaccine or a vaccine is administered to already-infected non-human animals. The response of a non-human animal(s) to a vaccine may be measured by monitoring of, and/or performing one or more assays on, the non-human animal(s) (or cells isolated therefrom) to determine the efficacy of the vaccine. The response of a non-human animal(s) to the vaccine is then compared with control animals, using one or more measures known in the art and/or described herein.

Vaccine efficacy may further be determined by viral neutralization assays. Briefly, non-human animals as described herein are immunized and serum is collected on various days post-immunization. Serial dilutions of serum are pre-incubated with a virus during which time antibodies in the serum that are specific for the virus will bind to it. The virus/serum mixture is then added to permissive cells to determine infectivity by a plaque assay or microneutralization assay. If antibodies in the serum neutralize the virus, there are fewer plaques or lower relative luciferase units compared to a control group.

Non-human animals as described herein produce human antibody variable regions and, therefore, provide an in vivo system for the production of human antibodies for use in diagnostic applications (e.g., immunology, serology, microbiology, cellular pathology, etc.). In various embodiments, non-human animals as described herein may be used to produce human antibody variable regions that bind relevant antigenic sites for identification of cellular changes such as, for example, expression of specific cell surface markers indicative of pathological changes. Such antibodies can be conjugated to various chemical entities (e.g., a radioactive tracer) and be employed in various in vivo and/or in vitro assays as desired.

Non-human animals as described herein provide an improved in vivo system for development and selection of human antibodies for use in oncology and/or infectious diseases. In various embodiments, non-human animals as described herein and control non-human animals (e.g., having a genetic modification that is different than as described herein or no genetic modification, i.e., wild-type) may be implanted with a tumor (or tumor cells) or infected with a virus (e.g., influenza, HIV, HCV, HPV, etc.). Following implantation of infection, non-human animals may be administered a candidate therapeutic. The tumor or virus may be allowed sufficient time to be established in one or more locations within the non-human animals prior to administration of a candidate therapeutic. Alternatively, and/or additionally, the immune response may be monitored in such non-human animals so as to characterize and select potential human antibodies that may be developed as a therapeutic.

### Pharmaceutical Compositions

In some embodiments, an antibody, a nucleic acid, or a therapeutically relevant portion thereof produced by a non-human animal disclosed herein or derived from an antibody, a nucleic acid, or a therapeutically relevant portion thereof produced by a non-human animal disclosed herein can be administered to a subject (e.g., a human subject). In some embodiments, a pharmaceutical composition includes an antibody produced by a non-human animal disclosed herein. In some embodiments, a pharmaceutical composition can include a buffer, a diluent, an excipient, or any combination thereof. In some embodiments, a composition, if desired, can also contain one or more additional therapeutically active substances.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

For example, a pharmaceutical composition provided herein may be provided in a sterile injectable form (e.g., a form that is suitable for subcutaneous injection or intravenous infusion). For example, in some embodiments, a pharmaceutical composition is provided in a liquid dosage form that is suitable for injection. In some embodiments, a pharmaceutical composition is provided as powders (e.g., lyophilized and/or sterilized), optionally under vacuum, which can be reconstituted with an aqueous diluent (e.g., water, buffer, salt solution, etc.) prior to injection. In some embodiments, a pharmaceutical composition is diluted and/or reconstituted in water, sodium chloride solution, sodium acetate solution, benzyl alcohol solution, phosphate buffered saline, etc. In some embodiments, a powder should be mixed gently with the aqueous diluent (e.g., not shaken).

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

In some embodiments, a pharmaceutical composition including an antibody produced by a non-human animal disclosed herein can be included in a container for storage or administration, for example, a vial, a syringe (e.g., an IV syringe), or a bag (e.g., an IV bag). A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100% (w/w) active ingredient.

A pharmaceutical composition may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

In some embodiments, a provided pharmaceutical composition comprises one or more pharmaceutically acceptable excipients (e.g., preservative, inert diluent, dispersing agent, surface active agent and/or emulsifier, buffering agent, etc.). In some embodiments, a pharmaceutical composition comprises one or more preservatives. In some embodiments, pharmaceutical compositions comprise no preservative.

In some embodiments, a pharmaceutical composition is provided in a form that can be refrigerated and/or frozen. In some embodiments, a pharmaceutical composition is provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of antibody compositions for longer than the specified time results in antibody degradation.

Liquid dosage forms and/or reconstituted solutions may comprise particulate matter and/or discoloration prior to administration. In some embodiments, a solution should not be used if discolored or cloudy and/or if particulate matter remains after filtration.

General considerations in the formulation and/or manufacture of pharmaceutical agents may be found, for example, in Remington: The Science and Practice of Pharmacy 21st ed., Lippincott Williams & Wilkins, 2005.

### Kits

The present disclosure further provides a pack or kit comprising one or more containers filled with at least one non-human animal, non-human cell, DNA fragment, targeting vector, or any combination thereof, as described herein. Kits may be used in any applicable method (e.g., a research method). Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, and/or (c) a contract that governs the transfer of materials and/or biological products (e.g., a non-human animal or non-human cell as described herein) between two or more entities and combinations thereof.

In some embodiments, a kit comprising a non-human animal, non-human cell, non-human tissue, immortalized cell, non-human ES cell, or non-human embryo as described herein is provided. In some embodiments, a kit comprising an amino acid (e.g., an antibody or fragment thereof) from a non-human animal, non-human cell, non-human tissue, immortalized cell, non-human ES cell, or non-human embryo as described herein is provided. In some embodiments, a kit comprising a nucleic acid (e.g., a nucleic acid encoding an antibody or fragment thereof) from a non-human animal, non-human cell, non-human tissue, immortalized cell, non-human ES cell, or non-human embryo as described herein is provided. In some embodiments, a kit comprising a sequence (amino acid and/or nucleic acid sequence) identified from a non-human animal, non-human cell, non-human tissue, immortalized cell, non-human ES cell, or non-human embryo as described herein is provided.

In some embodiments, a kit as described herein for use in the manufacture and/or development of a drug (e.g., an antibody or fragment thereof) for therapy or diagnosis is provided.

In some embodiments, a kit as described herein for use in the manufacture and/or development of a drug (e.g., an antibody or fragment thereof) for the treatment, prevention or amelioration of a disease, disorder or condition is provided.

Other features of certain embodiments will become apparent in the course of the following descriptions of exemplary embodiments, which are given for illustration and are not intended to be limiting thereof.

### EXAMPLES

### Example 1: Design and Construction of a Targeting Vectors for Generating a Rodent Expressing Only IgA Antibodies

This example illustrates exemplary methods of constructing a targeting vector for insertion into the genome of a non-human animal such as a rodent (e.g., a mouse) and subsequent homologous recombination. Furthermore, this example demonstrates production of a rodent whose germline genome comprises an engineered immunoglobulin heavy chain locus. In particular, this example demonstrates construction of a targeting vector for engineering an endogenous immunoglobulin heavy chain locus in a rodent so that the rodent expresses and/or produces only IgA antibodies from said immunoglobulin heavy chain locus in the germline genome of the non-human animal.

Design: The present example demonstrates construction of a targeting vector that, when introduced into a mouse cell, produces a deletion of the endogenous mouse IgM, IgD, IgG3, IgG1, IgG2b, IgG2a, and IgE genes on both alleles using VELOCIGENE technology (see, e.g., U.S. Pat. No. 6,586,251 and Valenzuela et al. (2003) High-throughput engineering of the mouse genome coupled with high-resolution expression analysis. Nat. Biotech. 21(6): 652-659, which are herein incorporated by reference in their entirety). The deletion includes a 165 kb region of mouse genomic sequence in the immunoglobulin heavy chain (IgH) locus that spans 200 bp prior to the IgM CH1 exon to 193 bp prior to the IgA CH1 exon on mouse chromosome 12, corresponding to coordinates Chr12:113,260,430-113,421,730 (GRCm38 assembly (see FIG. 1A). After introduction of the targeting vector and homologous recombination, the resulting mouse immunoglobulin heavy chain constant region comprises from 5′ to 3′ Eμ-Sμ-IgA-3′RR, where the mu intronic enhancer Eμ and mu switch region Sμ are directly 5′ of the immunoglobulin constant alpha gene sequence (see FIGS. 1B-1C). Without wishing to be bound by any theory, the resulting configuration, specifically the mu switch and enhancer regions directly upstream of the IgA constant region gene IgA antibody enhance IgA antibody production by way of driving IgA gene expression.

Generation: Mouse homology arms were made by PCR amplification using a modified bacterial artificial chromosome (BAC) vector originally based on BAC clone CT7-302a07 and an unmodified BAC clone BMQ-399K20 (Incyte Genomics/Invitrogen) as template for the 5′ arm and 3′ arm, respectively. Coordinates and primer sequences are indicated in Table 4 below:

TABLE 4
Primer coordinates and sequences

| Homology | Coordinates | length | | |
|---|---|---|---|---|
| arm | (GRCm38) | (bp) | 5′ primer sequence | 3′ primer sequence |
| 5′ homology | chr12: 113,422,931- | 1195 | ACAAAAGCTGGAG | AATTGGCATTATTT |
| arm | 113,424,130 | | CTCCACCGCGGTGG | AAAGTTAACCGGTG |
| | | | CGGCCGCTGAGCA | AACGTGACTTTGG |
| | | | AGAGTGAGTAGAG | AAGCCT (SEQ ID |
| | | | CT (SEQ ID NO: 1) | NO: 2) |
| 3′ homology | chr12: 113,258,009- | 2421 | ATAATGCCAATTAT | GTAATACGACTCAC |
| arm | 113,260,429 | | TTAAAGTTAGTCGA | TATAGGGCGAATTG |
| | | | CTGCAGGTCATGT | GGTACCCGTGGCA |
| | | | TCAAAGTC (SEQ ID | TCTTCTTCCCAGT |
| | | | NO: 3) | (SEQ ID NO: 4) |

Bold font represents sequence annealing to mouse template. Regular font represents sequence
added to carry out isothermal assembly.

To make the targeting vector (designated MAID9706), an isothermal assembly reaction was carried out using the PCR-amplified homology arms and a restriction enzyme-digested plasmid. The plasmid contained a hygromycin resistance cassette flanked by Rox recombinase sites (rox-hyg-rox cassette) and a vector backbone. The homology arms were inserted on either side between the resistance cassette and the vector backbone. After linearization, the final targeting vector contained from 5′ to 3′: the 5′ homology arm, the rox-hyg-rox cassette, and the 3′ homology arm (see FIGS. 1A-1B).

### Example 2: Design and Construction of a Targeting Vector for Generating a Rodent Expressing Only IgA, IgM, or IgD Antibodies

This example illustrates exemplary methods of constructing a targeting vector for insertion into the genome of a non-human animal such as a rodent (e.g., a mouse) and subsequent homologous recombination. Furthermore, this example demonstrates production of a rodent whose germline genome comprises an engineered immunoglobulin heavy chain locus. In particular, this example demonstrates construction of a targeting vector for engineering an endogenous immunoglobulin heavy chain locus in a rodent so that the rodent expresses and/or produces only IgA, IgM, or IgD antibodies from said immunoglobulin heavy chain locus in the germline genome of the non-human animal.

Design: The present example demonstrates construction of a targeting vector that, when introduced into a mouse cell, produces a deletion of endogenous mouse IgG3, IgG1, IgG2b, IgG2a, and IgE genes on both alleles using VELOCIGENE technology (see, e.g., U.S. Pat. No. 6,586,251 and Valenzuela et al. (2003)). The deletion includes an approximately 102 kb region of mouse genomic sequence in the IgH locus that spans from 2 kb prior to the IgG3 I exon to 2 kb prior to the IgA I exon on mouse chromosome 12, corresponding to coordinates Chr12:113,266,985-113,368,400 (GRCm38 assembly) (see FIG. 2A).

After introduction of the targeting vector and homologous recombination, the resulting mouse immunoglobulin heavy chain constant region comprises from 5′ to 3′ Eμ-Sμ-IgM-IgD-Sa-IgA-3′RR, where the IgA gene is driven by its cognate germline promoter and switch region (see FIGS. 2B-2C).

Generation: In detail, mouse homology arms were made by PCR amplification using BAC clone BMQ-70P8 and BMQ-399K20 (Incyte Genomics/Invitrogen) as template for the 5′ arm and 3′ arm, respectively. Coordinates and primer sequences are indicated in Table 5 below:

TABLE 5
Primer coordinates and sequences

| Homology | Coordinates | length | | |
|---|---|---|---|---|
| arm | (GRCm38) | (bp) | 5′ primer sequence | 3′ primer sequence |
| 5′ homology | chr12: 113,368,401- | 2435 | ACAAAAGCTGGAG | CCCGTCGACGAATT |
| arm | 113,370,836 | | CTCCACCGCGGTGG | CACCGGTATGGGG |
| | | | CGGCCGCAGACAC | TCTTCAGTACAAT |
| | | | TTTCATTGGGTCC | TCAG (SEQ ID NO: 6) |
| | | | CAG (SEQ ID NO: 5) | |
| 3′ homology | chr12: 113,264,546- | 2441 | CCCATACCGGTGAA | GTAATACGACTCAC |
| arm | 113,266,984 | | TTCGTCGACGGGG | TATAGGGCGAATTG |
| | | | AATGGTTATTCAC | GGTACCGCTAGCTG |
| | | | AAATG (SEQ ID NO: 7) | TCCCAGTTCAGCC |
| | | | | CAGAT (SEQ ID NO: 8) |

Bold font represents sequence annealing to mouse template. Regular font represents sequence
added to carry out isothermal assembly.

To make the targeting vector (designated MAID9707), an isothermal assembly reaction was carried out using the PCR-amplified homology arms and a restriction enzyme-digested plasmid. The homology arms were inserted in the multiple cloning sites of the vector backbone. Then, a hygromycin resistance cassette flanked by Rox recombinase sites (rox-hyg-rox cassette) was inserted by restriction digest and ligation between the two homology arms.

After linearization, the final targeting vector contained from 5′ to 3′: the 5′ homology arm, the rox-hyg-rox cassette, and the 3′ homology arm (FIGS. 2A-2B).

### Example 3: Generation of IgA Rodents

The present example demonstrates exemplary methods of producing genetically modified non-human animals such as rodents (e.g., a mice) using the targeting vector designed and constructed in Example 1, to generate genetically modified rodents that express and/or produce only IgA antibodies. Specifically, exemplary methods include engineering the rodent IgH locus by inserting the targeting vector of Example 1 into the genome of the rodent and deleting certain rodent IgH constant region gene segments through homologous recombination.

The targeting vector of Example 1 (MAID9706) was electroporated into mouse embryonic stem (ES) cells. The targeted ES cells contained previous modifications to both the heavy chain locus and the kappa chain locus comprising humanizations of 80 V_{H} genes, all D_{H} genes, all J_{H} genes, 40 Vκ genes, and all Jκ genes (e.g., U.S. Pat. Nos. 7,576,259, 7,659,442, 7,294,754, and 8,816,150, which are herein incorporated by reference in their entirety). Cas9 gRNAs targeting the region to be deleted were used to assist targeting. The gRNA sequences are indicated in Table 6 below:

TABLE 6
gRNA sequences

| | | Coordinates |
|---|---|---|
| Name | Sequence | (GRCm38) |
| MAID90089 | CTTCCAAAGTCACGTTCCTG | chr12: |
| | (SEQ ID NO: 9) | 113,422,928- |
| | | 113,422,947 |
| MAID90090 | CCTTGTGAAAGACTACCTGC | chr12: |
| | (SEQ ID NO: 10) | 113,260,427- |
| | | 113,260,446 |

Targeted homologous recombination resulted in a biallelic deletion of ˜165 kb of mouse sequence corresponding to coordinates Chr12:113,260,430-113,421,730 (GRCm38 assembly) and insertion of the rox-hyg-rox cassette. Successful biallelic integration was confirmed by a modification of allele (MOA) assay as described, e.g., in Valenzuela et al. (2003). Primers and probes used for the MOA assay for the loss of the intervening mouse sequence (LOA assay) and gain of inserted sequence (GOA) are depicted in Table 7 below, and are indicated schematically at appropriate locations in FIGS. 1A-1C:

TABLE 7
Primers and probes

| Name | Sequence | Coordinates (GRCm38) | Assay |
|---|---|---|---|
| VI1176mTU | CACCTGGAACTACCAGAACAAC | chr12: 113,422,586-113,422,612 | LOA |
| | ACTGA (SEQ ID NO: 11) | | |
| IgG1_226U | CCATGGTGACCCTGGGATGCCT | chr12: 113,330,442-113,330,463 | LOA |
| | (SEQ ID NO: 12) | | |
| VI1176mTD | CCTCTCGTCTTTGGGTCCACTGC | chr12: 113,260,808-113,260,830 | LOA |
| | (SEQ ID NO: 13) | | |
| Hyg | ACGAGCGGGTTCGGCCCATTC | N/A | GOA |
| | (SEQ ID NO: 14) | | |

After confirmation, the selection cassette was removed in ES cells via Dre recombinase, leaving behind one Rox recombination site in place (FIG. 1C). The resultant cassette-deleted alleles were designated MAID20797.

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008/0078000 A1, all of which are herein incorporated by reference in their entirety). The mouse embryo comprising the donor ES cells was incubated in vitro and then implanted into a surrogate mother to produce an F0 mouse fully derived from the donor ES cells. Mice with the proper MAID20797 alleles were identified by genotyping using the LOA assays described above. Resulting mice are either homozygous for MAID20797 allele or are bred to mice comprising a deletion of all or part of immunoglobulin locus such that the mouse only expresses antibodies comprising MAID20797 allele.

### Example 4: Generation of IgMDA Rodents

The present example demonstrates exemplary methods of producing genetically modified non-human animals such as rodents (e.g., a mice) using the targeting vector designed and constructed in Example 2, to generate genetically modified rodents that express and/or produce only IgM, IgD, or IgA antibodies. Specifically, exemplary methods include engineering the rodent IgH locus by inserting the targeting vector of Example 2 into the genome of the rodent and deleting certain rodent IgH constant region gene segments through homologous recombination.

The targeting vector of Example 2 (MAID9707) was electroporated into mouse embryonic stem (ES) cells. The targeted ES cells contained previous modifications to both the heavy chain locus and the kappa chain locus comprising humanizations of 80 V_{H} genes, all D_{H} genes, all J_{H} genes, 40 Vκ genes, and all Jκ genes (see, e.g., U.S. Pat. Nos. 7,576,259, 7,659,442, 7,294,754, and 8,816,150). Cas9 gRNAs targeting the region to be deleted were used to assist targeting. The gRNA sequences are indicated in Table 8 below:

TABLE 8
gRNA sequences

| | | Coordinates |
|---|---|---|
| Name | Sequence | (GRCm38) |
| MAID90091 | CTGACATCTCTGGCATTATG | chr12: |
| | (SEQ ID NO: 15) | 113,368,384- |
| | | 113,368,403 |
| MAID90092 | TGTGAATAACCATTCCCCAA | chr12: |
| | (SEQ ID NO: 16) | 113,266,967- |
| | | 113,266,986 |

Targeted homologous recombination resulted in a deletion of ˜102 kb of mouse sequence corresponding to coordinates Chr12:113,266,985-113,368,400 (GRCm38 assembly) and insertion of the rox-hyg-rox cassette on only one allele. The other allele was repaired through NHEJ without insertion of the cassette resulting in a deletion of ˜102 kb of mouse sequence corresponding to coordinates Chr12:113,266,719-113,368,439 (GRCm38 assembly). The NHEJ allele (designated MAID90091) was determined via Sanger sequencing of genomic DNA. Successful integration was confirmed by a modification of allele (MOA) assay as described, e.g., in Valenzuela et al. (2003). Primers and probes used for the MOA assay for the loss of the intervening mouse sequence (LOA assay) and gain of inserted sequence (GOA) are depicted in Table 9 below, and are indicated schematically at appropriate locations in FIGS. 2A-2C:

TABLE 9
Primers and probes

| Name | Sequence | Coordinates (GRCm38) | Assay |
|---|---|---|---|
| VI1177mTU2 | TCGGGCATGTGGATCAAGATATAGGT | chr12: 113,367,776-113,367,801 | LOA |
| | (SEQ ID NO: 17) | | |
| IgG1_226U | CCATGGTGACCCTGGGATGCCT | chr12: 113,330,442-113,330,463 | LOA |
| | (SEQ ID NO: 12) | | |
| VI1177mTD3 | TCCCACATAGAAGCAAATCCCACC | chr12: 113,267,187-113,267,210 | LOA |
| | (SEQ ID NO: 18) | | |
| Hyg | ACGAGCGGGTTCGGCCCATTC | N/A | GOA |
| | (SEQ ID NO: 14) | | |

After confirmation, the selection cassette was removed in ES cells via Dre recombinase, leaving behind one Rox recombination site in place (FIG. 2C). The resultant cassette-deleted allele was designated MAID20782.

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008/0078000 A1). The mouse embryo comprising the donor ES cells was incubated in vitro and then implanted into a surrogate mother to produce an F0 mouse fully derived from the donor ES cells. Resulting mice are then bred to homozygosity for the MAID20782 allele or are bred to mice comprising a deletion of all or part of immunoglobulin locus such that the mouse only expresses antibodies comprising MAID20782 allele. MAID20782 alleles were distinguished from MAID90091 alleles by tail genotyping using probes over the novel junctions, depicted in Table 10 below:

TABLE 10
Primers and probes

| Name | Forward Primer | Probe | Reverse Primer |
|---|---|---|---|
| 90091Jxn | CAGGGCGTGGAGTCACTGAT | AGGCCATAGCATAAA | AGGTGGGACCAGCA |
| | (SEQ ID NO: 19) | GTAACCCTACATAGA | GTTGAG (SEQ ID NO: 21) |
| | | (SEQ ID NO: 20) | |
| 20782Rox | GAGTCACTGATAGGC | TGAAATCTGAATTGT | GGTCACATTTGTGAA |
| | CATAGCATA (SEQ ID | ACTGAAGACC (SEQ | TAACCATTCC (SEQ ID |
| | NO: 22) | ID NO: 23) | NO: 24) |

### Example 5: Characterization of IgA and IgMDA Rodents

The present Examples examines immune system and antibody repertoire of exemplary IgA and IgMDA genetically modified rodents generated in the prior Examples, as compared to the VelocImmune Mice (VI3). Modified alleles used in the mice used in these examples are summarized schematically in FIG. 3. In these particular examples, mice designated as “het” or “heterozygous” in FIG. 3 were modified on the depicted alleles but were null on the second allele such that only the modified allele was expressed.

### Analysis of IgA in Stool and Serum of IgA and IgMDA Genetically Modified Mice

The present Example demonstrates, among other things, that genetically modified rodents generated in the previous Examples and described in the present disclosure produce exclusively IgA antibodies (IgA rodents) or exclusively IgA, IgM, or IgD antibodies (IgMDA rodents). Specifically, the present Example quantifies IgA and IgM antibodies in the stool and serum of IgA and IgMDA mice generated in the prior examples and VI3 mice described herein are used as a control.

Materials and Methods: Total stool IgA, serum IgA and IgM were measured by sandwich ELISA. For IgA detection, ELISA plates (Corning 3690) were coated with 1 g/mL goat anti-mouse IgA (Southern Biotech, AL) capture antibody overnight at 4° C. Plates were washed and blocked with 1% BSA in PBS for 2 h at room temperature. Diluted samples and standards were added and incubated overnight at 4° C. Captured IgA was detected by horseradish peroxidase (HRP)-conjugated goat anti-mouse IgA antibody (Sigma-Aldrich). Serum IgM immunoglobulin was detected with the following capture and detection antibody pairs: rat anti-mouse IgM (Southern Biotech, AL), goat anti-mouse IgM-HRP (Southern Biotech, AL). ELISA plates were developed by TMB microwell peroxidase substrate (KPL, Inc.) and quenched by 1 M H2SO4. Colorimetric reaction was measured at OD=450 nm by a Synergy H1 Multi-Mode Microplate Reader (BioTek Instruments, Inc.).

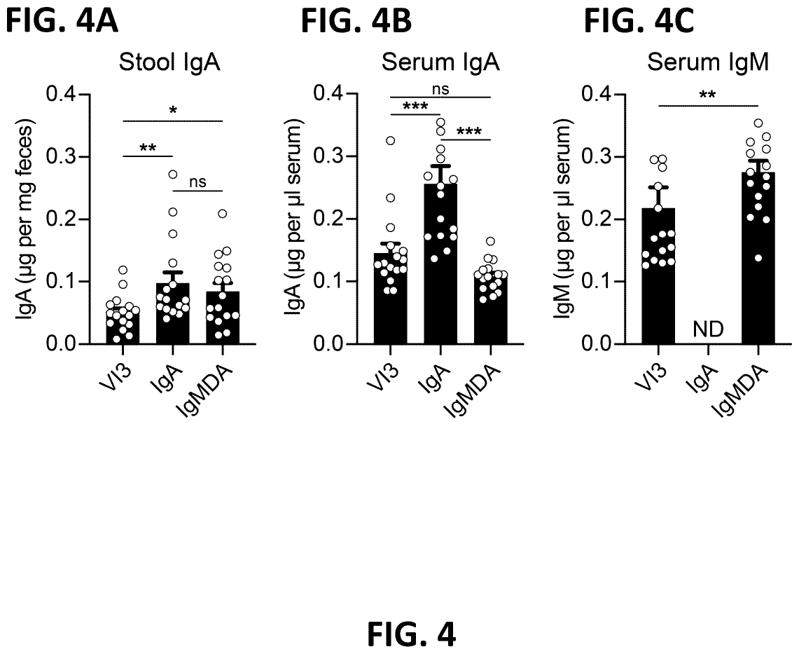

Results: Results from the ELISA are shown in FIGS. 4A-4C. Concentration of IgA antibodies in stool samples obtained from IgA, IgMDA, and VI3 mice (μg/mg feces) is shown in FIG. 4A. Concentration of IgA antibodies in serum samples obtained from IgA, IgMDA, and VI3 mice (μg/μl serum) is shown in FIG. 4B. Concentration of IgM antibodies in serum samples obtained from IgA, IgMDA, and VI3 mice (μg/μl serum) is shown in FIG. 4C. Each dot represents a mouse (10 weeks old); *p<0.05, **p<0.01, ***p<0.001, ns: not significant, determined by Mann-Whitney test; Data: mean+/−S.E.M. These results show that stool samples from IgA and IgMDA mice had significantly higher levels of IgA antibodies than the VI3 mice (FIG. 4A) and that serum samples from IgA mice had significantly higher levels of IgA antibodies than both the VI3 and IgMDA mice. Additionally, serum samples from IgA mice did not show detectable levels of IgM antibodies.

### Analysis of IgA-Coated Bacteria in Stool of IgA and IgMDA Genetically Modified Mice

IgA antibodies are known to be produced in the gut and prevent invasion of microorganisms such as bacteria from spreading to other systems in the body. The present Example examines the presence and percentage of IgA-coated bacteria present in stool samples obtained from IgA and IgMDA mice described in the previous Examples, using samples obtained from VI3 mice as a control. Presence of IgA-coated bacteria in stool indicate that IgA antibodies produced in the mice are present and functionally binding bacterial surface antigens.

Materials and Methods: IgA-coated fecal bacteria in stool samples obtained from IgA, IgMDA and VI3 mice were measured by flow cytometry. Briefly, mouse fecal pellets were dissolved in PBS, homogenized in vortex mixer and centrifuged at 4° C. to remove large particles. The supernatant was passed through a 40 μm sterile nylon filter and an aliquot of the bacteria suspension was collected for staining. Bacteria were washed in PBS/EDTA buffer for 3 times. Non-specific binding sites were first blocked with rat serum for 20 min at 4° C. Bacteria were then stained with monoclonal rat anti-mouse IgA antibody (eBioscience) for 30 min at 4° C. After washing, bacteria were stained with DAPI (Invitrogen). Samples were run through a BD LSR Fortessa cell analyzer and further analyzed by FlowJo software (Tree Star, Inc.).

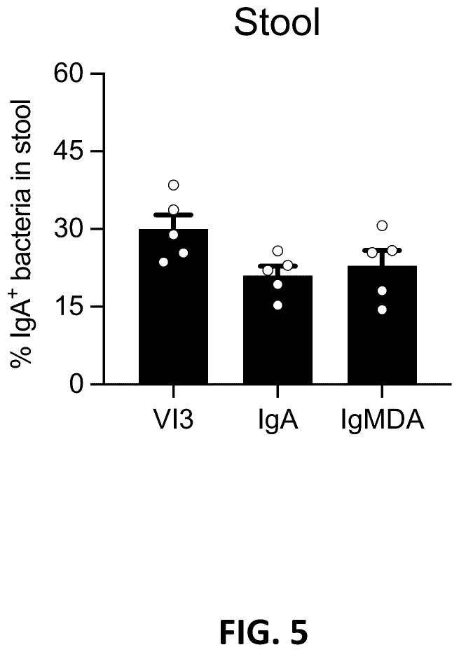

Results: FIG. 5 shows percentage of IgA-coated bacteria in the stool of VI3, IgA only (IgA) and IgMDA mice. Each dot represents the value for one mouse. Mann-Whitney test was used; *p<0.05. The results show that the stool samples obtained from the IgA mice had a lower percentage of IgA+ bacteria.

### Characterization of Immune Cells in IgA and IgMDA Genetically Modified Mice

The present Example examines T and B cells of IgA and IgMDA genetically modified mice compared to VI3 mice (containing all constant region gene segments, and capable of producing all antibody isotypes).

Materials and Methods: Spleen was excised from mouse and then mechanically processed by pushing them through a 40 μm strainer into Dulbecco's Modified Eagle Medium (DMEM) supplemented with 2% FBS. Filtered cells were spun down, washed and resuspended in DMEM/2% FBS. Isolated single cells were washed in PBS and incubated with Zombie Aqua dye (BioLegend) to distinguish dead cells. Non-specific binding to Fc receptors was blocked by anti-mouse CD16/32 antibody (BD Biosciences). Cells were stained in FACS buffer (PBS with 2% FBS and 2 mM EDTA) with antibodies for 30 min in dark at 4° C. The following antibodies were purchased from BioLegend if not indicated otherwise: anti-mouse CD3, anti-mouse CD19, anti-mouse IgA, anti-mouse IgM, anti-mouse kappa light chain, anti-mouse lambda light chain. Multi-parameter analysis was conducted with BD FACSymphony™ A5 and then analyzed with FlowJo software (Tree Star, Inc.).

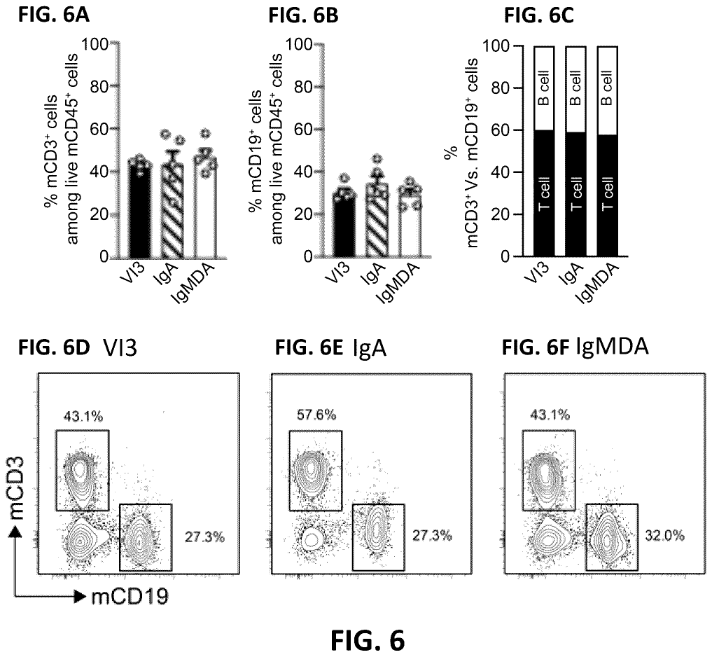

Results: FIGS. 6A-F show quantification of splenic T and B cells in IgA, IgMDA, and VI3 mice. FIG. 6A shows percentage of splenic T cells detected as represented by % CD3+ cells among live mCD45+ cells. FIG. 6B shows % of B cells detected as represented by % CD19+ cells among live mCD45+ cells. FIG. 6C shows ratio of splenic T:B cells detected as represented by % of mCD3+ vs. mCD19+ cells. Each dot represents a mouse. FIGS. 6D-6F show representative flow plots for CD19+ B cells and CD3+ T cells in VI3 mice (FIG. 6D), IgA mice (FIG. 6E), and IgMDA mice (FIG. 6F).

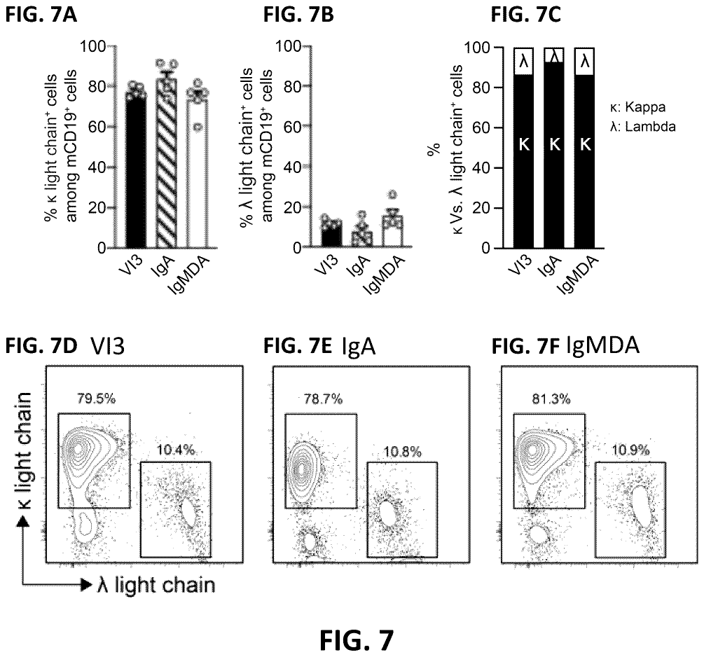

FIGS. 7A-F show quantification of kappa and lambda light chain antibodies among splenic B cells in IgA, IgMDA, and VI3 mice. FIG. 7A shows percentage of B cells with κ light chains detected as represented by % kappa (κ) light chain+ cells among mCD19+ cells (% of B cells with κ light chains). FIG. 7B shows percentage of B cells detected with λ light chains (% lambda (λ) light chain+ cells among mCD19+ cells). FIG. 7C shows the ratio of κ:λ antibodies detected as represented by % κ vs. λ light chain antibodies. Each dot represents a mouse. FIGS. 7D-7F show representative flow plots for κ light chain positive vs. λ light chain positive B cells in VI3 mice (FIG. 7D), IgA mice (FIG. 7E), and IgMDA mice (FIG. 7F).

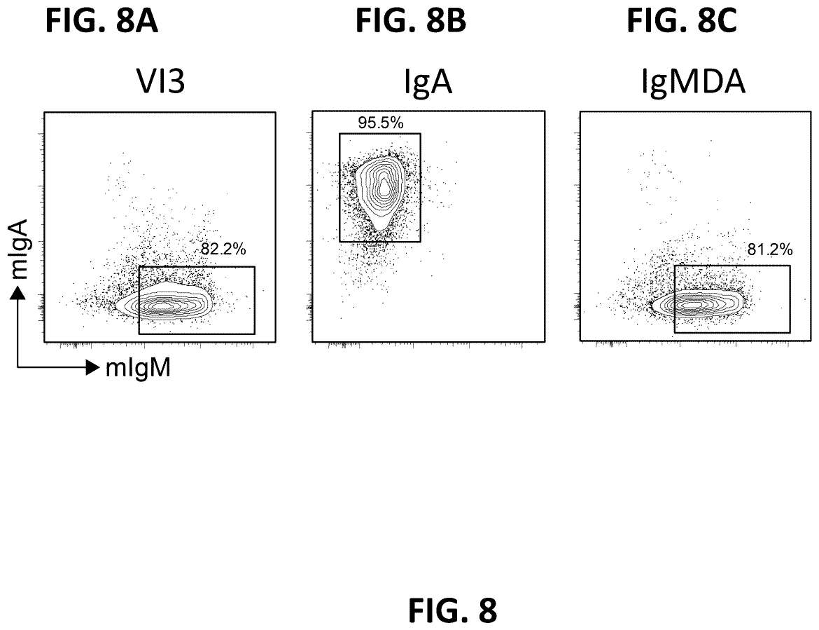

FIGS. 8A-C show representative flow plots for IgM+ and IgA+ B cells in VI3 mice (FIG. 8A), IgA mice (FIG. 8B), and IgMDA mice (FIG. 8C).

Collectively, these results indicate that engineering mice at their immunoglobulin heavy chain locus to remove particular constant region gene segments, thereby producing mice that only produce certain antibody isotypes, does not affect (i) the % and/or ratio of splenic T and B cells (FIGS. 6A-6F) and (ii) the % and/or ratio of antibodies with lambda vs kappa light chains (FIGS. 7A-7F). However, the IgA mice were shown to produce primarily IgA+ B cells, compared to VI3 and IgMDA, which were shown to produce predominantly IgM+ B cells (FIGS. 8A-8C 8).

### Example 6: Characterization of Immune Response in IgA and IgMDA Mice

The present Example examines immune system and antibody repertoire of exemplary IgA and IgMDA genetically modified rodents (e.g., mice) in response to antigen stimulation.

Methods: Titer of IgA antibodies specific for the viral antigen was detected by ELISA. Specifically, ELISA plates were coated with 2 μg/ml viral antigen protein that was used to immunize the mice. Plates were then washed and blocked with 1% BSA in PBS for 2 h at room temperature. Diluted serum samples from immunized mice were added and incubated overnight at 4° C. Captured viral antigen-specific IgA antibodies were detected by horseradish peroxidase (HRP)-conjugated goat anti-mouse IgA antibody (Sigma-Aldrich). ELISA plates were then developed by TMB microwell peroxidase substrate (KPL, Inc.) and quenched by 1 M H2SO4. Colorimetric reaction was measured at OD=450 nm by a Synergy H1 Multi-Mode Microplate Reader (BioTek Instruments, Inc.). Two times background OD value was used as cutoff to calculate titer.

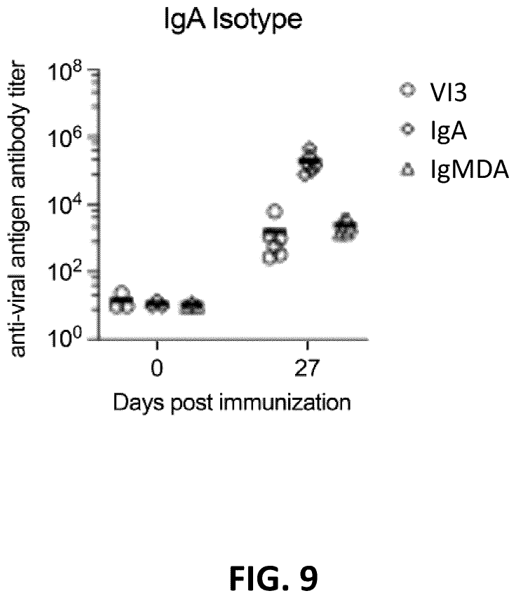

Results: FIG. 9 shows exemplary anti-viral antigen antibody titers with IgA isotype detected in serum of VI3, IgA, and IgMDA mice 27 days post immunization with viral antigen. These results show that the IgA mice had a higher titer of viral antigen-specific IgA antibodies than the VI3 or IgMDA mice, which had similar titers.

### Example 7: Characterization of Immune Response in IgA Mice after Antigen Stimulation

The present Example examines immune system and antibody repertoire of exemplary IgA genetically modified rodents (e.g., mice) in response to antigen stimulation.

Methods: IgA mice were primed with antigen via foot pad and boosted weekly three consecutive times. Bone marrow cells were harvested, and incubated with monomeric antigen for 30 mins. After washing twice with FACS buffer, cells were incubated with tetrameric antigen for 45 mins and followed by two washes. Incubation with tetramer was repeated once more. Cells were then stained for Live/Dead (Aqua), blocked with anti-Fc antibody and stained with a cocktail of antibodies that remove neutrophils, T cells, monocytes, basophils, and NK cells, and with anti-mouse CD19, anti-mouse CD138, anti-mouse CD267 (TACI), and anti-mouse IgA. After three washes, cells were fixed and analyzed with by flow cytometry (Symphony A3, BD). Plasma cells were gated as Live/Dead⁻(neutrophils, T cells, monocytes, basophils, and NK cell)⁻CD19⁻ CD138⁺CD267⁺. Antigen positive (Ag+) plasma cells were further gated as monomeric Ag+ cells among all plasma cells. All antigen positive plasma cells were checked to express surface IgA isotype.

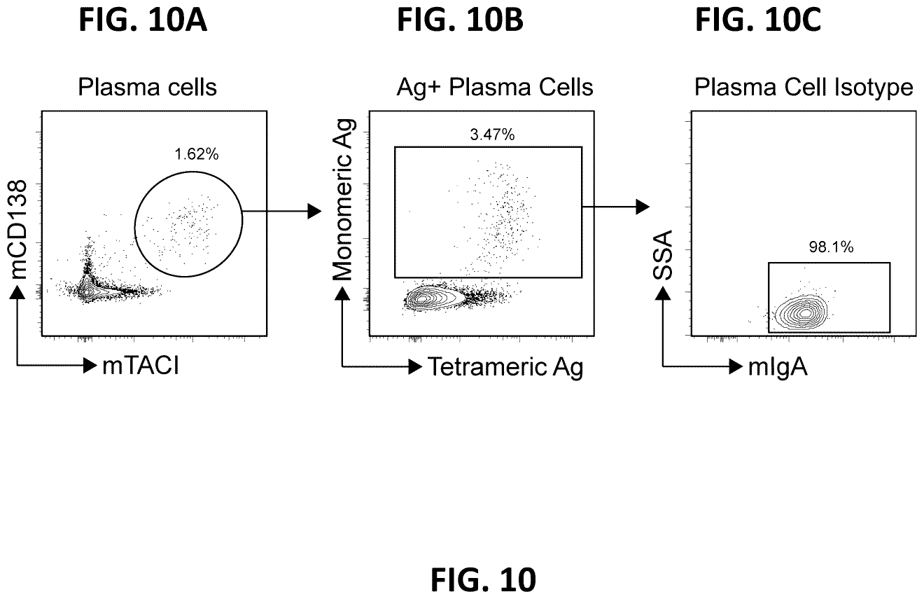

Results: FIGS. 10A-10C show representative flow plots of the plasma cell subpopulations obtained from bone marrow of IgA mice immunized with antigen and analyzed via flow cytometry. Specifically, FIG. 10A shows population of plasma cells (CD19⁻ CD138⁺CD267⁺). FIG. 10B shows the subpopulation of plasma cells that are antigen-specific (Ag+) plasma cells. FIG. 10C shows the subpopulation of Ag+ plasma cells that expressed surface IgA isotype. These results indicate that after immunization, IgA mice develop antigen-positive (Ag+) IgA plasma cells in bone marrow. Accordingly, the present disclosure provides genetically modified non-human animals that produce plasma cells that express IgA antibodies that are Ag+, and that Ag+ antibodies may be isolated from plasma cells produced by the genetically modified non-human animals.

### CERTAIN EMBODIMENTS

Embodiment 1. A genetically modified rodent whose germline genome comprises an engineered endogenous immunoglobulin heavy chain locus that comprises an immunoglobulin heavy chain constant region comprising a rodent immunoglobulin heavy chain alpha (Igha) constant region gene,
wherein the genetically modified rodent only produces immunoglobulin heavy chains that comprise a rodent Igha constant domain encoded by the rodent Igha constant region gene.

Embodiment 2. The genetically modified rodent of embodiment 1, wherein the rodent's germline genome further comprises one or more genetic alterations at the endogenous immunoglobulin heavy chain locus, wherein the one or more genetic alterations disrupt expression from rodent immunoglobulin heavy chain constant region genes: mu (Ighm), delta (Ighd), gamma (Ighg), and epsilon (Ighe) constant region genes.

Embodiment 3. The genetically modified rodent of embodiment 2, wherein the Ighg constant region gene comprises gamma 3 (Ighg3), gamma 1 (Ighg1), gamma 2a (Ighg2a), and gamma 2b (Ighg2b) constant region genes.

Embodiment 4. The genetically modified rodent of embodiment 2, wherein the Ighg constant region gene comprises gamma 3 (Ighg3), gamma 1 (Ighg1), gamma 2c (Ighg2c), and gamma 2b (Ighg2b) constant region genes.

Embodiment 5. The genetically modified rodent of any one of embodiments 2-4, wherein the one or more genetic alterations comprise one or more deletions, insertions, mutations, and/or inversions.

Embodiment 6. The genetically modified rodent of any one of embodiments 2-5, wherein the one or more genetic alterations comprises one or more deletions.

Embodiment 7. The genetically modified rodent of embodiment 5 or 6, wherein the one or more deletions remove all or part of each of the rodent immunoglobulin heavy chain constant region genes: Ighm, Ighd, Ighg, and Ighe constant region genes.

Embodiment 8. The genetically modified rodent of any one of embodiments 5-7, wherein the one or more deletions remove part of each of the rodent immunoglobulin heavy chain constant region genes: Ighm, Ighd, Ighg, and Ighe constant region genes.

Embodiment 9. The genetically modified rodent of any one of embodiments 5-7, wherein the one or more deletions remove all of the rodent immunoglobulin heavy chain constant region genes: Ighm, Ighd, Ighg, and Ighe constant region genes.

Embodiment 10. The genetically modified rodent of embodiment 9, wherein the one or more deletions remove a contiguous sequence spanning a region that is upstream of the rodent Ighm gene to a region that is upstream of the rodent Igha gene.

Embodiment 11. The genetically modified rodent of any one of embodiments 5-9, wherein the one or more deletions do not remove all or a part of the non-coding region between the rodent immunoglobulin heavy chain constant region genes: Ighm, Ighd, Ighg, and/or Ighe constant region genes.

Embodiment 12. The genetically modified rodent of any one of embodiments 5-11, wherein the one or more genetic alterations comprise one or more insertions.

Embodiment 13. The genetically modified rodent of embodiment 12, wherein the one or more insertions comprise an insertion of one or more repressor elements that inhibit expression of the rodent immunoglobulin heavy chain constant region genes: Ighm, Ighd, Ighg, and Ighe constant region genes.

Embodiment 14. The genetically modified rodent of any one of embodiments 5-13, wherein the one or more genetic alterations comprise one or more mutations.

Embodiment 15. The genetically modified rodent of embodiment 14, wherein the one or more mutations comprises a frameshift mutation that inhibits expression of rodent immunoglobulin heavy chain constant region genes: Ighm, Ighd, Ighg, and Ighe constant region genes.

Embodiment 16. The genetically modified rodent of any one of embodiments 1-15, wherein the engineered endogenous immunoglobulin heavy chain locus comprises in 5′ to 3′ order:
(i) a rodent intronic enhancer (Eμ);
(ii) a rodent switch mu region (Sμ); and
(iii) a rodent Igha constant region gene.

Embodiment 17. The genetically modified rodent of embodiment 16, wherein the engineered endogenous immunoglobulin heavy chain locus comprises a rodent 3′ IgH regulatory region (3′RR) that is 3′ of the rodent Igha constant region gene.

Embodiment 18. A genetically modified rodent whose germline genome comprises an engineered endogenous immunoglobulin heavy chain locus that comprises an immunoglobulin heavy chain constant region comprising rodent immunoglobulin heavy chain constant region genes Ighm, Ighd, and Igha,
wherein the genetically modified rodent only produces immunoglobulin heavy chains that comprise a rodent Igha, a rodent Ighm, or a rodent Ighd constant domain encoded by the rodent Igha constant region gene, the rodent Ighm constant region gene, or the rodent Ighd constant region gene, respectively.

Embodiment 19. The genetically modified rodent of embodiment 18, wherein the rodent's germline genome further comprises one or more genetic alterations at the endogenous immunoglobulin heavy chain locus, wherein the one or more genetic alterations disrupt expression from rodent immunoglobulin heavy chain constant region genes: Ighg and Ighe constant region genes.

Embodiment 20. The genetically modified rodent of embodiment 19, wherein the Ighg constant region gene comprises gamma 3 (Ighg3), gamma 1 (Ighg1), gamma 2a (Ighg2a), and gamma 2b (Ighg2b) constant region genes.

Embodiment 21. The genetically modified rodent of embodiment 19, wherein Ighg constant region gene comprises gamma 3 (Ighg3), gamma 1 (Ighg1), gamma 2c (Ighg2c), and gamma 2b (Ighg2b) constant region genes.

Embodiment 22. The genetically modified rodent of any one of embodiments 19-21, wherein the one or more genetic alterations comprise one or more deletions, insertions, mutations, and/or inversions.

Embodiment 23. The genetically modified rodent of any one of embodiments 19-22, wherein the one or more genetic alterations comprises one or more deletions.

Embodiment 24. The genetically modified rodent of embodiment 22 or 23, wherein the one or more deletions remove all or part of each of the rodent immunoglobulin heavy chain constant region genes: Ighg and Ighe constant region genes.

Embodiment 25. The genetically modified rodent of any one of embodiments 22-24, wherein the one or more deletions remove part of each of the rodent immunoglobulin heavy chain constant region genes: Ighg and Ighe constant region genes.

Embodiment 26. The genetically modified rodent of any one of embodiments 22-24, wherein the one or more deletions remove all of the rodent immunoglobulin heavy chain constant region genes: Ighg and Ighe constant region genes.

Embodiment 27. The genetically modified rodent of embodiment 26, wherein the one or more deletions remove a contiguous sequence spanning a region that is upstream of the Ighg constant region gene to a region that is upstream of the rodent Igha constant region gene.

Embodiment 28. The genetically modified rodent of any one of embodiments 22-26, wherein the one or more deletions do not remove one or more of the non-coding sequences adjacent to the rodent immunoglobulin heavy chain constant region genes: Ighg and Ighe constant region genes.

Embodiment 29. The genetically modified rodent of any one of embodiments 22-28, wherein the one or more genetic alterations comprise one or more insertions.

Embodiment 30. The genetically modified rodent of embodiment 29, wherein the one or more insertions comprise an insertion of one or more repressor elements that inhibit expression of the rodent immunoglobulin heavy chain constant region genes: Ighg and Ighe constant region genes.

Embodiment 31. The genetically modified rodent of any one of embodiments 22-30, wherein the one or more genetic alterations comprise one or more mutations.

Embodiment 32. The genetically modified rodent of embodiment 31, wherein the one or more mutations comprise a frameshift mutation that inhibits expression of rodent immunoglobulin heavy chain constant region genes: Ighg and Ighe constant region genes.

Embodiment 33. The genetically modified rodent of any one of embodiments 18-32, wherein the engineered endogenous immunoglobulin heavy chain locus comprises in 5′ to 3′ order:
(i) a rodent intronic enhancer (Eμ);
(ii) a rodent switch mu region (Sμ);
(iii) a rodent Ighm constant region gene;
(iv) a rodent Ighd constant region gene;
(v) a rodent switch alpha region (Sα); and
(vi) a rodent Igha constant region gene.

Embodiment 34. The genetically modified rodent of embodiment 33, wherein the engineered endogenous immunoglobulin heavy chain locus comprises a rodent 3′ IgH regulatory region 3′RR that is 3′ of the rodent Igha constant region gene.

Embodiment 35. The genetically modified rodent of any one of embodiments 1-34, wherein the engineered endogenous immunoglobulin heavy chain locus further comprises one or more rodent V_{H} gene segments, one or more rodent D_{H} gene segments, and one or more rodent J_{H} gene segments;
wherein the rodent V_{H}, D_{H}, and J_{H} gene segments are operably linked to the rodent immunoglobulin heavy chain constant region.

Embodiment 36. The genetically modified rodent of any one of embodiments 1-34, wherein the engineered endogenous immunoglobulin heavy chain locus comprises one or more non-rodent mammal V_{H} gene segments, one or more non-rodent mammal D_{H} gene segments, and one or more non-rodent mammal J_{H} gene segments;
wherein the non-rodent mammal V_{H}, D_{H}, and J_{H} gene segments are operably linked to the rodent immunoglobulin heavy chain constant region.

Embodiment 37. The genetically modified rodent of embodiment 36, wherein the non-rodent mammal is a cat, dog, cow, horse, primate, or human.

Embodiment 38. The genetically modified rodent of embodiment 37, wherein the one or more non-rodent mammal V_{H} gene segments, one or more non-rodent mammal D_{H} gene segments, and one or more non-rodent mammal J_{H} gene segments are or comprise one or more human V_{H} gene segments, one or more human D_{H} gene segments, and one or more human J_{H} gene segments.

Embodiment 39. The genetically modified rodent of embodiment 38, wherein the engineered endogenous immunoglobulin heavy chain locus comprises a contiguous human sequence that spans the one or more human V_{H} gene segments, one or more human D_{H} gene segments, and one or more human J_{H} gene segments;
wherein the contiguous human sequence is operably linked to the rodent immunoglobulin heavy chain constant region.

Embodiment 40. The genetically modified rodent of embodiment 38, wherein the engineered endogenous immunoglobulin heavy chain locus further comprises:
(i) one or more rodent V_{H} non-coding sequences, each of which is adjacent to the one or more human V_{H} gene segments, wherein the one or more V_{H} non-coding sequences naturally appears adjacent to a rodent V_{H} gene segment in an endogenous rodent immunoglobulin locus;
(ii) one or more rodent D_{H} non-coding sequences, each of which is adjacent to the one or more human D_{H} gene segments, wherein the one or more D_{H} non-coding sequences naturally appears adjacent to a rodent D_{H} gene segment in an endogenous rodent immunoglobulin locus;
(iii) one or more rodent J_{H} non-coding sequences, each of which is adjacent to the one or more human J_{H} gene segments, wherein the one or more J_{H} non-coding sequences naturally appears adjacent to a rodent J_{H} gene segment in an endogenous rodent immunoglobulin locus; or
(iv) any combination thereof.

Embodiment 41. The genetically modified rodent of any one of embodiments 38-40, wherein the one or more human V_{H} gene segments, one or more human D_{H} gene segments, and one or more human J_{H} gene segments are operably linked to the rodent immunoglobulin heavy chain constant region at the endogenous rodent immunoglobulin heavy chain locus.

Embodiment 42. The genetically modified rodent of any one of embodiments 1-17 and 36-41, wherein the engineered endogenous immunoglobulin heavy chain locus comprises in 5′ to 3′ order:
(i) one or more human V_{H} gene segments, one or more human D_{H} gene segments, and one or more human J_{H} gene segments;
(ii) a rodent intronic enhancer (Eμ);
(iii) a rodent switch mu region (Sμ);
(iv) a rodent Igha constant region gene; and optionally (v) a rodent 3′ IgH regulatory region 3′RR.

Embodiment 43. The genetically modified rodent of any one of embodiments 18-34 and 36-41, wherein the engineered endogenous immunoglobulin heavy chain locus comprises in 5′ to 3′ order:
(i) one or more human V_{H} gene segments, one or more human D_{H} gene segments, and one or more human J_{H} gene segments;
(ii) a rodent intronic enhancer (Eμ);
(iii) a rodent switch mu region (Sμ);
(iv) a rodent Ighm constant region gene;
(v) a rodent Ighd constant region gene;
(vi) a rodent switch alpha region (Sα);
(vii) a rodent Igha constant region gene; and
(viii) optionally a rodent 3′ IgH regulatory region 3′RR.

Embodiment 44. The genetically modified rodent of any one of embodiments 38-43, wherein the one or more human V_{H} gene segments, one or more human D_{H} gene segments, and one or more human J_{H} gene segments replace one or more rodent V_{H} gene segments, one or more rodent D_{H} gene segments, one or more rodent J_{H} gene segments, or any combination thereof.

Embodiment 45. The genetically modified rodent of any one of embodiments 38-43, wherein the one or more human V_{H} gene segments, the one or more human D_{H} gene segments, and the one or more human J_{H} gene segments are inserted downstream of one or more endogenous rodent V_{H} gene segments, one or more rodent D_{H} gene segments, one or more rodent J_{H} gene segments, or any combination thereof; and wherein the one or more endogenous rodent V_{H} gene segments, one or more rodent D_{H} gene segments, one or more rodent J_{H} gene segments, or any combination thereof are non-functional.

Embodiment 46. The genetically modified rodent of any one of embodiments 1-45, wherein the rodent is a mouse.

Embodiment 47. The genetically modified rodent of any one of embodiments 1-45, wherein the rodent is a rat.

Embodiment 48. The genetically modified rodent of any one of embodiments 1-47, wherein the rodent is homozygous for the engineered endogenous immunoglobulin heavy chain locus.

Embodiment 49. The genetically modified rodent of any one of embodiments 1-47, wherein the rodent is heterozygous for the engineered endogenous immunoglobulin heavy chain locus.

Embodiment 50. The genetically modified rodent of any one of embodiments 1-49, wherein the rodent Igha constant region gene is an endogenous rodent Igha constant region gene.

Embodiment 51. The genetically modified rodent of embodiment 50, wherein the rodent Igha constant region gene is an endogenous mouse Igha constant region gene.

Embodiment 52. The genetically modified rodent of embodiment 51, wherein the endogenous mouse Igha constant region gene comprises as Igha*01 allele.

Embodiment 53. The genetically modified rodent of embodiment 51, wherein the endogenous mouse Igha constant region gene comprises as Igha*02 allele.

Embodiment 54. The genetically modified rodent of any one of embodiments 38-53, wherein the one or more human V_{H} gene segments comprise V_{H}3-74, V_{H}3-73, V_{H}3-72, V_{H}2-70, V_{H}1-69, V_{H}3-66, V_{H}3-64, V_{H}4-61, V_{H}4-59, V_{H}1-58, V_{H}3-53, V_{H}5-51, V_{H}3-49, V_{H}3-48, V_{H}1-46, V_{H}1-45, V_{H}3-43, V_{H}4-39, V_{H}4-34, V_{H}3-33, V_{H}4-31, V_{H}3-30, V_{H}4-28, V_{H}2-26, V_{H}1-24, V_{H}3-23, V_{H}3-21, V_{H}3-20, V_{H}1-18, V_{H}3-15, V_{H}3-13, V_{H}3-11, V_{H}3-9, V_{H}1-8, V_{H}3-7, V_{H}2-5, V_{H}7-4-1, V_{H}4-4, V_{H}1-3, V_{H}1-2, V_{H}6-1, or any combination thereof.

Embodiment 55. The genetically modified rodent of any one of embodiments 38-54, wherein the one or more human D_{H} gene segments comprise D_{H}1-1, D_{H}2-2, D_{H}3-3, D_{H}4-4, D_{H}5-5, D_{H}6-6, D_{H}1-7, D_{H}2-8, D_{H}3-9, D_{H}3-10, D_{H}5-12, D_{H}6-13, D_{H}2-15, D_{H}3-16, D_{H}4-17, D_{H}6-19, D_{H}1-20, D_{H}2-21, D_{H}3-22, D_{H}6-25, D_{H}1-26, D_{H}7-27, or any combination thereof.

Embodiment 56. The genetically modified rodent of any one of embodiments 38-55, wherein the one or more human J_{H} gene segments comprise: J_{H}1, J_{H}2, J_{H}3, J_{H}4, J_{H}5, J_{H}6, or any combination thereof.

Embodiment 57. The genetically modified rodent of any one of embodiments 38-56, wherein the one or more human V_{H} gene segments, one or more human D_{H} gene segments, and one or more human J_{H} gene segments comprise all functional human V_{H} gene segments, all functional human D_{H} gene segments, and all functional human J_{H} gene segments.

Embodiment 58. The genetically modified rodent of any one of embodiments 38-56, wherein the one or more human V_{H} gene segments, one or more human D_{H} gene segments, and one or more human J_{H} gene segments comprises 80 V_{H} gene segments, all human D_{H} gene segments, and all human J_{H} gene segments.

Embodiment 59. The genetically modified rodent of any one of embodiments 38-58, wherein the one or more human V_{H} gene segments, one or more human D_{H} gene segments, and one or more human J_{H} gene segments comprises all V_{H} gene segments, all human D_{H} gene segments, and all human J_{H} gene segments.

Embodiment 60. The genetically modified rodent of any one of embodiments 1-59, wherein the genetically modified rodent is heterozygous for the engineered endogenous immunoglobulin heavy chain locus.

Embodiment 61. The genetically modified rodent of any one of embodiments 1-59, wherein the genetically modified rodent is hemizygous for the engineered endogenous immunoglobulin heavy chain locus.

Embodiment 62. The genetically modified rodent of any one of embodiments 1-59, wherein the genetically modified rodent is homozygous for the engineered endogenous immunoglobulin heavy chain locus.

Embodiment 63. The genetically modified rodent of any one of embodiments 1-62, wherein the engineered endogenous immunoglobulin heavy chain locus lacks a functional endogenous rodent Adam6 gene.

Embodiment 64. The genetically modified rodent of any one of embodiments 1-63, wherein the germline genome further comprises one or more nucleotide sequences encoding one or more rodent ADAM6 polypeptides, functional orthologs, functional homologs, or functional fragments thereof.

Embodiment 65. The genetically modified rodent of embodiment 64, wherein the one or more rodent ADAM6 polypeptides, functional orthologs, functional homologs, or functional fragments thereof are expressed.

Embodiment 66. The genetically modified rodent of embodiment 64 or 65, wherein the one or more nucleotide sequences encoding one or more rodent ADAM6 polypeptides, functional orthologs, functional homologs, or functional fragments thereof are included on the same chromosome as the engineered endogenous immunoglobulin heavy chain locus.

Embodiment 67. The genetically modified rodent of any one of embodiments 64-66, wherein the one or more nucleotide sequences encoding one or more rodent ADAM6 polypeptides, functional orthologs, functional homologs, or functional fragments thereof are included in the engineered endogenous immunoglobulin heavy chain locus.

Embodiment 68. The genetically modified rodent of any one of embodiments 64-67, wherein the one or more nucleotide sequences encoding one or more rodent ADAM6 polypeptides, functional orthologs, functional homologs, or functional fragments thereof are in place of a human Adam6 pseudogene.

Embodiment 69. The genetically modified rodent of any one of embodiments 64-68, wherein the one or more nucleotide sequences encoding one or more rodent ADAM6 polypeptides, functional orthologs, functional homologs, or functional fragments thereof replace a human Adam6 pseudogene.

Embodiment 70. The genetically modified rodent of any one of embodiments 64-69, wherein the one or more nucleotide sequences encoding one or more rodent ADAM6 polypeptides, functional orthologs, functional homologs, or functional fragments thereof are between a first human V_{H} gene segment and a second human V_{H} gene segment.

Embodiment 71. The genetically modified rodent of embodiment 70, wherein the first human V_{H} gene segment is V_{H}1-2 and the second human V_{H} gene segment is V_{H}6-1.

Embodiment 72. The genetically modified rodent of any one of embodiments 64-69, wherein the one or more nucleotide sequences encoding one or more rodent ADAM6 polypeptides, functional orthologs, functional homologs, or functional fragments thereof are between a human V_{H} gene segment and a human D_{H} gene segment.

Embodiment 73. The genetically modified rodent of any one of embodiments 1-72, wherein the germline genome of the rodent comprises an engineered endogenous immunoglobulin light chain locus comprising:
(a) one or more rodent or non-rodent mammal Vκ gene segments, and
(b) one or more rodent or non-rodent mammal Jκ gene segments,
wherein the one or more rodent or non-rodent mammal Vκ gene segments and the one or more rodent or non-rodent mammal Jκ gene segments are operably linked to a Cκ gene.

Embodiment 74. The genetically modified rodent of embodiments 73, wherein the non-rodent mammal is a cat, dog, cow, horse, primate, or human.

Embodiment 75. The genetically modified rodent of embodiment 74, wherein the one or more non-rodent mammal Vκ gene segments and the one or more non-rodent mammal Jκ gene segments are or comprise one or more human Vκ gene segments (e.g., Vκ3D-7, Vκ1D-8, Vκ1D-43, Vκ3D-11, Vκ3D-12, Vκ3D-13, Vκ3D-15, Vκ3D-16, Vκ3D-17, Vκ3D-20, Vκ6D-21, Vκ2D-26, Vκ2D-28, Vκ2D-29, Vκ2D-30, Vκ1D-33, Vκ1D-39, Vκ2D-40, Vκ2-40, Vκ1-39, Vκ1-33, Vκ2-33, Vκ2-29, Vκ2-28, Vκ1-27, Vκ2-24, Vκ6-21, Vκ3-20, Vκ1-17, Vκ1-16, Vκ3-15, Vκ1-13, Vκ1-12, Vκ3-11, Vκ1-9, Vκ1-8, Vκ1-6, Vκ1-5, Vκ5-2, and/or Vκ4-1 gene segment) and one or more human Jκ gene segments (e.g., Jκ1, Jκ2, Jκ3, Jκ4, and/or Jκ5 gene segment).

Embodiment 76. The genetically modified rodent of embodiment 75, wherein the engineered endogenous immunoglobulin light chain locus comprises a contiguous human sequence that spans the one or more human Vκ gene segments and one or more human Jκ gene segments;
wherein the contiguous human sequence spanning the one or more human Vκ gene segments and one or more human Jκ gene segments is operably linked to the endogenous rodent immunoglobulin light chain Cκ gene.

Embodiment 77. The genetically modified rodent of embodiment 75, wherein the engineered endogenous immunoglobulin light chain locus further comprises:
(i) one or more rodent Vκ non-coding sequences, each of which is adjacent to the one or more human Vκ gene segments, wherein the one or more Vκ non-coding sequences naturally appears adjacent to a rodent Vκ gene segment in an endogenous rodent immunoglobulin locus;
(ii) one or more rodent Jκ non-coding sequences, each of which is adjacent to the one or more human Jκ gene segments, wherein the one or more Jκ non-coding sequences naturally appears adjacent to a rodent Jκ gene segment in an endogenous rodent immunoglobulin locus; or
(iii) any combination thereof.

Embodiment 78. The genetically modified rodent of any one of embodiments 73-77, wherein the Cκ gene is a rodent Cκ gene.

Embodiment 79. The genetically modified rodent of embodiment 78, wherein the Cκ gene is an endogenous rodent Cκ gene.

Embodiment 80. The genetically modified rodent of any one of embodiments 75-79, wherein the one or more human Vκ gene segments and the one or more human Jκ gene segments replace one or more rodent Vκ gene segments and one or more rodent Jκ gene segments.

Embodiment 81. The genetically modified rodent of any one of embodiments 75-80, wherein the one or more human Vκ gene segments and the one or more human Jκ gene segments are inserted downstream of one or more endogenous rodent Vκ gene segments and one or more endogenous rodent Jκ gene segments; and wherein the one or more endogenous rodent Vκ gene and the one or more endogenous rodent Jκ gene segments are non-functional.

Embodiment 82. The genetically modified rodent of any one of embodiments 73-81, wherein the rodent is homozygous for the engineered endogenous immunoglobulin light chain locus.

Embodiment 83. The genetically modified rodent of any one of embodiments 73-81, wherein the rodent is heterozygous for the engineered endogenous immunoglobulin light chain locus.

Embodiment 84. The genetically modified rodent of any one of embodiments 73-81, further comprising an inactivated endogenous immunoglobulin κ light chain locus.

Embodiment 85. The genetically modified rodent of embodiment 84, wherein the genetically modified rodent is heterozygous for the inactivated endogenous immunoglobulin κ light chain locus.

Embodiment 86. The genetically modified rodent of embodiment 84, wherein the genetically modified rodent is homozygous for the inactivated endogenous immunoglobulin κ light chain locus.

Embodiment 87. The genetically modified rodent of any one of embodiments 73-86, wherein the endogenous Vκ gene segments and the endogenous Jκ gene segments are deleted in whole or in part.

Embodiment 88. The genetically modified rodent of any one of embodiments 73-87, wherein the rodent does not detectably express endogenous immunoglobulin κ light chain variable domains.

Embodiment 89. The genetically modified rodent of any one of embodiments 75-88, wherein the one or more human Vκ gene segments and the one or more human Jκ gene segments comprise 40 Vκ gene segments and all human Jκ gene segments.

Embodiment 90. The genetically modified rodent of any one of embodiments 75-89, wherein the one or more human Vκ gene segments and the one or more human Jκ gene segments comprise all functional human Vκ gene segments and all functional human Jκ gene segments.

Embodiment 91. The genetically modified rodent of any one of embodiments 75-90, wherein the one or more human Vκ gene segments and the one or more human Jκ gene segments comprise all human Vκ gene segments and all human Jκ gene segments.

Embodiment 92. The genetically modified rodent of any one of embodiments 75-88, wherein the one or more human Vκ gene segments comprises a single human Vκ gene segment and one or more human Jκ gene segments comprises a single human Jκ gene segment.

Embodiment 93. The genetically modified rodent of embodiment 92, wherein the single human Vκ gene segment and the single Jκ gene segment comprise a single rearranged Vκ/Jκ gene sequence.

Embodiment 94. The genetically modified rodent of embodiment 93, wherein all immunoglobulin κ light chains expressed by B cells of the genetically modified rodent include human immunoglobulin κ light chain variable domains expressed from the single rearranged human immunoglobulin κ light chain variable region or a somatically hypermutated version thereof.

Embodiment 95. The genetically modified rodent of any one of embodiments 1-94, wherein the germline genome of the rodent comprises an engineered endogenous immunoglobulin light chain locus comprising:
(a) one or more rodent or non-rodent mammal Vλ gene segments, and
(b) one or more rodent or non-rodent mammal Jλ gene segments,
wherein the one or more rodent or non-rodent mammal Vλ gene segments and one or more rodent or non-rodent mammal Jλ gene segments are operably linked to a rodent immunoglobulin light chain constant region gene.

Embodiment 96. The genetically modified rodent of embodiment 95, wherein the non-rodent mammal is a cat, dog, cow, horse, primate, or human.

Embodiment 97. The genetically modified rodent of embodiment 96, wherein the one or more non-rodent mammal Vλ gene segments and the one or more non-rodent mammal Jλ gene segments are or comprise one or more human Vλ gene segments and one or more human Jλ gene segments.

Embodiment 98. The genetically modified rodent of embodiment 97, wherein the engineered endogenous immunoglobulin light chain locus comprises a contiguous human sequence that spans the one or more human mammal Vλ gene segments and one or more human Jλ gene segments;
wherein the contiguous human sequence is operably linked to the endogenous rodent immunoglobulin light chain gene.

Embodiment 99. The genetically modified rodent of embodiment 97, wherein the engineered endogenous immunoglobulin light chain locus further comprises:
(i) one or more rodent Vλ non-coding sequences, each of which is adjacent to the one or more human Vλ gene segments, wherein the one or more Vλ non-coding sequences naturally appears adjacent to a rodent Vλ gene segment in an endogenous rodent immunoglobulin locus;
(ii) one or more rodent Jλ non-coding sequences, each of which is adjacent to the one or more human Jλ gene segments, wherein the one or more Jκ non-coding sequences naturally appears adjacent to a rodent Jλ gene segment in an endogenous rodent immunoglobulin locus; or
(iii) any combination thereof.

Embodiment 100. The genetically modified rodent of any one of embodiments 95-99, wherein the endogenous rodent immunoglobulin light chain constant region gene is a rodent Cλ gene.

Embodiment 101. The genetically modified rodent of any one of embodiments 95-99, wherein the endogenous rodent immunoglobulin light chain constant region gene is a rodent Cκ gene.

Embodiment 102. The genetically modified rodent of any one of embodiments 1-101, wherein the engineered endogenous immunoglobulin light chain locus is an engineered endogenous λ immunoglobulin light chain locus.

Embodiment 103. The genetically modified rodent of any one of embodiments 1-101, wherein the engineered endogenous immunoglobulin light chain locus is an engineered endogenous κ immunoglobulin light chain locus.

Embodiment 104. The genetically modified rodent of any one of embodiments 97-103, wherein the one or more human Vλ gene segments and the one or more human Jλ gene segments replace one or more rodent Vλ gene segments and one or more rodent Jλ gene segments.

Embodiment 105. The genetically modified rodent of any one of embodiments 97-103, wherein the one or more human Vλ gene segments and the one or more human Jλ gene segments are inserted downstream of one or more endogenous rodent Vλ gene segments and one or more endogenous rodent Jλ gene segments; and wherein the one or more endogenous rodent Vλ gene and the one or more endogenous rodent Jλ gene segments are non-functional.

Embodiment 106. The genetically modified rodent of any one of embodiments 95-105, wherein the rodent is homozygous for the engineered endogenous immunoglobulin light chain locus.

Embodiment 107. The genetically modified rodent of any one of embodiments 95-105, wherein the rodent is heterozygous for the engineered endogenous immunoglobulin light chain locus.

Embodiment 108. The genetically modified rodent of any one of embodiments 95-107, further comprising an inactivated endogenous immunoglobulin λ light chain locus.

Embodiment 109. The genetically modified rodent of embodiment 108, wherein the genetically modified rodent is heterozygous for the inactivated endogenous immunoglobulin λ light chain locus.

Embodiment 110. The genetically modified rodent of embodiment 108, wherein the genetically modified rodent is homozygous for the inactivated endogenous immunoglobulin λ light chain locus.

Embodiment 111. The genetically modified rodent of any one of embodiments 95-110, wherein the endogenous rodent Vλ gene segments and Jλ gene segments are deleted in whole or in part.

Embodiment 112. The genetically modified rodent of any one of embodiment 95-111, wherein the rodent does not detectably express endogenous immunoglobulin λ light chain variable domains.

Embodiment 113. The genetically modified rodent of any one of embodiments 97-112, wherein:
(i) the one or more human Vλ gene segments comprise Vλ5-52, Vλ1-51, Vλ9-49, Vλ1-47, Vλ7-46, Vλ5-45, Vλ1-44, Vλ7-43, Vλ1-40, Vλ5-39, Vλ5-37, Vλ1-36, Vλ3-27, Vλ3-25, Vλ2-23, Vλ3-22, Vλ3-21, Vλ3-19, Vλ3-16, Vλ2-14, Vλ3-12, Vλ2-11, Vλ3-10, Vλ3-9, Vλ2-8, Vλ4-3, Vλ3-1, or any combination thereof; and
(ii) the one or more human Jλ gene segments comprise Jλ1, Jλ2, Jλ3, Jλ6, Jλ7, or any combination thereof.

Embodiment 114. The genetically modified rodent of embodiment 113, wherein the one or more human Vλ gene segments comprise at least 12 human Vλ gene segments.

Embodiment 115. The genetically modified rodent of embodiment 113, wherein the one or more human Vλ gene segments comprise at least 28 human Vλ gene segments.

Embodiment 116. The genetically modified rodent of embodiment 113, wherein the one or more human Vλ gene segments comprise at least 40 human Vλ gene segments.

Embodiment 117. The genetically modified rodent of any one of embodiments 97-116, wherein the one or more human Vλ gene segments and the one or more human Jλ gene segments comprise all functional human Vλ gene segments and all functional human Jλ gene segments.

Embodiment 118. The genetically modified rodent of any one of embodiments 103-105, wherein the one or more human Vλ gene segments comprises a single human Vλ gene segment and one or more human Jλ gene segments comprises a single human Jλ gene segment.

Embodiment 119. The genetically modified rodent of embodiment 118, wherein the engineered endogenous immunoglobulin light chain locus comprises a single rearranged human immunoglobulin λ light chain variable region.

Embodiment 120. The genetically modified rodent of embodiment 119, wherein the single rearranged human immunoglobulin λ light chain variable region is operably linked to a rodent Cλ gene segment at the endogenous immunoglobulin κ light chain locus.

Embodiment 121. The genetically modified rodent of embodiment 120, wherein all immunoglobulin λ light chains expressed by B cells of the genetically modified rodent include human immunoglobulin λ light chain variable domains expressed from the single rearranged human immunoglobulin λ light chain variable region or a somatically hypermutated version thereof.

Embodiment 122. A method of making a genetically modified rodent, the method comprising genetically modifying the germline genome of a rodent so that the rodent comprises an immunoglobulin heavy chain constant region comprising a rodent Igha constant region gene and only produces immunoglobulin heavy chains that comprise a rodent Igha constant domain encoded by the rodent Igha constant region gene.

Embodiment 123. The method of embodiment 122, wherein modifying the germline genome of the rodent comprises introducing one or more genetic alterations at the endogenous immunoglobulin heavy chain locus, wherein the one or more genetic alterations disrupt expression from rodent immunoglobulin heavy chain constant region genes: Ighm, Ighd, Ighg, and Ighe constant region genes.

Embodiment 124. The method of embodiment 123, wherein the Ighg constant region gene comprises gamma 3 (Ighg3), gamma 1 (Ighg1), gamma 2a (Ighg2a) and gamma 2b (Ighg2b) constant region genes.

Embodiment 125. The method of embodiment 123, wherein Ighg constant region gene comprises gamma 3 (Ighg3), gamma 1 (Ighg1), gamma 2c (Ighg2c), and gamma 2b (Ighg2b) constant region genes.

Embodiment 126. The method of any one of embodiments 123-125, wherein the one or more genetic alterations comprise one or more deletions, insertions, mutations, and/or inversions.

Embodiment 127. The method of embodiment 126, wherein the one or more genetic alterations comprises one or more deletions.

Embodiment 128. The method of embodiment 126 or 127, wherein the one or more deletions remove all or part of each of the rodent immunoglobulin heavy chain constant region genes: Ighm, Ighd, Ighg, and Ighe constant region genes.

Embodiment 129. The method of any one of embodiments 126-128, wherein the one or more deletions remove part of each of the rodent immunoglobulin heavy chain constant region genes: Ighm, Ighd, Ighg, and Ighe constant region genes.

Embodiment 130. The method of any one of embodiments 126-128, wherein the one or more deletions remove all of each of the rodent immunoglobulin heavy chain constant region genes: Ighm, Ighd, Ighg and Ighe constant region genes.

Embodiment 131. The method of embodiment 130, wherein the one or more deletions remove a contiguous sequence spanning a region that is upstream of the rodent Ighm constant region gene to a region that is upstream of the rodent Igha constant region gene.

Embodiment 132. The method of any one of embodiments 127-130, wherein the one or more deletions do not remove all or a part of the non-coding region between the rodent immunoglobulin heavy chain constant region genes: Ighm, Ighd, Ighg, and Ighe constant region genes.

Embodiment 133. The method of any one of embodiments 126-132, wherein the one or more genetic alterations comprise one or more insertions.

Embodiment 134. The method of embodiment 133, wherein the one or more insertions comprise an insertion of one or more repressor elements that inhibit expression of the rodent immunoglobulin heavy chain constant region genes: Ighm, Ighd, Ighg, and Ighe constant region genes.

Embodiment 135. The method of any one of embodiments 126-134, wherein the one or more genetic alterations comprise one or more mutations.

Embodiment 136. The method of embodiment 135, wherein the one or more mutations comprise one or more frameshift mutations that inhibit expression of rodent immunoglobulin heavy chain constant region genes: Ighm, Ighd, Ighg, and Ighe constant region genes.

Embodiment 137. The method of any one of embodiments 122-136, wherein the engineered endogenous immunoglobulin heavy chain locus comprises in 5′ to 3′ order:
(i) a rodent intronic enhancer (Eμ);
(ii) a rodent switch mu region (Sμ); and
(iii) a rodent immunoglobulin heavy constant alpha (Igha) gene.

Embodiment 138. The method of embodiment 137, wherein the engineered endogenous immunoglobulin heavy chain locus comprises a rodent 3′ IgH regulatory region 3′RR that is 3′ of the rodent Igha constant region gene.

Embodiment 139. The method of any one of embodiments 122-138, wherein the genetically modified rodent is heterozygous for the engineered endogenous immunoglobulin heavy chain locus.

Embodiment 140. The method of any one of embodiments 122-138, wherein the genetically modified rodent is hemizygous for the engineered endogenous immunoglobulin heavy chain locus.

Embodiment 141. The method of any one of embodiments 122-138, wherein the genetically modified rodent is homozygous for the engineered endogenous immunoglobulin heavy chain locus.

Embodiment 142. A method of making a genetically modified rodent, the method comprising genetically modifying the germline genome of a rodent so that the rodent comprises an immunoglobulin heavy chain constant region comprising rodent immunoglobulin heavy chain constant region genes Ighm, Ighd, and Igha, and only produces immunoglobulin heavy chains that comprise a rodent Igha, a rodent Ighm, or a rodent Ighd constant domain encoded by the Igha constant region gene, the rodent Ighm constant region gene, or the rodent Ighd constant region gene, respectively.

Embodiment 143. The method of embodiment 142, wherein the rodent's germline genome further comprises one or more genetic alterations at the endogenous immunoglobulin heavy chain locus, wherein the one or more genetic alterations disrupt expression from rodent immunoglobulin heavy chain constant region genes: Ighg and Ighe constant region genes.

Embodiment 144. The method of embodiment 143, wherein the one or more genetic alterations comprise one or more deletions, insertions, mutations, and/or inversions.

Embodiment 145. The method of embodiment 143 or 144, wherein the one or more genetic alternations comprises one or more deletions.

Embodiment 146. The method of embodiment 144 or 145, wherein the one or more deletions remove all or part of each of the rodent immunoglobulin heavy chain constant region genes: Ighg and Ighe constant region genes.

Embodiment 147. The method of any one of embodiments 144-146, wherein the one or more deletions remove part of each of the rodent immunoglobulin heavy chain constant region genes: Ighg and Ighe constant region genes.

Embodiment 148. The method of any one of embodiments 144-146, wherein the one or more deletions remove all of each the rodent immunoglobulin heavy chain constant region genes: Ighg and Ighe constant region genes.

Embodiment 149. The method of embodiment 148, wherein the one or more deletions remove a contiguous sequence spanning a region that is upstream of the rodent Ighg3 constant region gene to a region that is upstream of the rodent Igha constant region gene.

Embodiment 150. The method of any one of embodiments 144-148, wherein the one or more deletions do not remove one or more of the non-coding sequences adjacent to the rodent immunoglobulin heavy chain constant region genes: Ighg and Ighe constant region genes.

Embodiment 151. The method of any one of embodiments 144-150, wherein the one or more genetic alterations comprise one or more insertions.

Embodiment 152. The method of embodiment 151, wherein the one or more insertions comprise an insertion of one or more repressor elements that inhibit expression of the rodent immunoglobulin heavy chain constant region genes: Ighg and Ighe constant region genes.

Embodiment 153. The method of any one of embodiments 144-152, wherein the one or more genetic alterations comprise one or more mutations.

Embodiment 154. The method of embodiment 153, wherein the one or more mutations comprise one or more frameshift mutations that inhibit expression of each of the rodent immunoglobulin heavy chain constant region genes: Ighg and Ighe constant region genes.

Embodiment 155. The method of any one of embodiments 142-154, wherein the genetically modified rodent comprises an engineered endogenous immunoglobulin heavy chain locus that comprises in 5′ to 3′ order:
(i) a rodent intronic enhancer (Eμ);
(ii) a rodent switch mu region (Sμ);
(iii) a rodent Ighm constant region gene;
(iv) a rodent Ighd constant region gene;
(v) a rodent switch alpha region (Sα);
(vi) a rodent Igha constant region gene; and
(vii) a rodent 3′ IgH regulatory region 3′RR.

Embodiment 156. The method of embodiment 155, wherein the engineered endogenous immunoglobulin heavy chain locus comprises a rodent 3′ IgH regulatory region 3′RR that is 3′ of the rodent Igha constant region gene.

Embodiment 157. The method of any one of embodiments 142-156, wherein the genetically modified rodent is heterozygous for the engineered endogenous immunoglobulin heavy chain locus.

Embodiment 158. The method of any one of embodiments 142-156, wherein the genetically modified rodent is hemizygous for the engineered endogenous immunoglobulin heavy chain locus.

Embodiment 159. The method of any one of embodiments 142-156, wherein the genetically modified rodent is homozygous for the engineered endogenous immunoglobulin heavy chain locus.

Embodiment 160. A method of producing an antibody, the method comprising the steps of:
(a) immunizing a genetically modified rodent according to any one of embodiments 1-17 and 36-121 with an antigen of interest;
(b) maintaining the genetically modified rodent under conditions sufficient for the genetically modified rodent to produce an immune response to the antigen of interest; and
(c) recovering from the genetically modified rodent:
   (i) an antibody that binds the antigen of interest,
   (ii) a nucleotide that encodes:
      (A) a human light variable domain of an antibody that binds the antigen of interest;
      (B) a human heavy chain variable domain of an antibody that binds the antigen of interest;
      (C) a human light chain of an antibody that binds the antigen of interest, or
      (D) a human a heavy chain of an antibody that binds the antigen of interest, or
   (iii) a cell (e.g., a B cell, e.g., a plasma cell) that expresses an antibody that binds the antigen of interest.

Embodiment 161. A method of producing an antibody, the method comprising the steps of:
(a) immunizing a genetically modified rodent according to any one of embodiments 18-34 and 36-121 with an antigen of interest;
(b) maintaining the genetically modified rodent under conditions sufficient for the genetically modified rodent to produce an immune response to the antigen of interest; and
(c) recovering from the genetically modified rodent:
   (i) an antibody that binds the antigen of interest,
   (ii) a nucleotide that encodes a human light or heavy chain variable domain, a light chain, or a heavy chain of an antibody that binds the antigen of interest, or
   (iii) a cell (e.g., a B cell, e.g., a plasma cell) that expresses an antibody that binds the antigen of interest.

Embodiment 162. A method of making an antibody, comprising:
(a) expressing a first nucleotide sequence that encodes an immunoglobulin heavy chain in a host cell, wherein the first nucleotide sequence includes a human heavy chain variable region sequence that was identified from a genetically modified rodent according to any one of embodiments 1-17 and 36-121;
(b) expressing a second nucleotide sequence that encodes an immunoglobulin light chain in a host cell;
(c) culturing the host cell so that immunoglobulin light chains and immunoglobulin heavy chains are expressed and form an antibody; and
(d) obtaining the antibody from the host cell or host cell culture.

Embodiment 163. A method of making an antibody, comprising:
(a) expressing a first nucleotide sequence that encodes an immunoglobulin heavy chain in a host cell, wherein the first nucleotide sequence includes a human heavy chain variable region sequence that was identified from a genetically modified rodent according to any one of embodiments 18-34 and 36-121;
(b) expressing a second nucleotide sequence that encodes an immunoglobulin light chain in a host cell;
(c) culturing the host cell so that immunoglobulin light chains and immunoglobulin heavy chains are expressed and form an antibody; and
(d) obtaining the antibody from the host cell or host cell culture.

Embodiment 164. A method of making a fully human antibody specific against an antigen comprising the steps of:
(a) immunizing a genetically modified rodent according to any one of embodiments 1-34 and 36-121 with the antigen;
(b) determining a nucleotide sequence that encodes a human heavy chain variable domain of an antibody that specifically binds the antigen and that was generated by the genetically modified rodent and/or determining a nucleotide sequence that encodes a human light chain variable domain of an antibody that specifically binds the antigen and that was generated by the genetically modified rodent; and
(c) expressing a fully human antibody by employing:
   (i) the nucleotide sequence encoding a human heavy chain variable domain of
(b) operably linked to a human heavy chain constant region gene, and/or
   (ii) the nucleotide sequence encoding a human light chain variable domain of
(e) operably linked to a human light chain constant region gene.

Embodiment 165. The method of embodiment 164, wherein the human heavy chain constant region gene is a human Ighg constant region chain.

Embodiment 166. A method of making a fully human antibody specific against an antigen comprising the steps of:
(a) expressing in a mammalian cell a fully human antibody comprising two human light chains and two human heavy chains, wherein each human light chain includes a human light chain variable domain encoded by a human light chain variable region and each human heavy chain includes a human heavy chain variable domain encoded by a human heavy chain variable region, wherein the nucleotide sequence of at least one human heavy or light chain variable region was obtained from a genetically modified rodent according to any one of embodiments 1-34 and 36-121; and
(b) obtaining the fully human antibody.

167. A method of making a fully human antibody specific against an antigen comprising the steps of:
(a) immunizing a genetically modified rodent according to any one of embodiments 1-34 and 26-121;
(b) determining a human heavy chain variable domain sequence of an antibody that specifically binds the antigen and that was generated by the genetically modified rodent and/or determining of a human light chain variable domain sequence of an antibody that specifically binds the antigen and that was generated by the genetically modified rodent; and
(c) expressing a fully human antibody by employing:
   (i) the human heavy chain variable domain sequence of (b) operably linked to a human heavy chain constant domain sequence, and/or
   (ii) the human light chain variable domain sequence of (b) operably linked to a human light chain constant domain sequence.

Embodiment 168. The method of embodiment 167, wherein employing the human heavy chain variable domain sequence of (b) operably linked to a human heavy chain constant domain sequence comprises expressing a nucleotide sequence that encodes the human heavy chain variable domain sequence of (b) and the human heavy chain constant domain sequence.

Embodiment 169. The method of embodiment 167 or 168, wherein employing the human light chain variable domain sequence of (b) operably linked to a human light chain constant domain sequence comprises expressing a nucleotide sequence that encodes the human light chain variable domain sequence of (b) and the human light chain constant domain sequence.

Embodiment 170. A method of making a fully human antibody specific against an antigen comprising the steps of:
(a) expressing in a mammalian cell said fully human antibody comprising two human light chains and two human heavy chains, wherein each human light chain includes a human light chain variable domain and each human heavy chain includes a human heavy chain variable domain, wherein the amino acid sequence of at least one human heavy or light chain variable domain was obtained from a rodent of any one of embodiments 1-34 and 36-121; and
(b) obtaining the fully human antibody.

Embodiment 171. A method of generating a human heavy chain or light chain variable domain sequence comprising the steps of:
(a) immunizing a genetically modified rodent of any one of embodiments 1-34 and 36-121; and
(b) determining a human heavy or light chain variable domain sequence of an antibody that specifically binds the antigen and that was generated by the genetically modified rodent.

Embodiment 172. The method of embodiment 171, wherein determining a human heavy or light chain variable domain sequence comprises determining a nucleotide sequence that encodes the human heavy or light chain variable domain sequence.

Embodiment 173. A method of making a fully human immunoglobulin heavy chain or a fully human immunoglobulin light chain comprising the steps of:
(a) immunizing a rodent of any one of embodiments 1-34 and 36-121 with an antigen;
(b) determining a human immunoglobulin heavy or light chain variable domain sequence of an antibody that specifically binds to the antigen and that was generated by the genetically modified rodent; and
(c) operably linking the human immunoglobulin heavy or light chain variable domain sequence to a human immunoglobulin heavy or light chain constant domain sequence, respectively, to form a fully human heavy chain or a fully human light chain.

Embodiment 174. The method of embodiments 173, wherein operably linking the human immunoglobulin heavy or light chain variable domain sequence to a human immunoglobulin heavy or light chain constant domain sequence, respectively, comprises operably linking a nucleotide sequence encoding the immunoglobulin human heavy or light chain variable domain sequence to a nucleotide sequence encoding the human immunoglobulin heavy or light chain constant domain sequence.

Embodiment 175. A method of generating a human immunoglobulin heavy or light chain variable region sequence comprising the steps of:
(a) immunizing a genetically modified rodent of any one of embodiments 1-34 and 36-121 with an antigen; and
(b) determining a human immunoglobulin heavy or light chain variable region sequence that encodes a human immunoglobulin heavy or light chain variable domain, respectively, of an antibody that specifically binds the antigen and that was generated by the genetically modified rodent.

Embodiment 176. A method of making a nucleotide sequence encoding a fully human immunoglobulin heavy chain or a fully human immunoglobulin light chain comprising the steps of:
(a) immunizing a genetically modified rodent of any one of embodiments 1-34 and 36-121 with an antigen;
(b) determining a human immunoglobulin heavy or light chain variable region sequence that encodes a human immunoglobulin heavy or light chain variable domain, respectively, of an antibody that specifically binds the antigen and that was generated by the genetically modified rodent; and
(c) operably linking the human immunoglobulin heavy or light chain variable region sequence to a human immunoglobulin heavy or light chain constant region gene, respectively, to form a nucleotide sequence encoding a fully human immunoglobulin heavy chain or a fully human immunoglobulin light chain.

Embodiment 177. A method of making a genetically modified rodent embryonic stem (ES) cell, the method comprising genetically modifying a rodent ES cell so that its genome comprises an engineered endogenous immunoglobulin heavy chain locus comprising only rodent Igha constant region gene.

Embodiment 178. A method of making a genetically modified rodent embryonic stem (ES) cell, the method comprising genetically modifying a rodent ES cell so that its genome comprises an engineered endogenous immunoglobulin heavy chain locus comprising only rodent Ighm, Ighd, and Igha constant region genes.

Embodiment 179. A rodent embryonic stem (ES) cell, whose genome comprises:
an engineered endogenous immunoglobulin heavy chain locus that comprises a rodent Igha constant region gene,
wherein the rodent ES cell genome further comprises one or more genetic alterations at the endogenous immunoglobulin heavy chain locus, wherein the one or more genetic alterations disrupt expression from rodent immunoglobulin heavy chain constant region genes: Ighm, Ighd, Ighg and Ighe constant region genes.

Embodiment 180. A rodent embryonic stem (ES) cell, whose genome comprises:
an engineered endogenous immunoglobulin heavy chain locus that comprises rodent Ighm, Ighd, and Igha constant region genes,
wherein the rodent ES cell genome further comprises one or more genetic alterations at the endogenous immunoglobulin heavy chain locus, wherein the one or more genetic alterations disrupt expression from rodent immunoglobulin heavy chain constant region genes: Ighg and Ighe constant region genes.

Embodiment 181. A rodent embryo generated from the rodent ES cell of embodiment 179 or 180.

Embodiment 182. An isolated rodent cell, whose genome comprises:
an engineered endogenous immunoglobulin heavy chain locus that comprises a rodent Igha constant region gene,
wherein the isolated rodent cell genome further comprises one or more genetic alterations at the endogenous immunoglobulin heavy chain locus, wherein the one or more genetic alterations disrupt expression from rodent immunoglobulin heavy chain constant region genes: Ighm, Ighd, Ighg, and Ighe constant region genes.

Embodiment 183. An isolated rodent cell, whose genome comprises:
an engineered endogenous immunoglobulin heavy chain locus that comprises rodent immunoglobulin heavy chain constant region genes: Ighm, Ighd, and Igha constant region genes,
wherein the isolated rodent cell genome further comprises one or more genetic alterations at the endogenous immunoglobulin heavy chain locus, wherein the one or more genetic alterations disrupt expression from rodent immunoglobulin heavy chain constant region genes: Ighg and Ighe constant region genes.

Embodiment 184. The isolated rodent cell of embodiment 182 or183, wherein the isolated rodent cell is a B cell (e.g., a plasma cell).

Embodiment 185. The isolated rodent cell of embodiment 184, wherein the cell is a plasma cell.

Embodiment 186. An immortalized cell generated from the isolated rodent cell of any one of embodiments 182-185.

Embodiment 187. A targeting vector comprising:
(i) a 5′ homology arm comprising a nucleotide sequence corresponding to a genomic target sequence that is upstream to a rodent Ighm constant region gene;
(ii) a selection marker; and
(iii) a 3′ homology arm comprising a nucleotide sequence corresponding to a genomic target sequence that is upstream of a rodent Igha constant region gene.

Embodiment 188. A targeting vector comprising:
(i) a 5′ homology arm comprising a nucleotide sequence corresponding to a genomic target sequence that is upstream to a rodent Ighg3 constant region gene;
(ii) a selection marker; and
(iii) a 3′ homology arm comprising a nucleotide sequence corresponding to a genomic target sequence that is upstream of a rodent Igha constant region gene.
wherein the genetically modified rodent only produces immunoglobulin heavy chains that comprise a rodent Igha constant domain encoded by the rodent Igha constant region gene.

1. A genetically modified rodent whose germline genome comprises an engineered endogenous immunoglobulin heavy chain locus that comprises an immunoglobulin heavy chain constant region comprising a rodent immunoglobulin heavy chain alpha (Igha) constant region gene,
wherein the genetically modified rodent only produces immunoglobulin heavy chains that comprise a rodent Igha constant domain encoded by the rodent Igha constant region gene.

2. The genetically modified rodent of claim 1, wherein the rodent's germline genome further comprises one or more genetic alterations at the endogenous immunoglobulin heavy chain locus, wherein the one or more genetic alterations disrupt expression from rodent immunoglobulin heavy chain constant region genes: mu (Ighm), delta (Ighd), gamma (Ighg), and epsilon (Ighe) constant region genes.

3-5. (canceled)

6. The genetically modified rodent of claim 2, wherein the one or more genetic alterations comprises one or more deletions.

7. The genetically modified rodent 6, wherein the one or more deletions remove all or part of each of the rodent immunoglobulin heavy chain constant region genes: Ighm, Ighd, Ighg, and Ighe constant region genes.

8. (canceled)

9. The genetically modified rodent of claim 1, wherein the engineered endogenous immunoglobulin heavy chain locus comprises in 5′ to 3′ order:
(i) a rodent intronic enhancer (Eμ);
(ii) a rodent switch mu region (Sμ); and
(iii) a rodent Igha constant region gene.

10-13. (canceled)

14. The genetically modified rodent of claim 1, wherein the engineered endogenous immunoglobulin heavy chain locus comprises in 5′ to 3′ order:
(i) one or more human V_{H} gene segments, one or more human D_{H} gene segments, and one or more human J_{H} gene segments;
(ii) a rodent intronic enhancer (Eμ);
(iii) a rodent switch mu region (Sμ);
(iv) a rodent Igha constant region gene; and optionally
(v) a rodent 3′ IgH regulatory region 3′RR.

15-27. (canceled)

28. The genetically modified rodent of claim 1, wherein the germline genome of the rodent comprises an engineered endogenous immunoglobulin light chain locus comprising:
(a) one or more non-rodent mammal Vκ gene segments, and
(b) one or more non-rodent mammal Jκ gene segments,
wherein the one or more non-rodent mammal Vκ gene segments and the one or more non-rodent mammal Jκ gene segments are operably linked to a Cκ gene.

29-37. (canceled)

38. The genetically modified rodent of claim 1, wherein the germline genome of the rodent comprises an engineered endogenous immunoglobulin light chain locus comprising:
(a) one or more non-rodent mammal Vλ gene segments, and
(b) one or more non-rodent mammal Jλ gene segments,
wherein the one or more non-rodent mammal Vλ gene segments and one or more non-rodent mammal Jλ gene segments are operably linked to a rodent immunoglobulin light chain constant region gene.

39-46. (canceled)

47. A genetically modified rodent whose germline genome comprises an engineered endogenous immunoglobulin heavy chain locus that comprises an immunoglobulin heavy chain constant region comprising rodent immunoglobulin heavy chain constant region genes Ighm, Ighd, and Igha,
wherein the genetically modified rodent only produces immunoglobulin heavy chains that comprise a rodent Igha, a rodent Ighm, or a rodent Ighd constant domain encoded by the rodent Igha constant region gene, the rodent Ighm constant region gene, or the rodent Ighd constant region gene, respectively.

48-52. (canceled)

53. A method of making a genetically modified rodent, the method comprising genetically modifying the germline genome of a rodent so that the rodent comprises an immunoglobulin heavy chain constant region comprising a rodent Igha constant region gene and only produces immunoglobulin heavy chains that comprise a rodent Igha constant domain encoded by the rodent Igha constant region gene.

54. A method of making a genetically modified rodent, the method comprising genetically modifying the germline genome of a rodent so that the rodent comprises an immunoglobulin heavy chain constant region comprising rodent immunoglobulin heavy chain constant region genes Ighm, Ighd, and Igha, and only produces immunoglobulin heavy chains that comprise a rodent Igha, a rodent Ighm, or a rodent Ighd constant domain encoded by the Igha constant region gene, the rodent Ighm constant region gene, or the rodent Ighd constant region gene, respectively.

55. A method of producing an antibody, the method comprising the steps of:
(a) immunizing a genetically modified rodent according to claim 1 with an antigen of interest;
(b) maintaining the genetically modified rodent under conditions sufficient for the genetically modified rodent to produce an immune response to the antigen of interest; and
(c) recovering from the genetically modified rodent:
(i) an antibody that binds the antigen of interest,
(ii) a nucleotide that encodes a human light or heavy chain variable domain, a light chain, or a heavy chain of an antibody that binds the antigen of interest, or
(iii) a cell that expresses an antibody that binds the antigen of interest.

56. A method of making an antibody, comprising:
(a) expressing a first nucleotide sequence that encodes an immunoglobulin heavy chain in a host cell, wherein the first nucleotide sequence includes a human heavy chain variable region sequence that was identified from a genetically modified rodent according to claim 1;
(b) expressing a second nucleotide sequence that encodes an immunoglobulin light chain in a host cell;
(c) culturing the host cell so that immunoglobulin light chains and immunoglobulin heavy chains are expressed and form an antibody; and
(d) obtaining the antibody from the host cell or host cell culture.

57. A method of making a fully human antibody specific against an antigen comprising the steps of:
(a) immunizing a genetically modified rodent according to claim 1;
(b) determining a nucleotide sequence that encodes a human heavy chain variable domain of an antibody that specifically binds the antigen and that was generated by the genetically modified rodent and/or determining a nucleotide sequence that encodes a human light chain variable domain of an antibody that specifically binds the antigen and that was generated by the genetically modified rodent; and
(c) expressing a fully human antibody by employing:
(i) the nucleotide sequence encoding a human heavy chain variable domain of
(d) operably linked to a human heavy chain constant region gene, and/or
(ii) the nucleotide sequence encoding a human light chain variable domain of
(e) operably linked to a human light chain constant region gene.

58. A method of making a fully human antibody specific against an antigen comprising the steps of:
(a) expressing in a mammalian cell a fully human antibody comprising two human light chains and two human heavy chains, wherein each human light chain includes a human light chain variable domain encoded by a human light chain variable region and each human heavy chain includes a human heavy chain variable domain encoded by a human heavy chain variable region, wherein the nucleotide sequence of at least one human heavy or light chain variable region was obtained from a genetically modified rodent according to claim 1; and
(b) obtaining the fully human antibody.

59. A method of making a fully human antibody specific against an antigen comprising the steps of:
(a) immunizing a genetically modified rodent according to claim 1;
(b) determining a human heavy chain variable domain sequence of an antibody that specifically binds the antigen and that was generated by the genetically modified rodent and/or determining of a human light chain variable domain sequence of an antibody that specifically binds the antigen and that was generated by the genetically modified rodent; and
(c) expressing a fully human antibody by employing:
(i) the human heavy chain variable domain sequence of (b) operably linked to a human heavy chain constant domain sequence, and/or
(ii) the human light chain variable domain sequence of (b) operably linked to a human light chain constant domain sequence.

60. A method of making a fully human antibody specific against an antigen comprising the steps of:
(a) expressing in a mammalian cell a fully human antibody comprising two human light chains and two human heavy chains, wherein each human light chain includes a human light chain variable domain and each human heavy chain includes a human heavy chain variable domain, wherein the amino acid sequence of at least one human heavy or light chain variable domain was obtained from a rodent of claim 1; and
(b) obtaining the fully human antibody.

61. A method of generating a human heavy chain or light chain variable domain sequence comprising the steps of:
(a) immunizing a genetically modified rodent of claim 1; and
(b) determining a human heavy or light chain variable domain sequence of an antibody that specifically binds the antigen and that was generated by the genetically modified rodent.

62. (canceled)

63. A method of making a fully human immunoglobulin heavy chain or a fully human immunoglobulin light chain comprising the steps of:
(a) immunizing a rodent of claim 1 with an antigen;
(b) determining a human immunoglobulin heavy or light chain variable domain sequence of an antibody that specifically binds to the antigen and that was generated by the genetically modified rodent; and
(c) operably linking the human immunoglobulin heavy or light chain variable domain sequence to a human immunoglobulin heavy or light chain constant domain sequence, respectively, to form a fully human heavy chain or a fully human light chain.

64. A method of generating a human immunoglobulin heavy or light chain variable region sequence comprising the steps of:
(a) immunizing a genetically modified rodent of claim 1 with an antigen; and
(b) determining a human immunoglobulin heavy or light chain variable region sequence that encodes a human immunoglobulin heavy or light chain variable domain, respectively, of an antibody that specifically binds the antigen and that was generated by the genetically modified rodent.

65. A method of making a nucleotide sequence encoding a fully human immunoglobulin heavy chain or a fully human immunoglobulin light chain comprising the steps of:
(a) immunizing a genetically modified rodent of claim 1 with an antigen;
(b) determining a human immunoglobulin heavy or light chain variable region sequence that encodes a human immunoglobulin heavy or light chain variable domain, respectively, of an antibody that specifically binds the antigen and that was generated by the genetically modified rodent; and
(c) operably linking the human immunoglobulin heavy or light chain variable region sequence to a human immunoglobulin heavy or light chain constant region gene, respectively, to form a nucleotide sequence encoding a fully human immunoglobulin heavy chain or a fully human immunoglobulin light chain.

66. A method of making a genetically modified rodent embryonic stem (ES) cell, the method comprising genetically modifying a rodent ES cell so that its genome comprises an engineered endogenous immunoglobulin heavy chain locus comprising a Igha constant region gene,
wherein the genetically modified rodent only produces immunoglobulin heavy chains that comprise a rodent Igha constant domain encoded by the rodent Igha constant region gene.

67. A rodent embryonic stem (ES) cell, whose genome comprises:
an engineered endogenous immunoglobulin heavy chain locus that comprises a rodent Igha constant region gene,
wherein the rodent ES cell genome further comprises one or more genetic alterations at the endogenous immunoglobulin heavy chain locus, wherein the one or more genetic alterations disrupt expression from rodent immunoglobulin heavy chain constant region genes: Ighm, Ighd, Ighg and Ighe constant region genes.

68. A rodent embryo generated from the rodent ES cell of claim 66.

69. An isolated rodent cell, whose genome comprises:
an engineered endogenous immunoglobulin heavy chain locus that comprises a rodent Igha constant region gene,
wherein the isolated rodent cell genome further comprises one or more genetic alterations at the endogenous immunoglobulin heavy chain locus, wherein the one or more genetic alterations disrupt expression from rodent immunoglobulin heavy chain constant region genes: Ighm, Ighd, Ighg, and Ighe constant region genes.

70. (canceled)

71. An immortalized cell generated from the isolated rodent cell of claim 69.

72. A targeting vector comprising:
(i) a 5′ homology arm comprising a nucleotide sequence corresponding to a genomic target sequence that is upstream to a rodent Ighm constant region gene;
(ii) a selection marker; and
(iii) a 3′ homology arm comprising a nucleotide sequence corresponding to a genomic target sequence that is upstream of a rodent Igha constant region gene.


SEQUENCE LISTING (24 sequences)

SEQ ID NO: 1 - DNA, 55 bp, synthetic construct
  source 1..55: mol_type = other DNA
acaaaagctggagctccaccgcggtggcggccgctgagcaagagtgagtagagct

SEQ ID NO: 2 - DNA, 47 bp, synthetic construct
  source 1..47: mol_type = other DNA
aattggcattatttaaagttaaccggtgaacgtgactttggaagcct

SEQ ID NO: 3 - DNA, 49 bp, synthetic construct
  source 1..49: mol_type = other DNA
ataatgccaattatttaaagttagtcgactgcaggtcatgttcaaagtc

SEQ ID NO: 4 - DNA, 54 bp, synthetic construct
  source 1..54: mol_type = other DNA
gtaatacgactcactatagggcgaattgggtacccgtggcatcttcttcccagt

SEQ ID NO: 5 - DNA, 56 bp, synthetic construct
  source 1..56: mol_type = other DNA
acaaaagctggagctccaccgcggtggcggccgcagacactttcattgggtcccag

SEQ ID NO: 6 - DNA, 44 bp, synthetic construct
  source 1..44: mol_type = other DNA
cccgtcgacgaattcaccggtatggggtcttcagtacaattcag

SEQ ID NO: 7 - DNA, 45 bp, synthetic construct
  source 1..45: mol_type = other DNA
cccataccggtgaattcgtcgacggggaatggttattcacaaatg

SEQ ID NO: 8 - DNA, 60 bp, synthetic construct
  source 1..60: mol_type = other DNA
gtaatacgactcactatagggcgaattgggtaccgctagctgtcccagttcagcccagat

SEQ ID NO: 9 - DNA, 20 bp, synthetic construct
  source 1..20: mol_type = other DNA
cttccaaagtcacgttcctg

SEQ ID NO: 10 - DNA, 20 bp, synthetic construct
  source 1..20: mol_type = other DNA
ccttgtgaaagactacctgc

SEQ ID NO: 11 - DNA, 27 bp, synthetic construct
  source 1..27: mol_type = other DNA
cacctggaactaccagaacaacactga

SEQ ID NO: 12 - DNA, 22 bp, synthetic construct
  source 1..22: mol_type = other DNA
ccatggtgaccctgggatgcct

SEQ ID NO: 13 - DNA, 23 bp, synthetic construct
  source 1..23: mol_type = other DNA
cctctcgtctttgggtccactgc

SEQ ID NO: 14 - DNA, 21 bp, synthetic construct
  source 1..21: mol_type = other DNA
acgagcgggttcggcccattc

SEQ ID NO: 15 - DNA, 20 bp, synthetic construct
  source 1..20: mol_type = other DNA
ctgacatctctggcattatg

SEQ ID NO: 16 - DNA, 20 bp, synthetic construct
  source 1..20: mol_type = other DNA
tgtgaataaccattccccaa

SEQ ID NO: 17 - DNA, 26 bp, synthetic construct
  source 1..26: mol_type = other DNA
tcgggcatgtggatcaagatataggt

SEQ ID NO: 18 - DNA, 24 bp, synthetic construct
  source 1..24: mol_type = other DNA
tcccacatagaagcaaatcccacc

SEQ ID NO: 19 - DNA, 20 bp, synthetic construct
  source 1..20: mol_type = other DNA
cagggcgtggagtcactgat

SEQ ID NO: 20 - DNA, 30 bp, synthetic construct
  source 1..30: mol_type = other DNA
aggccatagcataaagtaaccctacataga

SEQ ID NO: 21 - DNA, 20 bp, synthetic construct
  source 1..20: mol_type = other DNA
aggtgggaccagcagttgag

SEQ ID NO: 22 - DNA, 24 bp, synthetic construct
  source 1..24: mol_type = other DNA
gagtcactgataggccatagcata

SEQ ID NO: 23 - DNA, 25 bp, synthetic construct
  source 1..25: mol_type = other DNA
tgaaatctgaattgtactgaagacc

SEQ ID NO: 24 - DNA, 25 bp, synthetic construct
  source 1..25: mol_type = other DNA
ggtcacatttgtgaataaccattcc